SEQUENCE LISTING (3 sequences)

SEQ ID NO: 1 - DNA, 30 bp, synthetic construct
  source 1..30: mol_type = other DNA
  modified_base 15: mod_base = OTHER; note = deoxyuridine
accctgctgtgggcnggagaacaaggtgat

SEQ ID NO: 2 - DNA, 31 bp, synthetic construct
  source 1..31: mol_type = other DNA
atcaccttgttctccahgcccacagcagggt

SEQ ID NO: 3 - DNA, 29 bp, synthetic construct
  source 1..29: mol_type = other DNA
accctgctgtgggcggagaacaaggtgat

(19) United States
(12) Patent Application Publication
(10) Pub. No.: US 2026/0193238 A1
(43) Pub. Date: Jul. 9, 2026

(54) PHARMACEUTICAL COMPOUNDS AS PARP7 AND/OR PARP1 INHIBITORS

(71) Applicant: Duke Street Bio Limited, London (GB)

(72) Inventors: Phillip Cowley, London (GB); Alan Wise, London (GB)

(21) Appl. No.: 19/132,115

(22) PCT Filed: Nov. 28, 2023
(86) PCT No.: PCT/EP2023/083319

§ 371 Date: May 22, 2025

(30) Foreign Application Priority Data
Nov. 29, 2022 (GB) 2217888.3

(51) Int. Cl. C07D 403/14; A61K 31/501; A61K 31/502; A61K 31/506; A61K 31/5377; A61K 31/55; A61K 45/06; C07D 401/14; C07D 413/14; C07D 417/14; C07D 471/04; C07D 471/10; C07D 487/08; C07D 487/10

(52) U.S. Cl. CPC C07D 403/14; A61K 31/501; A61K 31/502; A61K 31/506; A61K 31/5377; A61K 31/55; A61K 45/06; C07D 401/14; C07D 413/14; C07D 417/14; C07D 471/04; C07D 471/10; C07D 487/08; C07D 487/10

(57) ABSTRACT

A compound which is a PARP1 and/or PARP7 inhibitor has a structure:

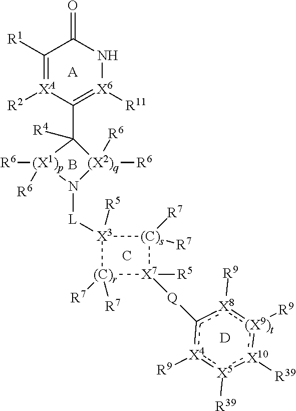

R¹, R³, R⁴, R¹⁰, R¹², and R¹⁴ are each independently H or an organic group. R², R⁵, R⁶, R⁷, R⁹, R¹¹ are each independently absent, H, or an organic group. One R³⁹ is an R⁹ and one R³⁹ is an R³. X¹, X2, X⁴, X⁵, X⁸, X⁹ and X¹⁰ are each independently C, N, O or S. X^{A} X³, X⁶, and X⁷ are each independently C or N. p+q is 2, 3, 4 or 5. r+s is 2, 3, 4 or 5. t is 0 or 1. Ring B is saturated or has one double bond. Ring D is aromatic. Q is a bond or a linker group.

L is selected from:

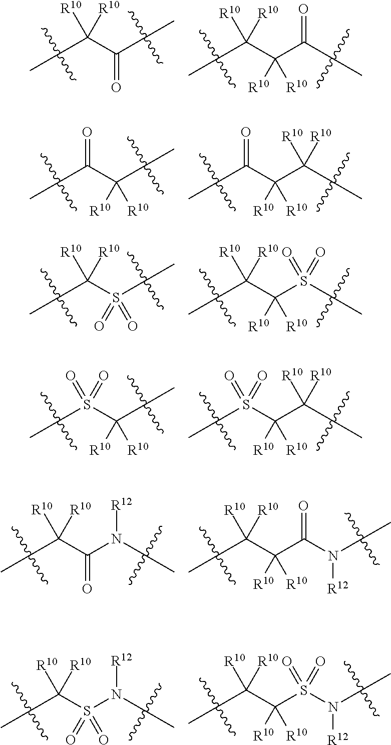

Alternatively, L and R⁵ together represent:

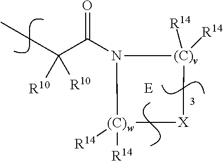

where v is 1 or 2 and w is 1 or 2.

### TECHNICAL FIELD

The present invention relates to PARP1 and/or PARP7 inhibitor compounds, and in particular to PARP1 and/or PARP7 inhibitor compounds for use in medicine. The inhibitors of the invention may be used in pharmaceutical compositions, and in particular pharmaceutical compositions for treating a cancer, an infectious disease, a central nervous system disease or disorder, a pain condition and other diseases, conditions and disorders. The invention also relates to methods of manufacture of such inhibitors, and methods of treatment using such inhibitors.

### BACKGROUND

### PARP1 Inhibitors

The family of poly(ADP-ribose) polymerases (PARPs) consists of 17 PARP proteins that catalyse the transfer of ADP-ribose to target proteins, a posttranslational process termed PARylation. Target protein modification by PARylation causes significant changes to function and as such PARPs play an important role in many cellular processes such as chromatin remodelling, transcription, replication, recombination, cell cycle progression and DNA damage repair (Kamaletdinova, T. et al. Cell. 2019; 8: 1625). PARP1 and 2 are the most widely studied PARP enzymes, primarily due to their role in DNA damage repair, in particular in the base excision repair (BER) process of DNA single-strand breaks (Ngoi, Y L. et al. Cancer J. 2021; 27: 521-528). PARP1 is activated by DNA damage breaks, and the subsequent PARylation of target proteins leads to recruitment of additional factors that initiate repair of DNA lesions. Auto-PARylation of PARP triggers the release of bound PARP from the DNA allowing other DNA repair proteins access to complete lesion repair. This highlights the critical role PARP plays in enabling a cancer cell to repair DNA damage caused by exogenous agents such as radiation therapy and chemotherapeutic agents. Hence, inhibition of PARP enzymes has been utilised as a strategy to selectively kill cancer cells that harbour genetic defects in complementary DNA damage repair pathways (Farmer, H. et al. Nature. 2005; 434: 917-921). This synthetic lethality approach has been demonstrated successfully in tumours with epigenetic modifications or deleterious mutations in BRCA1 and BRCA2, two functionally redundant tumour suppressor proteins involved in DNA double-strand break (DSB) repair by homologous recombination (HR) (Lord, C J. and Ashworth, A. Science. 2017; 355: 1152-1158). Such tumours with HR deficiency (HRD) are dependent on PARP function for survival—following PARP inhibition in these tumours, DSB breaks will be processed by alternative error-prone repair pathways leading to genomic instability and cancer cell death.

The inhibition of PARP can trap the inactivated PARP at the sites of DNA damage. This leads to replication fork stalling and subsequent collapse in S-phase when the fork reaches the site of the trapped PARP, resulting in the generation of genotoxic DNA double-strand breaks. It is believed that this PARP1-DNA trapping can lead to the selective death of cancer cells harbouring HRD (Farmer, H. et al. Nature. 2005; 434: 917-921).

This strategy has led to the successful approval of several PARP1 inhibitors for the treatment of cancers with HRD, such as in BRCA1/2-mutated breast, ovarian and prostate cancer, as well as in ovarian and prostate cancer harbouring genomic consequences of HRD, and ovarian cancer in the maintenance setting where platinum sensitivity acts as a surrogate for HRD (Fong, P C. et al. N. Engl. J. Med. 2009; 361: 123-134).

It has recently been shown that genomic instability, in the form of unrepaired DNA double-strand breaks or micronuclei disruption can trigger innate immune system activation via the cytosolic DNA sensor cyclic GMP-AMP synthase (cGAS), leading to generation of cyclic guanosine monophosphate-adenosine monophosphate (cGAMP) and induction of dimerization of Stimulator of interferon genes (STING). STING subsequently translocates from the endoplasmic reticulum to the Golgi where it recruits and activates TANK-binding kinase 1 (TBK1). TBK1 phosphorylates interferon regulatory transcription factor 3 (IRF3) which drives the production of type I interferons and supports the induction of an adaptive immune response (Zhu, Y. et al. Mol. Cancer. 2019, 18: 152).

For example, PARP1 inhibitor-induced STING pathway activation and anti-tumour immune responses have been demonstrated in multiple tumour models, providing rationale for exploiting combinations of PARP1 inhibitors with immunotherapies for improved therapeutic efficacy (Sen, T. et al. Cancer Discov. 2019; 9: 646-661). For example, the PARP1 inhibitor Olaparib was also recently shown to induce synthetic lethal effects in combination with a synthetic cyclic dinucleotide STING agonist in DNA damage repair deficient cancer cells and a BRCA-deficient breast cancer model (Pantelidou, C. et al. 2021: bioRxiv (DOI: 10.1101/2021.01.26.428337).

Overall, modulation of nucleic acid sensing pathways via multiple mechanisms has been shown to promote anti-tumour efficacy in a variety of cell and animal models thus demonstrating therapeutic potential for augmenting efficacy of immunotherapies and overcoming resistance to immune checkpoint blockade through use of PARP1 inhibitors. There are numerous clinical trials ongoing combining PARP1 inhibitors with immunotherapies (reviewed in Chabanon, R M, et al. Nat. Rev. Cancer. 2021; 21: 701-717).

Recently, PARP1 has also been shown to bind the Epstein Barr Virus (EBV) genome and that PARP1 inhibition can alter EBV chromatin structure and latent gene expression (Morgan, S M. et al. Nat. Commun. 2022; 13: 187). Hence, PARP1 inhibitors may play a role in cancers where EBV plays a contributing role such as Burkitt's lymphoma, Hodgkin's lymphoma, nasopharyngeal and gastrointestinal cancers. Interestingly, EBV has also been shown to be a causative factor in multiple sclerosis (MS) whereby EBV infection greatly increases the risk of subsequent MS (Bjornevik, K. et al. Science (2021); 375: 296-301).

First-generation PARP1 inhibitors generally demonstrate non-selective activity at PARP1 and 2. Haematological toxicities such as anaemia, neutropenia and thrombocytopenia are associated with clinical use of these molecules which restricts their use in combination with cytotoxic chemotherapies and other targeted agents due to dose-limiting cytopenias (LaFargue, C J. et al. Lancet Oncol. 2019, 20, e15-e28). Evidence from pre-clinical mouse studies strongly suggests that PARP2 inhibition is a major driver of these haematological toxicities, with PARP2 being particularly linked to erythrogenesis in mice (Farres, J. et al. Blood. 2013; 122: 44-54). In addition, PARP2 function has been shown to be dispensable for anti-tumour activity in HRD mouse cancer models (Ronson, G E. et al. Nat. Commun. 2018, 9: 746). Taken together, these data suggest an unmet medical need for the development of inhibitors with improved selectivity for PARP1 over PARP2 and other PARPs, thus providing expanded therapeutic utility (1) as single agents and (2) in combination with other anti-cancer agents.

To date, two PARP1-selective inhibitors, AZD5305 and AZD9574, have entered clinical development. AZD5305 was described as a potent PARP1 inhibitor and trapper with 500-fold selectivity over PARP2 and less off-target activity against secondary pharmacology targets than first-generation PARP inhibitors (Johannes, J W. et al. J. Med. Chem. 2021; 64: 14498-14512). Importantly, significantly less haematotoxicity was observed for AZD5305 in rodent models than with first-generation PARP inhibitors, confirming the reported pathogenic role of PARP2 in haematologic toxicity (Illuzzi, G. et al. Clin. Cancer Res. 2022; CCR-22-0301).

### PARP7 Inhibitors

Despite the remarkable clinical success of cancer immunotherapies over the last decade, most patients either fail to respond to immune checkpoint therapy or develop resistance. Cancer is a disease that uses a variety of methods to cloak itself from the immune system and to suppress the activity of immune cells in the tumour microenvironment. There is an urgent need to identify and develop complementary therapies that will broaden the population for whom immunomodulatory therapy delivers benefit. Monoclonal antibody-based therapeutics targeting immune checkpoints, most notably the PDL1-PD1 axis, are transforming approaches to the treatment of cancer.

These immune checkpoint inhibitors (ICIs) such as anti-PD1 and anti-PDL1 act by relieving checkpoint restraints on anti-tumour T cell responses. They work best against immunogenic, T-cell inflamed or hot tumours. In contrast, ICIs are poorly efficient in cold tumour microenvironments (TMEs) that are largely devoid of T cells and infiltrated by immunosuppressive cells. In hot TMEs, increased expression of type I interferons (IFN-I) and IFN-stimulated genes (ISGs), such as T-cell attracting chemokines, contribute to potent anti-tumour responses. One emerging therapeutic strategy to transform cold tumours into hot exploits the use of pattern recognition receptor (PRR) agonists. Indeed, combinations of ICIs with agonists of RIG-I Helicase, Toll-like receptor 9 (TLR9) or stimulator of interferon genes (STING) have now reached clinical evaluation.

The innate immune system provides a first line of host defence and plays a crucial role in initiating and driving the development of adaptive immune responses. The cytosolic DNA sensor cyclic GMP-AMP synthase (cGAS) can be activated by double stranded DNA arising from the genomes of invading pathogens and also by aberrant cytosolic levels of host DNA that are generated in tumour cells (Chen Q et al. Regulation and function of the cGAS-STING pathway of cytosolic DNA sensing. Nat Immunol. 17: 1142-9, (2016)). Activation of cGAS leads to the generation of cyclic guanosine monophosphate-adenosine monophosphate (cGAMP) which induces dimerization of Stimulator of interferon genes (STING). STING subsequently translocates from the endoplasmic reticulum to the Golgi where it recruits and activates TANK-binding kinase 1 (TBK1). TBK1 phosphorylates interferon regulatory transcription factor 3 (IRF3) which drives the production of type I interferons and supports the generation of immunity (Zhu Y et al. STING: a master regulator in the cancer-immunity cycle. Mol Cancer 18: 152 (2019)). As such, activation of the STING pathway has become of increasing interest to the cancer drug discovery community as a potential strategy to boost the development of adaptive immune responses to tumour cell neoantigens (Sivick K. E. et al. Magnitude of Therapeutic STING Activation Determines CD8+ T Cell-Mediated Anti-tumor Immunity. Cell Reports. 25: 3074, (2018)). Cytoplasmic DNA sensing has also been linked to inactivation of cellular proliferation providing an additional potential mechanistic axis that may contribute to control of tumorigenesis (Paludan S. R. et al. DNA-stimulated cell death: implications for host defence, inflammatory diseases and cancer. Nat Rev Immunol. 19: 141-153, (2019)).

Cancer cells can exhibit a chronic Interferon-stimulate gene (ISG) signature triggered by a STING-dependent pathway, which results in a unique primed cancer cell state that is sensitized to respond to aberrant nucleic acid accumulation (Liu H et al. Tumor-derived IFN triggers chronic pathway agonism and sensitivity to ADAR loss. Nat Medicine. 25: 95-102, 2019). It has recently been shown that genomic instability, in the form of unrepaired DNA double-strand breaks or micronuclei disruption can trigger STING-dependent anti-tumour responses. For example, use of chemotherapeutics can lead to higher levels of aberrant DNA in the cytosol which in turn can trigger cancer cell intrinsic STING signalling leading to anti-tumour immunity. Indeed the efficacy of the commonly used chemotherapeutic drug 5-fluorouracil (5-FU) was recently shown to depend on anti-tumor immunity triggered by the activation of cancer-cell intrinsic STING (Tian J et al. 5-Fluorouracil efficacy requires anti-tumor immunity triggered by cancer-cell-intrinsic STING. EMBO J. 40: e106065 (2021)). In addition, PARP inhibitor-induced STING pathway activation and anti-tumor immune responses have been demonstrated in multiple tumour models, providing rationale for exploiting combinations of PARP inhibitors with immunotherapies for improved therapeutic efficacy. For example the PARP inhibitor Olaparib was also recently shown to induce synthetic lethal effects in combination with a synthetic cyclic dinucleotide STING agonist in DNA damage repair deficient cancer cells and a BRCA-deficient breast cancer model (Pantelidou C et al. STING agonism enhances anti-tumor immune responses and therapeutic efficacy of PARP inhibition in BRCA-associated breast cancer. bioRxiv (2021), DOI: 10.1101/2021.01.26.428337). The authors hypothesize that STING agonism can enhance the therapeutic efficacy of PARP inhibitors in BRCA-associated triple-negative breast cancer (TNBC).

Overall, modulation of nucleic acid sensing pathways via multiple mechanisms has been shown to promote anti-tumour efficacy in a variety of cell and animal models thus demonstrating therapeutic potential for augmenting efficacy of immunotherapies and overcoming resistance to immune checkpoint blockade.

Poly-ADP-ribose polymerase 7 (PARP7, TIPARP, ARTD14), a member of the wider PARP enzyme family, modulates protein function by using nicotinamide adenine dinucleotide (NAD+) as a substrate to transfer an ADP-ribose monomer onto specific amino acid acceptor residues of target proteins (Gomez A et al. Characterisation of TCDD-inducible poly-ADP-ribose polymerase (TIPARP/ARTD14) catalytic activity. BiochemicalJournal. 475: 3827-3846, (2018)). PARP7 catalyses mono-ADP ribosylation (MARylation) of its target substrates and as such is a member of the mono(ADP-ribosyl) transferase (MART) enzymes, a subclass of the PARP family of enzymes (reviewed in-Challa L. et al. MARTs and MARylation in the Cytosol: Biological Functions, Mechanisms of Action, and Therapeutic Potential. Cells 10, 313 (2021)). PARP7 is a target gene of the Aryl Hydrocarbon Receptor (AHR) which is a ligand-activated transcription factor and member of the basic helix-loop-helix/Per-AHR nuclear translocator (ARNT)-Sim (PAS) protein family which plays a central role in controlling immune responses. Therefore, PARP7 has emerged as a critical regulator of the innate immune response. The PARP7 gene is amplified in a number of cancers, notably those of the upper aerodigestive tract (Vasbinder, M. M. et al. RBN-2397: A First-in-class PAPR7 inhibitor targeting a newly discovered cancer vulnerability in stress-signalling pathways. Cancer Res. 80: 16 suppl DDT02-01, (2020)). PARP7 has been reported to ADP ribosylate and inactivate the kinase domain of TBK1 resulting in suppression of a central pathway for interferon production (Yamada T et al. Constitutive aryl hydrocarbon receptor signalling constrains type I interferon-mediated antiviral innate defence. Nature Immunol. 17: 687-694, (2016)). The possibility of using PARP7 inhibitors in cancer therapy, especially in the treatment of lung squamous cell carcinoma, has been described in WO 2016/116602 A1. The discovery of a potent and selective inhibitor of PARP7, RBN-2397 has been recently reported (Vasbinder, M. M. et al. RBN-2397: A First-in-class PAPR7 inhibitor targeting a newly discovered cancer vulnerability in stress-signalling pathways. Cancer Res. 80: 16 suppl DDT02-01, (2020); Gozgit J et al. PARP7 negatively regulates the type I interferon response in cancer cells and its inhibition leads to tumour regression. Cancer Res. 80: 16 suppl 3405, (2020); Gozgit J et al. PARP7 negatively regulates the type I interferon response in cancer cells and its inhibition triggers antitumor immunity. Cancer Cell 39: 1-13, 2021). RBN-2397 potently inhibited proliferation in cancer cell lines with high baseline expression of interferon stimulated genes and restored type I interferon responses both in vitro and in vivo resulting in tumour regression and establishment of specific anti-tumour immunity in animal models. The direct anti-proliferative function of RBN-2397 on a subset of cancer cell types has been shown to be mediated via inhibition of autophagy, suppression of energy metabolism (both glycolysis and oxidative phosphorylation) which results in cell cycle arrest and senescence (Molina J PARP7 Inhibitor RBN-2397 Induces Tumoral IFN Signaling and Intrinsic Effects Resulting in Regressions in Mouse Models. AACR, 2022). WO 2019/212937 A1 describes pyridazinone compounds as inhibitors of PARP7 for use in the treatment of cancer. These observations provide a rational basis for generating novel agents to inhibit PARP7 and induce therapeutic anti-tumour responses.

In addition, there is therapeutic potential for use of a PARP7 inhibitor in canine cancer as notably recent data showed that intratumoral delivery of a STING agonist resulted in clinical responses in canine glioblastoma (Boudreau C E et al. Delivery of STING Agonist Results in Clinical Responses in Canine Glioblastoma. Clin Cancer Res (2021)).

Importantly, molecules that can inhibit both PARP1 and PARP7 can modulate multiple mechanisms controlling tumour development. These include direct tumour-intrinsic growth arrest and immune system activation in the tumour microenvironment via PARP7 and direct cancer cell killing in tumour harbouring loss-of-function mutations in DNA damage repair pathways via PARP1. Hence a dual acting PARP1 and PARP7 inhibitor may offer significant therapeutic benefit.

Having regard to the above, it is an aim of the present invention to provide PARP1 and/or PARP7 inhibitors, and in particular PARP1 and/or PARP7 inhibitors for use in medicine. It is a further aim to provide pharmaceutical compositions comprising such inhibitors, and in particular to provide compounds and pharmaceutical compositions for treating a cancer, an infectious disease, a central nervous system disease or disorder and other diseases, conditions and disorders. It is also an aim to provide methods of synthesis of the compounds.

### SUMMARY

One aspect provides a compound that is PARP7 and/or PARP1 inhibitor. The compound comprises the following formula:

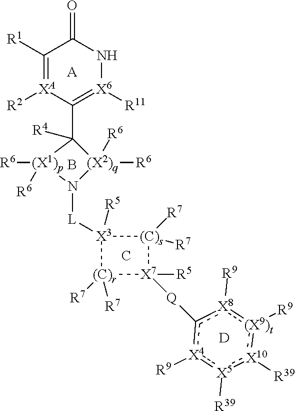

wherein:
   R¹ is independently selected from H and a substituted or unsubstituted organic group;
   X^{A} is selected from C and N;
      when X^{A} is C:
         R² is independently selected from H and a substituted or unsubstituted organic group, and wherein R¹ and R² may together form a 4 to 7 membered ring together with the atoms of ring A to which they are
      when X^{A} is N:
   R² is absent;
   X⁶ is independently selected from C and N;
   R¹¹ is present or absent depending on the valency of X⁶, and when present is independently selected from H and a substituted or unsubstituted organic group;
   p is selected from 0, 1, 2, 3, 4 and 5;
   q is selected from 0, 1 and 2, with the proviso that p+q is 2, 3, 4 or 5;
   each X¹ is independently selected from C, N, O and S;
   each X² is independently selected from C, N, O and S;
   ring B is a saturated ring or an unsaturated ring having exactly one double bond;
   R⁴ is independently selected from H and a substituted or unsubstituted organic group; each R⁶ is independently present or absent depending on the valency of the X¹ or X² atom to which it is attached, and each R⁶ is when present independently selected from H and a substituted or unsubstituted organic group;
   X³ and X⁷ are each independently selected from C and N;
   each bond between the X³ and/or the X⁷, and/or the carbon atoms in ring C may be a single bond or a double bond depending on the number of bonds to, and the valence of, the X³ atom, the X⁷ atom and the carbon atoms;
   each R⁵ is independently present or absent depending on the number of bonds to, and the valency of, the X³ or X⁷ atom to which that R⁵ is attached, and each R⁵ is when present independently selected from H and a substituted or unsubstituted organic group;
   each R⁷ is independently present or absent depending on the number of bonds to the carbon atom to which that R⁷ is attached, and each R⁷ is when present independently selected from H and a substituted or unsubstituted organic group;
   r is selected from 0, 1, 2, 3, 4 and 5;
   s is selected from 0, 1, 2, 3, 4 and 5, with the proviso that r+s is 2, 3, 4 or 5; each of X⁴, X⁵, X⁸, X⁹ and X¹⁰ is independently selected from C, N, O and S;
   t is 0 or 1;
   ring D is an aromatic ring, and includes 2 or 3 double bonds depending on the size of t and the nature of X⁴, X⁵, X⁸, X⁹ and X¹⁰;
   one of R³⁹ is an R⁹ and one of R³⁹ is an R³;
      R³ being independently selected from H and a substituted or unsubstituted organic group;
   each R⁹ is independently present or absent depending on the number of bonds to, and the valency of, the X⁴, X⁵, X⁸, X⁹ or X¹⁰ atom to which that R⁹ is attached, and each R⁹ is when present independently selected from H and a substituted or unsubstituted organic group;
   Q is a bond or a linker selected from —O—, —N(R¹³)—, and C(R¹⁵)₂,
      wherein R¹³ is H or a C1 to C3 alkyl group, and each R¹⁵ is independently selected from H and substituted or unsubstituted organic group; and
   L is selected from a group having one of the following structures:

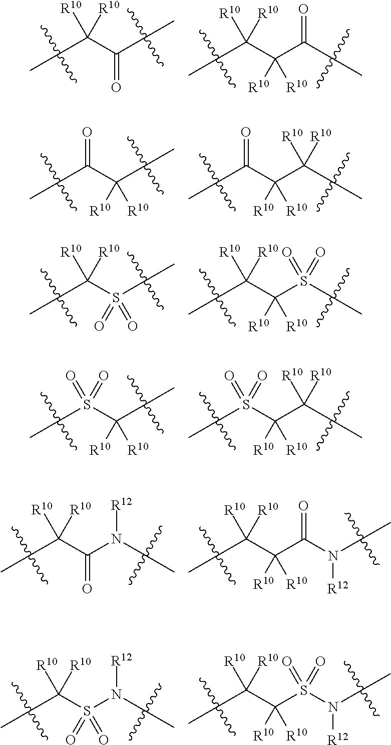

wherein:
   each R¹⁰ is independently selected from H and a substituted or unsubstituted organic group; and
   R¹² is selected from H and a substituted or unsubstituted organic group;
or L and R⁵ together represent:

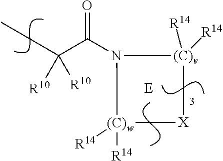

wherein:
   each R¹⁰ is independently selected from H and a substituted or unsubstituted organic group;
   v is 1 or 2;
   w is 1 or 2; and
   each R¹⁴ is independently selected from H and a substituted or unsubstituted organic group.

Optionally, X^{A} is C.

Q may be a bond or a linker selected from —O—, —NH—, —N(Me)-, —CH₂— and C═O. For example, Q may be a bond or a linker selected from —O—, —NH—, and —N(Me)-. Most preferably, Q is a bond.

Optionally, X⁶ is N and R¹¹ is absent.

Alternatively, X⁶ may be C. In such examples, R¹¹ may be selected from H, —CN, a C1 to C3 alkyl group, and a C1 to C3 haloalkyl group; optionally H; —CN; a methyl group, or an ethyl group. In particular, R¹¹ may be H.

Optionally, R⁴ is H or R⁴ and an R⁶ together represent a C1 to C3 alkylene group.

Optionally, no more than one X¹ is a heteroatom selected from N, O and S; each other X¹ being C. Typically, each X¹ is C.

Ring B may have a structure selected from the following:

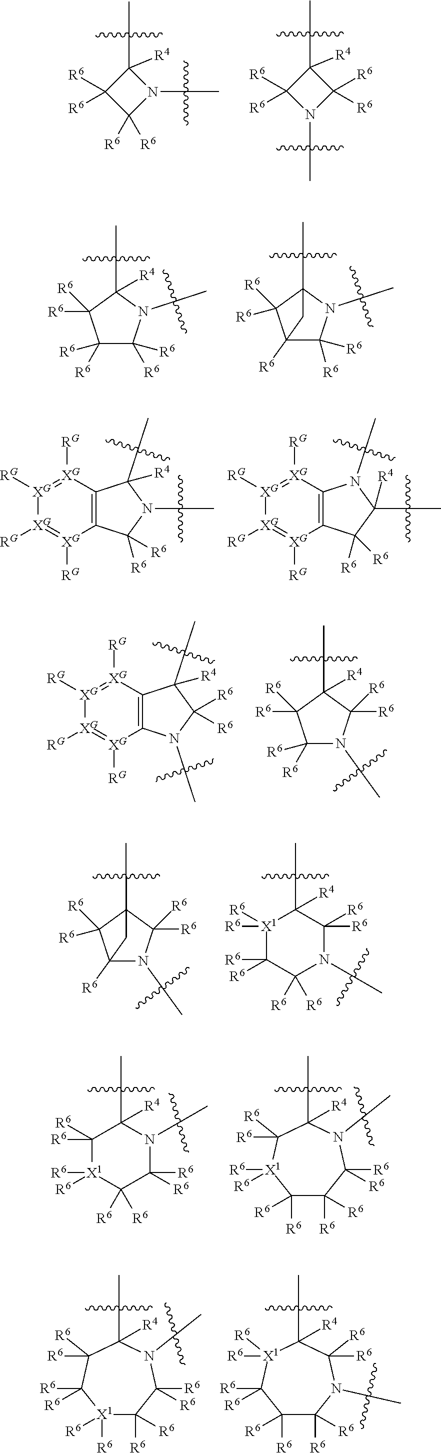

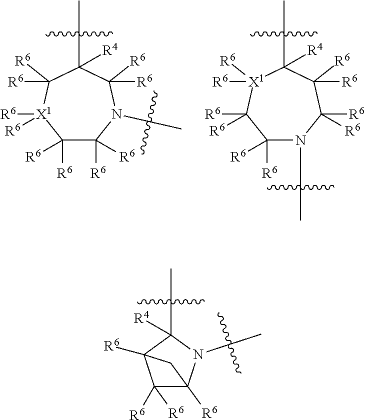

wherein:
   each X^{G} is independently selected from C and N;
      when an X^{G} is N, the corresponding R^{G} is absent;
      when an X^{G} is C, the corresponding R^{G} is independently selected from H and a substituted or unsubstituted organic group; and is optionally selected from H and F.

For example, ring B may have a structure selected from:

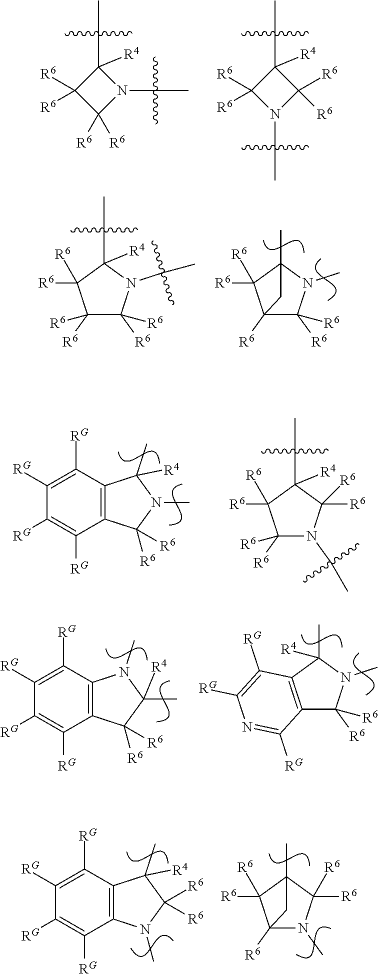

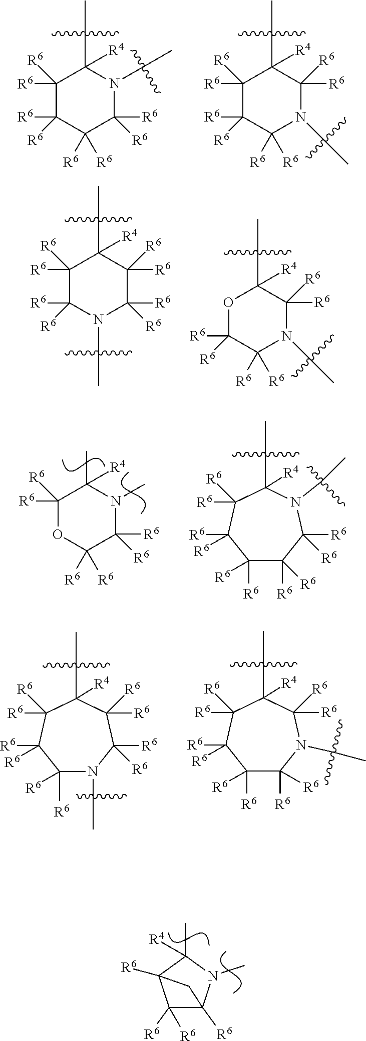

Optionally, each R⁶ is independently selected from H; F; —CH₃; a C1 to C4 alkoxy group, such as —OMe, —OEt, or

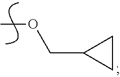

a C1 to C4 haloalkoxy group, such as —OCH₂CH₂F; —OPh; —OCH₂Ph; and

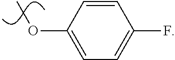

For instance, each R⁶ may be H.

Most preferably, ring B has a structure selected from:

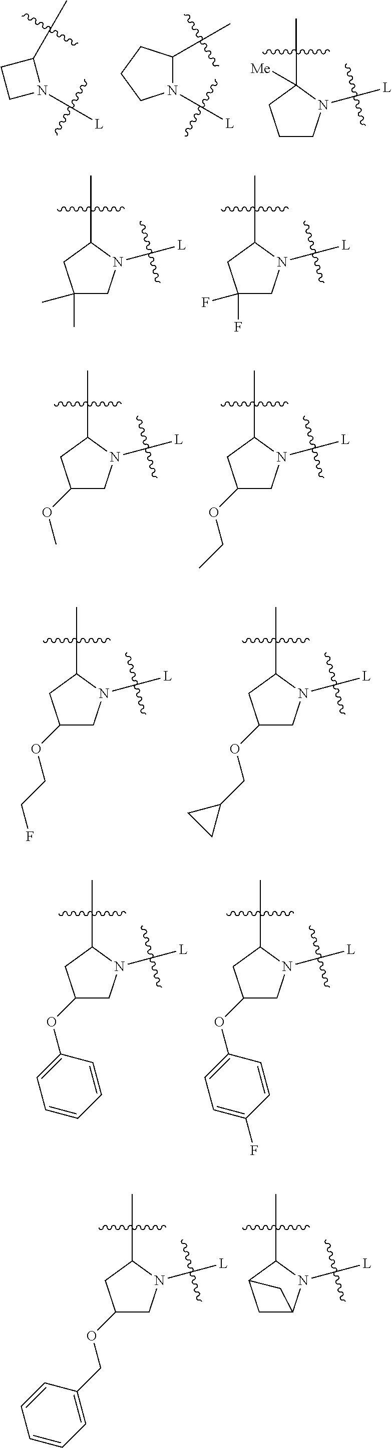

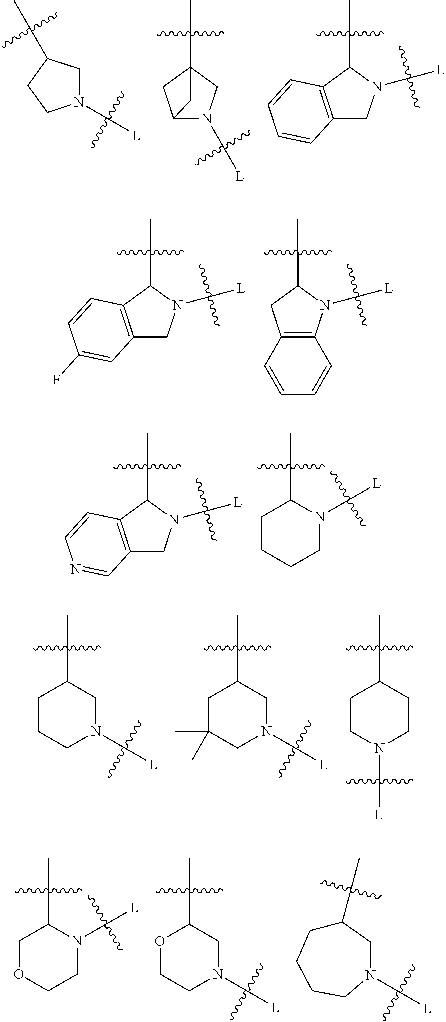

Typically, ring B is a saturated ring.

Optionally, each R¹⁰ is independently selected from:
H;
a halogen, such as —F, —Cl, —Br, and —I, and preferably —F;
a C₁-C₆ alkyl group;
a linear or branched C₁-C₆ halogenated alkyl group, preferably CF₃;
an —NH₂ group;
a C₁-C₆ amino group;
an —OH group;
a linear or branched C₁-C₆ alcohol group; and
a C₁-C₆ alkoxy group.

For example, each R¹⁰ may be H.

R¹² is optionally H or a methyl group.

L may be selected from:

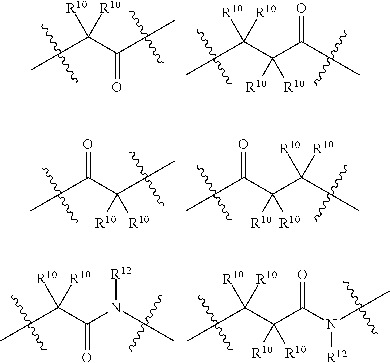

In particular, L may be selected from:

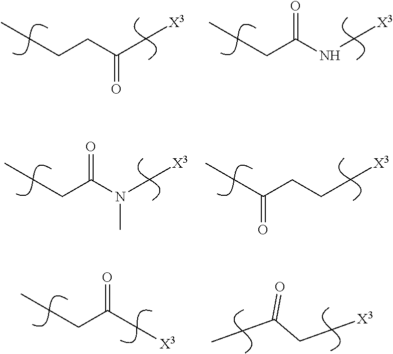

In accordance with another possibility, L may be:

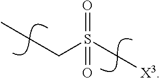

Alternatively, L and R⁵ may together represent:

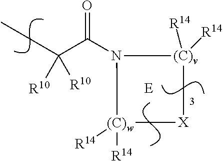

wherein each R¹⁴ is independently selected from H and a substituted or unsubstituted organic group, and ring E is spiro to ring C. Optionally, each R¹⁴ may be H.

For example, L and R⁵ may together represent:

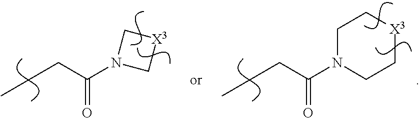

Ring C may be a 6-membered ring. In particular, r may be 2 and s may be 2.

Optionally, each R⁵ is H or absent; and each R⁷ is H or a member of a pair of R⁷ groups which together represent a C1 to C3 alkylene group.

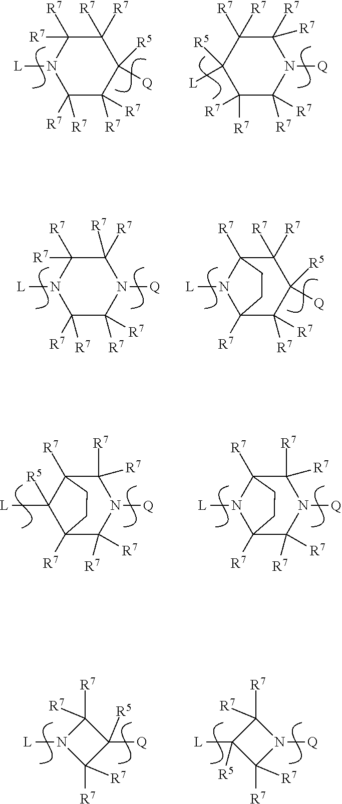

Ring C may have a structure selected from:

For example, ring C may have a structure selected from:

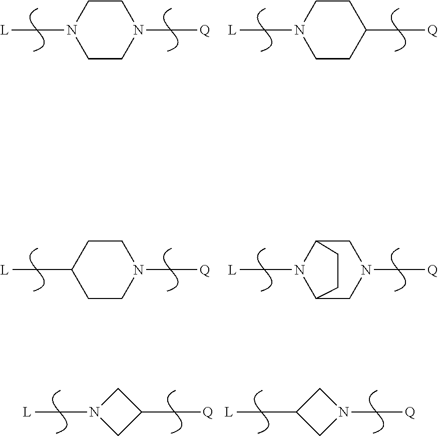

Ring D may have a structure selected from:

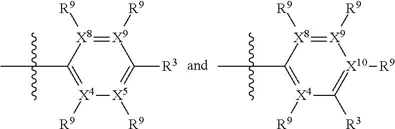

wherein X⁴, X⁵, X⁸, X⁹, and X¹⁰ are each independently selected from C and N.

Alternatively, ring D may have the following structure:

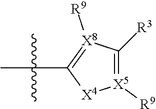

wherein X⁵ and X⁸ are each independently selected from C and N, and X⁴ is selected from O and S.

In accordance with another possibility, ring D may have the following structure:

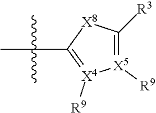

wherein X⁴ and X⁵ are each independently selected from C and N, and X⁸ is selected from O and S.

Ring D optionally has a structure selected from:

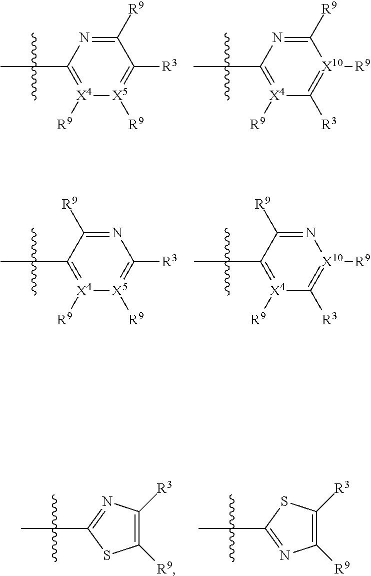

Further optionally, ring D may have a structure selected from:

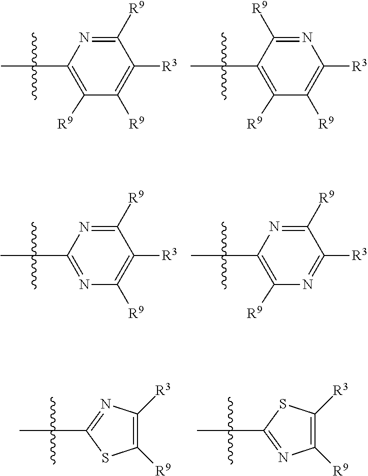

Alternatively, ring D may be selected from:

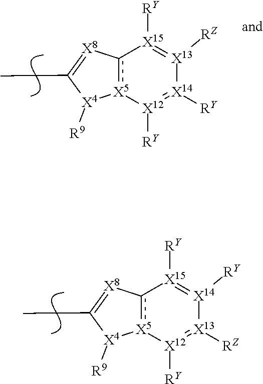

wherein:
   X⁴ is selected from N, O, and S,
   when X⁴ is N:
      R⁹ is H or a substituted or unsubstituted organic group;
   when X⁴ is O or S:
      R⁹ is absent;
   X⁵, X⁸, X¹², X¹³, X¹⁴, and X¹⁵ are each independently selected from C and N;
   each R^{Y} is:
      i) absent when the corresponding one of X¹², X¹⁴, and X¹⁵ is an N; or
      ii) independently selected from H and a substituted or unsubstituted organic group when the corresponding one of X¹², X¹⁴, and X¹⁵ is a C;
   R^{Z} is:
      i) absent when X¹³ is N; or
      ii) H or a substituted or unsubstituted organic group when X¹³ is C.

For example, ring D may be selected from:

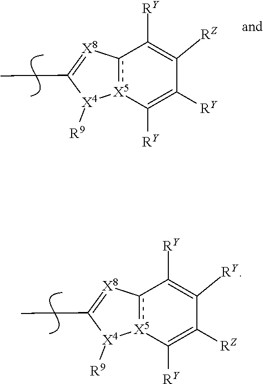

In such compounds, each R^{Y} is optionally H. Alternatively or additionally, R^{Z} may be selected from: H; —CN; —CF₃; and a halogen, such as Cl or F.

R³ may be selected from:
—F; —Cl; —CF₃; —CHF₂, —OCF₂H; —OCF₃; —CN;

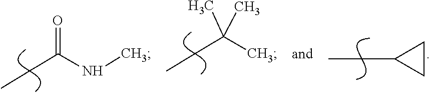

In particular, R³ may be Cl, —CF₃, —CHF₂, —OCHF₂, —CN, or

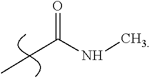

Each R⁹ independently may be H or an organic group selected from a C1-C3 alkyl group; a C1 to C3 fluoroalkyl group, such as CF₃; —CN; and a halogen group. Optionally, no more than one R⁹ is an organic group. Further optionally, each R⁹ may be H.

Ring D may be selected from:

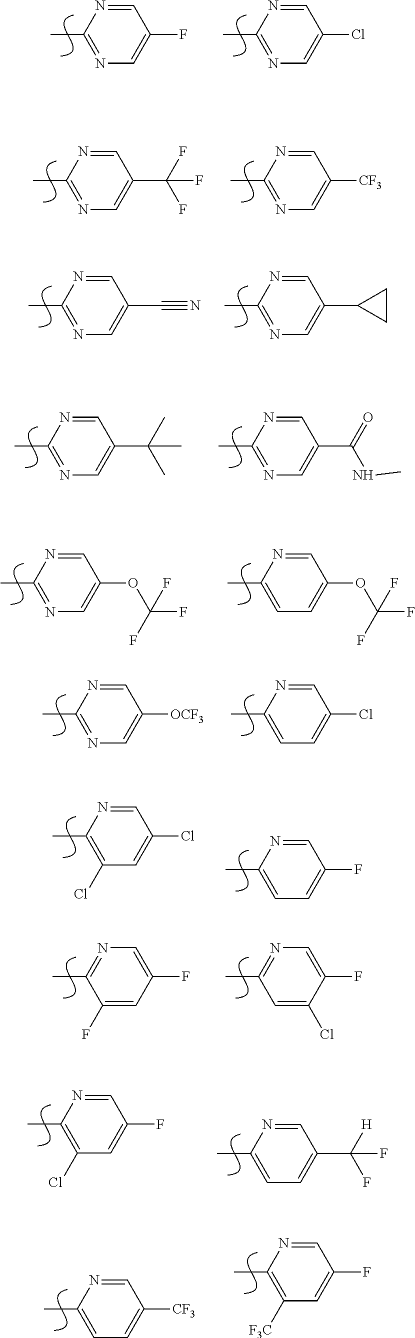

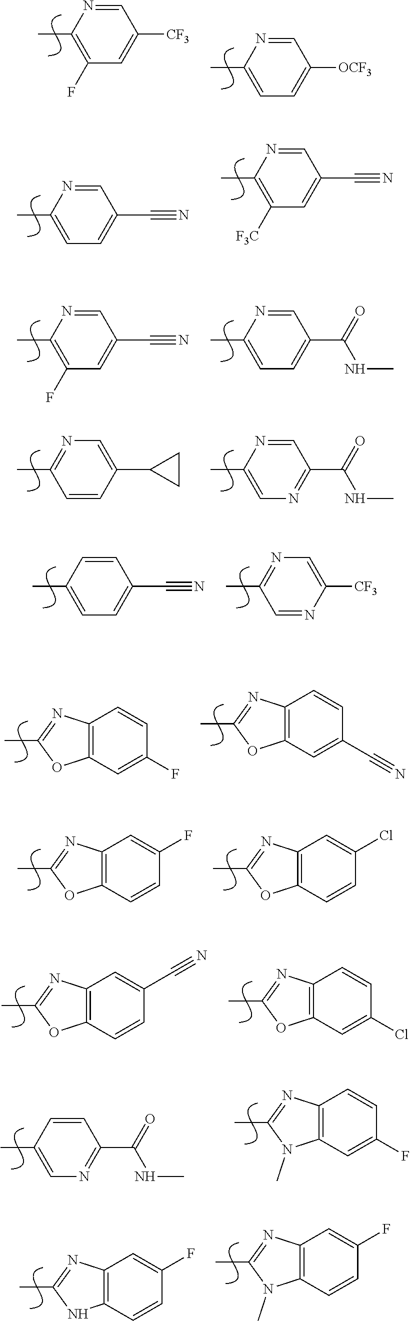

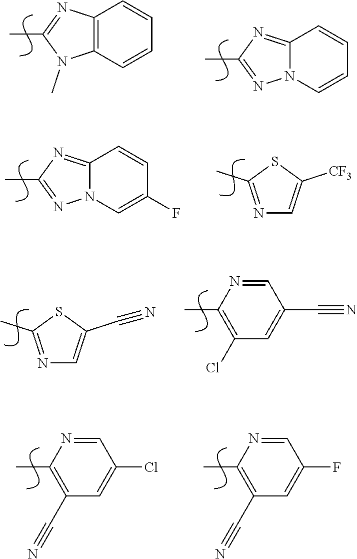

R¹ is optionally selected from:
a C1 to C6 alkyl, alkoxy or haloalkyl group;
a C3 to C6 cycloalkyl or heterocyclic group;
a halogen group; and
—CN.

Further optionally, R¹ may be selected from a —CH₃, —CH₂CH₃—, —CH₂F, —CHF₂, —CF₃, —F, —Cl, —CH₂CF₃, —CN, methoxy, isopropyl or cyclopropyl group. In particular, R¹ may be selected from —CH₃, —CH₂—CH₃, a cyclopropyl group, CF₃, Cl, or —CN.

R² may be H.

In many of the compounds provided herein, X^{A} is C, R² is H, and Q is a bond.

Examples of compounds provided herein include those of formula:

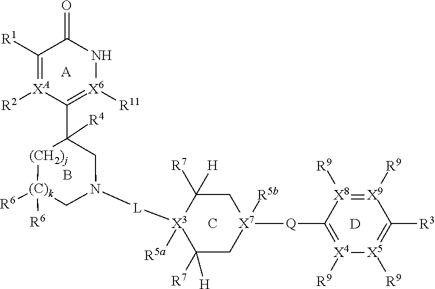

wherein:
   R¹ is selected from —CH₃, —CH₂—CH₃, a cyclopropyl group, CF₃, Cl, and —CN
   X^{A} is selected from C and N;
   when X^{A} is C:
      R² is selected from H, a halogen, a C1 to C3 alkyl group, and a C1 to C3 fluoroalkyl group;
      when X^{A} is N:
         R² is absent;
   i is 0, 1 or 2;
   j is 0, 1, or 2;
   k is 0 or 1;
      with the proviso that i, j and k sum to 1, 2, 3, or 4; and
   R⁴:
      i) is selected from H and methyl group, and each R⁶ is independently selected from H, F, a methyl group, a C1 to C4 alkoxy group, such as —OMe, —OEt, or

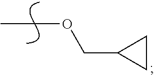

a C1 to C4 haloalkoxy group, such as —OCH₂CH₂F; —OPh; —OCH₂Ph; and

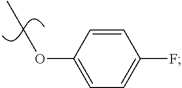

or
ii) is fused with one R⁶ to form a —CH₂— group bridging ring B, and the other R⁶ is H;
L is selected from:

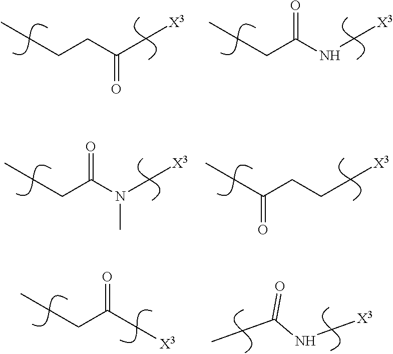

X³ and X⁷ are each selected from C and N, with the proviso that at least one of X³ and
X⁷ is N;
   when X³ is C, R^{5a} is H;
   when X³ is N, R^{5a} is absent;
   when X⁷ is C, R^{5b} is H and Q is a bond or a linker selected from —O—, —NH—, and —N(CH₃)—;
   when X⁷ is N, R^{5b} is absent and Q is a bond;
each R⁷ is H, or the R⁷ groups are fused to form a —CH₂—CH₂— group bridging ring C;
X⁴, X⁵, X⁸, and X⁹ are each selected from C and N, with the proviso that no more than two of X⁴, X⁵, X⁸, and X⁹ are N;
   when X⁴, X⁵, X⁸, or X⁹ is an N, the corresponding R⁹ is absent;
   when X⁴, X⁵, X⁸, or X⁹ is a C, the corresponding R⁹ is selected from H, F, Cl, —CN, and —CF₃; and
R³ is selected from —F; —Cl; —CF₃; —CHF₂, —OCF₂H; —OCF₃; —CN;

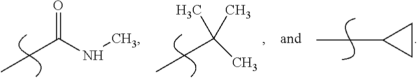

In such compounds, when R⁴ is H or Me:
i) each R⁶ may be H;
ii) each R⁶ may be Me;
iii) each R⁶ may be F; or
iv) one R⁶ may be H, and one R⁶ may be selected from F, a methyl group, a C1 to C4 alkoxy group, such as —OMe, —OEt, or

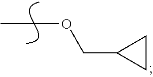

a C1 to C4 haloalkoxy group, such as —OCH₂CH₂F; —OPh; —OCH₂Ph; and

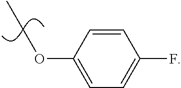

Other examples of compounds provided herein include those of formula:

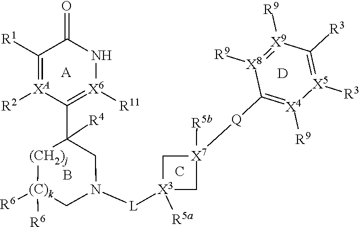

wherein:
   R¹ is selected from —CH₃, —CH₂—CH₃, a cyclopropyl group, CF₃, Cl, and —CN
   X^{A} is selected from C and N;
      when X^{A} is C:
         R² is selected from H, a halogen, a C1 to C3 alkyl group, and a C1 to C3 fluoroalkyl group;
      when X^{A} is N:
         R² is absent;
   i is 0, 1 or 2;
   j is 0, 1, or 2;
   k is 0 or 1;
      with the proviso that i, j and k sum to 1, 2, 3, or 4; and
   R⁴:
      i) is selected from H and methyl group, and each R⁶ is independently selected from H, F, a methyl group, a C1 to C4 alkoxy group, such as —OMe, —OEt, or

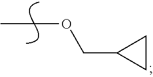

a C1 to C4 haloalkoxy group, such as —OCH₂CH₂F; —OPh; —OCH₂Ph; and

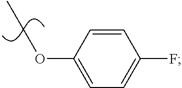

or
ii) is fused with one R⁶ to form a —CH₂— group bridging ring B, and the other R⁶ is H;
L is selected from:

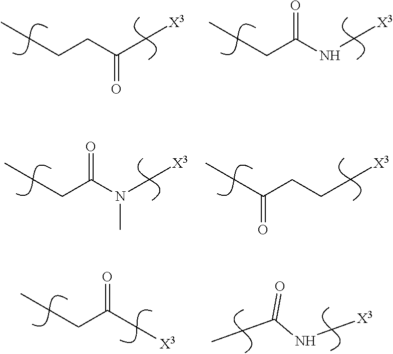

one of X³ and X⁷ is C, and the other of X³ and X⁷ is N;
   when X³ is C, R^{5a} is H;
   when X³ is N, R^{5a} is absent;
   when X⁷ is C, R^{5b} is H and Q is a bond or a linker selected from —O—, —NH—, and —N(CH₃)—;
   when X⁷ is N, R^{5b} is absent and Q is a bond;
X⁴, X⁵, X⁸, and X⁹ are each selected from C and N, with the proviso that no more than two of X⁴, X⁵, X⁸, and X⁹ are N;
   when X⁴, X⁵, X⁸, or X⁹ is an N, the corresponding R⁹ is absent;
   when X⁴, X⁵, X⁸, or X⁹ is a C, the corresponding R⁹ is selected from H, F, Cl, —CN, and —CF₃; and
R³ is selected from —F; —Cl; —CF₃; —CHF₂, —OCF₂H; —OCF₃; —CN;

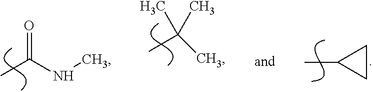

In such compounds, when R⁴ is H or Me:
i) each R⁶ may be H;
ii) each R⁶ may be Me;
iii) each R⁶ may be F; or
iv) one R⁶ may be H, and one R⁶ may be selected from F, a methyl group, a C1 to C4 alkoxy group, such as —OMe, —OEt, or

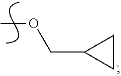

a C1 to C4 haloalkoxy group, such as —OCH₂CH₂F; —OPh; —OCH₂Ph; and

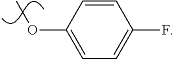

Further examples of compounds provided herein include those of formula:

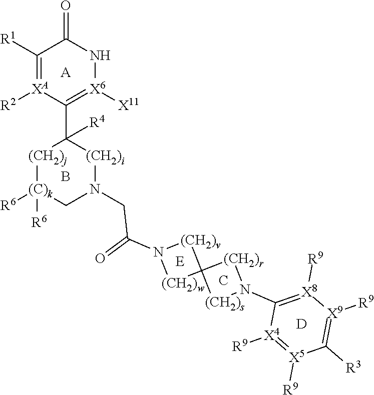

wherein:
   R¹ is selected from —CH₃, —CH₂—CH₃, a cyclopropyl group, CF₃, Cl, and —CN
   X^{A} is selected from C and N;
      when X^{A} is C:
         R² is selected from H, a halogen, a C1 to C3 alkyl group, and a C1 to C3 fluoroalkyl group;
      when X^{A} is N:
         R² is absent;
   i is 0, 1 or 2;
   j is 0, 1, or 2;
   k is 0 or 1;
      with the proviso that i, j and k sum to 1, 2, 3, or 4;
   R⁴:
      i) is selected from H and methyl group, and each R⁶ is independently selected from H, F, a methyl group, a C1 to C4 alkoxy group, such as —OMe, —OEt, or

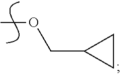

a C1 to C4 haloalkoxy group, such as —OCH₂CH₂F; —OPh; —OCH₂Ph; and

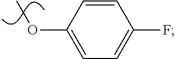

or
ii) is fused with one R⁶ to form a —CH₂— group bridging ring B, and the other R⁶ is H;
v is 1 or2;
w is 1 or 2;
r is 1 or 2;
s is 1 or 2;
X⁴, X⁵, X⁸, and X⁹ are each selected from C and N, with the proviso that no more than two of X⁴, X⁵, X⁸, and X⁹ are N;
   when X⁴, X⁵, X⁸, or X⁹ is an N, the corresponding R⁹ is absent;
   when X⁴, X⁵, X⁸, or X⁹ is a C, the corresponding R⁹ is selected from H, F, Cl, —CN, and —CF₃; and
R³ is selected from —F; —Cl; —CF₃; —CHF₂, —OCF₂H; —OCF₃; —CN;

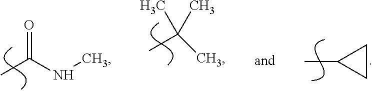

In such compounds, when R⁴ is H or Me:
i) each R⁶ may be H;
ii) each R⁶ may be Me;
iii) each R⁶ may be F; or
iv) one R⁶ may be H, and one R⁶ may be selected from F, a methyl group, a C1 to C4 alkoxy group, such as —OMe, —OEt, or

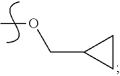

a C1 to C4 haloalkoxy group, such as —OCH₂CH₂F; —OPh; —OCH₂Ph; and

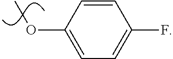

Further examples of compounds provided herein include those of the following formula:

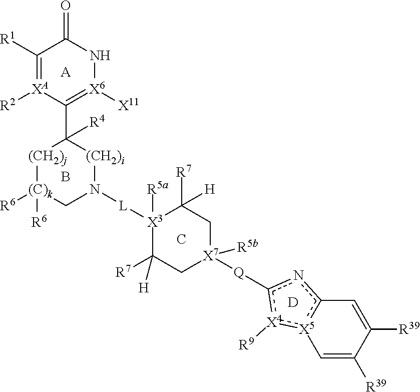

wherein:
   R¹ is selected from —CH₃, —CH₂—CH₃, a cyclopropyl group, CF₃, Cl, and —CN
   X^{A} is selected from C and N;
      when X^{A} is C:
         R² is selected from H, a halogen, a C1 to C3 alkyl group, and a C1 to C3 fluoroalkyl group;
      when X^{A} is N:
         R² is absent;
   i is 0, 1 or 2;
   j is 0, 1, or 2;
   k is 0 or 1;
      with the proviso that i, j and k sum to 1, 2, 3, or 4;
   R⁴:
      i) is selected from H and methyl group, and each R⁶ is independently selected from H, F, a methyl group, a C1 to C4 alkoxy group, such as —OMe, —OEt, or

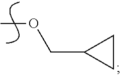

a C1 to C4 haloalkoxy group, such as —OCH₂CH₂F; —OPh; —OCH₂Ph; and

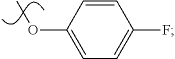

or
ii) is fused with one R⁶ to form a —CH₂— group bridging ring B, and the other R⁶ is H;
L is selected from:

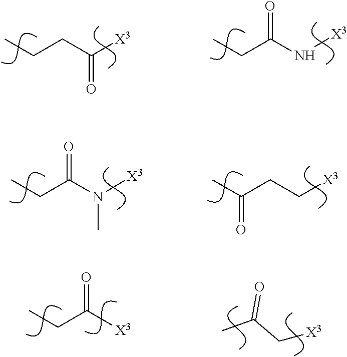

X³ and X⁷ are each selected from C and N, with the proviso that at least one of X³ and X⁷ is N;
   when X³ is C, R^{5a} is H;
   when X³ is N, R^{5a} is absent;
   when X⁷ is C, R^{5b} is H, and Q is a bond or a linker selected from —O—, —NH—, and —N(CH₃)—;
   when X⁷ is N, R^{5b} is absent, and Q is a bond;
each R⁷ is H, or the R⁷ groups are fused to form a —CH₂—CH₂— group bridging ring C;
ring D is an aromatic ring;
X⁴ is selected from N and O;
   when X⁴ is O, R⁹ is absent;
X⁵ is selected from C and N;
   when X⁴ is N and X⁵ is N, R⁹ is absent;
   when X⁴ is N and X⁵ is C, R⁹ is H or a methyl group;
each R³⁹ is selected from H, F, Cl, and —CN, with the proviso that at least one R³⁹ is H.

In such compounds, when R⁴ is H or Me:
i) each R⁶ may be H;
ii) each R⁶ may be Me;
iii) each R⁶ may be F; or
iv) one R⁶ may be H, and one R⁶ may be selected from F, a methyl group, a C1 to C4 alkoxy group, such as —OMe, —OEt, or

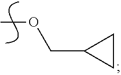

a C1 to C4 haloalkoxy group, such as —OCH₂CH₂F; —OPh; —OCH₂Ph; and

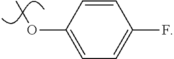

Other examples include compounds of formula:

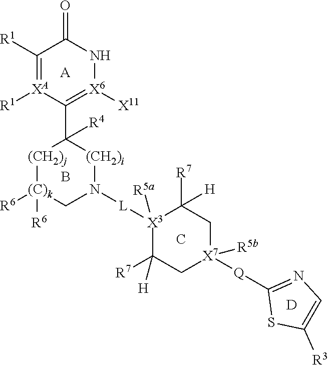

wherein:
   R¹ is selected from —CH₃, —CH₂—CH₃, a cyclopropyl group, CF₃, Cl, and —CN;
   X^{A} is selected from C and N;
      when X^{A} is C:
         R² is selected from H, a halogen, a C1 to C3 alkyl group, and a C1 to C3 fluoroalkyl group;
      when X^{A} is N:
         R² is absent;
   i is 0, 1 or 2;
   j is 0, 1, or 2;
   k is 0 or 1;
      with the proviso that i, j and k sum to 1, 2, 3, or 4;
   R⁴:
      i) is selected from H and methyl group, and each R⁶ is independently selected from H, F, a methyl group, a C1 to C4 alkoxy group, such as —OMe, —OEt, or

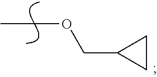

a C1 to C4 haloalkoxy group, such as —OCH₂CH₂F; —OPh; —OCH₂Ph; and

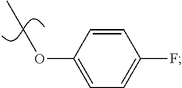

or
ii) is fused with one R⁶ to form a —CH₂— group bridging ring B, and the other R⁶ is H;
L is selected from:

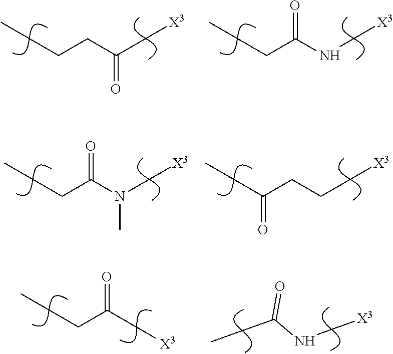

X³ and X⁷ are each selected from C and N, with the proviso that at least one of X³ and
X⁷ is N;
   when X³ is C, R^{5a} is H;
   when X³ is N, R^{5a} is absent;
   when X⁷ is C, R^{5b} is H and Q is a bond or a linker selected from —O—, —NH—, and —N(CH₃)—;
   when X⁷ is N, R^{5b} is absent and Q is a bond;
each R⁷ is H, or the R⁷ groups are fused to form a —CH₂—CH₂— group bridging ring C; and R³ is selected from —F; —Cl; —CF₃; —CHF₂; —OCF₂H; —OCF₃; —CN;

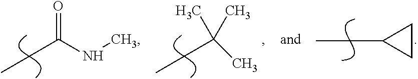

In such compounds, when R⁴ is H or Me:
i) each R⁶ may be H;
ii) each R⁶ may be Me;
iii) each R⁶ may be F; or
iv) one R⁶ may be H, and one R⁶ may be selected from F, a methyl group, a C1 to C4 alkoxy group, such as —OMe, —OEt, or

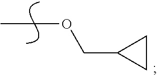

a C1 to C4 haloalkoxy group, such as —OCH₂CH₂F: —OPh: —OCH₂Ph; and

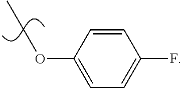

Still further examples include compounds of formula:

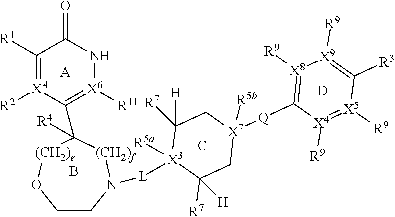

wherein:
   R¹ is selected from —CH₃, —CH₂—CH₃, a cyclopropyl group, CF₃, Cl, and —CN;
   X^{A} is selected from C and N;
      when X^{A} is C:
      R² is selected from H, a halogen, a C1 to C3 alkyl group, and a C1 to C3 fluoroalkyl group;
      when X^{A} is N:
         R² is absent;
   R⁴ is H or a methyl group;
   e is 1 and f is 0, or e is 0 and f is 1;
   L is selected from:

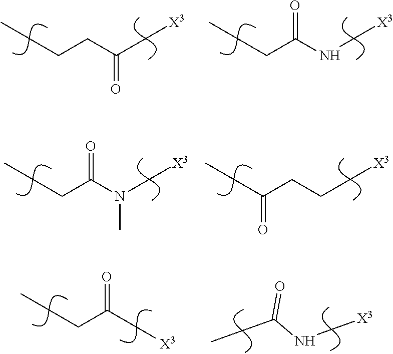

X³ and X⁷ are each selected from C and N, with the proviso that at least one of X³ and X⁷ is N;
   when X³ is C, R^{5a} is H;
   when X³ is N, R^{5a} is absent;
   when X⁷ is C, R^{5b} is H, and Q is a bond or a linker selected from —O—, —NH—, and —N(CH₃)—;
   when X⁷ is N, R^{5b} is absent, and Q is a bond;
each R⁷ is H, or the R⁷ groups are fused to form a —CH₂—CH₂— group bridging ring C;
X⁴, X⁵, X⁸, and X⁹ are each selected from C and N, with the proviso that no more than two of X⁴, X⁵, X⁸, and X⁹ are N;
   when X⁴, X⁵, X⁸, or X⁹ is an N, the corresponding R⁹ is absent;
   when X⁴, X⁵, X⁸, or X⁹ is a C, the corresponding R⁹ is selected from H, F, Cl, —CN, and —CF₃;
and
R³ is selected from —F; —Cl; —CF₃; —CHF₂; —OCF₂H; —OCF₃; —CN;

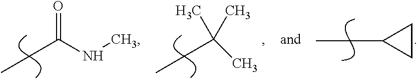

Additional examples include compounds of formula:

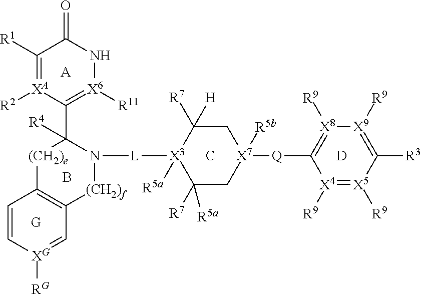

wherein:
   R¹ is selected from —CH₃, —CH₂—CH₃, a cyclopropyl group, CF₃, Cl, and —CN
   X^{A} is selected from C and N;
      when X^{A} is C:
         R² is selected from H, a halogen, a C1 to C3 alkyl group, and a C1 to C3 fluoroalkyl group;
      when X^{A} is N:
         R² is absent;
   R⁴ is H or a methyl group;
   e is 1 and f is 0, or e is 0 and f is 1;
   X^{G} is selected from C and N;
      when X^{G} is C, R^{G} is selected from H and F;
      when X^{G} is N, R^{G} is absent;
   L is selected from:

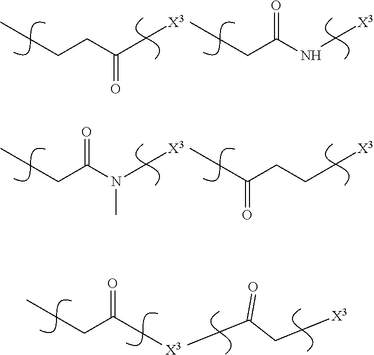

X³ and X⁷ are each selected from C and N, with the proviso that at least one of X³ and X⁷ is N;
   when X³ is C, R^{5a} is H;
   when X³ is N, R^{5a} is absent;
   when X⁷ is C, R^{5b} is H, and Q is a bond or a linker selected from —O—, —NH— and —N(CH₃)—;
   when X⁷ is N, R^{5b} is absent, and Q is a bond;
each R⁷ is H, or the R⁷ groups are fused to form a —CH₂—CH₂— group bridging ring C;
X⁴, X⁵, X⁸, and X⁹ are each selected from C and N, with the proviso that no more than two of X⁴, X⁵, X⁸, and X⁹ are N;
   when X⁴, X⁵, X⁸, or X⁹ is an N, the corresponding R⁹ is absent;
   when X⁴, X⁵, X⁸, or X⁹ is a C, the corresponding R⁹ is selected from H, F, Cl, —CN, and —CF₃;
and
R³ is selected from —F; —Cl; —CF₃; —CHF₂; —OCF₂H; —OCF₃; —CN;

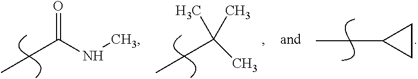

Other examples of compounds provided herein include those of formula:

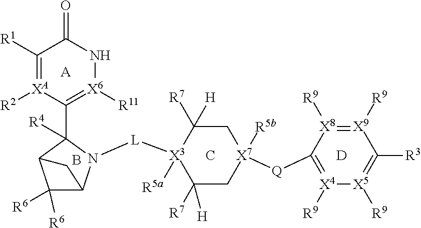

wherein:
   R¹ is selected from —CH₃, —CH₂—CH₃, a cyclopropyl group, CF₃, Cl, and —CN;
   X^{A} is selected from C and N;
      when X^{A} is C:
      R² is selected from H, a halogen, a C1 to C3 alkyl group, and a C1 to C3 fluoroalkyl group;
      when X^{A} is N:
         R² is absent;
   R⁴ is selected from H and methyl group;
   each R⁶ is independently selected from H, F, a methyl group, a C1 to C4 alkoxy group, such as —OMe, —OEt, or

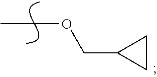

C1 to C4 haloalkoxy group, such as —OCH₂CH₂F; —OPh; —OCH₂Ph; and

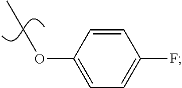

L is selected from:

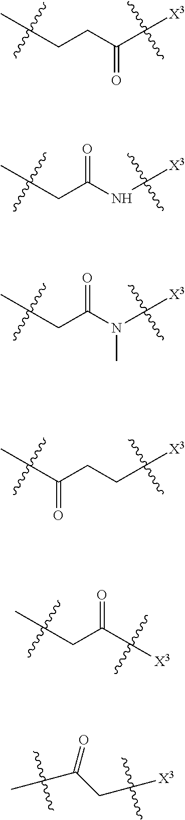

X³ and X⁷ are each selected from C and N, with the proviso that at least one of X³ and X⁷ is N;
   when X³ is C, R^{5a} is H;
   when X³ is N, R^{5a} is absent;
   when X⁷ is C, R^{5b} is H and Q is a bond or a linker selected from —O—, —NH—, and —N(CH₃)—;
   when X⁷ is N, R^{5b} is absent and Q is a bond;
each R⁷ is H, or the R⁷ groups are fused to form a —CH₂—CH₂— group bridging ring C;
X⁴, X⁵, X⁸, and X⁹ are each selected from C and N, with the proviso that no more than two of X⁴, X⁵, X⁸, and X⁹ are N;
   when X⁴, X⁵, X⁸, or X⁹ is an N, the corresponding R⁹ is absent;
   when X⁴, X⁵, X⁸, or X⁹ is a C, the corresponding R⁹ is selected from H, F, Cl, —CN, and —CF₃; and
R³ is selected from —F; —Cl; —CF₃; —CHF₂, —OCF₂H; —OCF₃; —CN;

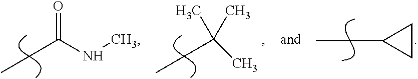

Optionally, in such compounds:
i) each R⁶ may be H;
ii) each R⁶ may be Me;
iii) each R⁶ may be F; or
iv) one R⁶ may be H, and one R⁶ may be selected from F, a methyl group, a C1 to C4 alkoxy group, such as —OMe, —OEt, or

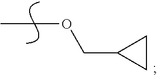

a C1 to C4 haloalkoxy group, such as —OCH₂CH₂F; —OPh; —OCH₂Ph; and

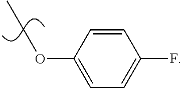

Provided are compounds having the following formula:

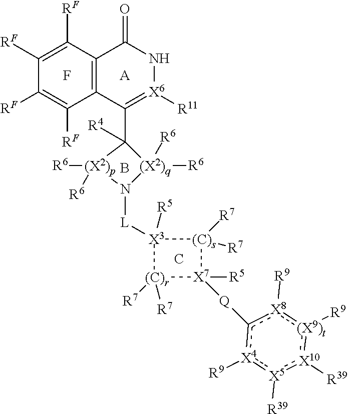

wherein:
   each R^{F} is independently selected from H and a substituted or unsubstituted organic group. Optionally, each R^{F} is H.

More specific compounds in the class defined immediately above are those of formula:

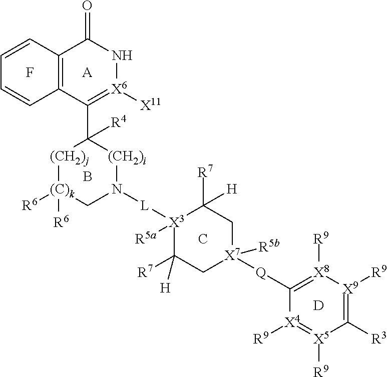

wherein:
   i is 0, 1 or 2;
   j is 0, 1, or 2;
   k is 0 or 1;
      with the proviso that i, j and k sum to 1, 2, 3, or 4;
   R⁴:
      i) is selected from H and methyl group, and each R⁶ is independently selected from H, F, a methyl group, a C1 to C4 alkoxy group, such as —OMe, —OEt, or

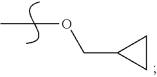

a C1 to C4 haloalkoxy group, such as —OCH₂CH₂F; —OPh; —OCH₂Ph; and

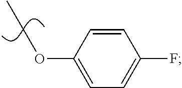

ii) is fused with one R⁶ to form a —CH₂— group bridging ring B, and the other R⁶ is H;
L is selected from:

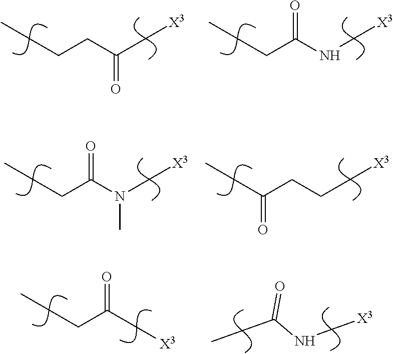

X³ and X⁷ are each selected from C and N, with the proviso that at least one of X³ and
X⁷ is N;
   when X³ is C, R^{5a} is H;
   when X³ is N, R^{5a} is absent;
   when X⁷ is C, R^{5b} is H, and Q is a bond or a linker selected from —O—, —NH— and —N(CH₃)—;
   when X⁷ is N, R^{5b} is absent, and Q is a bond;
each R⁷ is H, or the R⁷ groups are fused to form a —CH₂—CH₂— group bridging ring C;
X⁴, X⁵, X⁸, and X⁹ are each selected from C and N, with the proviso that no more than two of X⁴, X⁵, X⁸, and X⁹ are N;
   when X⁴, X⁵, X⁸, or X⁹ is an N, the corresponding R⁹ is absent;
   when X⁴, X⁵, X⁸, or X⁹ is a C, the corresponding R⁹ is selected from H, F, Cl, —CN, and —CF₃; and
R³ is selected from —F; —Cl; —CF₃; —CHF₂, —OCF₂H; —OCF₃; —CN;

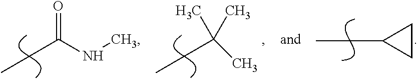

In such compounds, when R⁴ is H or Me:
i) each R⁶ may be H;
ii) each R⁶ may be Me;
iii) each R⁶ may be F; or
iv) one R⁶ may be H, and one R⁶ may be selected from F, a methyl group, a C1 to C4 alkoxy group, such as —OMe, —OEt, or

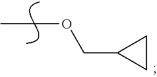

a C1 to C4 haloalkoxy group, such as —OCH₂CH₂F; —OPh; —OCH₂Ph; and

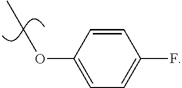

Where not otherwise specified, any of R⁴, R⁵, R⁶, R⁷ and R¹⁰ may be individually selected from H and the various organic groups identified in the detailed description (e.g. deuterium; a halogen; a nitrile group; a C1-C6 alkyl group, a C1-C6 alkyl-aryl group; a C1-C6 halogenated alkyl group; —NH₂; a primary, secondary, or tertiary C1-C6 amine group; an amino-aryl group; a cyclic amine or amido group; a cyclic C3-C8 alkyl group; an —OH group; a C1-C6 alcohol group; a C1-C6 carboxylic acid group; a carbonyl group; a C1-C6 carboxylic acid ester group; a C1-C6 amide group; a C1-C7 amino carbonyl group; a C1-C7 alkoxy or aryloxy group; an aminoalkoxy group; a sulphonyl group; an aminosulphonyl group; an aromatic group; or a heterocyclic group).

Where there are two R groups attached to the same atom, they may together form a group which is double bonded to that atom. For example, two R groups attached to the same atom may together represent a carbonyl group ═O, or an alkene group ═C(R′)₂ wherein each R′ group is the same or different and is H or an organic group, preferably H or a linear or branched C₁-C₆ alkyl group.

A pair of R⁶ groups attached to different atoms may together form a ring with ring B atoms. Alternatively or additionally, an R⁶ group and an R⁴ group may together form a ring with ring B atoms. Alternatively or additionally, a pair of R⁷ groups attached to different atoms may together form a ring with ring C atoms. Alternatively or additionally, an R⁷ group and an R⁵ group may together form a ring with ring B atoms.

When a pair of such groups forms a ring, each of the pair of R⁶ groups and/or the R⁶ and R⁴ groups and/or the pair of R⁷ groups and/or the R⁷ and R⁵ groups independently comprises (X¹¹)_{1 or 2},
wherein each X¹¹ is independently selected from C, N, O and S; and
each X¹¹ is independently unsubstituted or
   i) when X¹¹ is C: independently substituted with H or an organic group selected from a C₁-C₆ alkyl group, a halogen such as F, or hydroxyl; or
   ii) when X¹¹ is N: independently substituted with H or an organic group selected from a C₁-C₆ alkyl group or a C₁-C₆ amide group.

For example, R⁴, R⁵, R⁶, and R⁷ may each independently be selected from:
H;
deuterium;
a halogen, such as —F, —Cl, —Br, and —I, preferably F or Cl;
a C₁-C₆ alkyl group;
a linear or branched C₁-C₆ halogenated alkyl group, preferably CF₃;
an —OH group;
a linear or branched C₁-C₆ alcohol group;
an —NH₂ group;
a C₁-C₆ amino group;
a C₁-C₆ alkoxy group, such as —OMe or —OEt;
a C1-C6 haloalkoxy group, optionally a C1 to C6 fluoroalkoxy group such as —OCH₂CH₂F; an aryloxy group, such as —OPh;
a haloaryloxy group, such as

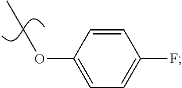

and
an arylalkoxy group, such as —OCH₂Ph.

Optionally, when a pair of R⁶ groups attached to different atoms together forms a ring with ring B atoms, and/or a pair of R⁷ groups attached to different atoms together forms a ring with ring C atoms, the pair of R⁶ groups and/or pair of R⁷ groups form a —CH₂— or —CH₂CH₂— group.

R¹² may be selected from H and the various example organic groups identified in the detailed description hereinbelow (a substituted or unsubstituted group selected from: a C₁-C₆ alkyl group; a C₁-C₆ alkyl-aryl group; a C₁-C₆ halogenated alkyl group; a cyclic amine or amido group; a cyclic C₃-C₈ alkyl group; a C₂-C₆ alcohol group; a C₂-C₆ carboxylic acid group; a carbonyl group; a C₁-C₆ carboxylic acid ester group; a C₁-C₆ amide group; a sulphonyl group; an aromatic group; and a heterocyclic group). Optionally, R¹² is selected from H, a C₁-C₆ alkyl group, and a linear or branched C₁-C₆ halogenated alkyl group.

The compound may comprise a formula selected from one of the following formulae G1 to G123:

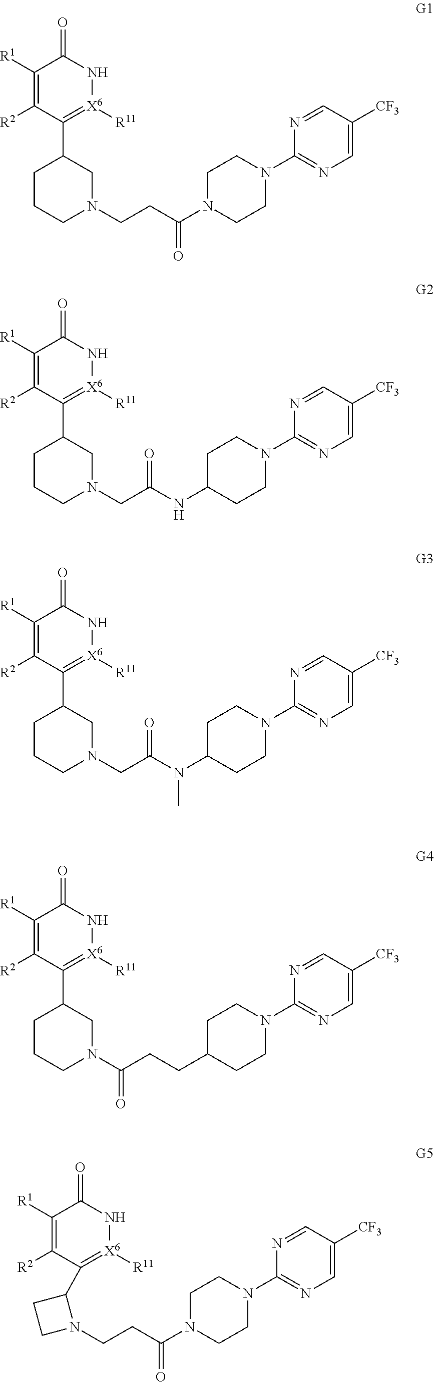

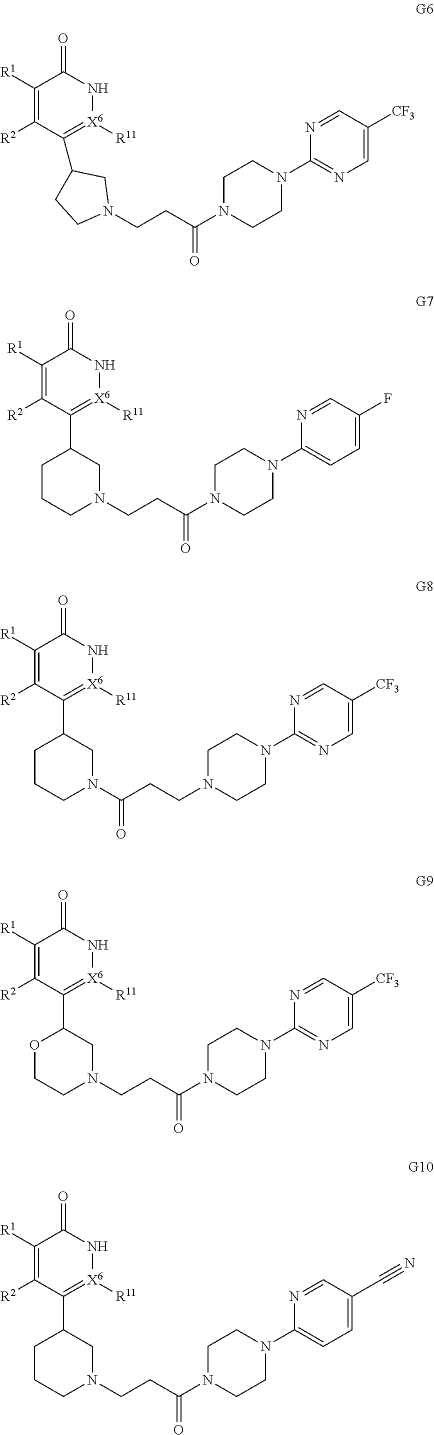

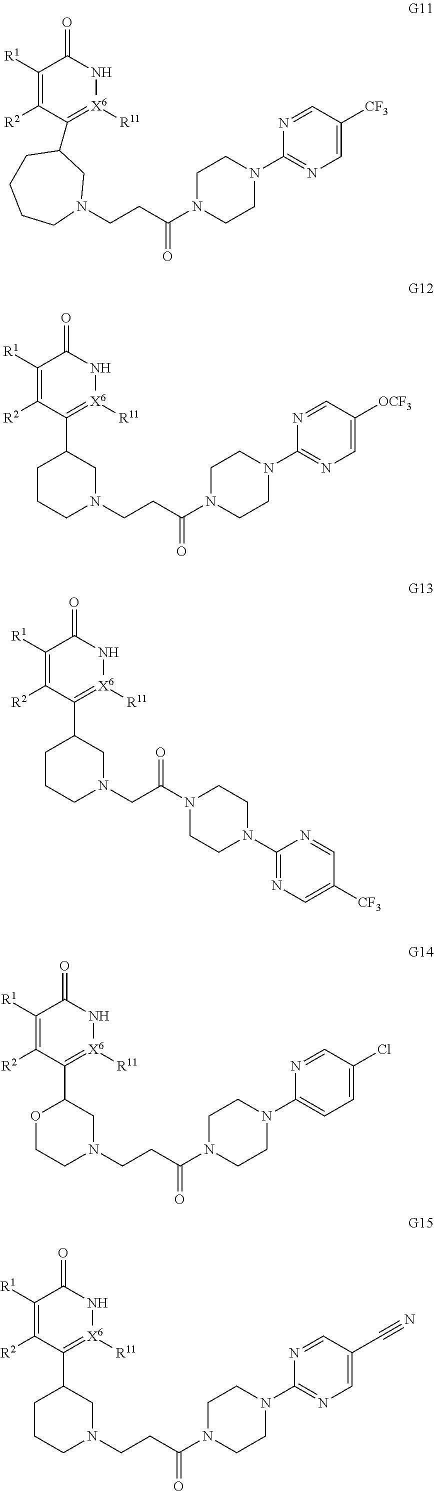

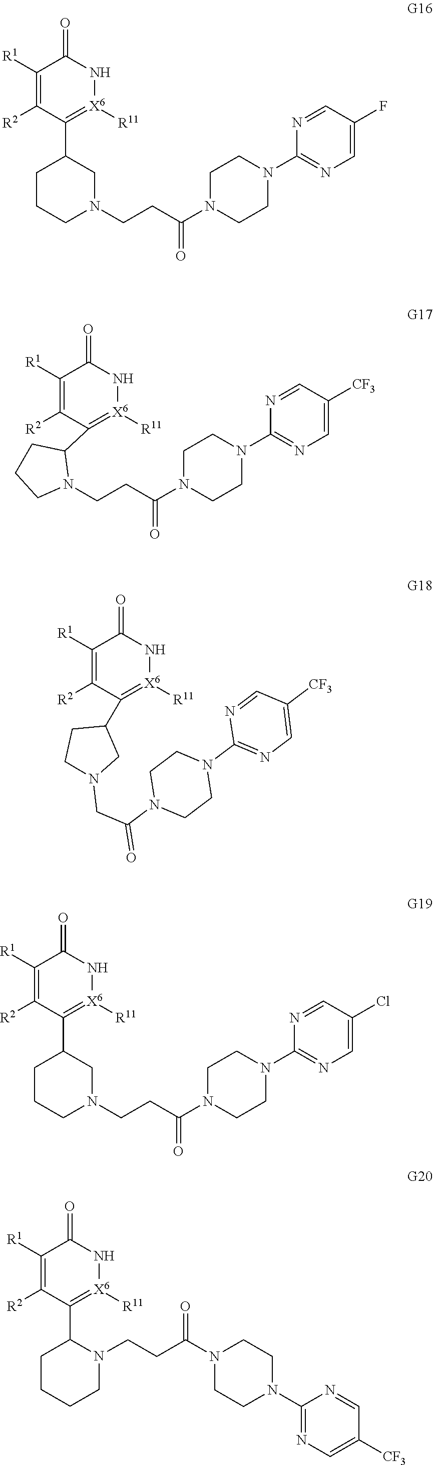

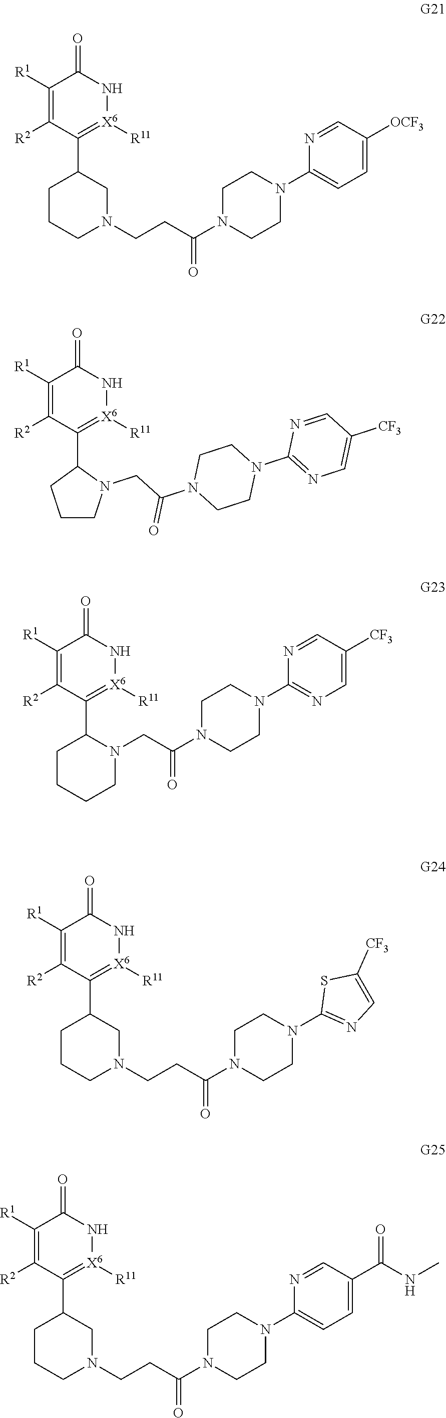

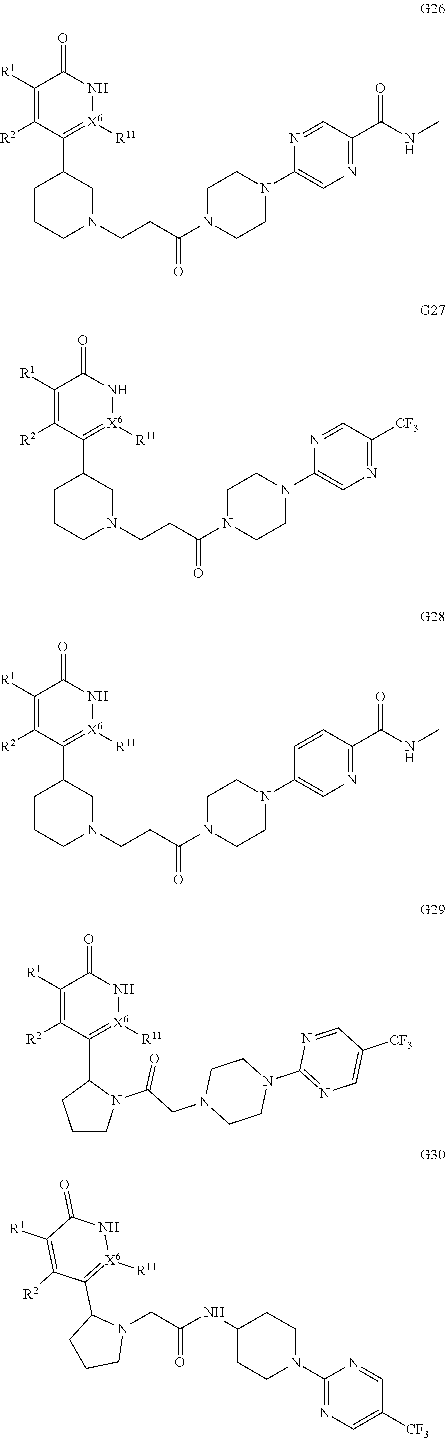

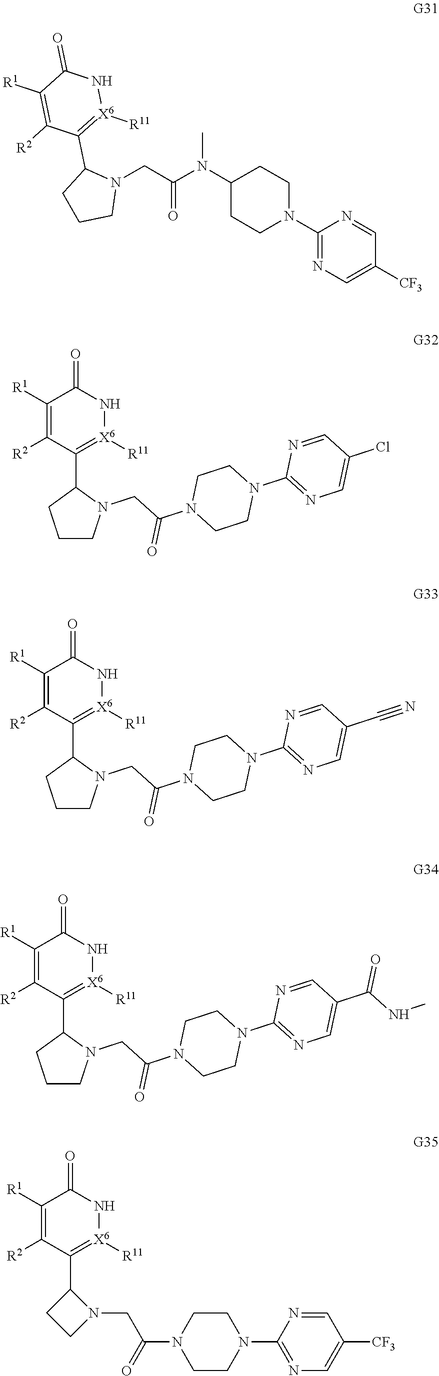

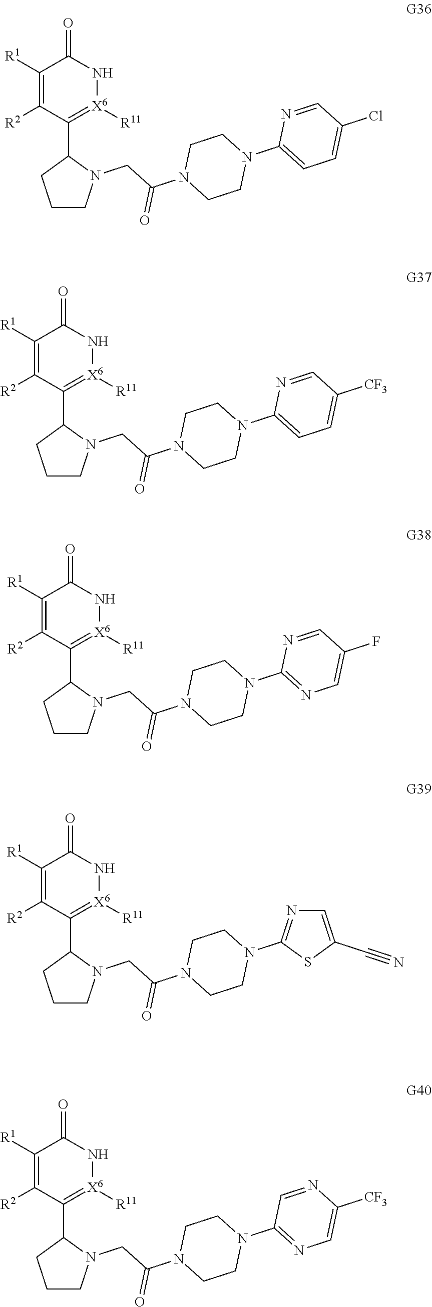

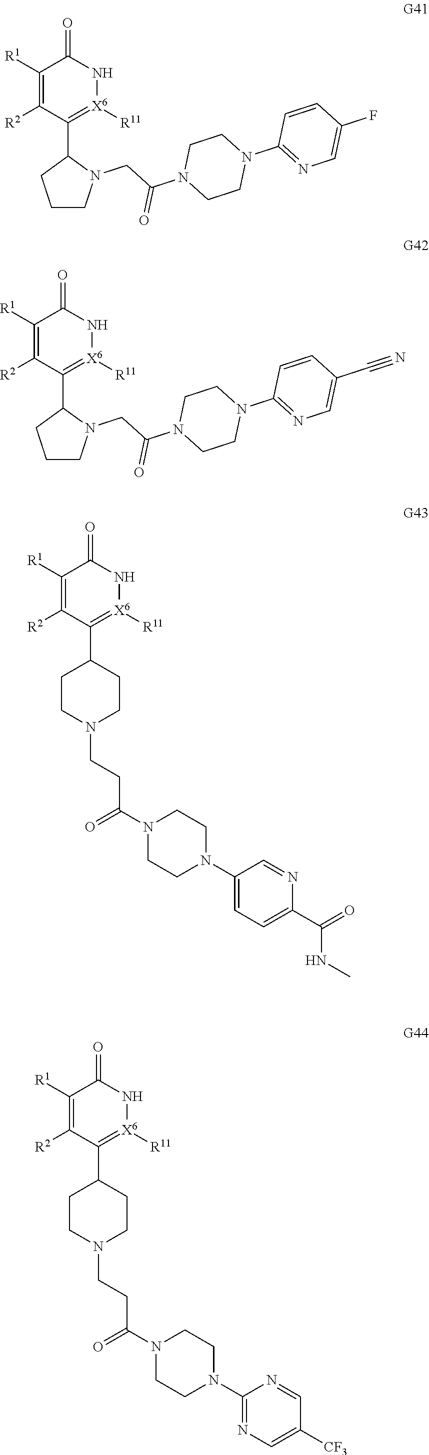

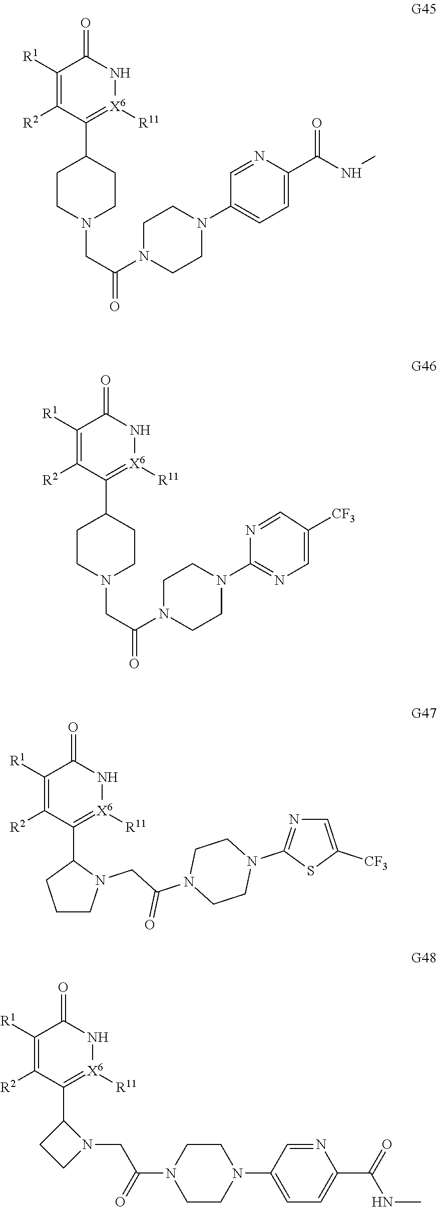

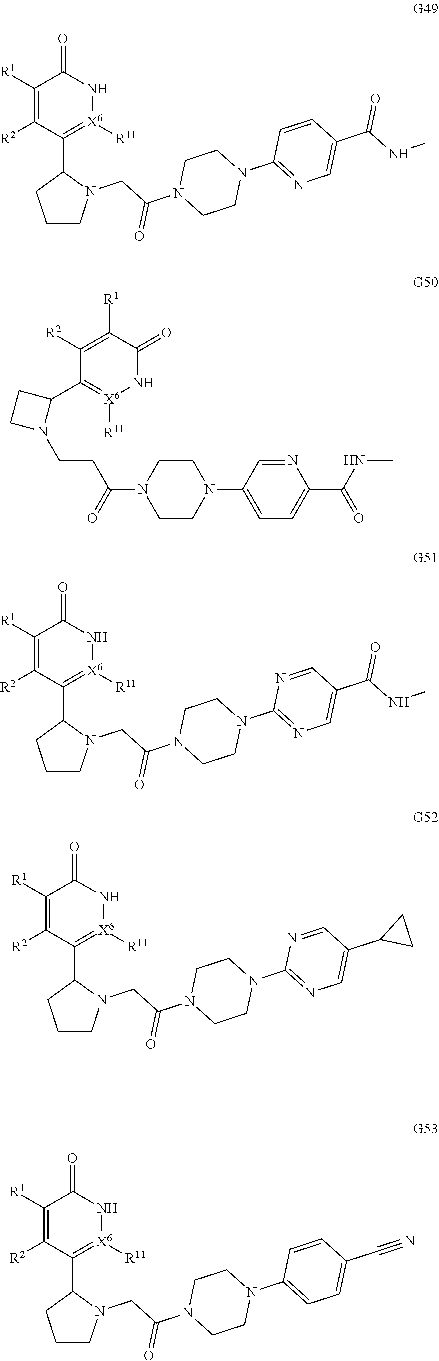

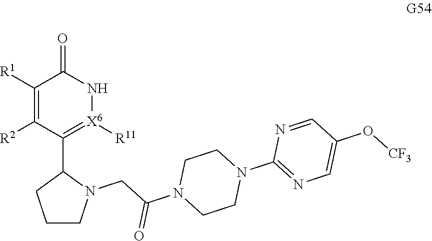

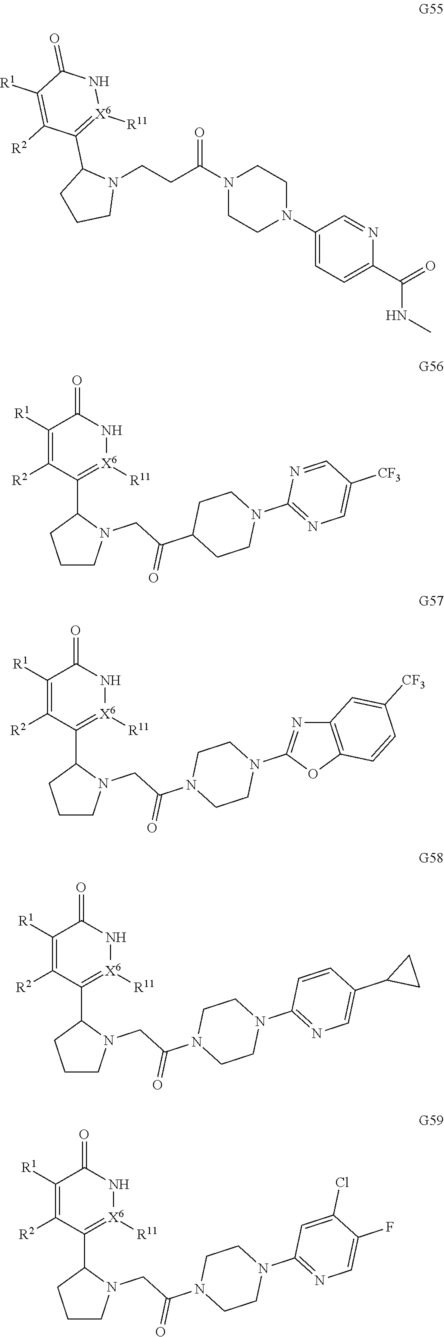

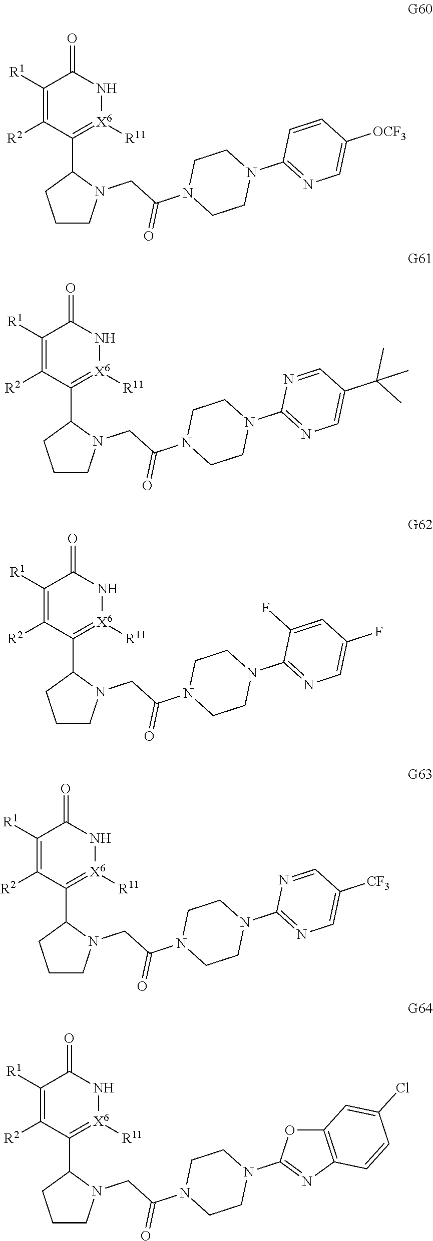

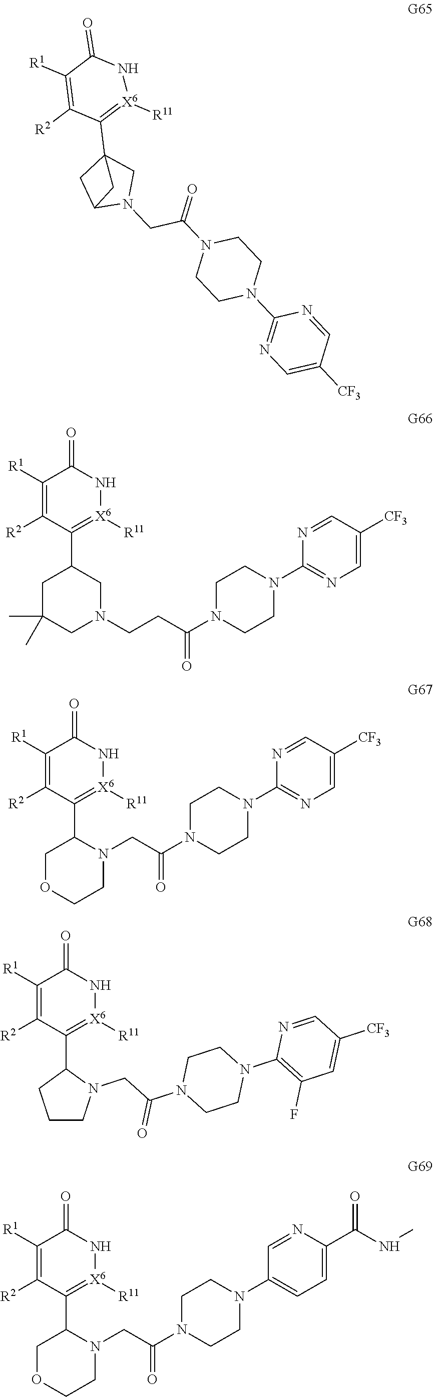

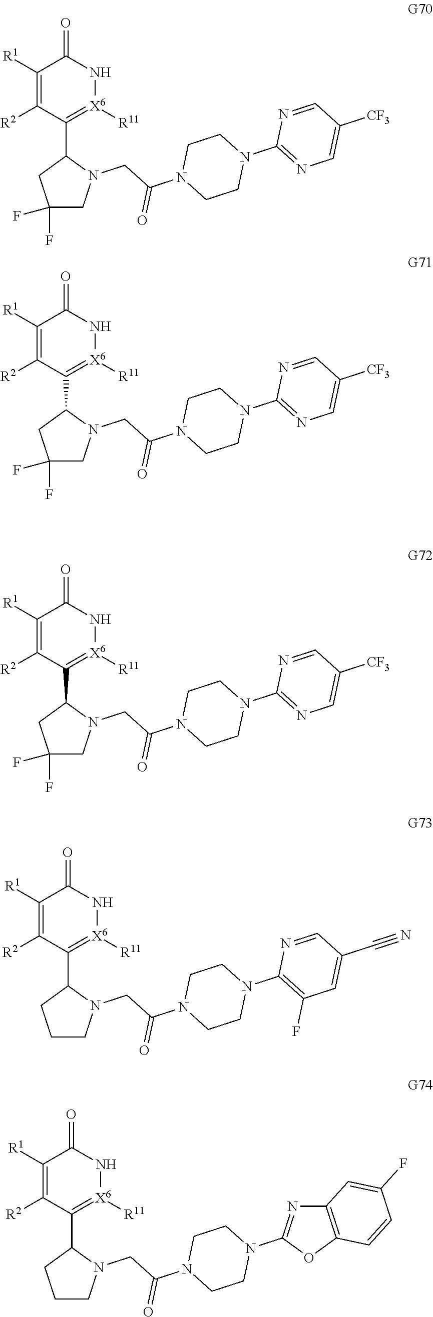

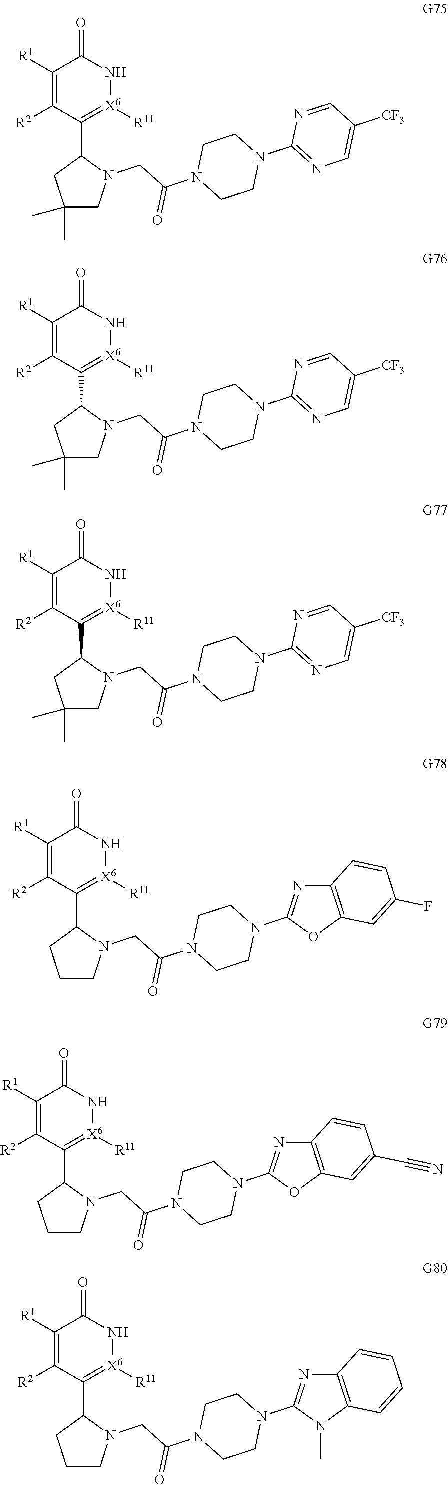

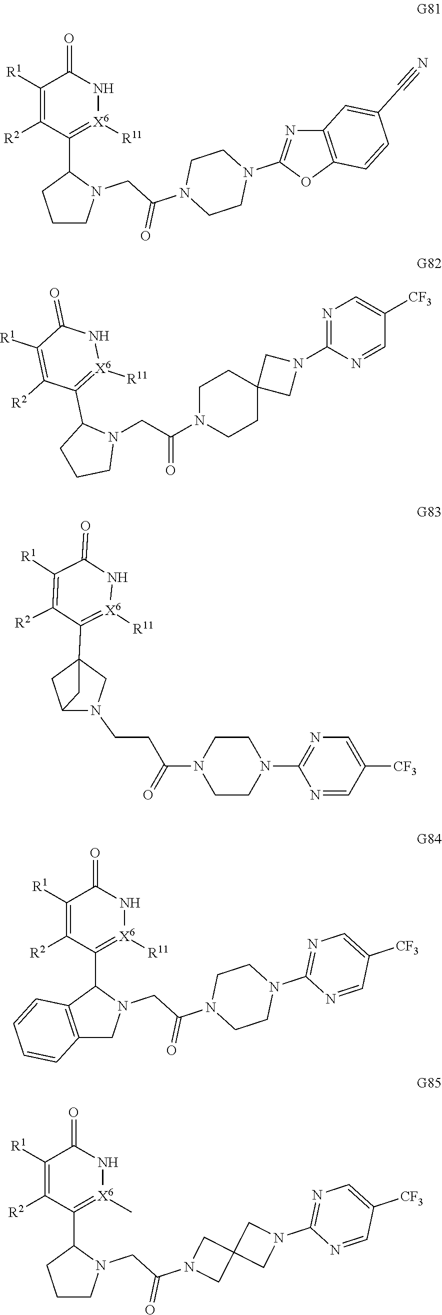

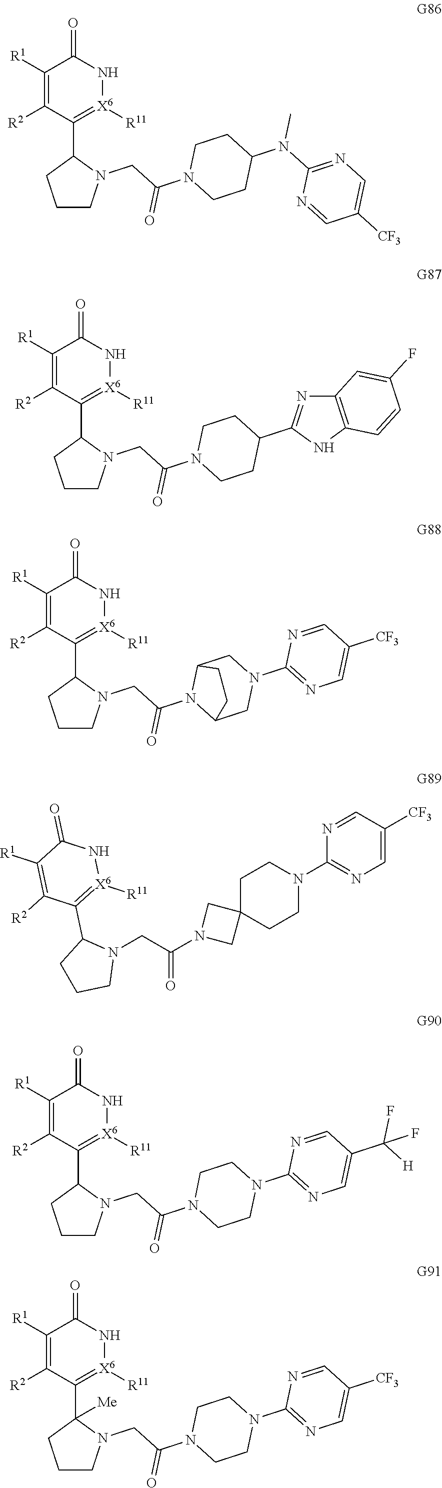

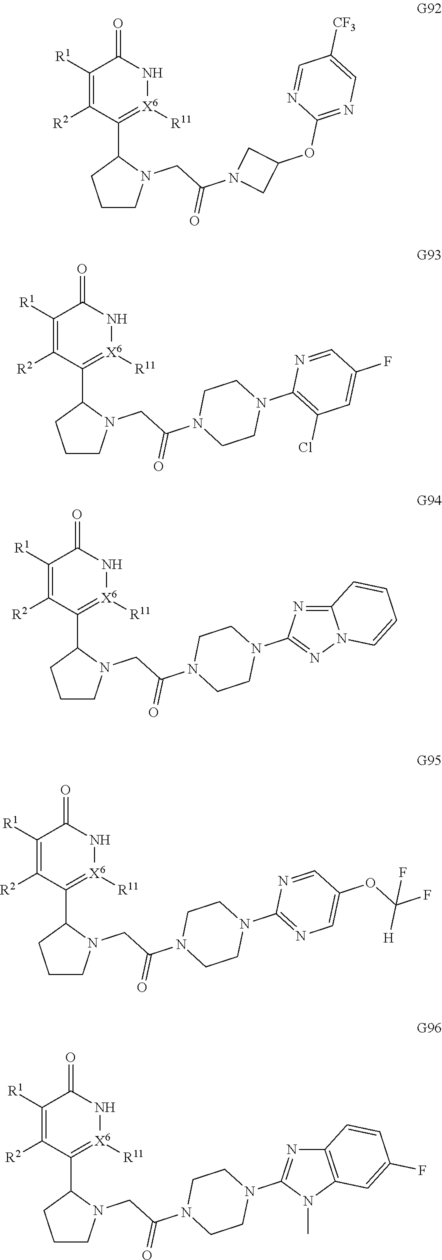

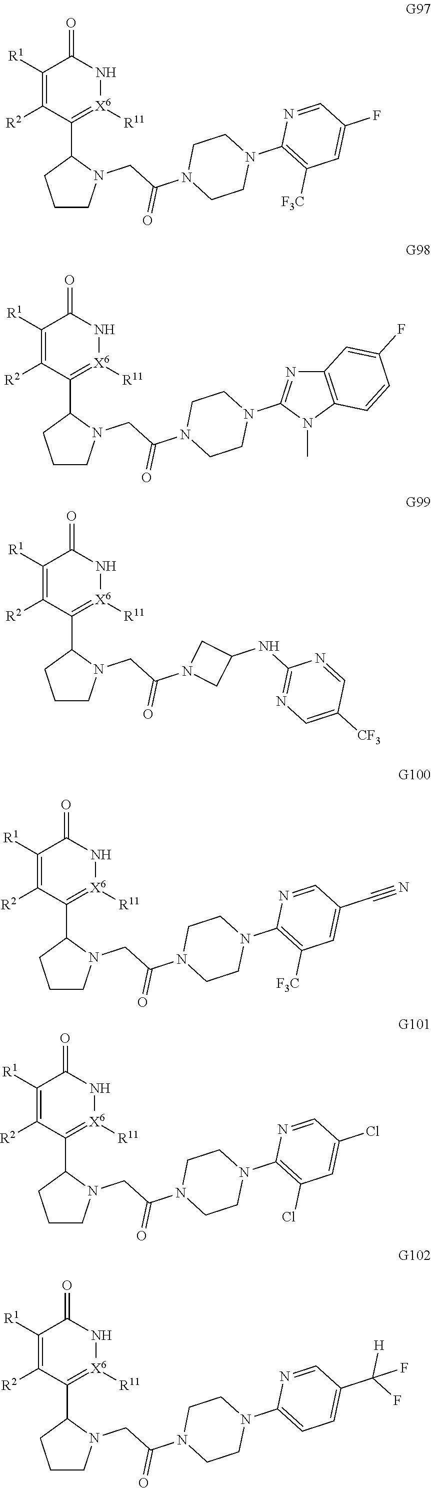

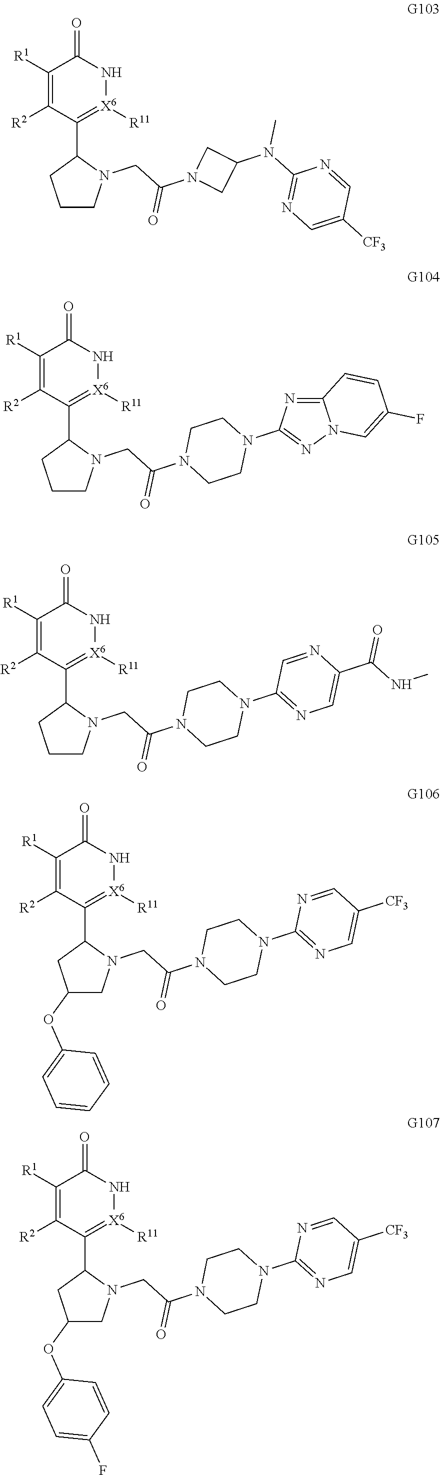

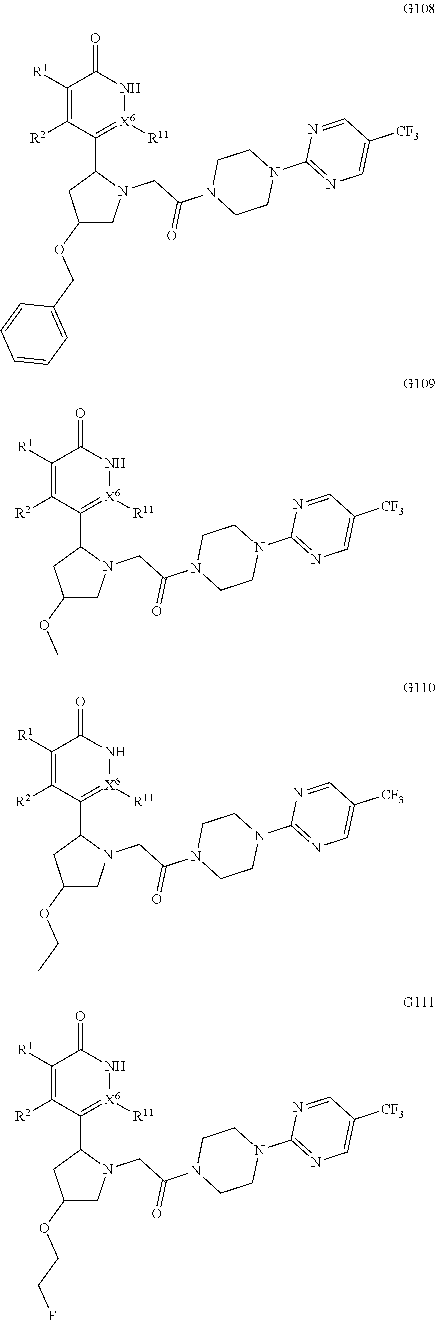

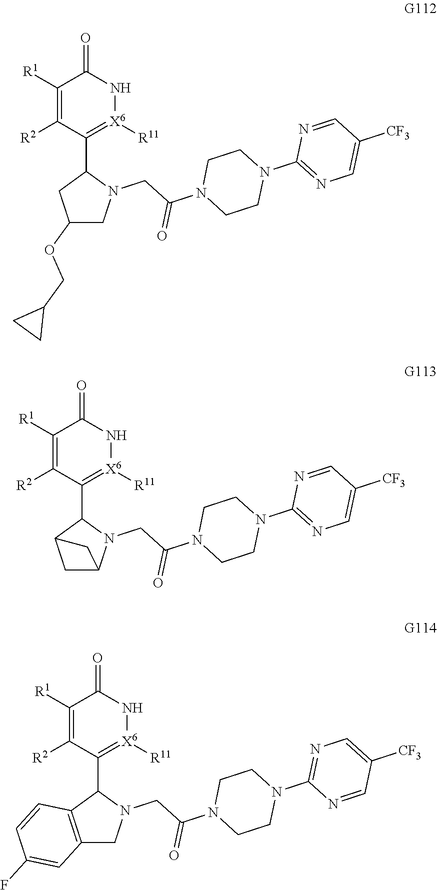

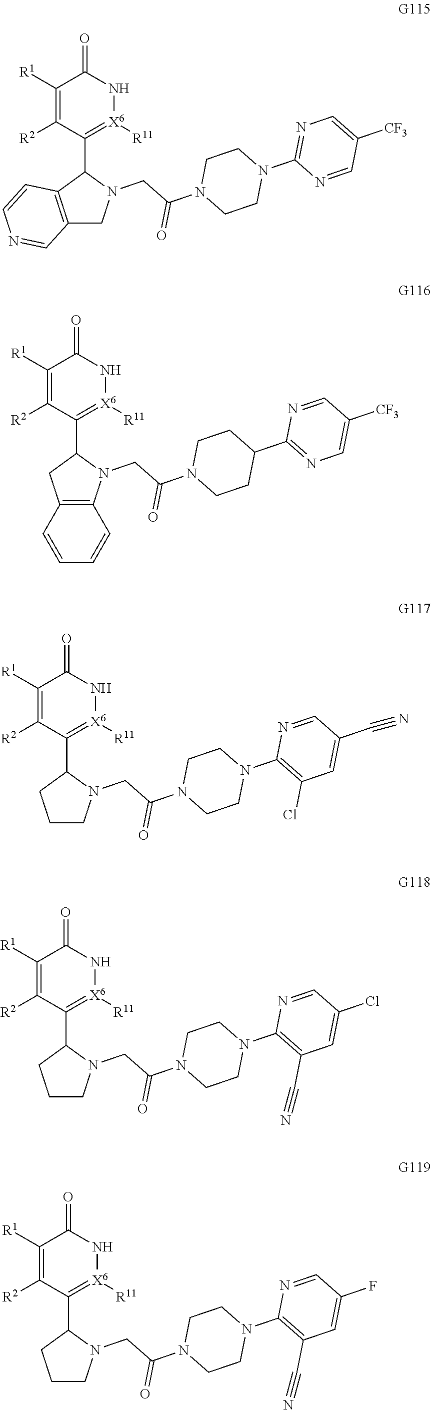

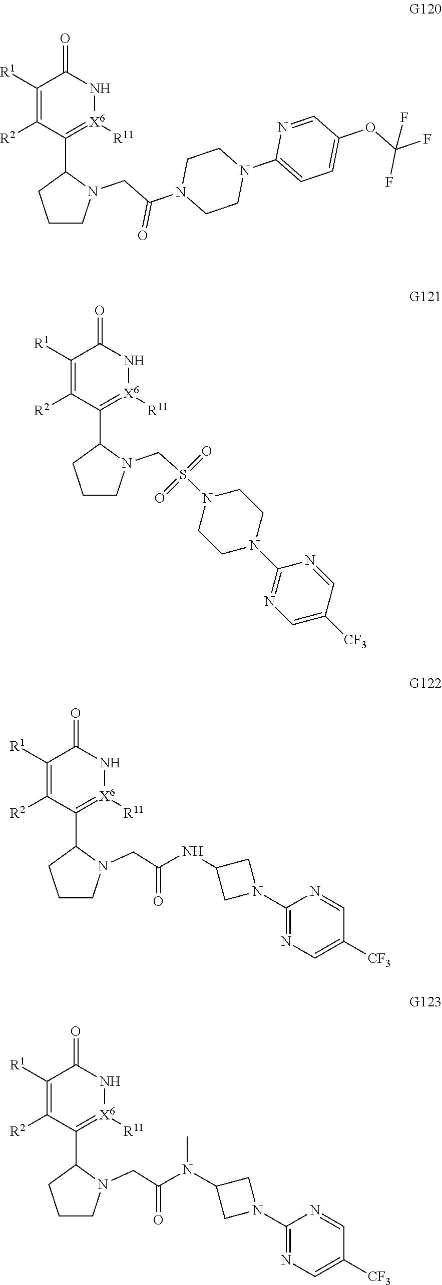

Specific examples of compounds provided herein are:

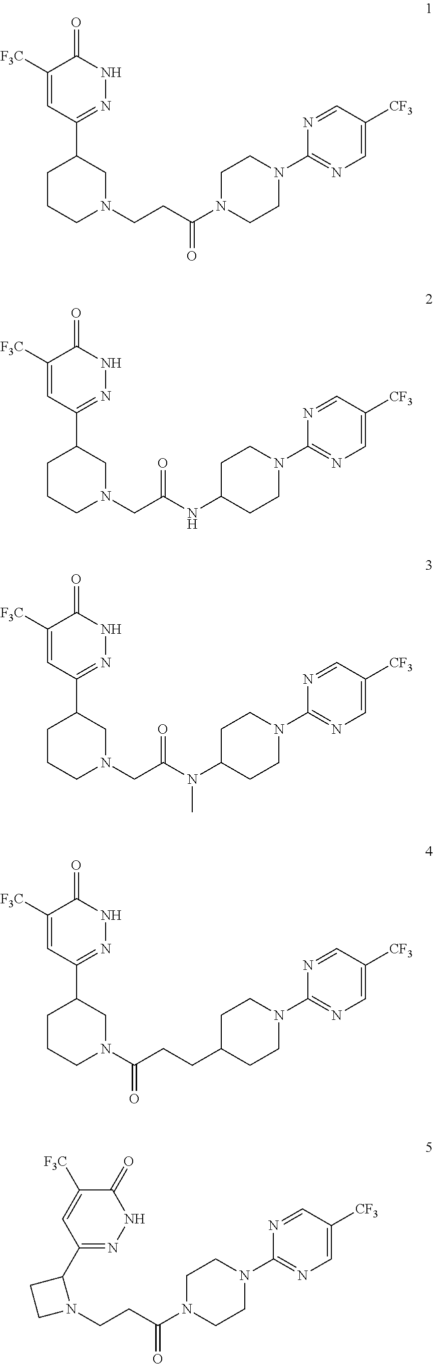 [SMILES: O=C(CCN1CCCC(c2cc(C(F)(F)F)c(=O)[nH]n2)C1)N1CCN(c2ncc(C(F)(F)F)cn2)CC1 ; O=C(CN1CCCC(c2cc(C(F)(F)F)c(=O)[nH]n2)C1)NC1CCN(c2ncc(C(F)(F)F)cn2)CC1 ; CN(C(=O)CN1CCCC(c2cc(C(F)(F)F)c(=O)[nH]n2)C1)C1CCN(c2ncc(C(F)(F)F)cn2)CC1 ; O=C(CCC1CCN(c2ncc(C(F)(F)F)cn2)CC1)N1CCCC(c2cc(C(F)(F)F)c(=O)[nH]n2)C1 ; O=C(CCN1CCC1c1cc(C(F)(F)F)c(=O)[nH]n1)N1CCN(c2ncc(C(F)(F)F)cn2)CC1]

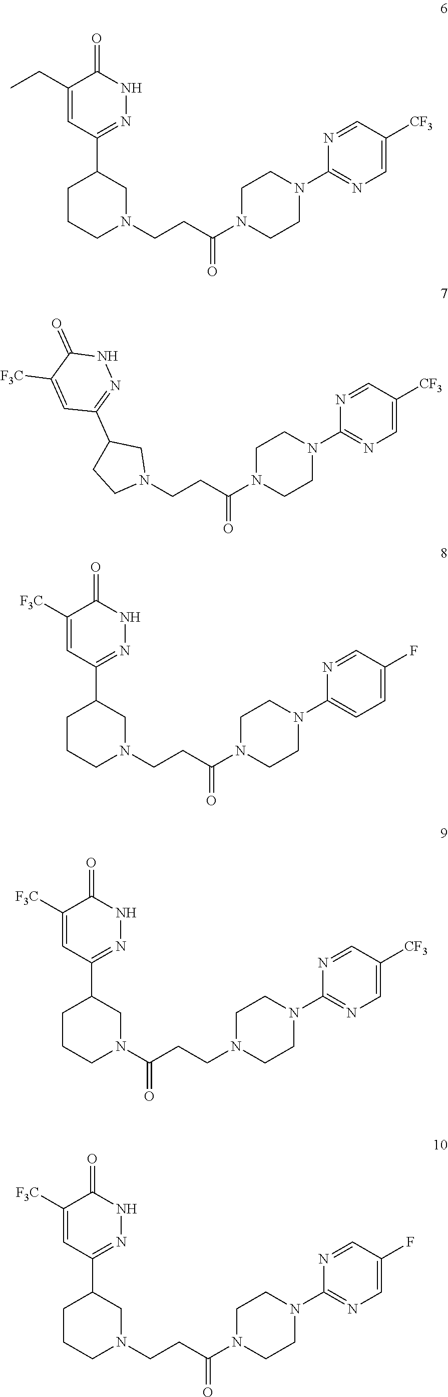 [SMILES: CCc1cc(C2CCCN(CCC(=O)N3CCN(c4ncc(C(F)(F)F)cn4)CC3)C2)n[nH]c1=O ; O=C(CCN1CCC(c2cc(C(F)(F)F)c(=O)[nH]n2)C1)N1CCN(c2ncc(C(F)(F)F)cn2)CC1 ; O=C(CCN1CCCC(c2cc(C(F)(F)F)c(=O)[nH]n2)C1)N1CCN(c2ccc(F)cn2)CC1 ; O=C(CCN1CCN(c2ncc(C(F)(F)F)cn2)CC1)N1CCCC(c2cc(C(F)(F)F)c(=O)[nH]n2)C1 ; O=C(CCN1CCCC(c2cc(C(F)(F)F)c(=O)[nH]n2)C1)N1CCN(c2ncc(F)cn2)CC1]

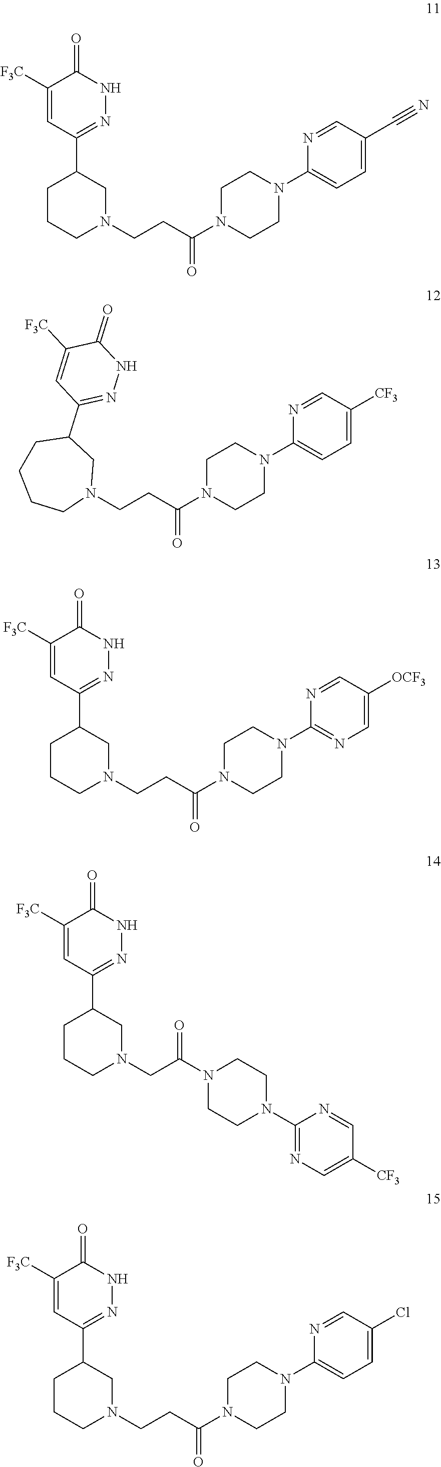 [SMILES: N#Cc1ccc(N2CCN(C(=O)CCN3CCCC(c4cc(C(F)(F)F)c(=O)[nH]n4)C3)CC2)nc1 ; O=C(CCN1CCCCC(c2cc(C(F)(F)F)c(=O)[nH]n2)C1)N1CCN(c2ccc(C(F)(F)F)cn2)CC1 ; O=C(CCN1CCCC(c2cc(C(F)(F)F)c(=O)[nH]n2)C1)N1CCN(c2ncc(OC(F)(F)F)cn2)CC1 ; O=C(CN1CCCC(c2cc(C(F)(F)F)c(=O)[nH]n2)C1)N1CCN(c2ncc(C(F)(F)F)cn2)CC1 ; O=C(CCN1CCCC(c2cc(C(F)(F)F)c(=O)[nH]n2)C1)N1CCN(c2ccc(Cl)cn2)CC1]

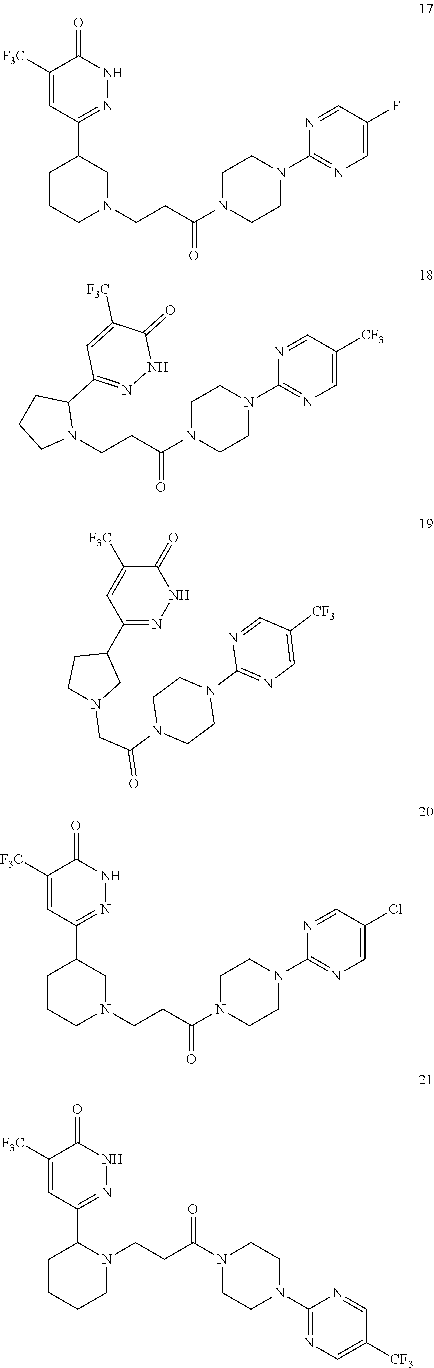 [SMILES: O=C(CCN1CCCC(c2cc(C(F)(F)F)c(=O)[nH]n2)C1)N1CCN(c2ncc(F)cn2)CC1 ; O=C(CCN1CCCC1c1cc(C(F)(F)F)c(=O)[nH]n1)N1CCN(c2ncc(C(F)(F)F)cn2)CC1 ; O=C(CN1CCC(c2cc(C(F)(F)F)c(=O)[nH]n2)C1)N1CCN(c2ncc(C(F)(F)F)cn2)CC1 ; O=C(CCN1CCCC(c2cc(C(F)(F)F)c(=O)[nH]n2)C1)N1CCN(c2ncc(Cl)cn2)CC1 ; O=C(CCN1CCCCC1c1cc(C(F)(F)F)c(=O)[nH]n1)N1CCN(c2ncc(C(F)(F)F)cn2)CC1]

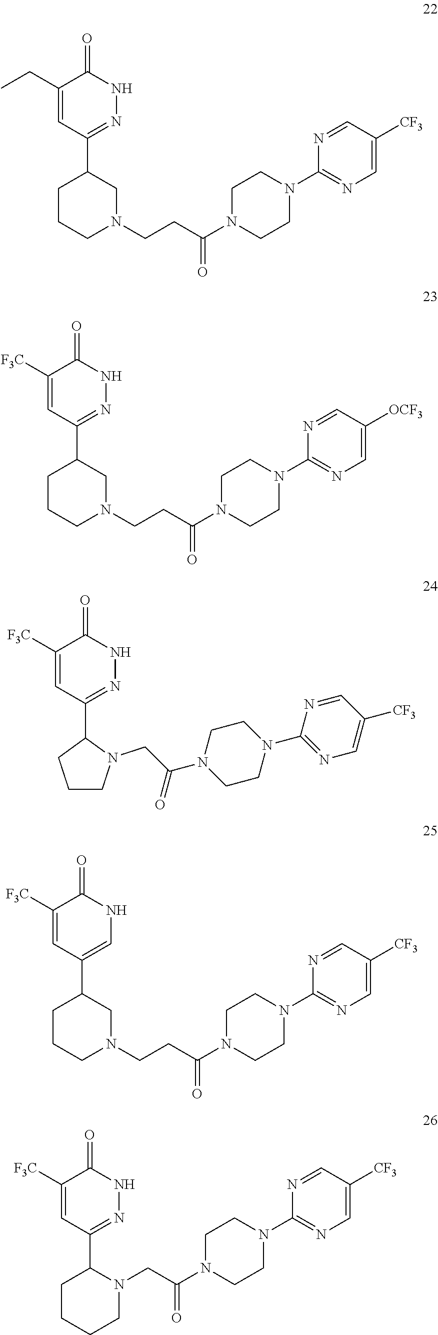 [SMILES: CCc1cc(C2CCCN(CCC(=O)N3CCN(c4ncc(C(F)(F)F)cn4)CC3)C2)n[nH]c1=O ; O=C(CCN1CCCC(c2cc(C(F)(F)F)c(=O)[nH]n2)C1)N1CCN(c2ncc(OC(F)(F)F)cn2)CC1 ; O=C(CN1CCCC1c1cc(C(F)(F)F)c(=O)[nH]n1)N1CCN(c2ncc(C(F)(F)F)cn2)CC1 ; O=C(CCN1CCCC(c2c[nH]c(=O)c(C(F)(F)F)c2)C1)N1CCN(c2ncc(C(F)(F)F)cn2)CC1 ; O=C(CN1CCCCC1c1cc(C(F)(F)F)c(=O)[nH]n1)N1CCN(c2ncc(C(F)(F)F)cn2)CC1]

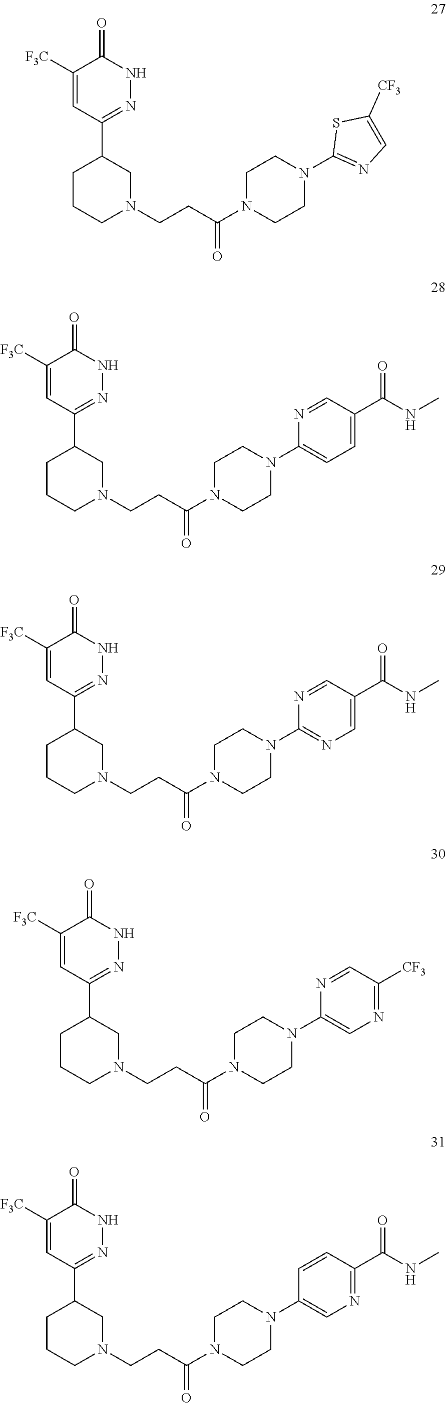 [SMILES: O=C(CCN1CCCC(c2cc(C(F)(F)F)c(=O)[nH]n2)C1)N1CCN(c2ncc(C(F)(F)F)s2)CC1 ; CNC(=O)c1ccc(N2CCN(C(=O)CCN3CCCC(c4cc(C(F)(F)F)c(=O)[nH]n4)C3)CC2)nc1 ; CNC(=O)c1cnc(N2CCN(C(=O)CCN3CCCC(c4cc(C(F)(F)F)c(=O)[nH]n4)C3)CC2)nc1 ; O=C(CCN1CCCC(c2cc(C(F)(F)F)c(=O)[nH]n2)C1)N1CCN(c2cnc(C(F)(F)F)cn2)CC1 ; CNC(=O)c1ccc(N2CCN(C(=O)CCN3CCCC(c4cc(C(F)(F)F)c(=O)[nH]n4)C3)CC2)cn1]

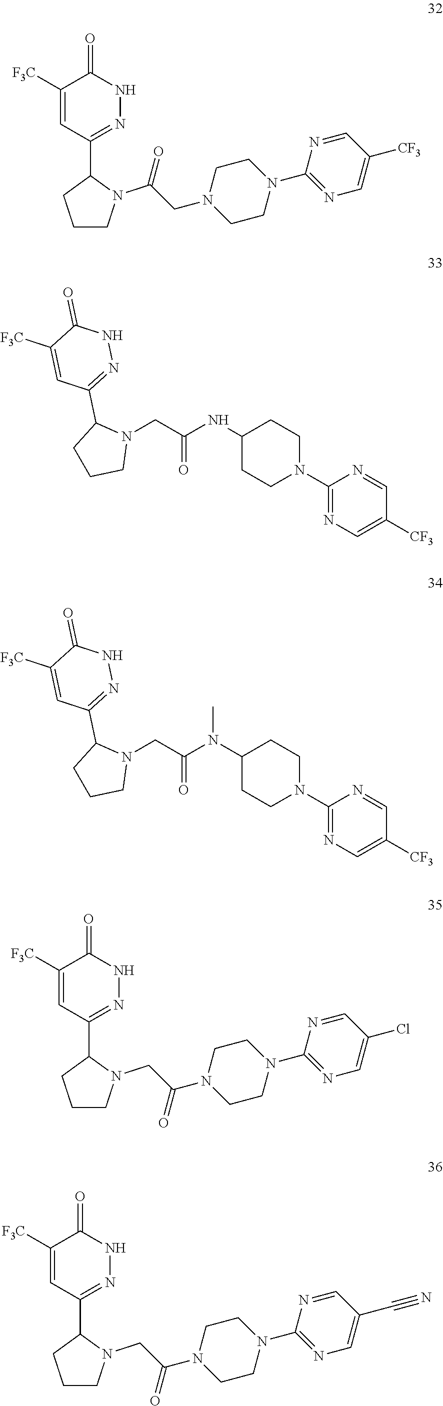 [SMILES: O=C(CN1CCN(c2ncc(C(F)(F)F)cn2)CC1)N1CCCC1c1cc(C(F)(F)F)c(=O)[nH]n1 ; O=C(CN1CCCC1c1cc(C(F)(F)F)c(=O)[nH]n1)NC1CCN(c2ncc(C(F)(F)F)cn2)CC1 ; CN(C(=O)CN1CCCC1c1cc(C(F)(F)F)c(=O)[nH]n1)C1CCN(c2ncc(C(F)(F)F)cn2)CC1 ; O=C(CN1CCCC1c1cc(C(F)(F)F)c(=O)[nH]n1)N1CCN(c2ncc(Cl)cn2)CC1 ; N#Cc1cnc(N2CCN(C(=O)CN3CCCC3c3cc(C(F)(F)F)c(=O)[nH]n3)CC2)nc1]

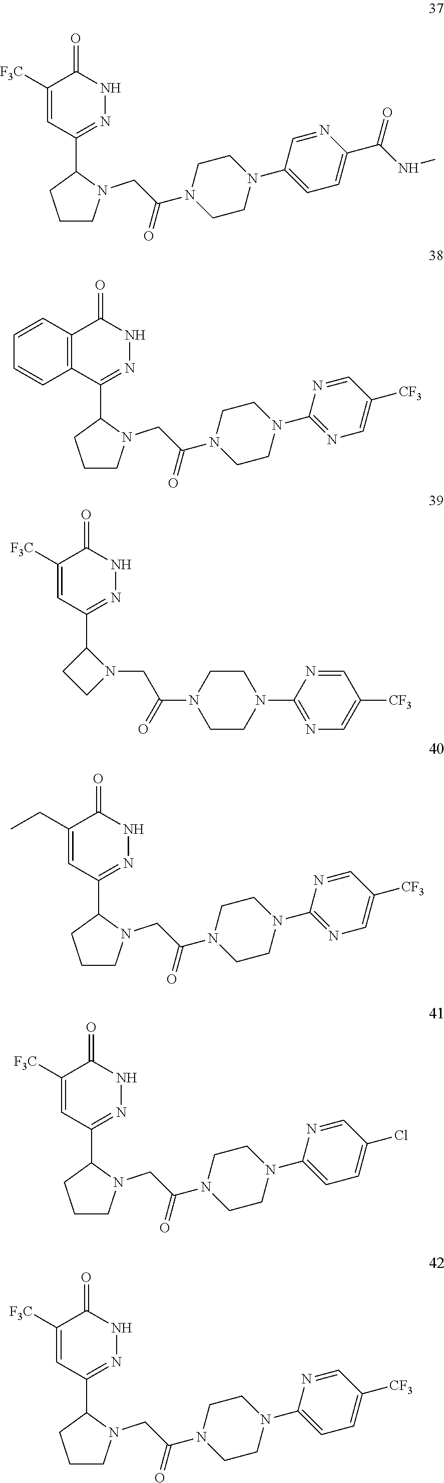

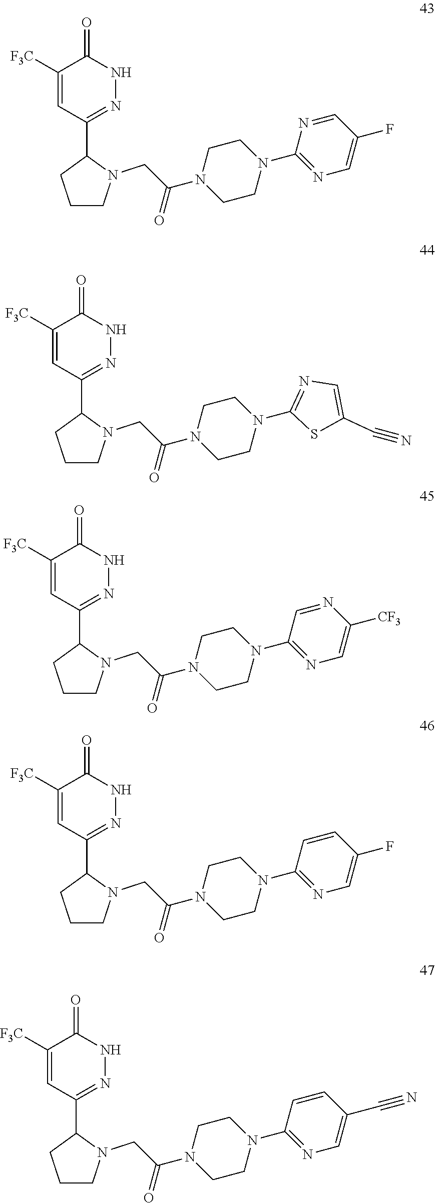 [SMILES: O=C(CN1CCCC1c1cc(C(F)(F)F)c(=O)[nH]n1)N1CCN(c2ncc(F)cn2)CC1 ; N#Cc1cnc(N2CCN(C(=O)CN3CCCC3c3cc(C(F)(F)F)c(=O)[nH]n3)CC2)s1 ; O=C(CN1CCCC1c1cc(C(F)(F)F)c(=O)[nH]n1)N1CCN(c2cnc(C(F)(F)F)cn2)CC1 ; O=C(CN1CCCC1c1cc(C(F)(F)F)c(=O)[nH]n1)N1CCN(c2ccc(F)cn2)CC1 ; N#Cc1ccc(N2CCN(C(=O)CN3CCCC3c3cc(C(F)(F)F)c(=O)[nH]n3)CC2)nc1]

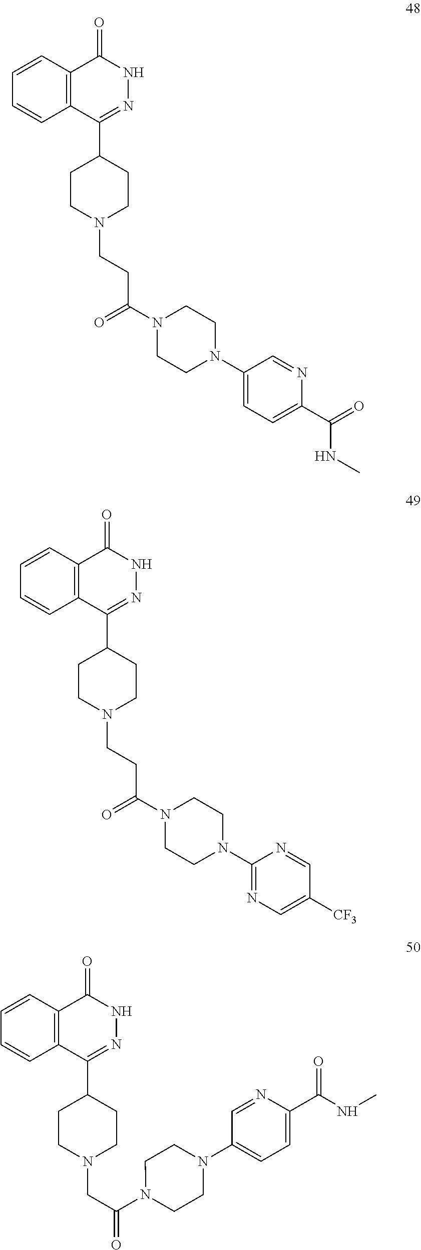 [SMILES: CNC(=O)c1ccc(N2CCN(C(=O)CCN3CCC(c4n[nH]c(=O)c5ccccc45)CC3)CC2)cn1 ; O=C(CCN1CCC(c2n[nH]c(=O)c3ccccc23)CC1)N1CCN(c2ncc(C(F)(F)F)cn2)CC1 ; CNC(=O)c1ccc(N2CCN(C(=O)CN3CCC(c4n[nH]c(=O)c5ccccc45)CC3)CC2)cn1]

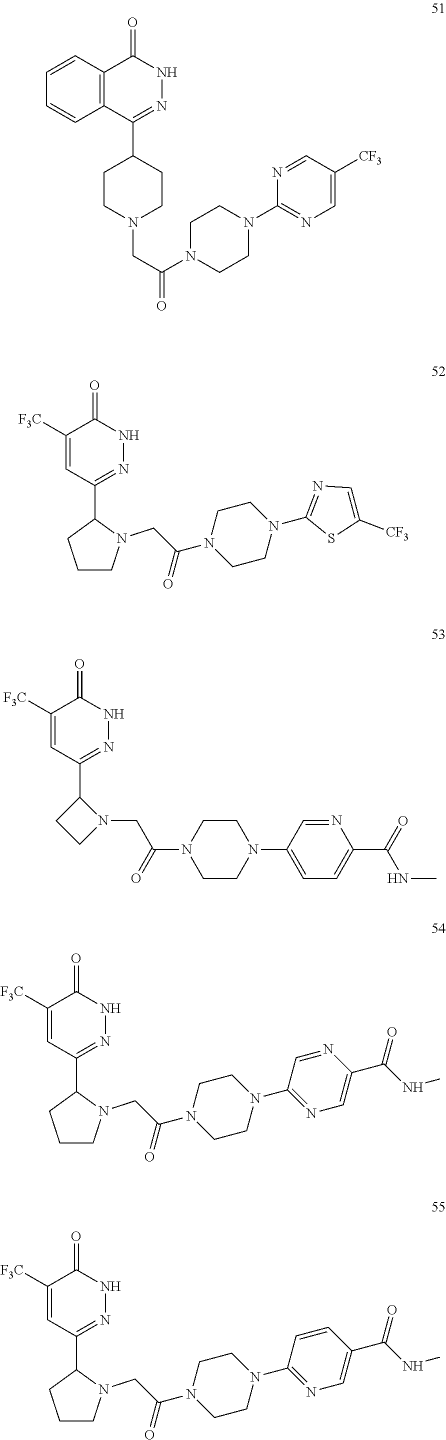 [SMILES: O=C(CN1CCC(c2n[nH]c(=O)c3ccccc23)CC1)N1CCN(c2ncc(C(F)(F)F)cn2)CC1 ; O=C(CN1CCCC1c1cc(C(F)(F)F)c(=O)[nH]n1)N1CCN(c2ncc(C(F)(F)F)s2)CC1 ; CNC(=O)c1ccc(N2CCN(C(=O)CN3CCC3c3cc(C(F)(F)F)c(=O)[nH]n3)CC2)cn1 ; CNC(=O)c1cnc(N2CCN(C(=O)CN3CCCC3c3cc(C(F)(F)F)c(=O)[nH]n3)CC2)cn1 ; CNC(=O)c1ccc(N2CCN(C(=O)CN3CCCC3c3cc(C(F)(F)F)c(=O)[nH]n3)CC2)nc1]

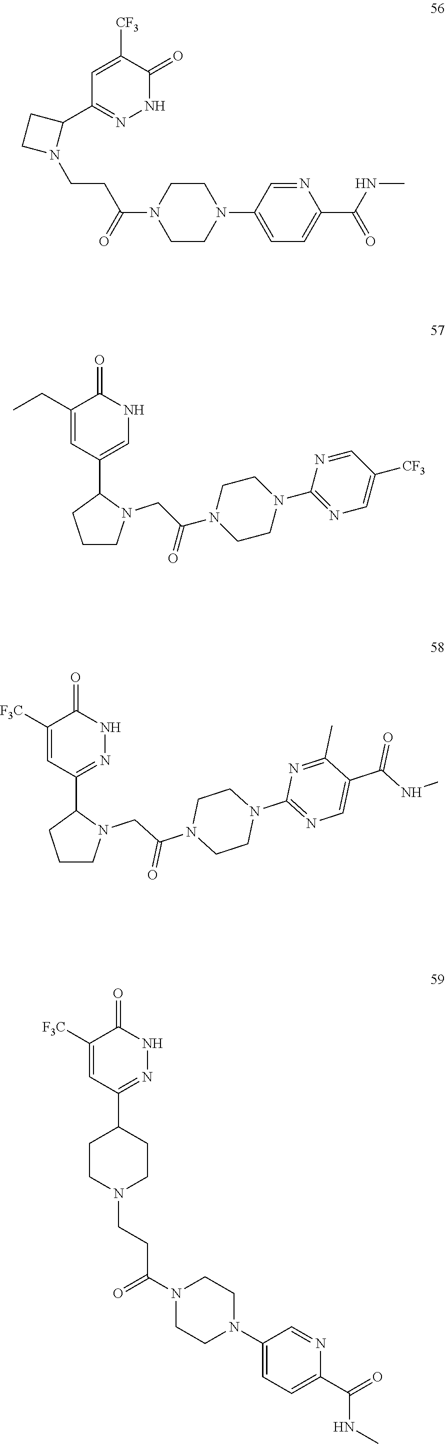

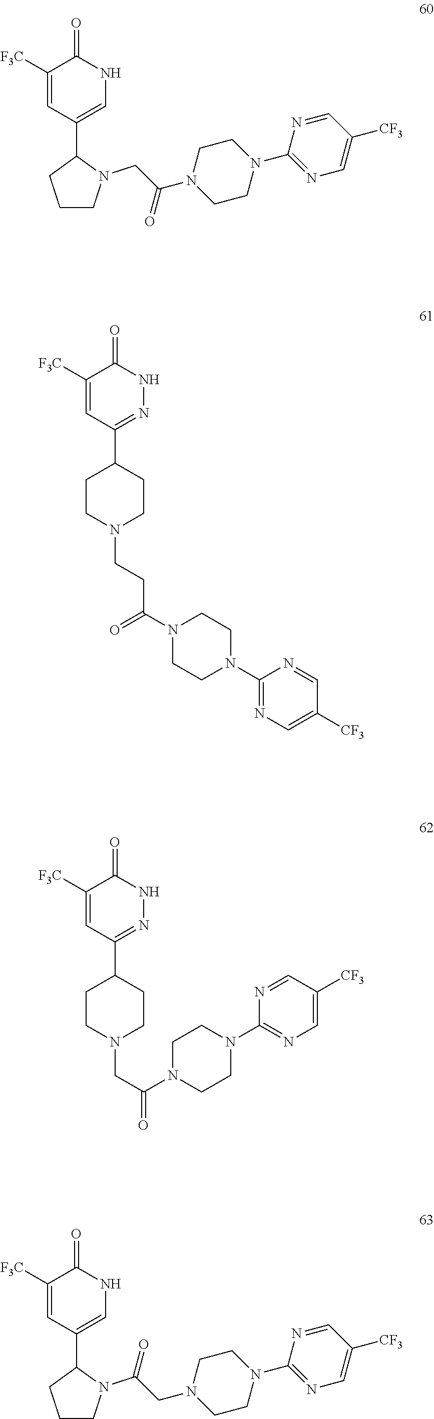

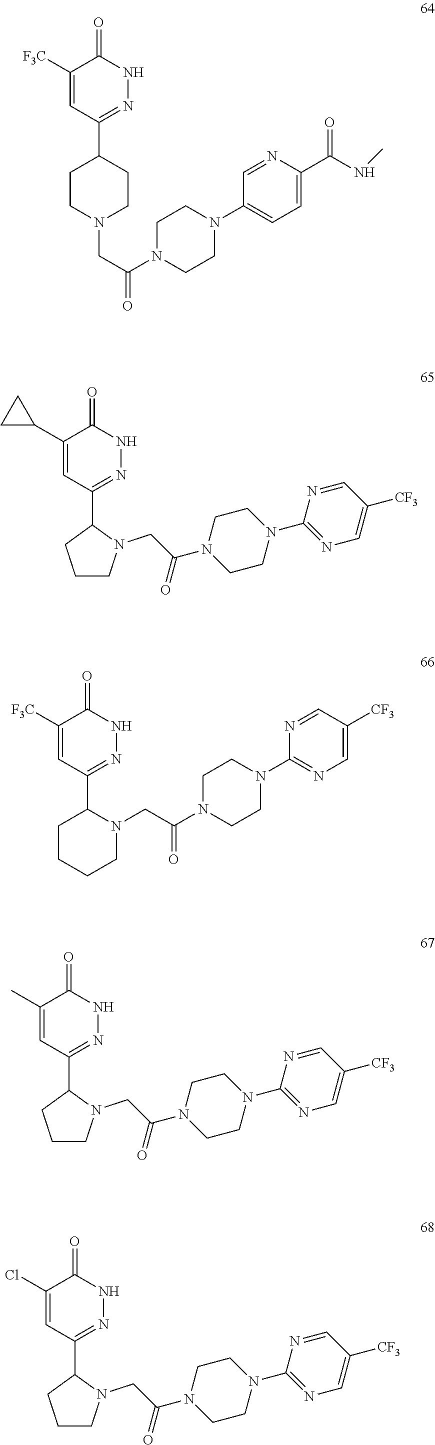

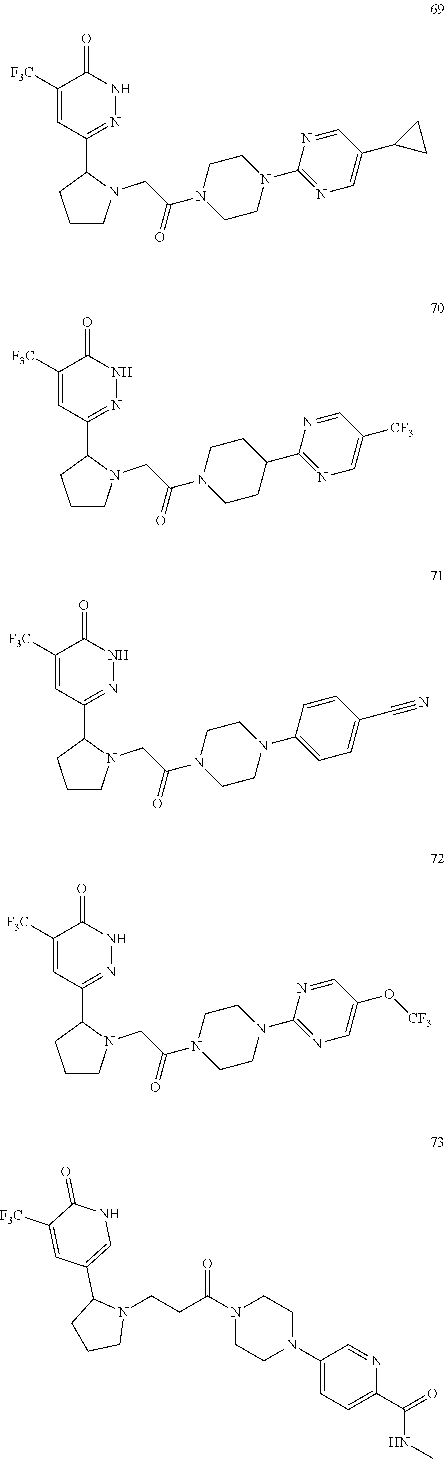 [SMILES: O=C(CN1CCCC1c1cc(C(F)(F)F)c(=O)[nH]n1)N1CCN(c2ncc(C3CC3)cn2)CC1 ; O=C(CN1CCCC1c1cc(C(F)(F)F)c(=O)[nH]n1)N1CCC(c2ncc(C(F)(F)F)cn2)CC1 ; N#Cc1ccc(N2CCN(C(=O)CN3CCCC3c3cc(C(F)(F)F)c(=O)[nH]n3)CC2)cc1 ; O=C(CN1CCCC1c1cc(C(F)(F)F)c(=O)[nH]n1)N1CCN(c2ncc(OC(F)(F)F)cn2)CC1 ; CNC(=O)c1ccc(N2CCN(C(=O)CCN3CCCC3c3c[nH]c(=O)c(C(F)(F)F)c3)CC2)cn1]

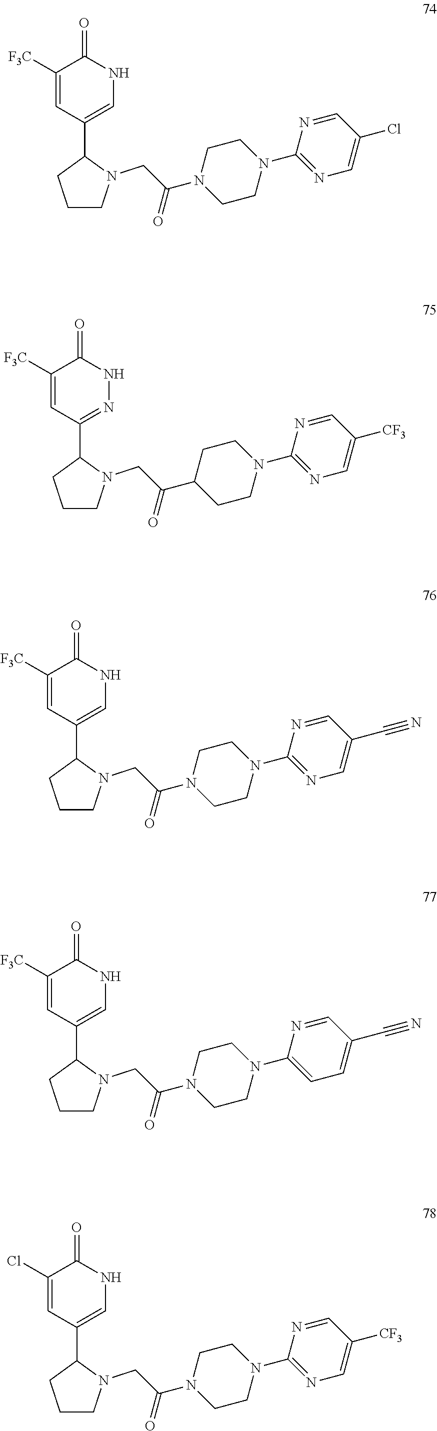 [SMILES: O=C(CN1CCCC1c1c[nH]c(=O)c(C(F)(F)F)c1)N1CCN(c2ncc(Cl)cn2)CC1 ; O=C(CN1CCCC1c1cc(C(F)(F)F)c(=O)[nH]n1)C1CCN(c2ncc(C(F)(F)F)cn2)CC1 ; N#Cc1cnc(N2CCN(C(=O)CN3CCCC3c3c[nH]c(=O)c(C(F)(F)F)c3)CC2)nc1 ; N#Cc1ccc(N2CCN(C(=O)CN3CCCC3c3c[nH]c(=O)c(C(F)(F)F)c3)CC2)nc1 ; O=C(CN1CCCC1c1c[nH]c(=O)c(Cl)c1)N1CCN(c2ncc(C(F)(F)F)cn2)CC1]

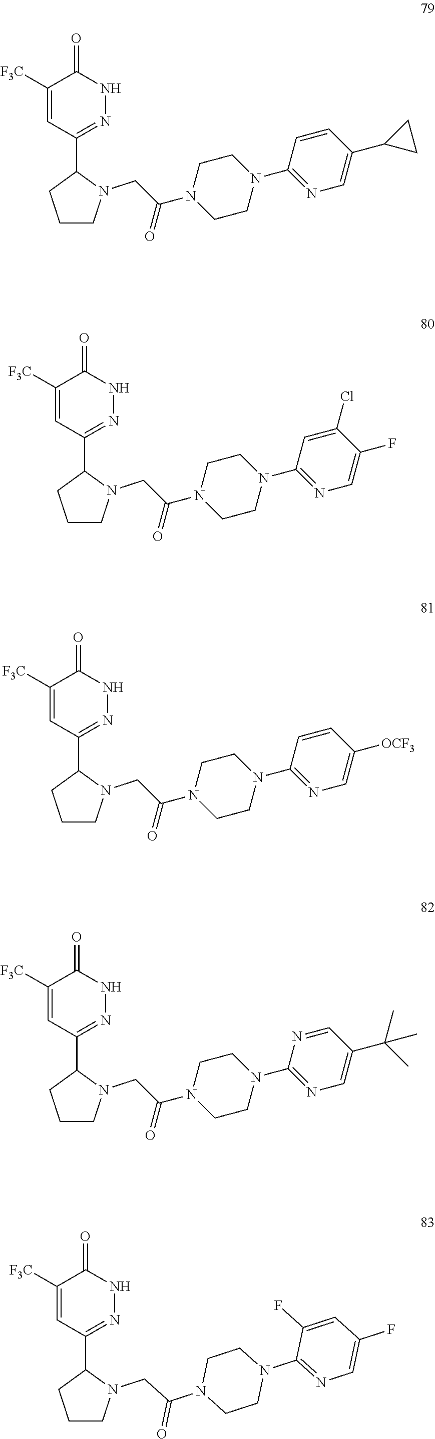 [SMILES: O=C(CN1CCCC1c1cc(C(F)(F)F)c(=O)[nH]n1)N1CCN(c2ccc(C3CC3)cn2)CC1 ; O=C(CN1CCCC1c1cc(C(F)(F)F)c(=O)[nH]n1)N1CCN(c2cc(Cl)c(F)cn2)CC1 ; O=C(CN1CCCC1c1cc(C(F)(F)F)c(=O)[nH]n1)N1CCN(c2ccc(OC(F)(F)F)cn2)CC1 ; CC(C)(C)c1cnc(N2CCN(C(=O)CN3CCCC3c3cc(C(F)(F)F)c(=O)[nH]n3)CC2)nc1 ; O=C(CN1CCCC1c1cc(C(F)(F)F)c(=O)[nH]n1)N1CCN(c2ncc(F)cc2F)CC1]

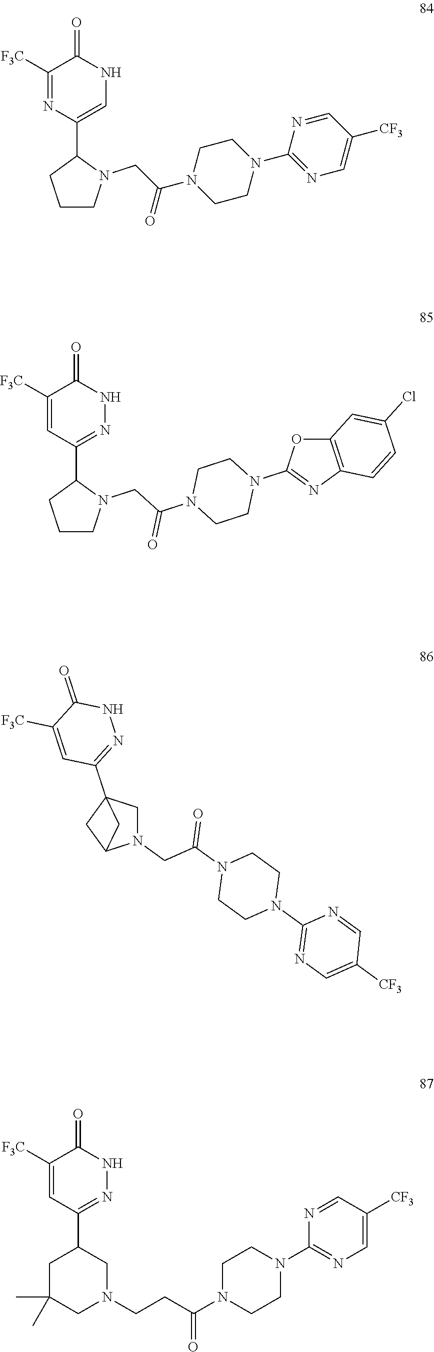

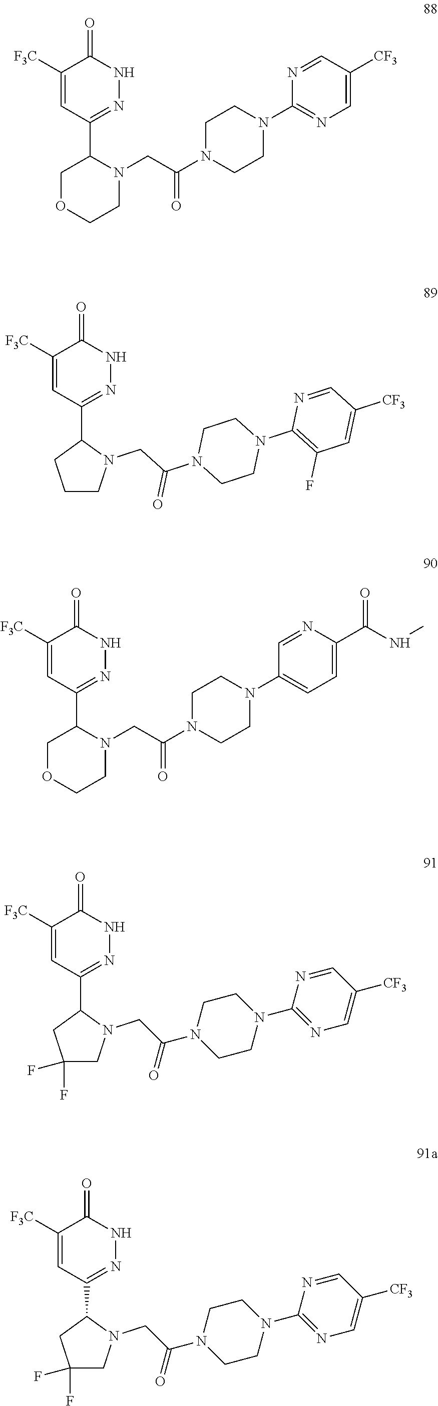 [SMILES: O=C(CN1CCOCC1c1cc(C(F)(F)F)c(=O)[nH]n1)N1CCN(c2ncc(C(F)(F)F)cn2)CC1 ; O=C(CN1CCCC1c1cc(C(F)(F)F)c(=O)[nH]n1)N1CCN(c2ncc(C(F)(F)F)cc2F)CC1 ; CNC(=O)c1ccc(N2CCN(C(=O)CN3CCOCC3c3cc(C(F)(F)F)c(=O)[nH]n3)CC2)cn1 ; O=C(CN1CC(F)(F)CC1c1cc(C(F)(F)F)c(=O)[nH]n1)N1CCN(c2ncc(C(F)(F)F)cn2)CC1 ; O=C(CN1CC(F)(F)C[C@@H]1c1cc(C(F)(F)F)c(=O)[nH]n1)N1CCN(c2ncc(C(F)(F)F)cn2)CC1]

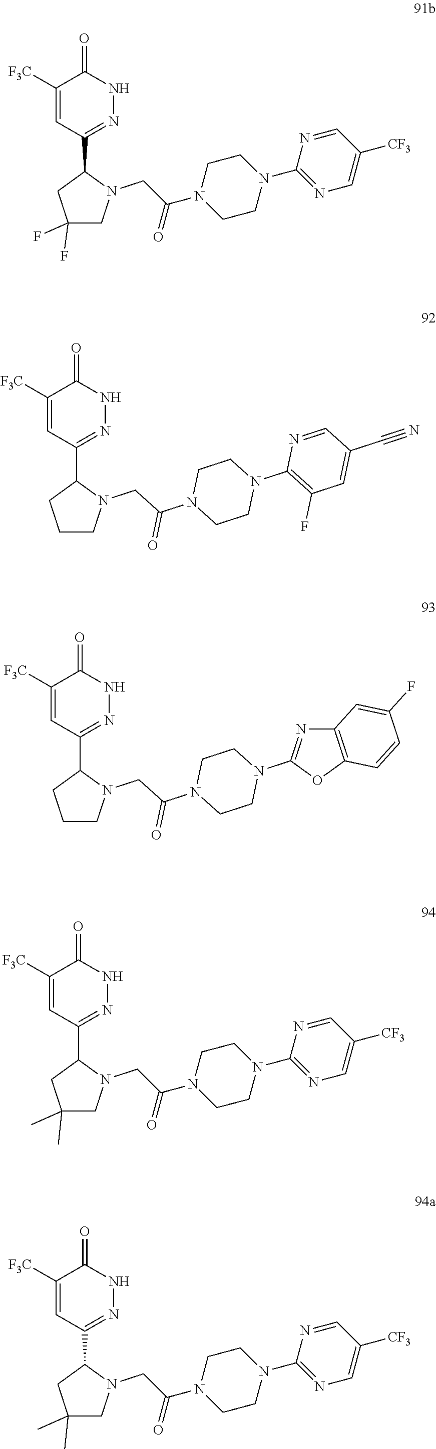 [SMILES: O=C(CN1CC(F)(F)C[C@H]1c1cc(C(F)(F)F)c(=O)[nH]n1)N1CCN(c2ncc(C(F)(F)F)cn2)CC1 ; N#Cc1cnc(N2CCN(C(=O)CN3CCCC3c3cc(C(F)(F)F)c(=O)[nH]n3)CC2)c(F)c1 ; O=C(CN1CCCC1c1cc(C(F)(F)F)c(=O)[nH]n1)N1CCN(c2nc3cc(F)ccc3o2)CC1 ; CC1(C)CC(c2cc(C(F)(F)F)c(=O)[nH]n2)N(CC(=O)N2CCN(c3ncc(C(F)(F)F)cn3)CC2)C1 ; CC1(C)C[C@H](c2cc(C(F)(F)F)c(=O)[nH]n2)N(CC(=O)N2CCN(c3ncc(C(F)(F)F)cn3)CC2)C1]

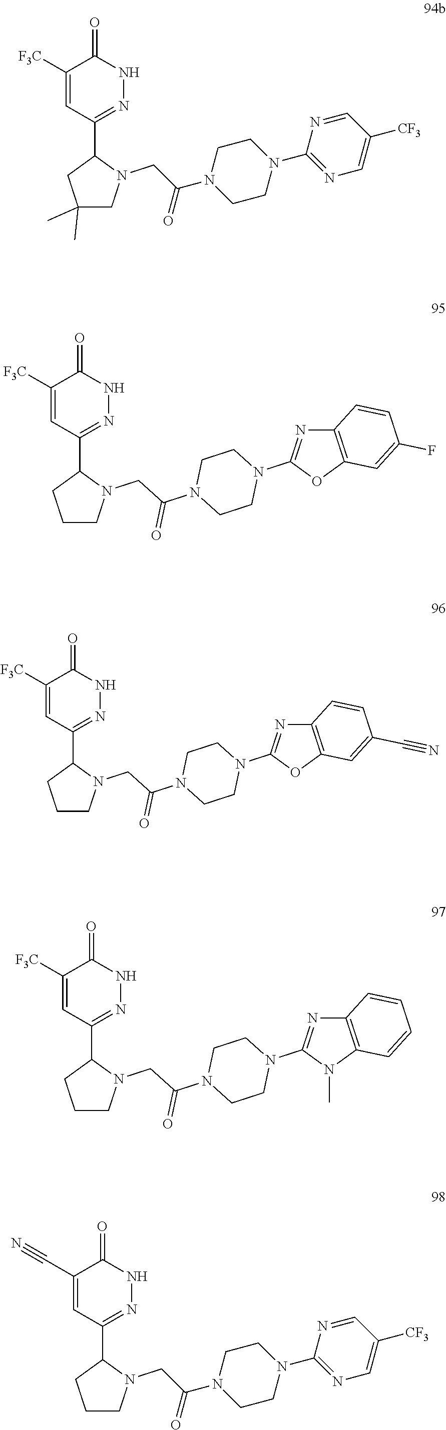 [SMILES: CC1(C)CC(c2cc(C(F)(F)F)c(=O)[nH]n2)N(CC(=O)N2CCN(c3ncc(C(F)(F)F)cn3)CC2)C1 ; O=C(CN1CCCC1c1cc(C(F)(F)F)c(=O)[nH]n1)N1CCN(c2nc3ccc(F)cc3o2)CC1 ; N#Cc1ccc2nc(N3CCN(C(=O)CN4CCCC4c4cc(C(F)(F)F)c(=O)[nH]n4)CC3)oc2c1 ; Cn1c(N2CCN(C(=O)CN3CCCC3c3cc(C(F)(F)F)c(=O)[nH]n3)CC2)nc2ccccc21 ; N#Cc1cc(C2CCCN2CC(=O)N2CCN(c3ncc(C(F)(F)F)cn3)CC2)n[nH]c1=O]

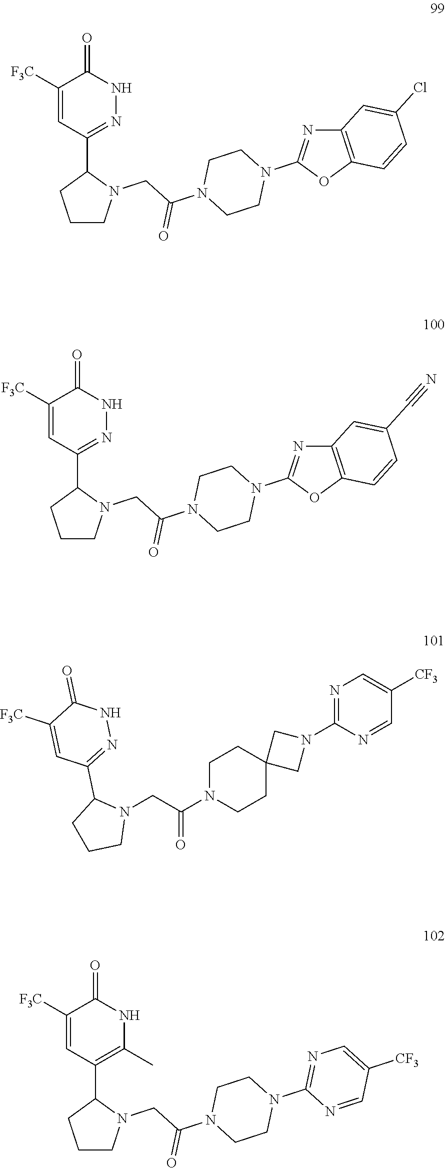 [SMILES: O=C(CN1CCCC1c1cc(C(F)(F)F)c(=O)[nH]n1)N1CCN(c2nc3cc(Cl)ccc3o2)CC1 ; N#Cc1ccc2oc(N3CCN(C(=O)CN4CCCC4c4cc(C(F)(F)F)c(=O)[nH]n4)CC3)nc2c1 ; O=C(CN1CCCC1c1cc(C(F)(F)F)c(=O)[nH]n1)N1CCC2(CC1)CN(c1ncc(C(F)(F)F)cn1)C2 ; Cc1[nH]c(=O)c(C(F)(F)F)cc1C1CCCN1CC(=O)N1CCN(c2ncc(C(F)(F)F)cn2)CC1]

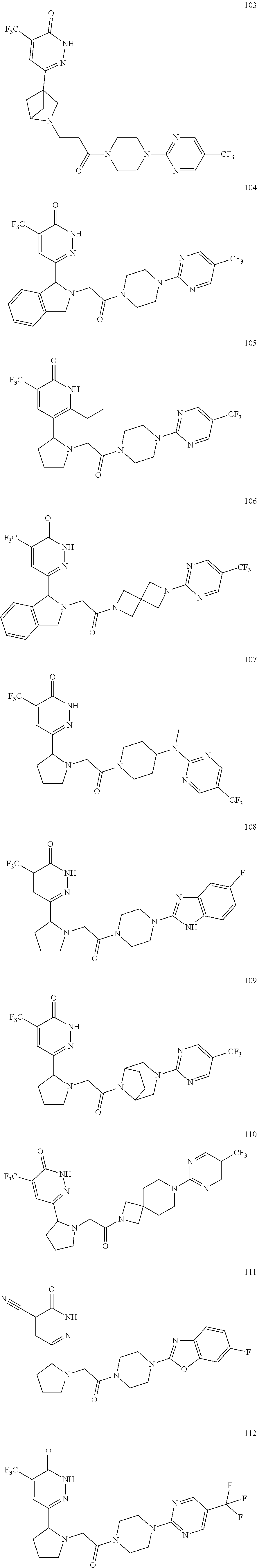

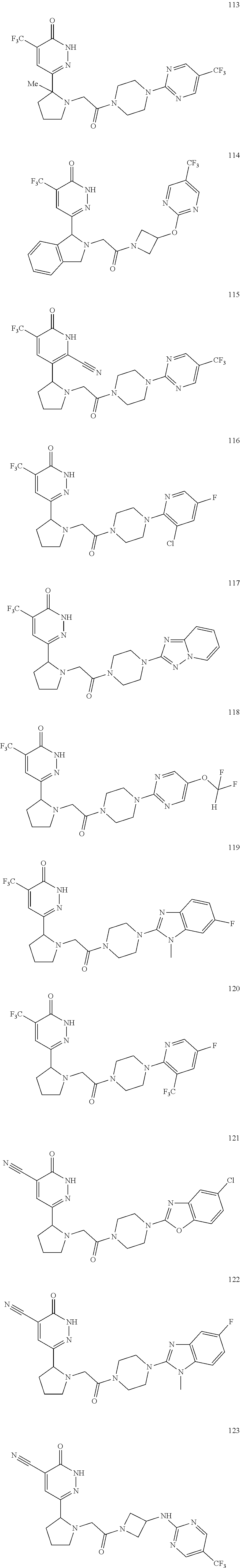

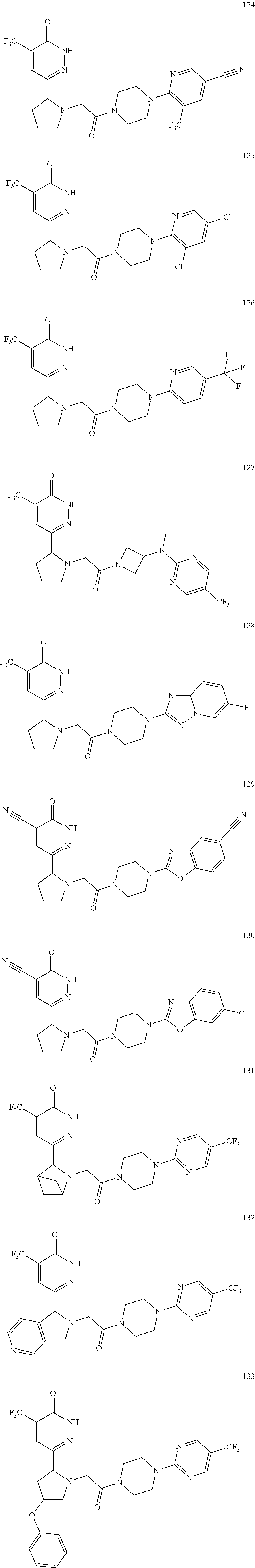

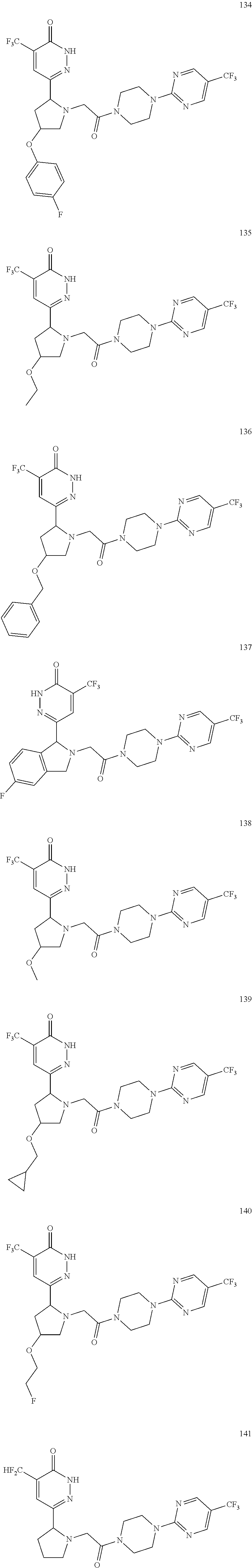

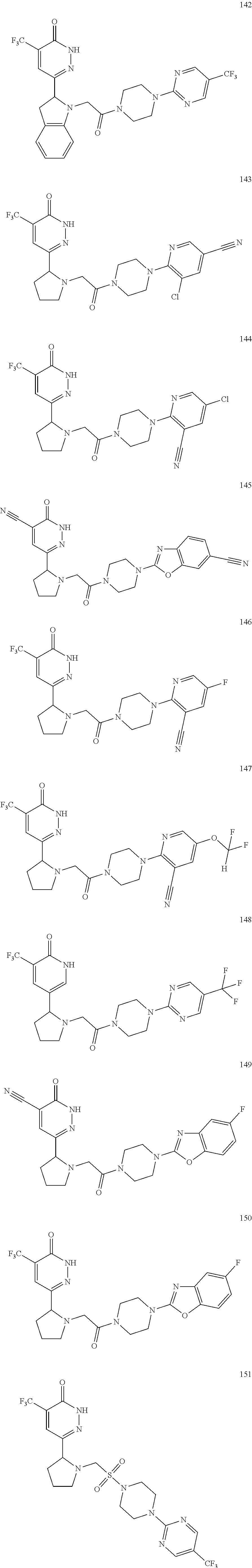

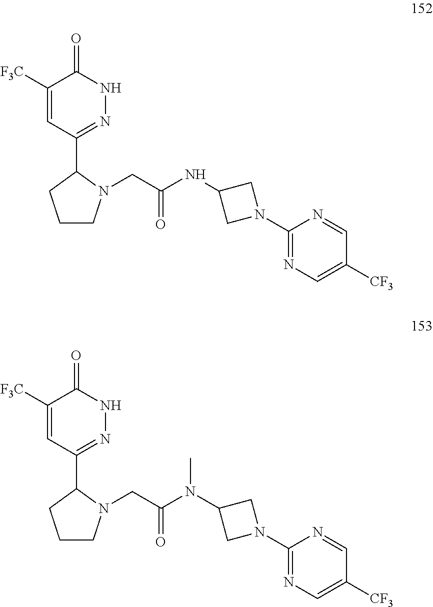 [SMILES: O=C(CN1CCCC1c1cc(C(F)(F)F)c(=O)[nH]n1)NC1CN(c2ncc(C(F)(F)F)cn2)C1 ; CN(C(=O)CN1CCCC1c1cc(C(F)(F)F)c(=O)[nH]n1)C1CN(c2ncc(C(F)(F)F)cn2)C1]

The compounds provided herein may be inhibitors of both PARP7 and PARP1.

The compound may be for use in medicine.

Another aspect provides pharmaceutical compositions and kits comprising a compound as defined herein.

Still another aspect provides a method of treating a disease and/or a condition and/or a disorder, which method comprises administering to a patient a compound, composition, kit as defined herein.

In another aspect, there is provided a method of synthesis of a compound as defined herein, which method includes conducting a reaction between:
i) a first reactant comprising ring A and ring B and
ii) a second reactant comprising group L,
so as to form the compound.

Optionally, the second reactant further comprises ring C, Q, and ring D.

### DETAILED DESCRIPTION

Accordingly, there is provided a compound, that is a PARP7 and/or a PARP1 inhibitor compound, which compound comprises the following formula:

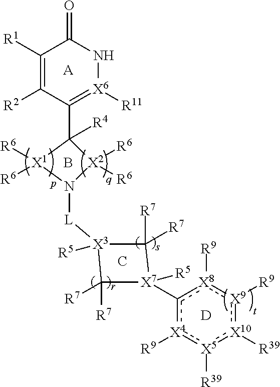

wherein, X⁶ is independently selected from C and N, R¹ is independently selected from H and a substituted or unsubstituted organic group, R² is independently selected from H and a substituted or unsubstituted organic group, and wherein R¹ and R² may together form a 4 to 7 membered ring together with the atoms of ring A to which they are attached, R¹¹ may be present or absent depending on the number of bonds to, and the valency of, the X⁶ atom to which it is attached, and is independently selected from H and a substituted or unsubstituted organic group;
X¹ is independently selected from C, N, O and S, X² is independently selected from C, N, O and S, and X¹ and X² may be the same or different; when there is more than one X¹, each X¹ may be the same or different; when there is more than one X², each X² may be the same or different; p is selected from 0, 1, 2, 3, 4 and 5; q is selected from 0, 1 and 2, such as 0 or 1; p+q may be 2, 3, 4 or 5; ring B is a saturated ring; R⁴ is independently selected from H and a substituted or unsubstituted organic group; each R⁶ may be the same or different and is independently selected from H and a substituted or unsubstituted organic group, and each R⁶ may be present or absent depending on the valency of the X¹ or X² atom to which it is attached;
X³ and X⁷ may be the same or different and are independently selected from C and N; each R⁵ may be the same or different and is independently selected from H and a substituted or unsubstituted organic group, and each R⁵ may be present or absent depending on the number of bonds to, and the valency of, the X³ or X⁷ atom to which they are attached; each bond between the X³ and/or the X⁷, and/or the carbon atoms in ring C may be a single bond or a double bond depending on the number of bonds to, and the valence of, the X³ atom, the X⁷ atom and the carbon atoms; each R⁷ may be present or absent, depending on the number of bonds to the atom to which they are attached, and each R⁷ may be the same or different and is independently selected from H and a substituted or unsubstituted organic group; r is selected from 0, 1, 2, 3, 4 and 5; s is selected from 0, 1, 2, 3, 4 and 5; and r+s may be 2, 3, 4 or 5;
each of X⁴, X⁵, X⁸, X⁹ and X¹⁰ may be the same or different and are independently selected from C, N, O and S; t is 0 or 1; ring D is an aromatic ring, and may comprise 2 or 3 double bonds depending on the size of t and the nature of X⁴, X⁵, X⁸, X⁹ and X¹⁰; one of R³⁹ is an R⁹ and one of R³⁹ is an R³; each R⁹ may be the same or different and is independently selected from H and a substituted or unsubstituted organic group, and each R⁹ may be present or absent depending on the number of bonds to, and the valency of, the X⁴, X⁵, X⁸, X⁹ or X¹⁰ atom to which it is attached; and R³ is independently selected from H and a substituted or unsubstituted organic group;
and wherein, L is selected from a group having one of the following structures:

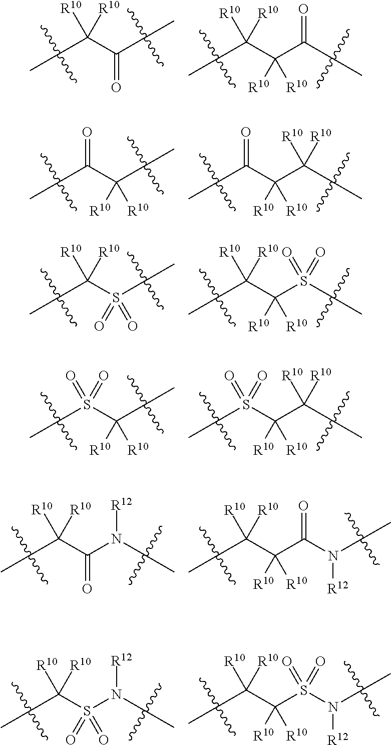

wherein each R¹⁰ may be the same or different and is independently selected from H and a substituted or unsubstituted organic group; and
wherein R¹² is selected from H and a substituted or unsubstituted organic group.

In general, where a substituent is possible, but not depicted in a formula, the number of substituents borne by any atom is the number required to maintain the valency for that atom. Similarly, in some cases a substituent has been depicted, but the number of such substituents (and their presence or absence) depends on the number of bonds to, and the valence of, the atom comprising the substituent. In those cases, both above and in the following, the number of substituents borne by the atom is again the number required to maintain the valency for that atom.

In the context of the present invention, maintaining the valency means ensuring that an atom has its normal (typically most common) valency in organic compounds (for example 2 for oxygen and sulphur, 3 for nitrogen and 4 for carbon). Nitrogen atoms may, in some instances, have 4 bonds, but in such cases they are typically positively charged such that the compound may have a counter-ion. Sulphur atoms may, in some instances, have a higher valency such as 6, for example when forming a sulphonyl group. Such compounds are also considered to be part of the invention. When there is a positive charge on a nitrogen, it will be clear that the nitrogen atom still maintains its normal valency of 3. For the avoidance of doubt, where the number of R groups may vary according to the choice of X group, it may vary as follows.

Each R⁶ may be the same or different, provided that for each X¹: R⁶ is absent when X¹ is O or S; one R⁶ is present when X¹ is N; and two R⁶ are present when X¹ is C. Each R⁶ may be the same or different, provided that for each X²: R⁶ is absent when X² is O or S; one R⁶ is present when X² is N; and two R⁶ are present when X² is C. Each R⁵ may be the same or different, provided that for each X³: R⁵ is absent when X³ is N; one R⁵ is present on X³ when X³ is C; and provided that for each X⁷: R⁵ is absent when X⁷ is N; and one R⁵ is present on X⁷ when X⁷ is C.

In ring D, the dotted lines indicate that the ring is aromatic and comprises a combination of both single and double bonds. Each R⁹ may be the same or different, provided that for each ring atom to which it is attached: R⁹ is absent when the ring atom is O or S; R⁹ is absent when the ring atom is N and is double bonded to an adjacent atom; one R⁹ is present when the ring atom is N and is not double bonded to an adjacent atom; one R⁹ is present when the ring atom is C and is double bonded to an adjacent atom.

In these compounds, and elsewhere herein, in some embodiments any R group may form a ring with any other R group on an adjacent and/or proximal atom, although in most embodiments this is not preferred, except where explicitly stated. Thus, in some embodiments the following substituents may together form a ring: R¹ with R²; R⁴ with R¹¹; R⁴ with R⁶; R⁶ with another R⁶; R⁵ with R⁷; R⁷ with another R⁷; and R⁹ with another R⁹. In the context of the present invention, an adjacent and/or proximal atom may mean another atom directly bonded to an atom (adjacent), or may be two atoms with only a single atom in between (proximal), or may mean two atoms close enough sterically to be capable of forming a ring (proximal). Preferably R groups attached to the same atom do not together form a ring, although this is not excluded.

In the present context the invention includes compounds in which a single R group on an atom, or two R groups on the same atom, form a group which is double bonded to that atom. Accordingly, an R group, or two R groups attached to the same atom, may together form a ═O group, or a ═C(R′)₂ group (wherein each R′ group is the same or different and is H or an organic group, preferably H or a straight or branched C₁-C₆ alkyl group). This is more typical in cases where the R groups are attached to a C atom, such that together they form a C═O group or a C═C(R′)₂ group. Thus in some cases a C ring atom in a ring may bear a ═O group.

In the present context, part of any structure present in brackets may be repeated the number of times given by the numbers next to the brackets (whether regular brackets or square brackets). For example, in the case of (C(R))_{0,1,2} or [C(R)]_{0,1,2} the C—R group may be absent, present once i.e. —C(R)—; or present twice i.e. —C(R)—C(R)—.

Further in the present context, where a structural component is depicted with a wavy line on a bond, that bond is the bond that attaches to another structural component of the compound.

In the context of the present invention, a compound is considered to be a PARP7 inhibitor if its presence is capable of preventing or reducing the ability of immobilised PARP7 to undergo auto-mono-ADP ribosylation (AutoMARylation) following incubation with biotinylated-NAD⁺ as compared to the same process in its absence. Typically, the compound is considered to be a PARP7 inhibitor if it has an IC₅₀<10 μM in a suitable assay. A suitable assay may be conducted using 10-30 nM PARP7 (amino acids 456-657), 2 μM biotin-NAD⁺ assay solution in 20 mM HEPES (pH 7.5), 100 mM NaCl, 2 mM DTT, 0.1% BSA (w/v), 0.02% Tween (v/v) assay buffer. MARylation may take place for 2-3 h at room temperature and may be detected using a dissociation-enhanced lanthanide fluorescence immunoassay (DELFIA) readout. This assay format has been recently utilised for screening for modulators of PARP7 and other MonoPARP enzymes (Wigle T. et al. Forced Self-Modification Assays as a Strategy to Screen MonoPARP Enzymes. SLAS Discovery. 25; 241-252 (2020)). A particularly suitable assay is described in the Examples below.

In the context of the present invention, a compound is considered to be a PARP1 inhibitor if its presence is capable of preventing or reducing the ability of immobilised PARP1 to undergo auto-poly-ADP ribosylation (AutoPARylation) following incubation with biotinylated-NAD+ as compared to the same process in its absence. Typically, the compound is considered to be a PARP1 inhibitor if it has an IC₅₀<10 μM in a suitable assay. A suitable assay may be conducted using 2 nM PARP1, 2 μM biotin-NAD⁺ assay solution in 20 mM HEPES (pH 7.5), 100 mM NaCl, 2 mM DTT, 0.1% BSA (w/v), 0.02% Tween (v/v) assay buffer. PARylation may take place for 2 h at room temperature and may be detected using a dissociation-enhanced lanthanide fluorescence immunoassay (DELFIA) readout. A particularly suitable assay is described in the Examples below. Preferably, the compound has an IC₅₀<1 μM, more preferably <100 nM and most preferably <10 nM in the PARP1 inhibitor assay.

In all of the embodiments of this invention (both above and below herein), the substituents (each of the R groups) are not especially limited, provided that they do not prevent the PARP7 and/or PARP1 inhibitory function from occurring. In all of the embodiments mentioned in connection with this invention, both above and in the following, the substituents are selected from H and an organic group. Thus, both above and in the following, the terms ‘substituent’ and ‘organic group’ are not especially limited and may be any functional group or any atom, especially any functional group or atom common in organic chemistry. Thus, ‘substituent’ and ‘organic group’ may have any of the following meanings.

The organic group may comprise any one or more atoms from any of groups IIIA, IVA, VA, VIA or VIIA of the Periodic Table, such as a B, Si, N, P, O, or S atom (e.g. OH, OR, NH₂, NHR, NR₂, SH, SR, SO₂R, SO₃H, PO₄H₂) or a halogen atom (e.g. F, Cl, Br or I) where R is a linear or branched lower hydrocarbon (1-6 C atoms) or a linear or branched higher hydrocarbon (7 C atoms or more, e.g. 7-40 C atoms).

The organic group preferably comprises a hydrocarbon group. The hydrocarbon group may comprise a straight chain, a branched chain or a cyclic group. Independently, the hydrocarbon group may comprise an aliphatic or an aromatic group. Also independently, the hydrocarbon group may comprise a saturated or unsaturated group.

When the hydrocarbon comprises an unsaturated group, it may comprise one or more alkene functionalities and/or one or more alkyne functionalities. When the hydrocarbon comprises a straight or branched chain group, it may comprise one or more primary, secondary and/or tertiary alkyl groups.

When the hydrocarbon comprises a cyclic group, it may comprise an aromatic ring, a non-aromatic ring, an aliphatic ring, a heterocyclic group, and/or fused ring derivatives of these groups. The ring may be fully saturated, partially saturated, or fully unsaturated. The cyclic group may thus comprise a benzene, naphthalene, anthracene, phenanthrene, phenalene, biphenylene, pentalene, indene, as-indacene, s-indacene, acenaphthylene, fluorene, fluoranthene, acephenanthrylene, azulene, heptalene, pyrrole, pyrazole, imidazole, 1,2,3-triazole, 1,2,4-triazole, tetrazole, pyrrolidine, furan, tetrahydrofuran, 2-aza-tetrahydrofuran, 3-aza-tetrahydrofuran, oxazole, isoxazole, furazan, 1,2,4-oxadiazol, 1,3,4-oxadiazole, thiophene, isothiazole, thiazole, thiolane, pyridine, pyridazine, pyrimidine, pyrazine, piperidine, 2-azapiperidine, 3-azapiperidine, piperazine, pyran, oxetan-2-yl, oxetan-3-yl, tetrahydropyran, 2-azapyran, 3-azapyran, 4-azapyran, 2-aza-tetrahydropyran, 3-aza-tetrahydropyran, morpholine, thiopyran, 2-azathiopyran, 3-azathiopyran, 4-azathiopyran, thiane, indole, indazole, benzimidazole, 4-azaindole, 5-azaindole, 6-azaindole, 7-azaindole, isoindole, 4-azaisoindole, 5-azaisoindole, 6-azaisoindole, 7-azaisoindole, indolizine, 1-azaindolizine, 2-azaindolizine, 3-azaindolizine, 5-azaindolizine, 6-azaindolizine, 7-azaindolizine, 8-azaindolizine, 9-azaindolizine, purine, carbazole, carboline, benzofuran, isobenzofuran, benzothiophene, isobenzothiophene, quinoline, cinnoline, quinazoline, quinoxaline, 5-azaquinoline, 6-azaquinoline, 7-azaquinoline, isoquinoline, phthalazine, 6-azaisoquinoline, 7-azaisoquinoline, pteridine, chromene, isochromene, acridine, phenanthridine, perimidine, phenanthroline, phenoxazine, xanthene, phenoxanthiin, and/or thianthrene, as well as regioisomers of the above groups. These groups may generally be attached at any point in the group, and also may be attached at a hetero-atom or at a carbon atom. In some instances particular attachment points are preferred, such as at 1-yl, 2-yl and the like, and these are specified explicitly where appropriate. All tautomeric ring forms are included in these definitions. For example, pyrrole is intended to include 1H-pyrrole, 2H-pyrrole and 3H-pyrrole.

The number of carbon atoms in the hydrocarbon group is not especially limited, but preferably the hydrocarbon group comprises from 1-40 C atoms. The hydrocarbon group may thus be a lower hydrocarbon (1-6 C atoms) or a higher hydrocarbon (7 C atoms or more, e.g. 7-40 C atoms). The lower hydrocarbon group may be a methyl, ethyl, propyl, butyl, pentyl or hexyl group or regioisomers of these, such as isopropyl, isobutyl, tert-butyl, etc. The number of atoms in the ring of the cyclic group is not especially limited, but preferably the ring of the cyclic group comprises from 3-10 atoms, such as 3, 4, 5, 6, 7, 8, 9 or 10 atoms.

The groups comprising heteroatoms described above, as well as any of the other groups defined above, may comprise one or more heteroatoms from any of groups IIIA, IVA, VA, VIA or VIIA of the Periodic Table, such as a B, Si, N, P, O, or S atom or a halogen atom (e.g. F, Cl, Br or I). Thus the substituent may comprise one or more of any of the common functional groups in organic chemistry, such as hydroxy groups, carboxylic acid groups, ester groups, ether groups, aldehyde groups, ketone groups, amine groups, amide groups, imine groups, thiol groups, thioether groups, sulphate groups, sulphonic acid groups, sulphonyl groups, and phosphate groups etc. The substituent may also comprise derivatives of these groups, such as carboxylic acid anhydrides and carboxylic acid halides.

In addition, any substituent may comprise a combination of two or more of the substituents and/or functional groups defined above.

In a typical embodiment, the invention provides a compound, that is a PARP7 and/or a PARP1 inhibitor compound, which compound comprises the following formula:

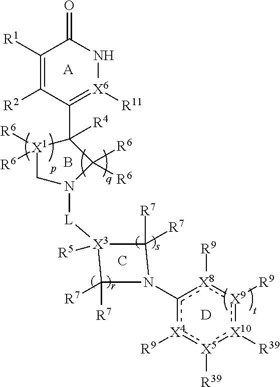

wherein:
   p is selected from 0, 1, 2, 3, and 4;
   q is selected from 0, 1 and 2, preferably from 0 and 1;
   p+q may be 1, 2, 3 or 4;
   ring C is a saturated ring; and
   wherein X⁶, R¹, R², R¹¹, X¹, R⁴, R⁶, X³, R⁷, r, s, X⁴, X⁵, X⁸, X⁹, X¹⁰, R⁹, R³⁹, t and L are as defined herein.

In typical embodiments, the invention provides a compound as defined above, wherein ring B is selected from a group having one of the following structures:

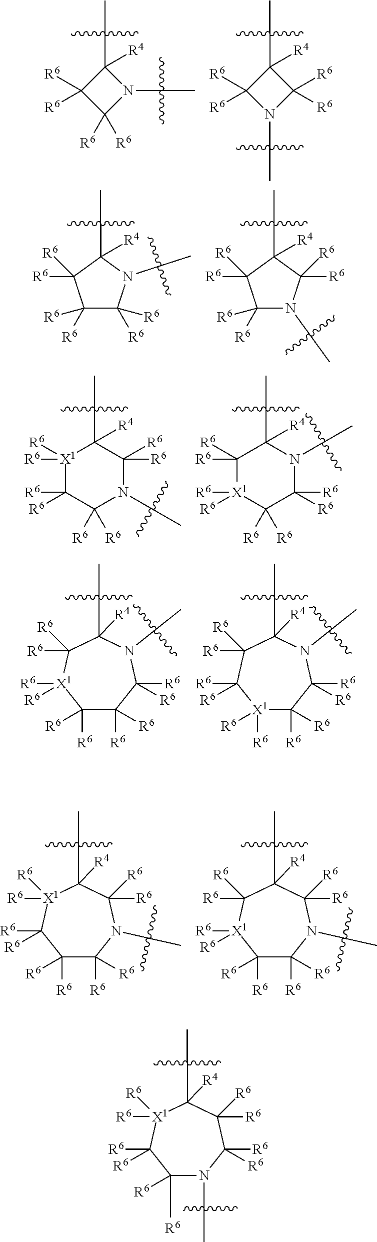

wherein X¹ is independently selected from C, N, O and S, and R⁴ and each R⁶ are as defined herein.

Preferably, ring B is selected from a group having one of the following structures:

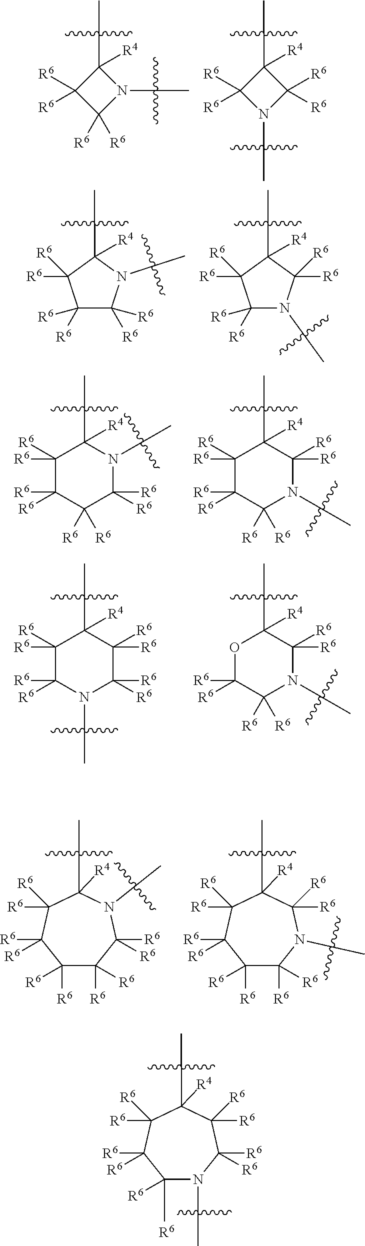

wherein R⁴ and each R⁶ are as defined herein.

In typical embodiments, the invention provides a compound as defined above, wherein ring D is selected from a group having one of the following structures:

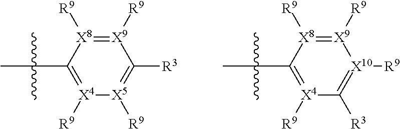

wherein, X⁴, X⁵, X⁸, and X⁹, are independently selected from C and N; and R⁹ and R³ are as defined herein;
or wherein ring D is selected from the following structure:

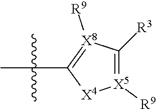

wherein, X⁵ and X⁸ are independently selected from C and N, X⁴ is independently selected from O and S, and R⁹ and R³ are as defined herein;
or wherein ring D is selected from the following structure:

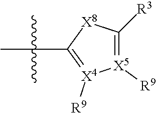

wherein, X⁴ and X⁵ are independently selected from C and N, X⁸ is independently selected from O and S; and R⁹ and R³ are as defined herein.

Preferably, ring D is selected from one of the following structures:

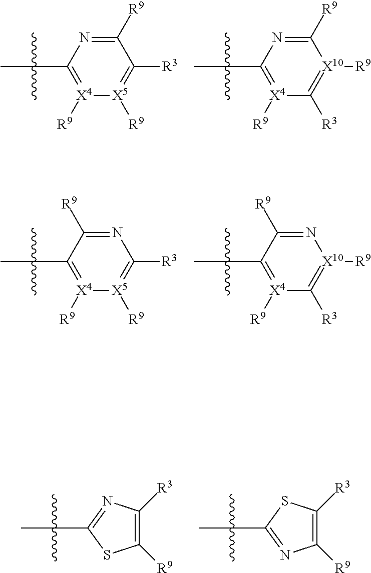

wherein, X⁴, X⁵ and X¹⁰ are independently selected from C and N; and R⁹ and R³ are as defined herein.

More preferably, ring D is selected from one of the following structures:

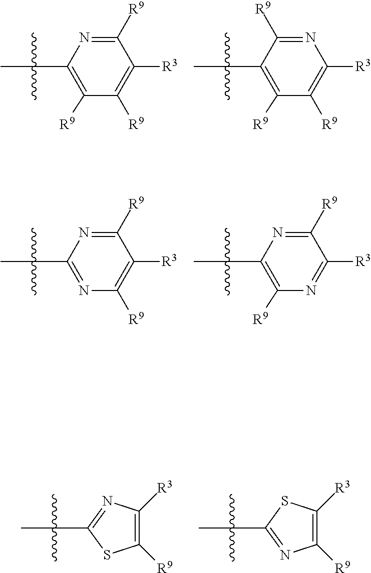

wherein R⁹ and R³ are as defined herein.

The R groups referred to in the compounds and structures herein will now be described in more detail.

As has been mentioned, the number of R substituents a ring atom will depend on its valency. Thus, it will be apparent in all of the embodiments of the invention, both above and below, that when a ring atom has three ring bonds (either 3 single bonds or a single bond and a double bond), it will have no substituents if it is N and 1 substituent (H or an organic group as defined herein) if it is C, and when a ring atom has two ring bonds (2 single bonds), it will have 1 substituent (H or an organic group as defined herein) if it is N and 2 substituents if it is C (each independently chosen from H or an organic group as defined herein). Of course, if the ring atom is O there will not be any substituents. If the ring atom is S it may have no substituents, or it may be a sulphonyl group.

As has been mentioned, in all of the embodiments of this invention (both above and below herein), the substituent is not especially limited, provided that it does not prevent the PARP7 and/or PARP1 inhibitory function from occurring. However, in typical embodiments, the substituents may be selected independently as follows.

In typical embodiments, R³ is selected from a halogenated C1 to C3 alkyl group, a halogenated C1 to C3 alkoxy group, a halogen group, a —CN group, and an amide group, and is preferably a —CF₃, —OCF₃, —F, —Cl, —CN, or a —CONHR group, where R is a lower alkyl group, preferably a C1 to C6 alkyl group.

In further typical embodiments, R⁴, R⁵, R⁶, R⁷ and R¹⁰ are each independently selected from H and a group selected from the following groups:
deuterium;
a halogen
   (such as —F, —Cl, —Br and —I);
a nitrile group;
a substituted or unsubstituted linear or branched C₁-C₆ alkyl group
   (such as Me, Et, Pr, i-Pr, n-Bu, i-Bu, t-Bu, pentyl and hexyl);
a substituted or unsubstituted linear or branched C₁-C₆ alkyl-aryl group
   (such as —CH₂Ph, —CH₂(2,3 or 4)F-Ph, —CH₂(2,3 or 4)Cl-Ph, —CH₂(2,3 or 4)Br-Ph, —CH₂(2,3 or 4)I-Ph, —CH₂CH₂Ph, —CH₂CH₂CH₂Ph, —CH₂CH₂CH₂CH₂Ph, —CH₂CH₂CH₂CH₂CH₂Ph, and —CH₂CH₂CH₂CH₂CH₂CH₂Ph);
a substituted or unsubstituted linear or branched C₁-C₆ halogenated alkyl group
   (such as —CH₂F, —CH₂Cl, —CH₂Br, —CH₂I, —CHF₂, —CF₃, —CCl₃—CBr₃, —Cl₃, —CH₂CH₂F, —CH₂CF₃, —CH₂CCl₃, —CH₂CBr₃, and —CH₂Cl₃);
—NH₂ or a substituted or unsubstituted linear or branched primary secondary or tertiary C₁-C₆ amine group
   (such as —NMeH, —NMe₂, —NEtH, —NEtMe, —NEt₂, —NPrH, —NPrMe, —NPrEt, —NPr₂, —NBuH, —NBuMe, —NBuEt, —CH₂—NH₂, —CH₂—NMeH, —CH₂—NMe₂, —CH₂—NEtH, —CH₂—NEtMe, —CH₂—NEt₂, —CH₂—NPrH, —CH₂—NPrMe, and —CH₂—NPrEt);
a substituted or unsubstituted amino-aryl group
   (such as —NH-Ph, —NH-(2,3 or 4)F-Ph, —NH-(2,3 or 4)Cl-Ph, —NH-(2,3 or 4)Br-Ph, —NH-(2,3 or 4)1-Ph, —NH-(2,3 or 4)Me-Ph, —NH-(2,3 or 4)Et-Ph, —NH-(2,3 or 4)Pr-Ph, —NH-(2,3 or 4)Bu-Ph, NH-(2,3 or 4)OMe-Ph, —NH-(2,3 or 4)OEt-Ph, —NH-(2,3 or 4)OPr-Ph, —NH-(2,3 or 4)OBu-Ph, —NH-2,(3,4,5 or 6)F₂-Ph, —NH-2,(3,4,5 or 6)Cl₂-Ph, —NH-2,(3,4,5 or 6)Br₂-Ph, —NH-2,(3,4,5 or 6)F₂-Ph, —NH-2,(3,4,5 or 6)Me₂-Ph, —NH-2,(3,4,5 or 6)Et₂-Ph, —NH-2,(3,4,5, or 6)Pr₂-Ph, —NH-2,(3,4,5 or 6)Bu₂-Ph),
a substituted or unsubstituted cyclic amine or amido group
   (such as pyrrolidin-1-yl, pyrrolidin-2-yl, pyrrolidin-3-yl, piperidin-1-yl, piperidin-2-yl, piperidin-3-yl, piperidin-4-yl, morpholin-2-yl, morpholin-3-yl, morpholin-4-yl, 2-keto-pyrrolidinyl, 3-keto-pyrrolidinyl, 2-keto-piperidinyl, 3-keto-piperidinyl, and 4-keto-piperidinyl);
a substituted or unsubstituted cyclic C₃-C₈ alkyl group
   (such as cyclopropyl, cyclobutyl, cyclopentyl, cyclohexyl, cycloheptyl and cyclooctyl);
an —OH group or a substituted or unsubstituted linear or branched C₁-C₆ alcohol group
   (such as —CH₂OH, —CH₂CH₂OH, —CH(CH₃)CH₂OH, —C(CH₃)₂OH, —CH₂CH₂CH₂OH, —CH₂CH₂CH₂CH₂OH, —CH(CH₃)CH₂CH₂OH, —CH(CH₃)CH(CH₃)OH, —CH(CH₂CH₃)CH₂OH, —C(CH₃)₂CH₂OH, —CH₂CH₂CH₂CH₂CH₂OH, and —CH₂CH₂CH₂CH₂CH₂CH₂OH);
a substituted or unsubstituted linear or branched C₁-C₆ carboxylic acid group
   (such as —COOH, —CH₂COOH, —CH₂CH₂COOH, —CH₂CH₂CH₂COOH, —CH₂CH₂CH₂CH₂COOH, and —CH₂CH₂CH₂CH₂CH₂COOH);
a substituted or unsubstituted linear or branched carbonyl group
   (such as —(CO)Me, —(CO)Et, —(CO)Pr, —(CO)iPr, —(CO)nBu, —(CO)iBu, —(CO)tBu, —(CO)Ph, —(CO)CH₂Ph, —(CO)CH₂OH, —(CO)CH₂OCH₃, —(CO)CH₂NH₂, —(CO)CH₂NHMe, —(CO)CH₂NM e₂, —(CO)-cyclopropyl, —(CO)-1,3-epoxypropan-2-yl; —(CO)NH₂, —(CO)NHMe, —(CO)NMe₂, —(CO)NHEt, —(CO)NEt₂, —(CO)-pyrollidine-N-yl, —(CO)-morpholine-N-yl, —(CO)-piperazine-N-yl, —(CO)—N-methyl-piperazine-N-yl, —(CO)NHCH₂CH₂OH, —(CO)NHCH₂CH₂OMe, —(CO)NHCH₂CH₂NH₂, —(CO)NHCH₂CH₂NHMe, and —(CO)NHCH₂CH₂NMe₂);
a substituted or unsubstituted linear or branched C₁-C₆ carboxylic acid ester group
   (such as —COOMe, —COOEt, —COOPr, —COO-i-Pr, —COO-n-Bu, —COO-i-Bu, —COO-t-Bu, —CH₂COOMe, —CH₂CH₂COOMe, —CH₂CH₂CH₂COOMe, and —CH₂CH₂CH₂CH₂COOMe);
a substituted or unsubstituted linear or branched C₁-C₆ amide group
   (such as —CO—NH₂, —CO—NMeH, —CO—NMe₂, —CO-NEtH, —CO-NEtMe, —CO-NEt₂, —CO—NPrH, —CO—NPrMe, and —CO-NPrEt);
a substituted or unsubstituted linear or branched C₁-C₇ amino carbonyl group
   (such as —NH—CO-Me, —NH—CO-Et, —NH—CO—Pr, —NH—CO-Bu, —NH—CO-pentyl, —NH—CO-hexyl, —NH—CO-Ph, —NMe-CO-Me, —NMe-CO-Et, —NMe-CO—Pr, —NMe-CO-Bu, —NMe-CO-pentyl, —NMe-CO-hexyl, —NMe-CO-Ph;
a substituted or unsubstituted linear or branched C1-C7 alkoxy or aryloxy group
   (such as —OMe, —OEt, —OPr, —O-i-Pr, —O-n-Bu, —O-i-Bu, —O-t-Bu, —O-pentyl, —O-hexyl, —OCH₂F, —OCHF₂, —OCF₃, —OCH₂Cl, —OCHCl₂, —OCCl₃, —O-Ph, —O—CH₂-Ph, —O—CH₂-(2,3 or 4)-F-Ph, —O—CH₂-(2,3 or 4)-Cl-Ph, —CH₂OMe, —CH₂OEt, —CH₂OPr, —CH₂OBu, —CH₂CH₂OMe, —CH₂CH₂CH₂OMe, —CH₂CH₂CH₂CH₂OMe, and —CH₂CH₂CH₂CH₂CH₂OMe);
a substituted or unsubstituted linear or branched aminoalkoxy group
   (such as —OCH₂NH₂, —OCH₂NHMe, —OCH₂NMe₂, —OCH₂NHEt, —OCH₂NEt₂, —OCH₂CH₂NH₂, —OCH₂CH₂NHMe, —OCH₂CH₂NMe₂, —OCH₂CH₂NHEt, and —OCH₂CH₂NEt₂;
a substituted or unsubstituted sulphonyl group
   (such as —SO₂Me, —SO₂Et, —SO₂Pr, —SO₂iPr, —SO₂Ph, —SO₂-(2,3 or 4)-F-Ph, —SO₂— cyclopropyl, —SO₂CH₂CH₂OCH₃), —SO₂NH₂, —SO₂NHMe, —SO₂NMe₂, —SO₂NHEt, —SO₂NEt₂, —SO₂-pyrrolidine-N-yl, —SO₂-morpholine-N-yl, —SO₂NHCH₂OMe, and —SO₂NHCH₂CH₂OMe;
a substituted or unsubstituted aminosulphonyl group
   (such as —NHSO₂Me, —NHSO₂Et, —NHSO₂Pr, —NHSO₂iPr, —NHSO₂Ph, —NHSO₂-(2,3 or 4)-F-Ph, —NHSO₂-cyclopropyl, —NHSO₂CH₂CH₂OCH₃);
a substituted or unsubstituted aromatic group
   (such as Ph-, 2-F-Ph-, 3-F-Ph-, 4-F-Ph-, 2-Cl-Ph-, 3-Cl-Ph-, 4-Cl-Ph-, 2-Br-Ph-, 3-Br-Ph-, 4-Br-Ph-, 2-I-Ph-, 3-I-Ph, 4-I-Ph-, 2,(3,4,5 or 6)-F₂-Ph-, 2,(3,4,5 or 6)-Cl₂-Ph-, 2,(3,4,5 or 6)-Br₂-Ph-, 2,(3,4,5 or 6)-F₂-Ph-, 2,(3,4,5 or 6)-Me₂-Ph-, 2,(3,4,5 or 6)-Et₂-Ph-, 2,(3,4,5 or 6)-Pr₂-Ph-, 2,(3,4,5 or 6)-Bu₂-Ph-, 2,(3,4,5 or 6)-(CN)₂-Ph-, 2,(3,4,5 or 6)-(NO₂)₂-Ph-, 2,(3,4,5 or 6)-(NH₂)₂-Ph-, 2,(3,4,5 or 6)-(MeO)₂-Ph-, 2,(3,4,5 or 6)-(CF₃)₂-Ph-, 3,(4 or 5)-F₂-Ph-, 3,(4 or 5)-Cl₂-Ph-, 3,(4 or 5)-Br₂-Ph-, 3,(4 or 5)-F₂-Ph-, 3,(4 or 5)-Me₂-Ph-, 3,(4 or 5)-Et₂-Ph-, 3,(4 or 5)-Pr₂-Ph-, 3,(4 or 5)-Bu₂-Ph-, 3,(4 or 5)-(CN)₂-Ph-, 3,(4 or 5)-(NO₂)₂-Ph-, 3,(4 or 5)-(NH₂)₂-Ph-, 3,(4 or 5)-(MeO)₂-Ph-, 3,(4 or 5)-(CF₃)₂-Ph-, 2-Me-Ph-, 3-Me-Ph-, 4-Me-Ph-, 2-Et-Ph-, 3-Et-Ph-, 4-Et-Ph-, 2-Pr-Ph-, 3-Pr-Ph-, 4-Pr-Ph-, 2-Bu-Ph-, 3-Bu-Ph-, 4-Bu-Ph-, 2-(CN)-Ph-, 3-(CN)-Ph-, 4-(CN)-Ph-, 2-(NO₂)-Ph-, 3-(NO₂)-Ph-, 4-(NO₂)-Ph-, 2-(NH₂)-Ph-, 3-(NH₂)-Ph-, 4-(NH₂)-Ph-, 2-MeO-Ph-, 3-MeO-Ph-, 4-MeO-Ph-, 2-(NH₂—CO)-Ph-, 3-(NH₂—CO)-Ph-, 4-(NH₂—CO)-Ph-, 2-CF₃-Ph-, 3-CF₃-Ph-, 4-CF₃-Ph-, 2-CF₃O-Ph-, 3-CF₃O-Ph-, and 4-CF₃O-Ph-);
a saturated or unsaturated, substituted or unsubstituted, heterocyclic group including an aromatic heterocyclic group and/or a non-aromatic heterocyclic group
   (such as pyrrole-1-yl, pyrrole-2-yl, pyrrole-3-yl, pyrazole-1-yl, pyrazole-3-yl, pyrazole-4-yl, pyrazole-5-yl, imidazole-1-yl, imidazole-2-yl, imidazole-4-yl, imidazole-5-yl, 1,2,3-triazole-1-yl, 1,2,3-triazole-4-yl, 1,2,3-triazole-5-yl, 1,2,4-triazole-1-yl, 1,2,4-triazole-3-yl, 1,2,4-triazole-5-yl, pyridin-2-yl, pyridin-3-yl, pyridin-4-yl, pyridazine-3-yl, pyridazine-4-yl, pyrimidin-2-yl, pyrimidin-4-yl, pyrimidin-5-yl, pyrimidin-6-yl, pyrazine-2-yl, pyrrolidine-1-yl, pyrrolidine-2-yl, pyrrolidine-3-yl, piperidine-1-yl, piperidine-2-yl, piperidine-3-yl, piperidine-4-yl, 2-azapiperidine-1-yl, 2-azapiperidine-3-yl, 2-azapiperidine-4-yl, 3-azapiperidine-1-yl, 3-azapiperidine-2-yl, 3-azapiperidine-4-yl, 3-azapiperidine-5-yl, piperazine-1-yl, piperazine-2-yl, furan-2-yl, furan-3-yl, pyran-2-yl, pyran-3-yl, pyran-4-yl, 2-azapyran-2-yl, 2-azapyran-3-yl, 2-azapyran-4-yl, 2-azapyran-5-yl, 2-azapyran-6-yl, 3-azapyran-2-yl, 3-azapyran-4-yl, 3-azapyran-5-yl, 3-azapyran-6-yl, 4-azapyran-2-yl, 4-azapyran-3-yl, 4-azapyran-4-yl, 4-azapyran-5-yl, 4-azapyran-6-yl, oxetan-2-yl, oxetan-3-yl, tetrahydrofuran-2-yl, tetrahydrofuran-3-yl, 2-aza-tetrahydrofuran-2-yl, 2-aza-tetrahydrofuran-3-yl, 2-aza-tetrahydrofuran-4-yl, 2-aza-tetrahydrofuran-5-yl, 3-aza-tetrahydrofuran-2-yl, 3-aza-tetrahydrofuran-3-yl, 3-aza-tetrahydrofuran-4-yl, 3-aza-tetrahydrofuran-5-yl, tetrahydropyran-2-yl, tetrahydropyran-3-yl, tetrahydropyran-4-yl, 2-aza-tetrahydropyran-2-yl, 2-aza-tetrahydropyran-3-yl, 2-aza-tetrahydropyran-4-yl, 2-aza-tetrahydropyran-5-yl, 2-aza-tetrahydropyran-6-yl, 3-aza-tetrahydropyran-2-yl, 3-aza-tetrahydropyran-3-yl, 3-aza-tetrahydropyran-4-yl, 3-aza-tetrahydropyran-5-yl, 3-aza-tetrahydropyran-6-yl, morpholine-2-yl, morpholine-3-yl, morpholine-4-yl, thiophen-2-yl, thiophen-3-yl, isothiazole-3-yl, isothiazole-4-yl, isothiazole-5-yl, thiazole-2-yl, thiazole-4-yl, thiazole-5-yl, thiopyran-2-yl, thiopyran-3-yl, thiopyran-4-yl, 2-azathiopyran-2-yl, 2-azathiopyran-3-yl, 2-azathiopyran-4-yl, 2-azathiopyran-5-yl, 2-azathiopyran-6-yl, 3-azathiopyran-2-yl, 3-azathiopyran-4-yl, 3-azathiopyran-5-yl, 3-azathiopyran-6-yl, 4-azathiopyran-2-yl, 4-azathiopyran-3-yl, 4-azathiopyran-4-yl, 4-azathiopyran-5-yl, 4-azathiopyran-6-yl, thiolane-2-yl, thiolane-3-yl, thiane-2-yl, thiane-3-yl, thiane-4-yl, oxazol-2-yl, oxazol-4-yl, oxazol-5-yl, isoxazol-3-yl, isoxazol-4-yl, isoxazol-5-yl, furazan-3-yl, (1,3,4-oxadiazol)-2-yl, (1,3,4-oxadiazol)-5-yl, (1,2,4-oxadiazol)-3-yl, (1,2,4-oxadiazol)-5-yl; and tetrazole-1-yl, tetrazole-2-yl, tetrazole-5-yl);
where there are two R groups attached to the same atom, they may together form a group which is double bonded to that atom, (such as a carbonyl group (═O) or an alkene group (═C(R′)₂) wherein each R′ group is the same or different and is H or an organic group, preferably H or a straight or branched C₁-C6 alkyl group);
wherein, a pair of R⁶ groups attached to different atoms may together form a ring with ring B atoms, and/or an R⁶ group and an R⁴ group may together form a ring with ring B atoms, and/or a pair of R⁷ groups attached to different atoms may together form a ring with ring C atoms, and/or an R⁷ group and an R⁵ group may together form a ring with ring B atoms, optionally wherein each of the pair of R⁶ groups and/or the R⁶ and R⁴ groups and/or the pair of R⁷ groups and/or the R⁷ and R⁵ groups independently comprises (X¹¹)_{1 or 2}, wherein each X¹¹ may be the same or different and is independently selected from C, N, O and S; and wherein each X¹¹ is independently unsubstituted or (i) independently substituted with H or an organic group selected from a C₁-C₆ alkyl group, a halogen such as F, or hydroxyl, when X¹¹ is C; and
(ii) independently substituted with H or an organic group selected from a C₁-C₆ alkyl group or a C₁-C₆ amide group, when X¹¹ is N.

Preferably, R⁴, R⁵, R⁶, and R⁷ are each independently selected from H, deuterium, a halogen (such as —F, —Cl, —Br, and —I, preferably F or Cl), a substituted or unsubstituted C₁-C₆ alkyl group, a substituted or unsubstituted linear or branched C₁-C₆ halogenated alkyl group (preferably CF₃), an —OH group or a substituted or unsubstituted linear or branched C₁-C₆ alcohol group, an —NH₂ group or a substituted or unsubstituted C₁-C₆ amino group and a substituted or unsubstituted C₁-C₆ alkoxy group; and wherein, when a pair of R⁶ groups attached to different atoms together forms a ring with ring B atoms, and/or a pair of R⁷ groups attached to different atoms together forms a ring with ring C atoms, each of the pair of R⁶ groups and/or pair of R⁷ groups independently comprises —CH₂— or —CH₂CH₂—.

Preferably, R⁹ is selected from H, a C₁-C₃ alkyl group, a C₁ to C₃ fluoroalkyl group and a halogen group.

Preferably, R¹⁰ is selected from H, a halogen (such as —F, —Cl, —Br, and —I, preferably —F), a substituted or unsubstituted C₁-C₆ alkyl group, a substituted or unsubstituted linear or branched C₁-C₆ halogenated alkyl group (preferably CF₃), an —NH₂ group or a substituted or unsubstituted C₁-C₆ amino group, an —OH group or a substituted or unsubstituted linear or branched C₁-C₆ alcohol group and a substituted or unsubstituted C₁-C₆ alkoxy group.

In further typical embodiments, R¹² is selected from H and a group selected from the following groups:
a substituted or unsubstituted linear or branched C₁-C₆ alkyl group
   (such as Me, Et, Pr, i-Pr, n-Bu, i-Bu, t-Bu, pentyl and hexyl);
a substituted or unsubstituted linear or branched C₁-C₆ alkyl-aryl group
   (such as —CH₂Ph, —CH₂(2,3 or 4)F-Ph, —CH₂(2,3 or 4)Cl-Ph, —CH₂(2,3 or 4)Br-Ph, —CH₂(2,3 or 4)I-Ph, —CH₂CH₂Ph, —CH₂CH₂CH₂Ph, —CH₂CH₂CH₂CH₂Ph, —CH₂CH₂CH₂CH₂CH₂Ph, and —CH₂CH₂CH₂CH₂CH₂CH₂Ph);
a substituted or unsubstituted linear or branched C₁-C₆ halogenated alkyl group
   (such as —CH₂F, —CF₃, —CH₂CH₂F and —CH₂CF₃);
a substituted or unsubstituted cyclic amine or amido group
   (such as pyrrolidin-3-yl, piperidin-3-yl, piperidin-4-yl, 2-keto-pyrrolidinyl, 3-keto-pyrrolidinyl, 2-keto-piperidinyl, 3-keto-piperidinyl, and 4-keto-piperidinyl);
a substituted or unsubstituted cyclic C₃-C₈ alkyl group
   (such as cyclopropyl, cyclobutyl, cyclopentyl, cyclohexyl, cycloheptyl and cyclooctyl);
a substituted or unsubstituted linear or branched C₂-C₆ alcohol group
   (such as —CH₂CH₂OH, —CH(CH₃)CH₂OH, —C(CH₃)₂OH, —CH₂CH₂CH₂OH, —CH₂CH₂CH₂CH₂OH, —CH(CH₃)CH₂CH₂OH, —CH(CH₃)CH(CH₃)OH, —CH(CH₂CH₃)CH₂OH, —C(CH₃)₂CH₂OH, —CH₂CH₂CH₂CH₂CH₂OH, and —CH₂CH₂CH₂CH₂CH₂CH₂OH);
a substituted or unsubstituted linear or branched C₂-C₆ carboxylic acid group
   (such as —CH₂COOH, —CH₂CH₂COOH, —CH₂CH₂CH₂COOH, —CH₂CH₂CH₂CH₂COOH, and —CH₂CH₂CH₂CH₂CH₂COOH);
a substituted or unsubstituted linear or branched carbonyl group
   (such as —(CO)Me, —(CO)Et, —(CO)Pr, —(CO)-i_Pr, —(CO)-n-Bu, —(CO)-i-Bu, —(CO)-t-Bu, —(CO)Ph, —(CO)CH₂Ph, —(CO)CH₂OH, —(CO)CH₂OCH₃, —(CO)CH₂NH₂, —(CO)CH₂NHMe, —(CO)CH₂NMe₂, —(CO)-cyclopropyl, —(CO)-1,3-epoxypropan-2-yl; —(CO)NH₂, —(CO)NHMe, —(CO)NMe₂, —(CO)NHEt, —(CO)NEt₂, —(CO)-pyrollidine-N-yl, —(CO)-morpholine-N-yl, —(CO)-piperazine-N-yl, —(CO)—N-methyl-piperazine-N-yl, —(CO)NHCH₂CH₂OH, —(CO)NHCH₂CH₂OMe, —(CO) NHCH₂CH₂NH₂, —(CO)NHCH₂CH₂NHMe, and —(CO)NHCH₂CH₂NMe₂;
a substituted or unsubstituted linear or branched C₁-C₆ carboxylic acid ester group
   (such as —COOMe, —COOEt, —COOPr, —COO-i-Pr, —COO-n-Bu, —COO-i-Bu, —COO-t-Bu, —CH₂COOMe, —CH₂CH₂COOMe, —CH₂CH₂CH₂COOMe, and —CH₂CH₂CH₂CH₂COOMe);
a substituted or unsubstituted linear or branched C₁-C₆ amide group
   (such as —CO—NH₂, —CO—NMeH, —CO—NMe₂, —CO-NEtH, —CO-NEtMe, —CO-NEt₂, —CO—NPrH, —CO—NPrMe, and —CO-NPrEt);
a substituted or unsubstituted sulphonyl group
   (such as —SO₂Me, —SO₂Et, —SO₂Pr, —SO₂iPr, —SO₂Ph, —SO₂-(2,3 or 4)-F-Ph, —SO₂— cyclopropyl, —SO₂CH₂CH₂OCH₃, —SO₂NH₂, —SO₂NHMe, —SO₂NMe₂, —SO₂NHEt, —SO₂NEt₂, —SO₂-pyrrolidine-N-yl, —SO₂-morpholine-N-yl, —SO₂NHCH₂OMe, and —SO₂NHCH₂CH₂OMe);
a substituted or unsubstituted aromatic group
   (such as Ph-, 2-F-Ph-, 3-F-Ph-, 4-F-Ph-, 2-Cl-Ph-, 3-Cl-Ph-, 4-Cl-Ph-, 2-Br-Ph-, 3-Br-Ph-, 4-Br-Ph-, 2-I-Ph-, 3-I-Ph, 4-I-Ph-, 2,(3,4,5 or 6)-F₂-Ph-, 2,(3,4,5 or 6)-Cl₂-Ph-, 2,(3,4,5 or 6)-Br₂-Ph-, 2,(3,4,5 or 6)-I₂-Ph-, 2,(3,4,5 or 6)-Me₂-Ph-, 2,(3,4,5 or 6)-Et₂-Ph-, 2,(3,4,5 or 6)-Pr₂-Ph-, 2,(3,4,5 or 6)-Bu₂-Ph-, 2,(3,4,5 or 6)-(CN)₂-Ph-, 2,(3,4,5 or 6)-(NO₂)₂-Ph-, 2,(3,4,5 or 6)-(NH₂)₂-Ph-, 2,(3,4,5 or 6)-(MeO)₂-Ph-, 2,(3,4,5 or 6)-(CF₃)₂-Ph-, 3,(4 or 5)-F₂-Ph-, 3,(4 or 5)-Cl₂-Ph-, 3,(4 or 5)-Br₂-Ph-, 3,(4 or 5)-I₂-Ph-, 3,(4 or 5)-Me₂-Ph-, 3,(4 or 5)-Et₂-Ph-, 3,(4 or 5)-Pr₂-Ph-, 3,(4 or 5)-Bu₂-Ph-, 3,(4 or 5)-(CN)₂-Ph-, 3,(4 or 5)-(NO₂)₂-Ph-, 3,(4 or 5)-(NH₂)₂-Ph-, 3,(4 or 5)-(MeO)₂-Ph-, 3,(4 or 5)-(CF₃)₂-Ph-, 2-Me-Ph-, 3-Me-Ph-, 4-Me-Ph-, 2-Et-Ph-, 3-Et-Ph-, 4-Et-Ph-, 2-Pr-Ph-, 3-Pr-Ph-, 4-Pr-Ph-, 2-Bu-Ph-, 3-Bu-Ph-, 4-Bu-Ph-, 2-(CN)-Ph-, 3-(CN)-Ph-, 4-(CN)-Ph-, 2-(NO₂)-Ph-, 3-(NO₂)-Ph-, 4-(NO₂)-Ph-, 2-(NH₂)-Ph-, 3-(NH₂)-Ph-, 4-(NH₂)-Ph-, 2-MeO-Ph-, 3-MeO-Ph-, 4-MeO-Ph-, 2-(NH₂—CO)-Ph-, 3-(NH₂—CO)-Ph-, 4-(NH₂—CO)-Ph-, 2-CF₃-Ph-, 3-CF₃-Ph-, 4-CF₃-Ph-, 2-CF₃O-Ph-, 3-CF₃O-Ph-, and 4-CF₃O-Ph-); and
a substituted or unsubstituted saturated or unsaturated, substituted or unsubstituted, heterocyclic group including an aromatic heterocyclic group and/or a non-aromatic heterocyclic group
   (such as pyrrole-2-yl, pyrrole-3-yl, pyrazole-3-yl, pyrazole-4-yl, pyrazole-5-yl, imidazole-2-yl, imidazole-4-yl, imidazole-5-yl, 1,2,3-triazole-4-yl, 1,2,3-triazole-5-yl, 1,2,4-triazole-3-yl, 1,2,4-triazole-5-yl, pyridin-2-yl, pyridin-3-yl, pyridin-4-yl, pyridazine-3-yl, pyridazine-4-yl, pyrimidin-2-yl, pyrimidin-4-yl, pyrimidin-5-yl, pyrimidin-6-yl, pyrazine-2-yl, pyrrolidine-2-yl, pyrrolidine-3-yl, piperidine-2-yl, piperidine-3-yl, piperidine-4-yl, 2-azapiperidine-3-yl, 2-azapiperidine-4-yl, 3-azapiperidine-2-yl, 3-azapiperidine-4-yl, 3-azapiperidine-5-yl, piperazine-2-yl, furan-2-yl, furan-3-yl, pyran-2-yl, pyran-3-yl, pyran-4-yl, 2-azapyran-3-yl, 2-azapyran-4-yl, 2-azapyran-5-yl, 2-azapyran-6-yl, 3-azapyran-2-yl, 3-azapyran-4-yl, 3-azapyran-5-yl, 3-azapyran-6-yl, 4-azapyran-2-yl, 4-azapyran-3-yl, 4-azapyran-5-yl, 4-azapyran-6-yl, tetrahydrofuran-2-yl, tetrahydrofuran-3-yl, 2-aza-tetrahydrofuran-3-yl, 2-aza-tetrahydrofuran-4-yl, 2-aza-tetrahydrofuran-5-yl, 3-aza-tetrahydrofuran-2-yl, 3-aza-tetrahydrofuran-4-yl, 3-aza-tetrahydrofuran-5-yl, tetrahydropyran-2-yl, oxetan-3-yl, tetrahydropyran-3-yl, tetrahydropyran-4-yl, 2-aza-tetrahydropyran-3-yl, 2-aza-tetrahydropyran-4-yl, 2-aza-tetrahydropyran-5-yl, 2-aza-tetrahydropyran-6-yl, 3-aza-tetrahydropyran-2-yl, 3-aza-tetrahydropyran-4-yl, 3-aza-tetrahydropyran-5-yl, 3-aza-tetrahydropyran-6-yl, morpholine-2-yl, morpholine-3-yl, thiophen-2-yl, thiophen-3-yl, isothiazole-3-yl, isothiazole-4-yl, isothiazole-5-yl, thiazole-2-yl, thiazole-4-yl, thiazole-5-yl, thiopyran-2-yl, thiopyran-3-yl, thiopyran-4-yl, 2-azathiopyran-3-yl, 2-azathiopyran-4-yl, 2-azathiopyran-5-yl, 2-azathiopyran-6-yl, 3-azathiopyran-2-yl, 3-azathiopyran-4-yl, 3-azathiopyran-5-yl, 3-azathiopyran-6-yl, 4-azathiopyran-2-yl, 4-azathiopyran-3-yl, 4-azathiopyran-5-yl, 4-azathiopyran-6-yl, thiolane-2-yl, thiolane-3-yl, thiane-2-yl, thiane-3-yl, thiane-4-yl, oxazol-2-yl, oxazol-4-yl, oxazol-5-yl, isoxazol-3-yl, isoxazol-4-yl, isoxazol-5-yl, furazan-3-yl, (1,3,4-oxadiazol)-2-yl, (1,3,4-oxadiazol)-5-yl, (1,2,4-oxadiazol)-3-yl, (1,2,4-oxadiazol)-5-yl; and tetrazole-5-yl).

Preferably, R¹² is selected from H, a substituted or unsubstituted C₁-C₆ alkyl group or a substituted or unsubstituted linear or branched C₁-C₆ halogenated alkyl group.

In some embodiments, the present invention provides a PARP7 and/or PARP1 inhibitor compound which comprises a formula selected from any of following general formulae G1 to G28 as defined hereinabove, wherein R¹, R², X⁶ and R¹¹ are as defined herein.

In further typical embodiments, R¹ is selected from a lower (C1 to C6) alkyl, alkoxy or haloalkyl group, a substituted or unsubstituted C3 to C6 cycloalkyl or heterocyclic group and a halogen group, preferably a —CH₃, —CH₂CH₃—, —CH₂F, —CHF₂, —CF₃, —F, —CH₂CF₃, —CN, methoxy, isopropyl or cyclopropyl group.

In further typical embodiments, R² is selected from H, a C1-C3 alkyl group, a C1 to C3 fluoroalkyl group and a halogen group.

In further typical embodiments, R¹¹ is selected from H, a C1-C3 alkyl group and a C1 to C3 haloalkyl group.

In some embodiments, the present invention provides a PARP7 and/or PARP1 inhibitor compound which comprises a formula selected from any of formulae 1 to 32 as defined hereinabove.

### Stereochemistry; Compound Numbering

The compounds of the present invention have been described in detail above in terms of their structures. For the avoidance of doubt, any compounds for use in the invention may comprise compounds or compositions in accordance with their structure as follows:
an isolated enantiomer, or
a mixture of two or more enantiomers, or
a mixture of two or more diastereomers, and/or epimers, or
a racemic mixture, or
one or more tautomers;
of each structure.

The above numbered compounds represent more than one enantiomeric structure which may have PARP7 and/or PARP1 inhibitory activity as a racemic mixture and/or as a separated enantiomer(s). In the examples below, a compound with a suffix “a” (eg 10a) represents an enantiomer eluted as a first fraction when a racemic mixture of the two enantiomers is applied to a Daicel CHIRALPAK chiral chromatography column. In the examples below, a compound with a suffix “b” (eg 10b) represents an enantiomer eluted as a second fraction when a racemic mixture of the two enantiomers is applied to a Daicel CHIRALPAK chiral chromatography column. In the examples below, a compound which bears a suffix “rac” represents a racemic mixture of the enantiomers.

The suffixes cis and trans describe the stereochemistry across ring B, unless otherwise stated. The suffix ‘cis-1’ refers to the first cis isomer to be eluted from a chiral column; ‘cis-2’ refers to the second cis isomer to be eluted, etc.

### Medical Uses

The compounds described herein may be provided for use in medicine. In the context of the present invention, the medicinal use is not especially limited, provided that it is a use which is facilitated by the PARP7 and/or PARP1 inhibitory effect of the compound. Thus, the compounds of the invention may be for use in any disease, condition or disorder that may be prevented, ameliorated or treated using a PARP7 and/or PARP1 inhibitor. Typically, this comprises a disease condition and/or a disorder selected from: a cancer, an infectious disease, a central nervous system disease or disorder, and a pain condition.

When the disease, condition or disorder is a cancer, it is not especially limited, provided that the cancer is one which may be treated, prevented or ameliorated by using a PARP7 and/or PARP1 inhibitor. Thus the cancer may be a cancer selected from: a solid or liquid tumour including cancer of the eye, brain (such as gliomas, glioblastomas, medullablastomas, craniopharyngioma, ependymoma, and astrocytoma), spinal cord, kidney, mouth, lip, throat, oral cavity, nasal cavity, small intestine, colon, parathyroid gland, gall bladder, head and neck, breast, bone, bile duct, cervix, heart, hypopharyngeal gland, lung, bronchus, liver, skin, ureter, urethra, testicles, vagina, anus, laryngeal gland, ovary, thyroid, oesophagus, nasopharyngeal gland, pituitary gland, salivary gland, prostate, pancreas, adrenal glands; an endometrial cancer, oral cancer, melanoma, neuroblastoma, gastric cancer, an angiomatosis, a hemangioblastoma, a pheochromocytoma, a pancreatic cyst, a renal cell carcinoma, Wilms' tumour, squamous cell carcinoma, sarcoma, osteosarcoma, Kaposi sarcoma, rhabdomyosarcoma, hepatocellular carcinoma, PTEN Hamartoma-Tumor Syndromes (PHTS) (such as Lhermitte-Duclos disease, Cowden syndrome, Proteus syndrome, and Proteus-like syndrome), leukaemias and lymphomas (such as acute lymphoblastic leukaemia, chronic lymphocytic leukaemia, acute myelogenous leukaemia, chronic myelogenous leukaemia, hairy cell leukaemia, T-cell prolymphocytic leukaemia (T-PLL), large granular lymphocytic leukaemia, adult T-cell leukaemia, juvenile myelomonocytic leukaemia, Hodgkin lymphoma, non-Hodgkin lymphoma, mantle lymphoma, follicular lymphoma, primary effusion lymphoma, AIDS-related lymphoma, diffuse B cell lymphoma, Burkitt lymphoma, and cutaneous T-cell lymphoma), nasopharyngeal and gastrointestinal cancers, preferably wherein the cancer is a cancer selected from oesophageal, head and neck, non-small cell lung cancer, squamous cell cancer of the lung, breast, acute myeloid leukaemia (AML), a small-cell lung cancer, a melanoma, an ovarian cancer, a colorectal cancer, a pancreatic cancer, an endometrial cancer, and a skin papilloma.

In some cases the cancer is deficient in a DNA damage response repair pathway, such as Homologous Recombination dependent DNA Double Strand Break DNA repair activity.

In some cases, the cancer is deficient in BRCA1 and/or BRCA2 function.

Advantageously, the compounds are both PARP7 and PARP1 inhibitors. When such dual inhibitors are used in medicine, particularly in the treatment of cancer, more than one mode of inhibitory action may be exhibited. This enables the modulation of multiple mechanisms controlling tumour development in cancer. These include direct tumour-intrinsic growth arrest and immune system activation in the tumour microenvironment via PARP7 and direct cancer cell killing in tumours harbouring loss-of-function mutations in DNA damage repair pathways via PARP1. The DNA damage promoted via PARP1 inhibition in DNA damage response impaired tumours may also result in increased levels of aberrant double-stranded DNA (dsDNA) in the cytosol. This aberrant cytosolic nucleic acid may enhance signalling through the cGAS-STING pathway working in combination with PARP7 inhibition to augment the tumour-intrinsic induction of type I interferons and subsequent immune system activation in the tumour microenvironment.

When the disease is an infectious disease, it is not especially limited, provided that the disease is one which may be treated, prevented or ameliorated by using a PARP7 inhibitor. However, typically the infectious disease is selected from a bacterial infection and a viral infection, preferably a respiratory infection, immune system infection, gut infection and sepsis. Such viral respiratory infections include influenza and coronavirus infections, particularly influenza A and SARS-CoV-2 infections.

When the disease, condition or disorder is a central nervous system disease, condition or disorder, it is not especially limited, provided that the disease, condition or disorder is one which may be treated, prevented or ameliorated by using a PARP7 inhibitor. However, the central nervous system disease, condition or disorder is typically selected from amyotrophic lateral sclerosis (AML), Huntington's disease, Alzheimer's disease, pain, a psychiatric disorder, multiple sclerosis, Parkinson's disease, and HIV related neurocognitive decline.

When the disease, condition or disorder is a pain condition it is not especially limited, provided that the condition is one which may be treated, prevented or ameliorated by using a PARP7 inhibitor. Typically, the pain condition is nociceptive pain or neuropathic pain and may be a chronic pain condition such as cancer-associated pain and peripheral neuropathy.

### Pharmaceutical Compositions

The present invention also provides a pharmaceutical composition comprising a compound as defined above. Whilst the pharmaceutical composition is not especially limited, typically the composition further comprises a pharmaceutically acceptable additive and/or excipient. In the pharmaceutical composition, the compound as defined above may be present in the form described above, but may alternatively be in a form suitable for improving bioavailability, solubility, and/or activity, and/or may be in a form suitable for improving formulation. Thus, the compound may be in the form of a pharmaceutically acceptable salt, hydrate, acid, ester, or other alternative suitable form. Typically, the composition is for treating a disease, condition or disorder as defined above. In some instances, the compound may be present in the composition as a pharmaceutically acceptable salt, or other alternative form of the compound, in order to ameliorate pharmaceutical formulation.

In some embodiments the pharmaceutical composition is a composition for treating a cancer, further comprising a further agent for treating cancer. The further agent for treating cancer is not especially limited, provided that it affords some utility for cancer treatment. However, typically the further agent for treating cancer is selected from anti-microtubule agents, platinum coordination complexes, alkylating agents, antibiotic agents, topoisomerase II inhibitors, antimetabolites, topoisomerase I inhibitors, senolytic agents, hormones and hormone analogues, signal transduction pathway inhibitors, DNA damage repair pathway inhibitors, anti-androgen therapies, antibody-drug conjugates (ADCs), non-receptor tyrosine kinase angiogenesis inhibitors, immunotherapeutic agents (such as an anti-tumour vaccine, an oncolytic virus, an immune stimulatory antibody such as anti-CTLA4, anti-PD1, anti-PDL-1, anti-OX40, anti-41BB, anti-CD27, anti-CD40, anti-LAG3, anti-TIM3, and anti-GITR, a novel adjuvant, a peptide, a cytokine, a chimeric antigen receptor T cell therapy (CAR-T), a small molecule immune modulator such as an IDO or TDO inhibitor or a pattern recognition receptor agonist such as a STING, TLR-9 or RIG-1 Helicase agonist, tumour microenvironment modulators), anti-angiogenic agents, receptor tyrosine kinase inhibitors, cell growth inhibitors such as Ras and Raf inhibitors, proapoptotic agents and cell cycle signalling inhibitors.

### Pharmaceutical Kits

In still further embodiments the invention provides a pharmaceutical kit for treating a cancer, which pharmaceutical kit comprises:
(a) a compound as defined above; and
(b) a further agent for treating cancer; preferably wherein the further agent for treating cancer is selected from anti-microtubule agents, platinum coordination complexes, alkylating agents, antibiotic agents, topoisomerase II inhibitors, antimetabolites, topoisomerase I inhibitors, senolytic agents, hormones and hormone analogues, signal transduction pathway inhibitors, DNA damage repair pathway inhibitors, non-receptor tyrosine kinase angiogenesis inhibitors, anti-androgen therapies, antibody-drug conjugates, immunotherapeutic agents (such as an anti-tumour vaccine, an oncolytic virus, an immune stimulatory antibody such as anti-CTLA4, anti-PD1, anti-PDL-1, anti-OX40, anti-41BB, anti-CD27, anti-CD40, anti-LAG3, anti-TIM3, and anti-GITR, a novel adjuvant, a peptide, a cytokine, a chimeric antigen receptor T cell therapy (CAR-T), a small molecule immune modulator such as a pattern recognition receptor agonist such as a STING, TLR-9 or RIG-1 Helicase agonist, tumour microenvironment modulators, and anti-angiogenic agents), receptor tyrosine kinase inhibitors, cell growth inhibitors such as Ras and Raf inhibitors, proapoptotic agents and cell cycle signalling inhibitors;
wherein the compound and the further agent are suitable for administration simultaneously, sequentially or separately.

### Methods of Treatment

Further provided by the invention is a method of treating a disease and/or a condition and/or a disorder, which method comprises administering to a patient (or subject) a compound, or a composition, or a kit as defined above. The method is typically a method for treating any disease condition or disorder mentioned herein. In typical embodiments, the method is a method for treating a cancer. Preferably such a method comprises administering to a patient (or subject) a compound or a composition as defined above and a further agent for treating cancer as defined above. The compound or composition and the further agent may administered simultaneously, sequentially or separately, depending upon the agents and patients involved, and the type of cancer indicated.

Typically, in all embodiments of the invention, both above and below, the patient (or subject) is an animal, typically a mammal, including canines and felines, and more typically a human.

### Methods of Synthesis

Further provided by the invention is a method of synthesis of a compound as defined above, which method comprises conducting a reaction between (i) a first reactant comprising ring A and ring B and (ii) a second reactant comprising group L, so as to form the PARP7 and/or PARP1 inhibitor compound.

Preferably, the second reactant comprises an L group precursor bearing a reactive group, which method comprises joining the N atom of ring B to the L group precursor, for example through alkylation reaction or Michael addition.

Typically, the reaction between the first reactant and the second reactant forms an intermediate and (iii) a third reactant comprising ring C and ring D is reacted with the intermediate to form the PARP7 and/or PARP1 inhibitor compound.

Preferably, the nitrogen of ring A bears a protecting group in place of H, which protecting group is removed so as to form the PARP7 and/or PARP1 inhibitor compound, typically after the above reactions have been completed.

The skilled person may select the reaction conditions, with reference to known synthesis techniques depending on the appropriate starting materials. In some embodiments, the method comprises one or more additional substitution steps. Exemplary syntheses are shown in the Examples herein.

### General Definitions

Typically, the above formulae (and all formulae herein) are shown in non-stereoisomeric form. For the avoidance of doubt, throughout the present disclosure a single formula is intended to represent all possible stereoisomers of a particular structure, including all possible isolated enantiomers corresponding to the formula, all possible mixtures of enantiomers corresponding to the formula, all possible mixtures of diastereomers corresponding to the formula, all possible mixtures of epimers corresponding to the formula and all possible racemic mixtures corresponding to the formula. In addition to this, the above formulae (and all formulae herein) are intended to represent all tautomeric forms equivalent to the corresponding formula.

The term “comprises” as used throughout the description and claims herein means “includes or consists of”. The term denotes the inclusion of at least the features following the term and does not exclude the inclusion of other features which have not been explicitly mentioned. The term may also denote an entity which consists only of the features following the term.

Where it is said that a compound “comprises” a certain formula, it may equivalently be said that the compound “is according to” that formula, or “has” that formula.

Where it is said that an organic group is “substituted or unsubstituted”, the unsubstituted alternative is preferred in the absence of an explicit statement to the contrary.

Where it is said that an R group “corresponds to” an X group, it is meant that the R group is, when present, attached to that X group. For example, in the formula immediately below R² corresponds to X^{A}, and R¹¹ corresponds to X⁶:

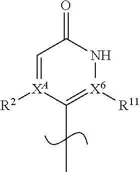

When X^{A} is N, the corresponding R group (i.e., R²) is absent.

### Examples

The invention will now be described in more detail, by way of example only, with reference to the following specific embodiments.

### Exemplary Syntheses of Compounds of the Invention

The compounds of the invention may be synthesised using readily available starting materials and known reactions. Exemplary syntheses of various compounds are shown below:

### 1 Synthesis of 6-(1-(3-oxo-3-(4-(5-(trifluoromethyl)pyrimidin-2-yl)piperazin-1-yl)propyl)piperidin-3-yl)-4-(trifluoromethyl)pyridazin-3(2H)-one (Compound 1)

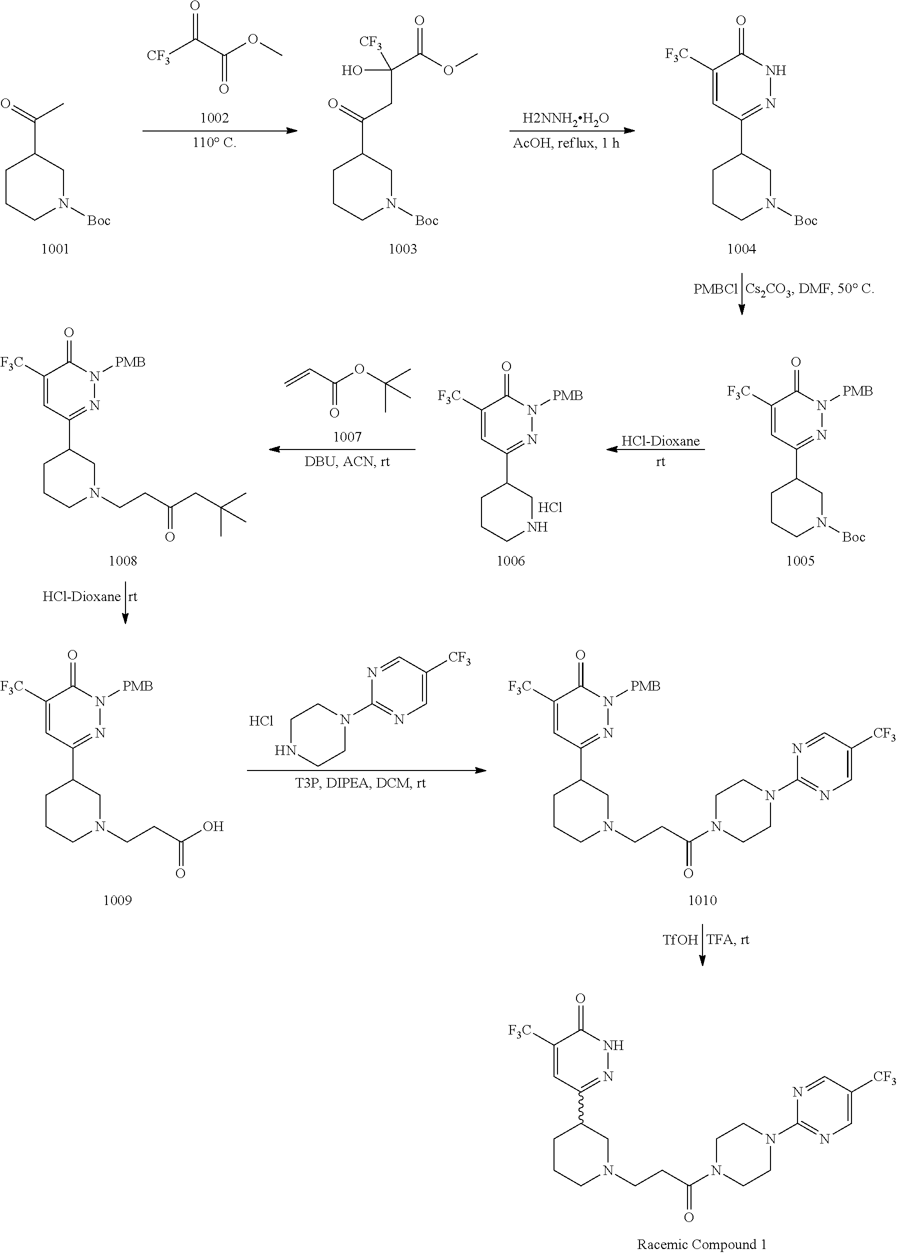

### Preparation of tert-butyl 3-(4,4,4-trifluoro-3-hydroxy-3-(methoxycarbonyl)butanoyl) piperidine-1-carboxylate (1003)

To tert-butyl 3-acetylpiperidine-1-carboxylate 1001 (700 mg, 3.1 mmol) at room temperature was added methyl 3,3,3-trifluoro-2-oxopropanoate 1002 (4.8 g, 31 mmol). The reaction mixture was stirred at 110° C. for 48 h under N₂. The reaction solution was concentrated under reduced pressure. The residue was purified by silica gel column (eluting with PE/EtOAc=90:10 to 70:30) to obtain tert-butyl 3-(4,4,4-trifluoro-3-hydroxy-3-(methoxycarbonyl)butanoyl) piperidine-1-carboxylate 1003 (500 mg, 70% purity, 29% yield) as yellow oil.

LCMS (ESI) calcd for C₁₆H₂₄F₃NO₆ [M-55]⁺ m/z 328.16, found 327.95.

### Preparation of tert-butyl 3-(6-oxo-5-(trifluoromethyl)-1,6-dihydropyridazin-3-yl)piperidine-1-carboxylate (1004)

To a solution of tert-butyl 3-(4,4,4-trifluoro-3-hydroxy-3-(methoxycarbonyl)butanoyl) piperidine-1-carboxylate 1003 (1 g, 0.0026 mol) in AcOH (10 mL) was added H₂NNH₂·H₂O (80% wt, 1.30 g, 0.032 mol) at room temperature. The mixture was kept stirring at 80° C. for 1 h. The resulting mixture was concentrated under reduced pressure. The residue was purified by flash chromatography (eluting with PE/EtOAc=100:0 to 80:20) to give tert-butyl 3-(6-oxo-5-(trifluoromethyl)-1,6-dihydropyridazin-3-yl)piperidine-1-carboxylate 1004 (0.45 g, 80% purity, 38% yield) as a yellow oil.

LCMS (ESI) calcd for C₁₅H₂₀F₃N₃O₃[M+H]⁺ m/z 348.15, found 348.20.

### Preparation of tert-butyl 3-(1-(4-methoxybenzyl)-6-oxo-5-(trifluoromethyl)-1,6-dihydropyridazin-3-yl)piperidine-1-carboxylate (1005)

To a solution of tert-butyl tert-butyl 3-(6-oxo-5-(trifluoromethyl)-1,6-dihydropyridazin-3-yl)piperidine-1-carboxylate 1004 (450 mg, 1.29 mmol) in DMF (15 mL) were added Cs₂CO₃ (2.53 g, 3.88 mmol) and PMBCl (404 mg, 2.58 mmol) at room temperature successively. The mixture was stirred at 50° C. for 1 h. The resulting mixture was diluted with water and extracted with EtOAc (20 mL×3). The combined organic layer was dried over Na₂SO₄ and concentrated under reduced pressure. The residue was purified by flash chromatography (eluting with PE/EtOAc=100:0 to 50:50) to give tert-butyl 3-(1-(4-methoxybenzyl)-6-oxo-5-(trifluoromethyl)-1,6-dihydropyridazin-3-yl)piperidine-1-carboxylate 1005 (230 mg, 90% purity, 34% yield) as a yellow oil.

LCMS (ESI) calcd for C₂₃H₂₈F₃N₃O₄[M-55]⁺ m/z 412.20, found 412.05.

### Preparation of 2-(4-methoxybenzyl)-6-(piperidin-3-yl)-4-(trifluoromethyl)pyridazin-3(2H)-one hydrochloride (1006)

A solution of tert-butyl 3-(1-(4-methoxybenzyl)-6-oxo-5-(trifluoromethyl)-1,6-dihydropyridazin-3-yl)piperidine-1-carboxylate 1005 (630 mg, 1.34 mmol) in 4 M HCl/dioxane (10 mL) was stirred at rt for 0.5 h. The reaction mixture was concentrated under reduced pressure to afford 2-(4-methoxybenzyl)-6-(piperidin-3-yl)-4-(trifluoromethyl)pyridazin-3(2H)-one hydrochloride 1006 (400 mg, 90% purity, 72% yield) as a yellow solid.

LCMS (ESI) calcd for C₁₈H₂₀F₃N₃O₂[M+H]⁺ m/z 368.15, found 368.05.

### Preparation of tert-butyl 3-(3-(1-(4-methoxybenzyl)-6-oxo-5-(trifluoromethyl)-1,6-dihydropyridazin-3-yl)piperidin-1-yl)propanoate (1008)

To a solution of 2-(4-methoxybenzyl)-6-(piperidin-3-yl)-4-(trifluoromethyl)pyridazin-3(2H)-one hydrochloride 1006 (380 mg, 1.03 mmol) in ACN (30 mL) at room temperature were added tert-butyl acrylate 1007 (400 mg, 3.20 mmol) and DBU (80 mg, 0.52 mmol). The reaction mixture was stirred at rt for 18 h. The reaction solution was quenched with water and extracted with EtOAc (20 mL×3). The organic phase was concentrated under reduced pressure and the residue was purified by flash chromatography (eluting with PE/EtOAc=100:0 to 70:30) to give tert-butyl 3-(3-(1-(4-methoxybenzyl)-6-oxo-5-(trifluoromethyl)-1,6-dihydropyridazin-3-yl)piperidin-1-yl)propanoate 1008 (400 mg, 80% purity, 62% yield) as a yellow oil.

LCMS (ESI) calcd for C₂₅H₃₂F₃N₃O₄[M+H]⁺ m/z 496.23, found 496.30.

### Preparation of 3-(3-(1-(4-methoxybenzyl)-6-oxo-5-(trifluoromethyl)-1,6-dihydropyridazin-3-yl)piperidin-1-yl)propanoic acid (1009)

A solution of tert-butyl 3-(3-(1-(4-methoxybenzyl)-6-oxo-5-(trifluoromethyl)-1,6-dihydropyridazin-3-yl)piperidin-1-yl)propanoate 1008 (400 mg, 0.80 mmol) in HCl-dioxane (4 M, 15 mL) was stirred at room temperature for 2 h under N₂ atmosphere. The reaction solution was dried under reduced pressure and purified on a Biotage Isolera One (C18 column, eluting with 60% to 90% MeCN/H₂O containing 0.1% formic acid) to obtain 3-(3-(1-(4-methoxybenzyl)-6-oxo-5-(trifluoromethyl)-1,6-dihydropyridazin-3-yl)piperidin-1-yl)propanoic acid 1009 (250 mg, 90% purity, 63% yield) as white solid.

L CMS (ESI) calcd for C₂₁H₂₄F₃N₃O₄[M+H]⁺ m/z 440.17, found 440.15.

### Preparation of 2-(4-methoxybenzyl)-6-(1-(3-oxo-3-(4-(5-(trifluoromethyl)pyrimidin-2-yl)piperazin-1-yl)propyl)piperidin-3-yl)-4-(trifluoromethyl)pyridazin-3(2H)-one (1010)

To a solution of 3-(3-(1-(4-methoxybenzyl)-6-oxo-5-(trifluoromethyl)-1,6-dihydropyridazin-3-yl)piperidin-1-yl)propanoic acid 1009 (250 mg, 0.57 mmol) in DCM (20 mL) were added 2-(piperazin-1-yl)-5-(trifluoromethyl)pyrimidine hydrochloride (183 mg, 0.68 mmol), T3P (50% wt in EtOAc, 724 mg, 1.20 mmol), DIPEA (220 mg, 1.70 mmol) at room temperature successively. The mixture was kept stirring at room temperature for 1 h. The resulting mixture was diluted with water and extracted with DCM (20 mL×3). The combined organic layer was dried over Na₂SO₄ and concentrated under reduced pressure. The residue was purified by flash chromatography (eluting with DCM/MeOH=100:0 to 97:3) to give 2-(4-methoxybenzyl)-6-(1-(3-oxo-3-(4-(5-(trifluoromethyl)pyrimidin-2-yl)piperazin-1-yl)propyl)piperidin-3-yl)-4-(trifluoromethyl)pyridazin-3(2H)-one 1010 (300 mg, 90% purity, 72% yield) as a white solid.

LCMS (ESI) calcd for C₃₀H₃₃F₆N₇O₃[M+H]⁺ m/z 654.25, found 654.30.

### Preparation of 6-(1-(3-oxo-3-(4-(5-(trifluoromethyl)pyrimidin-2-yl)piperazin-1-yl)propyl)piperidin-3-yl)-4-(trifluoromethyl)pyridazin-3(2H)-one (racemic Compound 1)

To a solution of 2-(4-methoxybenzyl)-6-(1-(3-oxo-3-(4-(5-(trifluoromethyl)pyrimidin-2-yl)piperazin-1-yl)propyl)piperidin-3-yl)-4-(trifluoromethyl)pyridazin-3(2H)-one 1010 (300 mg, 0.46 mmol) in TFA (10 mL) was added TfOH (689 mg, 4.6 mmol) at room temperature. The reaction mixture was stirred at room temperature for 0.5 h. The reaction solution pH was adjusted to 7-8 with saturated aqueous NaHCO₃ at 0° C. The solution was extracted with EtOAc (10 mL×3). The combined organic phases were concentrated and purified by flash chromatography (eluting with DCM/MeOH=100:0 to 97:3) to obtain 6-(1-(3-oxo-3-(4-(5-(trifluoromethyl)pyrimidin-2-yl)piperazin-1-yl)propyl)piperidin-3-yl)-4-(trifluoromethyl)pyridazin-3(2H)-one racemic Compound 1 (150 mg, 99% purity, 61% yield) as a white solid.

LCMS (ESI) calcd for C₂₂H₂₅F₆N₇O₂[M+H]⁺ m/z 534.20, found 534.15.

### Chiral resolution of 6-(1-(3-oxo-3-(4-(5-(trifluoromethyl)pyrimidin-2-yl)piperazin-1-yl)propyl)piperidin-3-yl)-4-(trifluoromethyl)pyridazin-3(2H)-one (racemic Compound 1)

Racemic Compound 1 was separated by SFC (Column: Daicel Chiralpak AD-H SFC, 250 mm×20 mm I.D., 5 μmm; Mobile phase: CO₂/MeOH [0.1% NH₃]=65/35) and concentrated under reduced pressure to afford the first fraction as Compound 1a (37.3 mg, 99% purity, 100% ee, white solid) and the second fraction as Compound 1b (41.7 mg, 99% purity, 100% ee, white solid).

### Compound 1a

¹H NMR (400 MHz, DMSO-d₆, ppm) δ: 13.53 (s, 1H), 8.73 (s, 2H), 7.96 (s, 1H), 3.89-3.78 (m, 4H), 3.60-3.53 (m, 4H), 3.03-2.96 (m, 1H), 2.85-2.79 (s, 2H), 2.63-2.54 (m, 4H), 2.11-1.96 (m, 2H), 1.90-1.82 (m, 1H), 1.72-1.65 (m, 1H), 1.58-1.36 (m, 2H).

LCMS (ESI) calcd for C₂₂H₂₅F₆N₇O₂[M+H]⁺ m/z 534.20, found 534.25.

### Compound 1b

¹H NMR (400 MHz, DMSO-d₆, ppm) δ: 13.53 (s, 1H), 8.73 (s, 2H), 7.96 (s, 1H), 3.89-3.78 (m, 4H), 3.61-3.53 (m, 4H), 3.03-2.95 (m, 1H), 2.87-2.77 (m, 2H), 2.64-2.54 (m, 4H), 2.13-1.97 (m, 2H), 1.91-1.82 (m, 1H), 1.72-1.63 (m, 1H), 1.58-1.34 (m, 2H).

LCMS (ESI) calcd for C₂₂H₂₅F₆N₇O₂[M+H]⁺ m/z 534.20, found 534.30.

### 2 Synthesis of racemic 6-(1-(3-oxo-3-(4-(5-(trifluoromethyl)pyrimidin-2-yl)piperazin-1-yl)propyl)pyrrolidin-3-yl)-4-(trifluoromethyl)pyridazin-3(2H)-one (Compound 5rac)

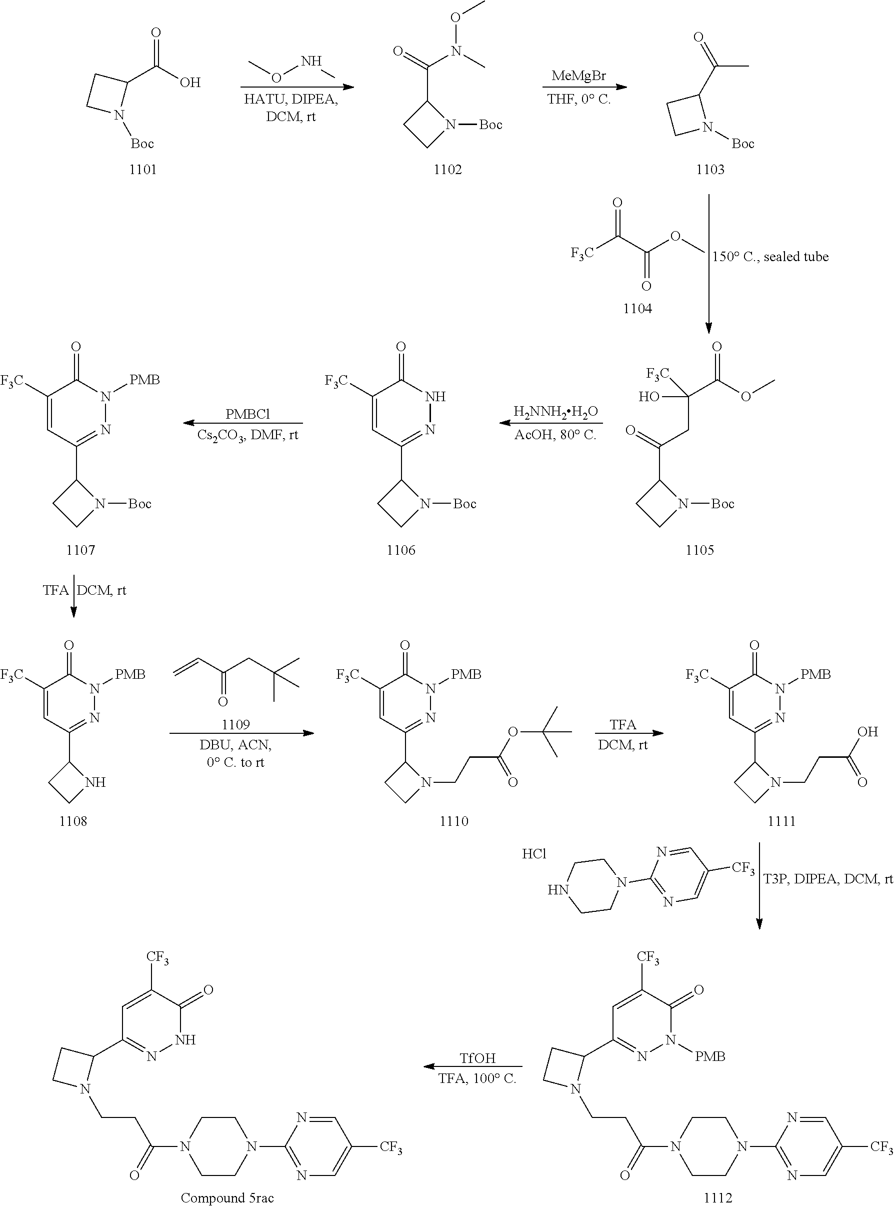

### Preparation of tert-butyl 2-(methoxy(methyl)carbamoyl)azetidine-1-carboxylate (1102)

To a solution of 1-(tert-butoxycarbonyl)azetidine-2-carboxylic acid 1101 (2 g, 0.0099 mol) and N,O-dimethylhydroxylamine hydrochloride (1.93 g, 0.0198 mol) in DCM (50 mL) were added DIPEA (6.4 g, 0.0495 mol) and HATU (3.76 g, 0.0099 mol) at rt. The mixture was stirred at rt for an additional 3 h. The resulting mixture was concentrated under reduced pressure. The residue was purified by flash chromatography (eluting with EtOAc/PE, 10% to 30%) to give tert-butyl 2-(methoxy(methyl)carbamoyl)azetidine-1-carboxylate 1102 (2 g, 80% purity, 65% yield) as a yellow oil.

LCMS (ESI) calcd for C₁₁H₂₀N₂O₄[M+H]⁺ m/z 245.15, found 245.00.

### Preparation of tert-butyl 2-acetylazetidine-1-carboxylate (1103)

To a solution of tert-butyl 2-(methoxy(methyl)carbamoyl)azetidine-1-carboxylate 1102 (2.0 g, 0.0055 mol) in THF (50 mL) was added MeMgBr (3 M in THF, 5.5 mL, 0.0165 mmol) at 0° C. and stirred at 0° C. for additional 30 min. The mixture was quenched with saturated NH₄Cl solution and then extracted with EtOAc (200 mL×3). The combined organic layers were concentrated under reduced pressure. The residue was purified by flash chromatography (eluting with EtOAc/PE, 30% to 60%) to obtain tert-butyl 2-acetylazetidine-1-carboxylate 1103 (1.4 g, 70% purity, 89% yield) as a yellow oil.

LCMS (ESI) calcd for C₁₀H₁₇NO₃ [M-56+H]⁺ m/z 144.06, found 144.05.

### Preparation of tert-butyl 2-(4,4,4-trifluoro-3-hydroxy-3-(methoxycarbonyl)butanoyl)azetidine-1-carboxylate (1105)

A mixture of tert-butyl 2-acetylazetidine-1-carboxylate 1103 (1.4 g, 0.007 mol) and methyl 3,3,3-trifluoro-2-oxopropanoate 1104 (1.65 g, 0.0105 mol) was stirred at 150° C. for 1.5 h in a sealed tube. The resulting mixture was concentrated under reduced pressure to give crude tert-butyl 2-(4,4,4-trifluoro-3-hydroxy-3-(methoxycarbonyl)butanoyl)azetidine-1-carboxylate 1105 (0.8 g, 80% purity, 25% yield) as a black solid.

LCMS (ESI) calcd for C₁₄H₂₀F₃NO₆ [M+Na]⁺ m/z 378.11, found 378.10.

### Preparation of tert-butyl 2-(6-oxo-5-(trifluoromethyl)-1,6-dihydropyridazin-3-yl)azetidine-1-carboxylate (1106)

To a solution of tert-butyl 2-(4,4,4-trifluoro-3-hydroxy-3-(methoxycarbonyl)butanoyl)azetidine-1-carboxylate 1105 (800 mg, 2.24 mmol) in AcOH (20 mL) was added H₂NNH₂·H₂O (80% wt, 437 mg, 6.72 mmol) in one portion. The reaction mixture was heated with stirring at 80° C. for 3 h. Most of the solvent was removed by evaporation under reduced pressure. The resulting solution was extracted with EtOAc (50 mL×3). The combined organic layers were washed with brine, dried over Na₂SO₄ and concentrated under reduced pressure. The residue was purified by flash chromatography (eluting with EtOAc/PE, 20% to 40%) to obtain tert-butyl 2-(6-oxo-5-(trifluoromethyl)-1,6-dihydropyridazin-3-yl)azetidine-1-carboxylate 1106 (800 mg, 50% purity, 34% yield) as a yellow oil.

LCMS (ESI) calcd for C₁₃H₁₆F₃N₃O₃[M-56+H]⁺ m/z 264.06, found 263.90.

### Preparation of tert-butyl 2-(1-(4-methoxybenzyl)-6-oxo-5-(trifluoromethyl)-1,6-dihydropyridazin-3-yl)azetidine-1-carboxylate (1107)

To a solution of tert-butyl 2-(6-oxo-5-(trifluoromethyl)-1,6-dihydropyridazin-3-yl)azetidine-1-carboxylate 1106 (800 mg, 2.48 mmol) and Cs₂CO₃ (1.6 mg, 4.97 mmol) in DMF (20 mL) was added PMBCl (775 mg, 4.97 mmol). The mixture was stirred at rt for 1 hours. The reaction mixture was quenched with water and extracted with EtOAc (50 mL×3). The combined organic layers were washed by brine, dried over Na₂SO₄, and concentrated under reduced pressure. The residue was purified by flash chromatography (eluting with EtOAc/PE, 20% to %) to give tert-butyl 2-(1-(4-methoxybenzyl)-6-oxo-5-(trifluoromethyl)-1,6-dihydropyridazin-3-yl)azetidine-1-carboxylate 1107 (500 mg, 90% purity, 65% yield) as a yellow oil.

LCMS (ESI) calcd for C₂₁H₂₄F₃N₃O₄[M-56+H]⁺ m/z 384.11, found 384.05.

### Preparation of 6-(azetidin-2-yl)-2-(4-methoxybenzyl)-4-(trifluoromethyl)pyridazin-3(2H)-one (1108)

To a solution of tert-butyl 2-(1-(4-methoxybenzyl)-6-oxo-5-(trifluoromethyl)-1,6-dihydropyridazin-3-yl)azetidine-1-carboxylate 1107 (500 mg, 1.133 mmol) in DCM (15 mL) was added TFA (5 mL) dropwise at rt. The mixture was stirred at room temperature for 1 hour then concentrated under reduced pressure. The residue was partitioned between EtOAc and 1 M NaOH. The separated organic layer was washed with water, dried over anhydrous Na₂SO₄ and concentrated under vacuum to afford 6-(azetidin-2-yl)-2-(4-methoxybenzyl)-4-(trifluoromethyl)pyridazin-3(2H)-one 1108 (400 mg, 90% purity, 93% yield).

LCMS (ESI) calcd for C₁₆H₁₆F₃N₃O₂[M+Na]⁺ m/z 362.11, found 361.75.

### Preparation of tert-butyl 3-(2-(1-(4-methoxybenzyl)-6-oxo-5-(trifluoromethyl)-1,6-dihydropyridazin-3-yl)azetidin-1-yl)propanoate (1110)

To a solution of 6-(azetidin-2-yl)-2-(4-methoxybenzyl)-4-(trifluoromethyl)pyridazin-3(2H)-one 1108 (400 mg, 1.2 mmol) in ACN (20 mL) was added DBU (715 mg, 4.7 mmol) and tert-butyl acrylate 1109 (603 mg, 4.7 mmol) dropwise at 0° C. The mixture was stirred at rt for 2 hours. The resulting mixture was diluted with water (100 mL) and extracted with EtOAc (50 mL×3). The combined organic phases were washed with brine, dried over sodium sulfate, concentrated, and purified by silica gel column chromatography (eluting with EtOAc/PE, 40% to 60%) to give tert-butyl 3-(2-(1-(4-methoxybenzyl)-6-oxo-5-(trifluoromethyl)-1,6-dihydropyridazin-3-yl)azetidin-1-yl)propanoate 1110 (60 mg, 90% purity, 9% yield) as a yellow oil.

LCMS (ESI) calcd for C₂₃H₂₈F₃N₃O₄[M+H]⁺ m/z 468.21, found 468.20.

### Preparation of 3-(2-(1-(4-methoxybenzyl)-6-oxo-5-(trifluoromethyl)-1,6-dihydropyridazin-3-yl)azetidin-1-yl)propanoic acid (1111)

To a solution of tert-butyl 3-(2-(1-(4-methoxybenzyl)-6-oxo-5-(trifluoromethyl)-1,6-dihydropyridazin-3-yl)azetidin-1-yl)propanoate 1110 (60 mg, 0.128 mmol) in DCM (4 mL) was added TFA (1 mL) dropwise at rt. The mixture was stirred at room temperature for 30 min then concentrated under reduced pressure to afford 3-(2-(1-(4-methoxybenzyl)-6-oxo-5-(trifluoromethyl)-1,6-dihydropyridazin-3-yl)azetidin-1-yl)propanoic acid 1111 (50 mg, 90% purity, 66% yield) as a yellow oil.

LCMS (ESI) calcd for C₁₉H₂₀F₃N₃O₄[M+H]⁺ m/z 412.14, found 412.10.

### Preparation of 2-(4-methoxybenzyl)-6-(1-(3-oxo-3-(4-(5-(trifluoromethyl)pyrimidin-2-yl)piperazin-1-yl)propyl)azetidin-2-yl)-4-(trifluoromethyl)pyridazin-3(2H)-one (1112)

To a solution of 3-(2-(1-(4-methoxybenzyl)-6-oxo-5-(trifluoromethyl)-1,6-dihydropyridazin-3-yl)azetidin-1-yl)propanoic acid 1111 (50 mg, 0.12 mmol) in DCM (5 mL) were added 2-(piperazin-1-yl)-5-(trifluoromethyl)pyrimidine hydrochloride (49 mg, 0.18 mmol), DIPEA (78 mg, 0.61 mmol) and T3P (50% in EtOAc, 154 mg, 0.24 mmol) at room temperature successively. The mixture was kept stirring at room temperature for 1 h. The resulting mixture was diluted with water and extracted with DCM (30 mL×3). The combined organic layers were dried over Na₂SO₄ and concentrated under reduced pressure. The residue was purified by flash chromatography (eluting with MeOH/DCM, 3% to 5%) to give 2-(4-methoxybenzyl)-6-(1-(3-oxo-3-(4-(5-(trifluoromethyl)pyrimidin-2-yl)piperazin-1-yl)propyl)azetidin-2-yl)-4-(trifluoromethyl) pyridazin-3(2H)-one 1112 (40 mg, 90% purity, 47% yield) as a yellow oil.

LCMS (ESI) calcd for C₂₈H₂₉F₆N₇O₃[M+H]⁺ m/z 626.23, found 626.25.

### Preparation of 6-(1-(3-oxo-3-(4-(5-(trifluoromethyl)pyrimidin-2-yl)piperazin-1-yl)propyl)pyrrolidin-3-yl)-4-(trifluoromethyl)pyridazin-3(2H)-one (Compound 5rac)

To a solution of 2-(4-methoxybenzyl)-6-(1-(3-oxo-3-(4-(5-(trifluoromethyl)pyrimidin-2-yl)piperazin-1-yl)propyl)azetidin-2-yl)-4-(trifluoromethyl) pyridazin-3(2H)-one 1112 (40 mg, 0.064 mmol) in TFA (3 mL) was added TfOH (1 mL) dropwise at rt. The reaction mixture was heated at 100° C. for 5 min. The pH of the resulting mixture was adjusted to around 8.0 by progressively adding saturated NaHCO₃ solution at 0° C., and then extracted with DCM (50 mL×3). The combined organic layers were dried over Na₂SO₄ and concentrated under reduced pressure. The residue was purified by C18 column (eluting with 40% to 60% MeCN/H₂O containing 0.1% formic acid) and prep-TLC (MeOH/DCM, 1/20) to give 6-(1-(3-oxo-3-(4-(5-(trifluoromethyl)pyrimidin-2-yl)piperazin-1-yl)propyl)azetidin-2-yl)-4-(trifluoromethyl)pyridazin-3(2H)-one Compound 5rac (5.3 mg, 90% purity, 14% yield) as a white solid.

¹H NMR (400 MHz, DMSO-d₆, ppm) δ: 13.52 (s, 1H), 8.73 (s, 2H), 8.06 (s, 1H), 4.05 (t, J=8.0 Hz, 1H), 3.85-3.74 (m, 4H), 3.56-3.46 (m, 4H), 3.39-3.36 (m, 2H), 2.96-2.88 (m, 1H), 2.82-2.73 (m, 1H), 2.67-2.60 (m, 1H), 2.34-2.32 (m, 1H), 2.24-2.12 (m, 2H).

LCMS (ESI) calcd for C₂₀H₂₁F₆N₇O₂[M+H]⁺ m/z 506.17, found 506.00.

### 3. Synthesis of 4-ethyl-6-(1-(3-oxo-3-(4-(5-(trifluoromethyl)pyrimidin-2-yl)piperazin-1-yl)propyl)piperidin-3-yl)pyridazin-3(2H)-one (Compound 6)

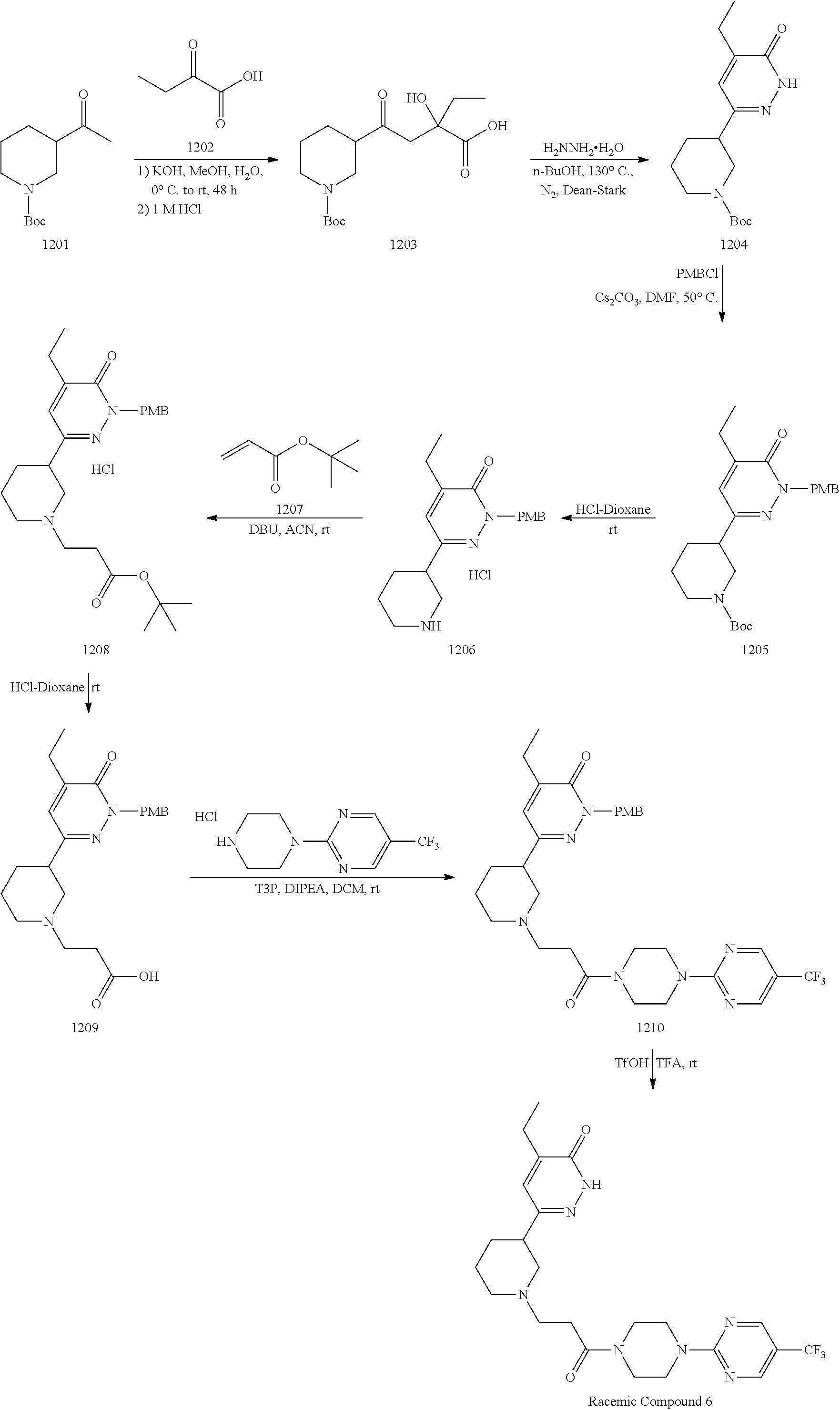

### Preparation of 4-(1-(tert-butoxycarbonyl)piperidin-3-yl)-2-ethyl-2-hydroxy-4-oxobutanoic acid (1203)

To a solution of 2-oxobutanoic acid 1202 (1.53 g, 15.0 mmol) in MeOH (50 mL) was added KOH (5.6 g, 110 mmol) at 0° C. and stirred for additional 1 h. Then tert-butyl 3-acetylpiperidine-1-carboxylate 1201 (2.50 g, 11.0 mmol) was added at 0° C. The mixture was warmed to rt and stirred for 48 h. The resulting mixture was adjusted pH to 2 with 1 M HCl and extracted with DCM (200 mL×3). The combined organic layer was dried over Na₂SO₄ and concentrated under reduced pressure. The residue was purified by gel silica column (eluting with DCM/MeOH=90:10) to give 4-(1-(tert-butoxycarbonyl)piperidin-3-yl)-2-ethyl-2-hydroxy-4-oxobutanoic acid 1203 (2.8 g, 50% purity, 38% yield) as yellow oil.

LCMS (ESI) calcd for C₁₆H₂₇NO₆ [M+Na]⁺ m/z 352.18, found 351.95.

### Preparation of tert-butyl 3-(5-ethyl-6-oxo-1,6-dihydropyridazin-3-yl)piperidine-1-carboxylate (1204)

To a solution of 4-(1-(tert-butoxycarbonyl)piperidin-3-yl)-2-ethyl-2-hydroxy-4-oxobutanoic acid 1203 (2.80 g, 8.47 mmol) in n-BuOH (50 mL) at room temperature was added H₂NNH₂·H₂O (80% wt, 1.06 g, 17.0 mmol). The reaction mixture was stirred at 130° C. for 5 h with a Dean-Stark trap. After cooling to room temperature, the reaction mixture was concentrated and purified by gel silica column (eluting with DCM/MeOH=95:5) to give tert-butyl 3-(5-ethyl-6-oxo-1,6-dihydropyridazin-3-yl)piperidine-1-carboxylate 1204 (600 mg, 90% purity, 20% yield) as white solid.

LCMS (ESI) calcd for C₁₆H₂₅N₃O₃ [M+H]⁺ m/z 308.19, found 308.10.

### Preparation of tert-butyl 3-(5-ethyl-1-(4-methoxybenzyl)-6-oxo-1,6-dihydropyridazin-3-yl)piperidine-1-carboxylate (1205)

To a solution of tert-butyl 3-(5-ethyl-6-oxo-1,6-dihydropyridazin-3-yl)piperidine-1-carboxylate 1204 (600 mg, 1.95 mmol) and Cs₂CO₃ (1.27 g, 3.89 mmol) in DMF (20 mL) was added PMBCl (365 mg, 2.34 mmol) in one portion at rt. The reaction mixture was then stirred at 50° C. for 1 h. After cooling to room temperature, the reaction mixture was poured into cold water and then extracted with EtOAc (50 mL×4). The combined organic layer was concentrated under reduced pressure. The residue was purified by gel silica column (eluting with PE/EtOAc=100:0 to 20:80) to give tert-butyl 3-(5-ethyl-1-(4-methoxybenzyl)-6-oxo-1,6-dihydropyridazin-3-yl)piperidine-1-carboxylate 1205 (550 mg, 90% purity, 59% yield) as yellow oil.

LCMS (ESI) calcd for C₂₄H₃₃N₃O₄ [M+Na]⁺ m/z 450.25, found 450.15.

### Preparation of 4-ethyl-2-(4-methoxybenzyl)-6-(piperidin-3-yl)pyridazin-3(2H)-one hydrochloride (1206)

A solution of tert-butyl 3-(5-ethyl-1-(4-methoxybenzyl)-6-oxo-1,6-dihydropyridazin-3-yl)piperidine-1-carboxylate 1205 (550 mg, 1.28 mmol) in HCl-dioxane (4 M, 22 mL) was stirred at rt for 2 h. The reaction mixture was concentrated to obtain 4-ethyl-2-(4-methoxybenzyl)-6-(piperidin-3-yl)pyridazin-3(2H)-one hydrochloride 1206 (350 mg, 90% purity, 67% yield) as a white solid.

LCMS (ESI) calcd for C₁₉H₂₅N₃O₂ [M+H]⁺ m/z 328.19, found. 328.10

### Preparation of tert-butyl 3-(3-(5-ethyl-1-(4-methoxybenzyl)-6-oxo-1,6-dihydropyridazin-3-yl)piperidin-1-yl)propanoate (1208)

To a solution of 4-ethyl-2-(4-methoxybenzyl)-6-(piperidin-3-yl)pyridazin-3(2H)-one hydrochloride 1206 (350 mg, 0.96 mmol) and tert-butyl acrylate 1207 (246 mg, 1.92 mmol) in ACN (10 mL) was added DBU (292 mg, 1.92 mmol) at rt. The mixture was kept stirring at room temperature for 1 h. The resulting mixture was diluted with water and extracted with DCM (30 mL×3). The combined organic layer was dried over Na₂SO₄ and concentrated under reduced pressure to give tert-butyl 3-(3-(5-ethyl-1-(4-methoxybenzyl)-6-oxo-1,6-dihydropyridazin-3-yl)piperidin-1-yl)propanoate 1208 (285 mg, 90% purity, 58% yield) as a yellow solid.

LCMS (ESI) calcd for C₂₆H₃₇N₃O₄ [M+H]⁺ m/z 456.28, found 456.15.

### Preparation of 3-(3-(5-ethyl-1-(4-methoxybenzyl)-6-oxo-1,6-dihydropyridazin-3-yl)piperidin-1-yl)propanoic acid (1209)

A solution of tert-butyl 3-(3-(5-ethyl-1-(4-methoxybenzyl)-6-oxo-1,6-dihydropyridazin-3-yl)piperidin-1-yl)propanoate 1208 (285 mg, 0.626 mmol) in HCl-dioxane (4 M, 10 mL) was stirred at rt for 2 h. The reaction mixture was concentrated to obtain 3-(3-(5-ethyl-1-(4-methoxybenzyl)-6-oxo-1,6-dihydropyridazin-3-yl)piperidin-1-yl)propanoic acid 1209 (185 mg, 90% purity, 66% yield) as a yellow oil.

LCMS (ESI) calcd for C₂₂H₂₉N₃O₄ [M+H]⁺ m/z 400.22, found 400.15.

### Preparation of 4-ethyl-2-(4-methoxybenzyl)-6-(1-(3-oxo-3-(4-(5-(trifluoromethyl)pyrimidin-2-yl)piperazin-1-yl)propyl)piperidin-3-yl)pyridazin-3(2H)-one (1210)

3-(3-(5-ethyl-1-(4-methoxybenzyl)-6-oxo-1,6-dihydropyridazin-3-yl)piperidin-1-yl)propanoic acid 1209 (185 mg, 0.463 mmol), DIPEA (298 mg, 2.32 mmol) and T3P (50% in EtOAc, 589 mg, 0.926 mmol) were added successively to a solution of 2-(piperazin-1-yl)-5-(trifluoromethyl)pyrimidine hydrochloride (124 mg, 0.463 mmol) in DCM (10 mL) at rt. After stirring at rt for 30 min, the reaction mixture was diluted with water (15 mL) and then extracted with DCM (20 mL×3). The combined organic layers were washed with water and concentrated under reduced pressure. The residue was purified by flash silica chromatography (eluting with MeOH/DCM, 0 to 5%) to 4-ethyl-2-(4-methoxybenzyl)-6-(1-(3-oxo-3-(4-(5-(trifluoromethyl)pyrimidin-2-yl)piperazin-1-yl)propyl)piperidin-3-yl)pyridazin-3(2H)-one 1210 (125 mg, 99% purity, 39% yield) as a white solid.

LCMS (ESI) calcd for C₃₁H₃₈F₃N₇O₃[M+H]⁺ m/z 614.30, found 614.45.

### Preparation of 4-ethyl-6-(1-(3-oxo-3-(4-(5-(trifluoromethyl)pyrimidin-2-yl)piperazin-1-yl)propyl)piperidin-3-yl)pyridazin-3(2H)-one (racemic Compound 6)

TfOH (1 mL) was added to a solution of 4-ethyl-2-(4-methoxybenzyl)-6-(1-(3-oxo-3-(4-(5-(trifluoromethyl)pyrimidin-2-yl)piperazin-1-yl)propyl)piperidin-3-yl)pyridazin-3(2H)-one 1210 (125 mg, 0.204 mmol) in TFA (5 mL). The mixture was stirred at room temperature for 10 min. Then the solution was adjusted to pH 7-8 with saturated aqueous NaHCO₃ at 0° C. and extracted with DCM (50 mL×3). The combined organic layers were washed with brine, dried over Na₂SO₄ and concentrated under reduced pressure. The residue was purified by C18 column (Agela 40 g, mobile phase: ACN—H₂O (0.1% FA), gradient: 25-45) to give 4-ethyl-6-(1-(3-oxo-3-(4-(5-(trifluoromethyl)pyrimidin-2-yl)piperazin-1-yl)propyl)piperidin-3-yl)pyridazin-3(2H)-one racemic Compound 6 (35 mg, 97% purity, 33% yield) as a white solid.

### Chiral resolution of 4-ethyl-6-(1-(3-oxo-3-(4-(5-(trifluoromethyl)pyrimidin-2-yl)piperazin-1-yl)propyl)piperidin-3-yl)pyridazin-3(2H)-one (racemic Compound 6)

Racemic compound 6 was separated by SFC (Column: DAICEL AS-H 20 mm×250 mm I.D., 5 μm; Mobile phase: CO₂/MeOH [0.1% NH₃]=75/25) and concentrated under reduced pressure to afford the first fraction as compound 6a (12.5 mg, 98% purity, 100% ee, light yellow solid) and the second fraction as compound 6b (13.5 mg, 99% purity, 98% ee, light yellow solid).

### Compound 6a

¹H NMR (400 MHz, DMSO-d₆, ppm) δ: 12.68 (s, 1H), 8.81-8.67 (m, 2H), 7.25 (s, 1H), 3.91-3.76 (m, 4H), 3.62-3.51 (m, 4H), 3.01-2.93 (m, 1H), 2.89-2.80 (m, 1H), 2.73-2.66 (m, 1H), 2.65-2.54 (m, 4H), 2.46-2.39 (m, 2H), 2.05-1.93 (m, 2H), 1.88-1.78 (m, 1H), 1.73-1.64 (m, 1H), 1.58-1.46 (m, 1H), 1.42-1.33 (m, 1H), 1.11 (t, J=7.6 Hz, 3H).

LCMS (ESI) calcd for C₂₃H₃₀F₃N₇O₂[M+H]⁺ m/z 494.24, found 494.20.

### Compound 6b

¹H NMR (400 MHz, DMSO-d₆, ppm) δ: 12.68 (s, 1H), 8.78-8.67 (m, 2H), 7.25 (s, 1H), 3.91-3.76 (m, 4H), 3.61-3.51 (m, 4H), 3.03-2.92 (m, 1H), 2.90-2.81 (m, 1H), 2.71-2.65 (m, 1H), 2.65-2.54 (m, 4H), 2.45-2.40 (m, 2H), 2.03-1.95 (m, 2H), 1.88-1.79 (m, 1H), 1.75-1.64 (m, 1H), 1.57-1.47 (m, 1H), 1.42-1.33 (m, 1H), 1.11 (t, J=7.6 Hz, 3H).

LCMS (ESI) calcd for C₂₃H₃₀F₃N₇O₂ [M+H]⁺ m/z 494.24, found 494.20.

### 4. Synthesis of 6-(1-(3-oxo-3-(4-(5-(trifluoromethyl)pyrimidin-2-yl)piperazin-1-yl)propyl)pyrrolidin-2-yl)-4-(trifluoromethyl)pyridazin-3(2H)-one (Compound 18)

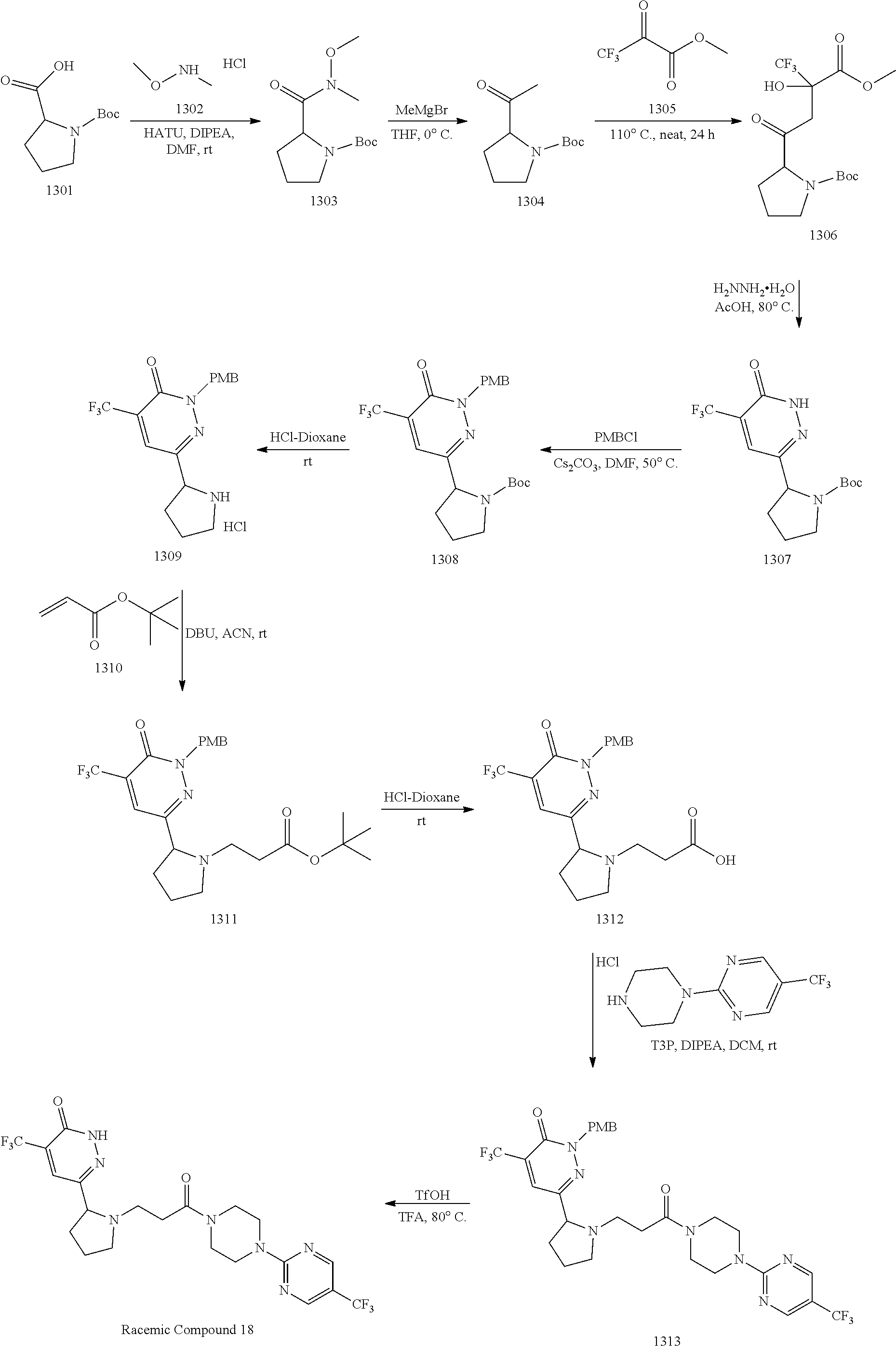

### Preparation of tert-butyl 2-(methoxy(methyl)carbamoyl)pyrrolidine-1-carboxylate (1303)

To a solution of (tert-butoxycarbonyl)proline 1301 (7 g, 32.4 mmol) in DMF (200 mL) were added N,O-dimethylhydroxylamine hydrochloride 1302 (4.7 g, 48.6 mmol), DIPEA (16.7 g, 129.6 mmol) and HATU (24.6 g, 64.8 mmol) at rt successively. The reaction mixture was stirred at rt for 1 h. The reaction mixture was poured into water. The aqueous layer was extracted with EtOAc (1000 mL×3). The combined organic layers were washed with brine (500 mL×3), dried over Na₂SO₄ and concentrated under reduced pressure. The residue was purified by flash column chromatography (eluting with PE/DCM=100:0 to 0:100) to afford tert-butyl 2-(methoxy(methyl)carbamoyl)pyrrolidine-1-carboxylate 1303 (8.5 g, 85% purity, 86% yield) as a colorless oil.

LCMS (ESI) calcd for C₁₂H₂₂N₂O₄ [M+H]⁺ m/z 259.16, found 259.08.

### Preparation of tert-butyl 2-acetylpyrrolidine-1-carboxylate (1304)

To a solution of tert-butyl 2-(methoxy(methyl)carbamoyl)pyrrolidine-1-carboxylate 1303 (8.5 g, 32.8 mmol) in THF (150 mL) was added MeMgBr (33 mL, 98.4 mmol, 3M in THF) at 0° C. under N₂. The reaction mixture was stirred at 0° C. for 1 h. The reaction was quenched with aq. NH₄Cl, the aqueous layer was extracted with EtOAc (500 mL×3). The combined organic layers were washed with brine (200 mL), dried over Na₂SO₄ and concentrated under reduced pressure. The residue was purified by flash column chromatography (eluting with PE/DCM=100:0 to 10:90) to afford tert-butyl 2-acetylpyrrolidine-1-carboxylate 1304 (6.8 g, 75% purity, 72% yield) as a yellow oil.

LCMS (ESI) calcd for C₁₁H₁₉NO₃ [M+Na]⁺ m/z 236.14, found 236.20.

### Preparation of tert-butyl 2-(4,4,4-trifluoro-3-hydroxy-3 (methoxycarbonyl)butanoyl)pyrrolidine-1-carboxylate (1306)

A solution of tert-butyl 2-acetylpyrrolidine-1-carboxylate 1304 (6.8 g, 31.7 mmol) and methyl 3,3,3-trifluoro-2-oxopropanoate 1305 (24.9 g, 158.5 mmol) was stirred at 110° C. for 24 h in a sealed tube. The reaction solution was purified by flash column chromatography (eluting with PE/EtOAc=100:0 to 50:50) to afford tert-butyl 2-(4,4,4-trifluoro-3-hydroxy-3-(methoxycarbonyl)butanoyl)pyrrolidine-1-carboxylate 1306 (8.5 g, 75% purity, 54% yield) as a yellow oil.

LCMS (ESI) calcd for C₁₅H₂₂F₃NO₆ [M-55]⁺ m/z 314.14, found 313.98.

### Preparation of tert-butyl 2-(6-oxo-5-(trifluoromethyl)-1,6-dihydropyridazin-3-yl)pyrrolidine-1-carboxylate (1307)

To a solution of tert-butyl 2-(4,4,4-trifluoro-3-hydroxy-3-(methoxycarbonyl)butanoyl)pyrrolidine-1-carboxylate 1306 (8.5 g, 22.9 mol) in AcOH (150 mL) was added H₂NNH₂·H₂O (80% wt, 4.6 g, 114.5 mmol) at rt. The reaction mixture was stirred at 80° C. for 2 h. The resulting solution was concentrated under reduced pressure and the residue was purified by flash column chromatography (eluting with DCM/MeOH=100:0 to 96:4) to afford tert-butyl 2-(6-oxo-5-(trifluoromethyl)-1,6-dihydropyridazin-3-yl)pyrrolidine-1-carboxylate 1307 (6.8 g, 85% purity, 75% yield) as a yellow oil.

LCMS (ESI) calcd for C₁₄H₁₈F₃N₃O₃[M+H]⁺ m/z 334.13, found 334.00.

### Preparation of tert-butyl 2-(1-(4-methoxybenzyl)-6-oxo-5-(trifluoromethyl)-1,6-dihydropyridazin-3-yl)pyrrolidine-1-carboxylate (1308)

To a solution of tert-butyl 2-(6-oxo-5-(trifluoromethyl)-1,6-dihydropyridazin-3-yl)pyrrolidine-1-carboxylate 1307 (5.0 g, 15.0 mmol) in DMF (50 mL) was added PMBCl (4.7 g, 30.0 mmol) and Cs₂CO₃ (14.7 g, 45.0 mmol) successively at rt. The reaction mixture was stirred at 50° C. for 1 h. The reaction mixture was poured into water. The aqueous layer was extracted with EtOAc (150 mL×3). The combined organic layers were washed with brine (150 mL×3), dried over Na₂SO₄ and concentrated under reduced pressure. The residue was purified by flash column chromatography (eluting with PE/EtOAc=100:0 to 60:40) to afford tert-butyl 2-(1-(4-methoxybenzyl)-6-oxo-5-(trifluoromethyl)-1,6-dihydropyridazin-3-yl)pyrrolidine-1-carboxylate 1308 (4.6 g, 90% purity, 60% yield) as a yellow oil.

LCMS (ESI) calcd for C₂₂H₂₆F₃N₃O₄[M+H]⁺ m/z 454.19, found 454.05.

### Preparation of 2-(4-methoxybenzyl)-6-(pyrrolidin-2-yl)-4-(trifluoromethyl)pyridazin-3(2H)-one hydrochloride (1309)

A solution of tert-butyl 2-(1-(4-methoxybenzyl)-6-oxo-5-(trifluoromethyl)-1,6-dihydropyridazin-3-yl)pyrrolidine-1-carboxylate 1308 (4.5 g, 9.9 mmol) in HCl-1,4-dioxane (4 M, 50 mL) was stirred at rt for 1 h. The reaction solution was concentrated under reduced pressure to give 2-(4-methoxybenzyl)-6-(pyrrolidin-2-yl)-4-(trifluoromethyl)pyridazin-3(2H)-one hydrochloride 1309 (3.2 g, 90% purity, 74% yield) as a yellow solid.

LCMS (ESI) calcd for C₁₇H₁₈F₃N₃O₂[M+H]⁺ m/z 354.14, found 354.00.

### Preparation of tert-butyl 3-(2-(1-(4-methoxybenzyl)-6-oxo-5-(trifluoromethyl)-1,6-dihydropyridazin-3-yl)pyrrolidin-1-yl)propanoate (1311)

To a solution of 2-(4-methoxybenzyl)-6-(pyrrolidin-2-yl)-4-(trifluoromethyl)pyridazin-3(2H)-one hydrochloride 1309 (550 mg, 1.56 mmol) in ACN (30 mL) was added DBU (474 mg, 3.11 mmol) and tert-butyl acrylate 1310 (513 mg, 3.11 mmol) successively at rt. The reaction mixture was stirred at rt for 16 h. The reaction solution was concentrated under reduced pressure. The residue was purified by flash column chromatography (eluting with PE/EtOAc=100:0 to 70:30) to afford tert-butyl 3-(2-(1-(4-methoxybenzyl)-6-oxo-5-(trifluoromethyl)-1,6-dihydropyridazin-3-yl)pyrrolidin-1-yl)propanoate 1311 (350 mg, 85% purity, 39% yield) as a yellow oil.

LCMS (ESI) calcd for C₂₄H₃₀F₃N₃O₄[M+H]⁺ m/z 482.22, found 482.19.

### Preparation of 3-(2-(1-(4-methoxybenzyl)-6-oxo-5-(trifluoromethyl)-1,6-dihydropyridazin-3-yl)pyrrolidin-1-yl)propanoic acid (1312)

A solution of tert-butyl 3-(2-(1-(4-methoxybenzyl)-6-oxo-5-(trifluoromethyl)-1,6-dihydropyridazin-3-yl)pyrrolidin-1-yl)propanoate 1311(350 mg, 0.73 mmol) in HCl-Dioxane (4 M, 15 mL) was stirred at rt for 2 h. The reaction solution was concentrated under reduced pressure to obtain 3-(2-(1-(4-methoxybenzyl)-6-oxo-5-(trifluoromethyl)-1,6-dihydropyridazin-3-yl)pyrrolidin-1-yl)propanoic acid 1312 (180 mg, 85% purity, 49% yield) as a yellow oil.

LCMS (ESI) calcd for C₂₀H₂₂F₃N₃O₄[M+H]⁺ m/z 426.16, found 425.95.

### Preparation of 2-(4-methoxybenzyl)-6-(1-(3-oxo-3-(4-(5-(trifluoromethyl)pyrimidin-2-yl)piperazin-1-yl)propyl)pyrrolidin-2-yl)-4-(trifluoromethyl)pyridazin-3(2H)-one (1313)

To a solution of 3-(2-(1-(4-methoxybenzyl)-6-oxo-5-(trifluoromethyl)-1,6-dihydropyridazin-3-yl)pyrrolidin-1-yl)propanoic acid 1312 (180 mg, 0.42 mmol) and 2-(piperazin-1-yl)-5-(trifluoromethyl)pyrimidine hydrochloride (171 mg, 0.63 mmol) in DCM (20 mL) were added DIPEA (218 mg, 1.69 mmol) and T3P (50% wt in EtOAc, 538 mg, 0.85 mmol) at rt successively. The reaction mixture was stirred at rt for 1 h. The reaction solution was quenched with water (20 mL) and extracted with DCM (30 mL×2). The organic phase was concentrated and purified by flash column chromatography (eluting with DCM/MeOH=100:0 to 95:5) to afford 2-(4-methoxybenzyl)-6-(1-(3-oxo-3-(4-(5-(trifluoromethyl)pyrimidin-2-yl)piperazin-1-yl)propyl)pyrrolidin-2-yl)-4-(trifluoromethyl)pyridazin-3(2H)-one 1313 (190 mg, 90% purity, 63% yield) as a white solid.

LCMS (ESI) calcd for C₂₉H₃₁F₆N₇O₃[M+H]⁺ m/z 640.24, found 640.28.

### Preparation of 6-(1-(3-oxo-3-(4-(5-(trifluoromethyl)pyrimidin-2-yl)piperazin-1-yl)propyl)pyrrolidin-2-yl)-4-(trifluoromethyl)pyridazin-3(2H)-one (racemic Compound 18)

To a solution of 2-(4-methoxybenzyl)-6-(1-(3-oxo-3-(4-(5-(trifluoromethyl)pyrimidin-2-yl)piperazin-1-yl)propyl)pyrrolidin-2-yl)-4-(trifluoromethyl)pyridazin-3(2H)-one 1313 (190 mg, 0.30 mmol) in TFA (5 mL) was added TfOH (0.3 mL) at rt. The reaction solution was stirred at 80° C. for 15 min. The mixture was adjusted to pH=8-9 with saturated aqueous NaHCO₃ at 0° C., then the aqueous layer was extracted with EtOAc (20 mL×3). The combined organic layers were concentrated under reduced pressure. The residue was purified by flash column chromatography (eluting with DCM/MeOH=100:0 to 94:6) to afford 6-(1-(3-oxo-3-(4-(5-(trifluoromethyl)pyrimidin-2-yl)piperazin-1-yl)propyl)pyrrolidin-2-yl)-4-(trifluoromethyl)pyridazin-3(2H)-one racemic Compound 18 (85 mg, 95% purity, 52% yield) as a white solid.

### Chiral resolution of 6-(1-(3-oxo-3-(4-(5-(trifluoromethyl)pyrimidin-2-yl)piperazin-1-yl)propyl)pyrrolidin-2-yl)-4-(trifluoromethyl)pyridazin-3(2H)-one (racemic Compound 18)

Racemic Compound 18 was separated by SFC (Column: Daicel Chiralpak IH; 20 mm×250 mm I.D., 5 umm; Mobile phase: CO₂/MeOH [0.1% NH₃ (7 M Solution in MeOH)]=70/30) and concentrated under reduced pressure to afford the first fraction as Compound 18a (6.0 mg, 98% purity, 100% ee, white solid) and the second fraction as Compound 18b (8.2 mg, 99% purity, 97% ee, white solid).

### Compound 18a

¹H NMR (400 MHz, DMSO-d₆, ppm) δ: 13.50 (s, 1H), 8.73 (s, 2H), 7.73 (s, 1H), 3.89-3.71 (m, 4H), 3.63-3.42 (m, 4H), 3.39-3.35 (m, 1H), 3.31-3.23 (m, 2H), 2.83-2.71 (m, 1H), 2.44-2.35 (m, 2H), 2.31-2.19 (m, 1H), 2.15-2.02 (m, 1H), 1.92-1.77 (m, 2H), 1.76-1.63 (m, 1H).

LCMS (ESI) calcd for C₂₁H₂₃F₆N₇O₂[M+H]⁺ m/z 520.18, found 520.20.

### Compound 18b

¹H NMR (400 MHz, DMSO-d₆, ppm) δ: 13.51 (s, 1H), 8.73 (s, 2H), 7.74 (s, 1H), 3.87-3.75 (m, 4H), 3.57-3.45 (m, 4H), 3.37 (s, 1H), 3.31-3.23 (m, 2H), 2.81-2.73 (m, 1H), 2.44-2.35 (m, 2H), 2.31-2.22 (m, 1H), 2.15-2.03 (m, 1H), 1.90-1.76 (m, 2H), 1.76-1.64 (m, 1H).

LCMS (ESI) calcd for C₂₁H₂₃F₆N₇O₂[M+H]⁺ m/z 520.18, found 520.25.

### 5. Synthesis of 3-ethyl-5-(1-(3-oxo-3-(4-(5-(trifluoromethyl)pyrimidin-2-yl)piperazin-1-yl)propyl)piperidin-3-yl)pyridin-2(1H)-one (Compound 22)

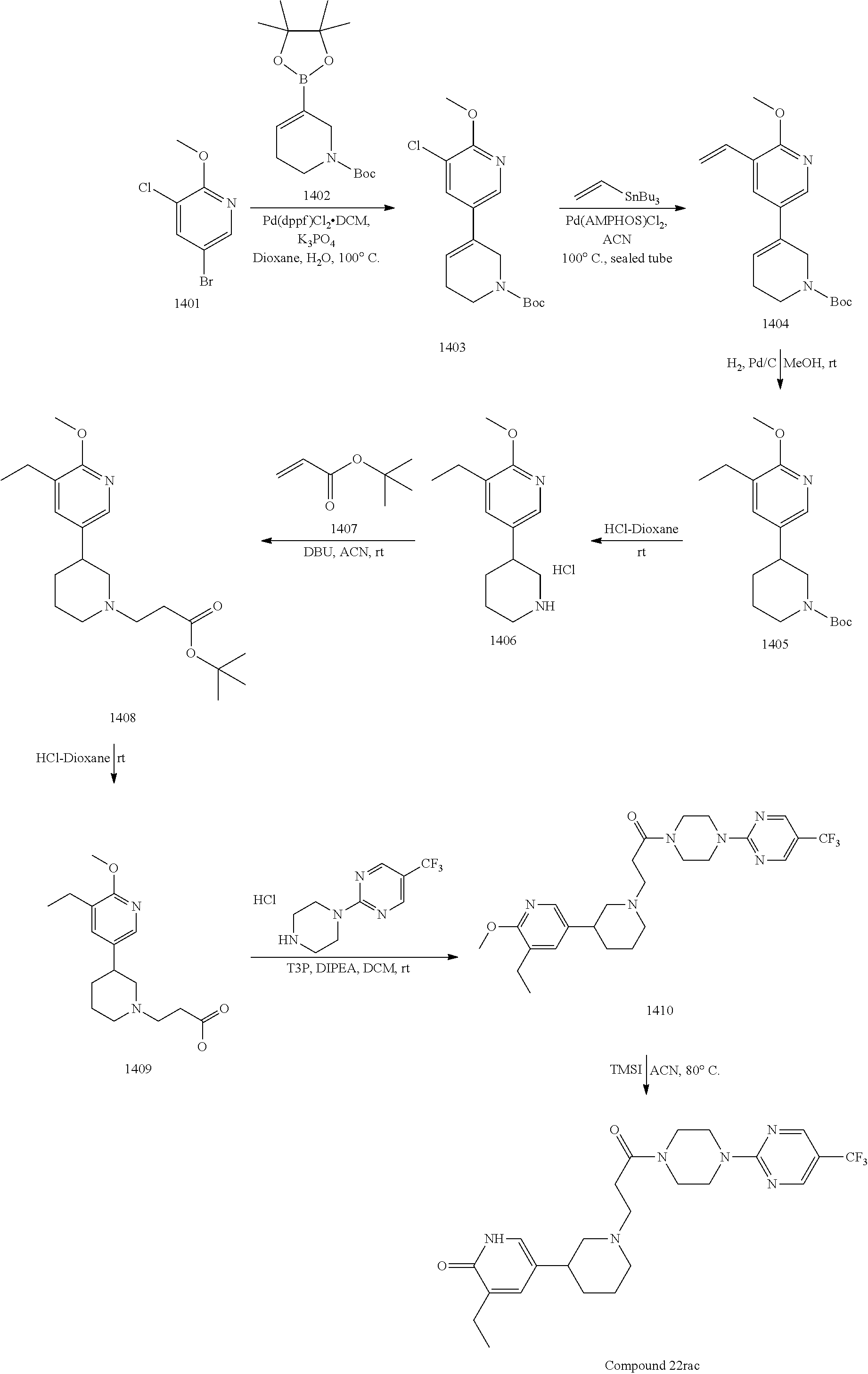

### Preparation of tert-butyl 5′-chloro-6′-methoxy-5,6-dihydro-[3,3′-bipyridine]-1(2H)-carboxylate (1403)

A mixture of 5-bromo-3-chloro-2-methoxypyridine 1401 (2.0 g, 0.009 mol), tert-butyl 5-(4,4,5,5-tetramethyl-1,3,2-dioxaborolan-2-yl)-3,6-dihydropyridine-1(2H)-carboxylate 1402 (3.1 g, 0.010 mol), Pd(dppf)Cl₂-DCM (0.7 g, 0.001 mol) and K₃PO₄ (3.8 g, 0.018 mol) in dioxane:H₂O=3:1 (100 mL) was heated at 100° C. for 2 h. After cooling to rt, the mixture was filtered, and the filtrate was extracted with EtOAc (50 mL×3). The combined organic layer was washed with brine, dried over Na₂SO₄, concentrated, and purified with flash silica chromatography (eluting with EtOAc/PE, 0 to 18%) to obtain tert-butyl 5′-chloro-6′-methoxy-5,6-dihydro-[3,3′-bipyridine]-1(2H)-carboxylate 1403 (1.9 g, 90% purity, 57% yield) as a yellow oil.

LCMS (ESI) calcd for C₁₆H₂₁ClN₂O₃ [M+H]⁺ m/z 325.13, found 325.01.

### Preparation of tert-butyl 6′-methoxy-5′-vinyl-5,6-dihydro-[3,3′-bipyridine]-1(2H)-carboxylate (1404)

A mixture of tert-butyl 5′-chloro-6′-methoxy-5,6-dihydro-[3,3′-bipyridine]-1(2H)-carboxylate 1403 (1.0 g, 0.003 mol), tributyl(vinyl)stannane (2.0 g, 0.006 mol) and Pd(AMPHOS)Cl₂ (220 mg, 0.300 mmol) in ACN (20 mL) was heated at 100° C. for 12 h in a sealed tube. After cooling to rt, the mixture was concentrated and purified with flash silica chromatography (eluting with EtOAc/PE, 0 to 14%) to obtain tert-butyl 6′-methoxy-5′-vinyl-5,6-dihydro-[3,3′-bipyridine]-1(2H)-carboxylate 1404 (800 mg, 76% purity, 61% yield) as a yellow oil.

LCMS (ESI) calcd for C₁₈H₂₄N₂O₃ [M+H]⁺ m/z 317.19, found 317.08.

### Preparation of tert-butyl 3-(5-ethyl-6-methoxypyridin-3-yl)piperidine-1-carboxylate (1405)

A mixture of tert-butyl 6′-methoxy-5′-vinyl-5,6-dihydro-[3,3′-bipyridine]-1(2H)-carboxylate 1404 (800 mg, 2.520 mmol) and Pd/C (800 mg) in MeOH (20 mL) was degassed with H₂ and stirred at rt for 3 h under H₂ atmosphere. The resulting mixture was filtered to remove Pd/C and the filtrate was concentrated to obtain tert-butyl 3-(5-ethyl-6-methoxypyridin-3-yl)piperidine-1-carboxylate 1405 (700 mg, 80% purity, 69% yield) as a yellow oil.

LCMS (ESI) calcd for C₁₈H₂₈N₂O₃[M+H]⁺ m/z 321.22, found 321.10.

### Preparation of 3-ethyl-2-methoxy-5-(piperidin-3-yl)pyridine hydrochloride (1406)

A solution of tert-butyl 3-(5-ethyl-6-methoxypyridin-3-yl)piperidine-1-carboxylate 1405 (700 mg, 2.178 mmol) in HCl-dioxane (10 mL, 4 M) was stirred at rt for 30 min and concentrated to obtain 3-ethyl-2-methoxy-5-(piperidin-3-yl)pyridine hydrochloride 1406 (500 mg, 60% purity, 53% yield) as a yellow oil.

LCMS (ESI) calcd for C₁₃H₂₀N₂O [M+H]⁺ m/z 221.16, found 221.00.

### Preparation of tert-butyl 3-(3-(5-ethyl-6-methoxypyridin-3-yl)piperidin-1-yl)propanoate (1408)

To a solution of 3-ethyl-2-methoxy-5-(piperidin-3-yl)pyridine hydrochloride 1406 (500 mg, 1.947 mmol) in ACN (20 mL) were added tert-butyl acrylate 1407 (499 mg, 3.895 mmol) and DBU (593 mg, 3.895 mmol). The mixture was stirred at rt for 1 h, concentrated and purified with flash silica chromatography (eluting with EtOAc/PE, 0 to 23%) to obtain tert-butyl 3-(3-(5-ethyl-6-methoxypyridin-3-yl)piperidin-1-yl)propanoate 1408 (400 mg, 80% purity, 47% yield) as a yellow oil.

LCMS (ESI) calcd for C₂₀H₃₂N₂O₃ [M+H]⁺ m/z 349.25, found 349.21.

### Preparation of 3-(3-(5-ethyl-6-methoxypyridin-3-yl)piperidin-1-yl)propanoic acid (1409)

A solution of tert-butyl 3-(3-(5-ethyl-6-methoxypyridin-3-yl)piperidin-1-yl)propanoate 1408 (400 mg, 1.148 mmol) in HCl-dioxane (10 mL, 4 mol/L) was stirred at rt for 1 h and concentrated to obtain 3-(3-(5-ethyl-6-methoxypyridin-3-yl)piperidin-1-yl)propanoic acid 1409 (300 mg, 70% purity, 62% yield) as a yellow oil.

LCMS (ESI) calcd for C₁₆H₂₄N₂O₃ [M+H]⁺ m/z 293.19, found 293.00.

### Preparation of 3-(3-(5-ethyl-6-methoxypyridin-3-yl)piperidin-1-y)-1-(4-(5-(trifluoromethyl)pyrimidin-2-yl)piperazin-1-yl)propan-1-one (1410)

To a solution of 3-(3-(5-ethyl-6-methoxypyridin-3-yl)piperidin-1-yl)propanoic acid 1409 (200 mg, 0.684 mmol) in DCM (20 mL) were added 2-(piperazin-1-yl)-5-(trifluoromethyl)pyrimidine hydrochloride (220 mg, 0.821 mmol), DIPEA (353 mg, 2.736 mmol) and T3P (871 mg, 1.368 mmol, 50% in EtOAc) at rt. The mixture was stirred at rt for 30 min, concentrated and purified by flash silica chromatography (eluting with MeOH/DCM, 0 to 6%) to obtain 3-(3-(5-ethyl-6-methoxypyridin-3-yl)piperidin-1-yl)-1-(4-(5-(trifluoromethyl)pyrimidin-2-yl)piperazin-1-yl)propan-1-one 1410 (200 mg, 70% purity, 40% yield) as a colorless oil.

LCMS (ESI) calcd for C₂₅H₃₃F₃N₆O₂[M+H]⁺ m/z 507.27, found 507.30.

### Preparation of 3-ethyl-5-(1-(3-oxo-3-(4-(5-(trifluoromethyl)pyrimidin-2-yl)piperazin-1-yl)propyl)piperidin-3-yl)pyridin-2(1H)-one (Compound 22rac)

TMSI (118 mg, 0.592 mmol) was added to a solution of 3-(3-(5-ethyl-6-methoxypyridin-3-yl)piperidin-1-yl)-1-(4-(5-(trifluoromethyl)pyrimidin-2-yl)piperazin-1-yl)propan-i-one 1410 (100 mg, 0.197 mmol) in ACN (10 mL) at rt. The reaction mixture was heated at 80° C. for 1 h. After cooling to rt, the mixture was washed with water and extracted with DCM (30 mL×3). The combined organic layer was washed with brine, dried over Na₂SO₄, and concentrated under reduced pressure. The residue was purified with prep-HPLC (Column: YMC 5 μm C₁₈ 150×20 mm, mobile phase: ACN—H₂O (0.1% FA), gradient: 25-35-85), flash silica chromatography (eluting with MeOH/DCM, 0 to 10%), prep-TLC (MeOH/DCM=10:1) and SFC (Column: chiralpak-OD-H 250 mm×20 mm I.D., 5 μm; Mobile phase: CO₂/MeOH (0.1% NH₃)=60/40) to obtain 3-ethyl-5-(1-(3-oxo-3-(4-(5-(trifluoromethyl)pyrimidin-2-yl)piperazin-1-yl)propyl)piperidin-3-yl)pyridin-2(1H)-one compound 22rac (2.9 mg, 97% purity, 2% yield) as a white solid.

¹H NMR (400 MHz, DMSO-d₆, ppm) δ: 11.30 (s, 1H), 8.74 (d, J=0.4 Hz, 2H), 7.23 (d, J=2.4 Hz, 1H), 7.05-6.99 (m, 1H), 3.94-3.74 (m, 4H), 3.64-3.50 (m, 4H), 2.93-2.76 (m, 2H), 2.64-2.53 (m, 4H), 2.48-2.43 (m, 1H), 2.37 (q, J=7.4 Hz, 2H), 2.04-1.88 (m, 2H), 1.76-1.63 (m, 2H), 1.58-1.45 (m, 1H), 1.35-1.27 (m, 1H), 1.08 (t, J=7.4 Hz, 3H).

LCMS (ESI) calcd for C₂₄H₃₁F₃N₆O₂[M+H]⁺ m/z 493.25, found 493.00.

### 6. Synthesis of 6-(1-(2-oxo-2-(4-(5-(trifluoromethyl)pyrimidin-2-yl)piperazin-1-yl)ethyl)pyrrolidin-2-yl)-4-(trifluoromethyl)pyridazin-3(2H)-one (Compounds 24a and 24b)

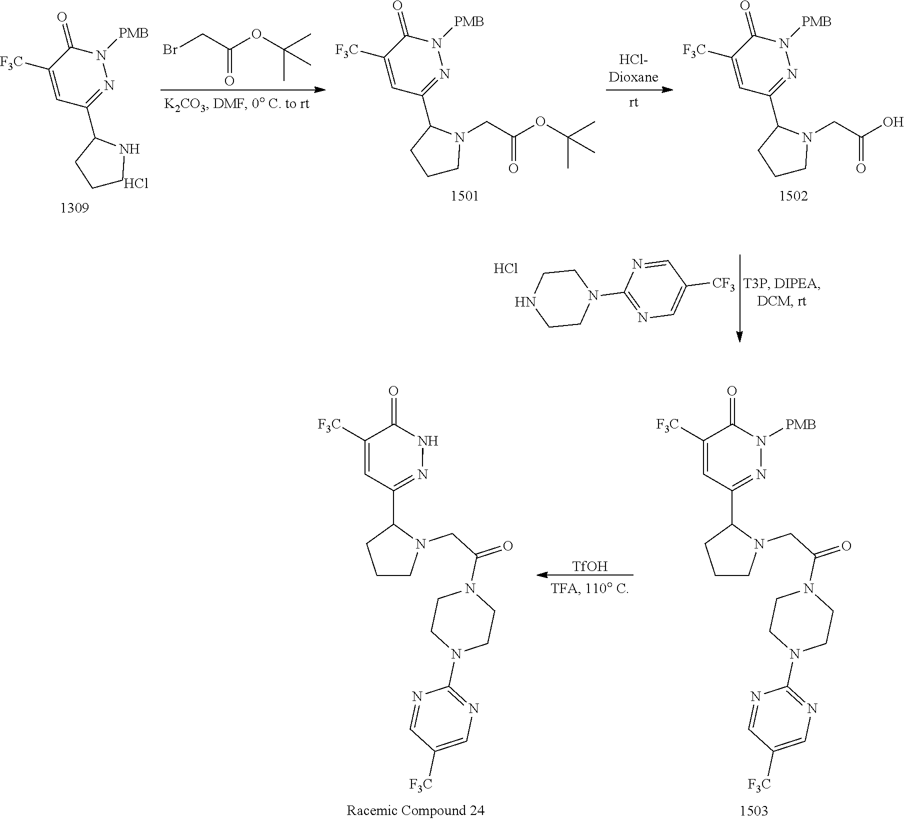

### Preparation of tert-butyl 2-(2-(1-(4-methoxybenzyl)-6-oxo-5-(trifluoromethyl)-1,6-dihydropyridazin-3-yl)pyrrolidin-1-yl)acetate (1501)

To a solution of 2-(4-methoxybenzyl)-6-(pyrrolidin-2-yl)-4-(trifluoromethyl)pyridazin-3(2H)-one hydrochloride 1309 (1.0 g, 2.8 mmol) in DMF (40 mL) was added K₂CO₃ (1.2 g, 8.4 mmol) at 0° C. Tert-butyl 2-bromoacetate (820 mg, 4.2 mmol) was added dropwise at 0° C. The reaction mixture was warmed to rt and stirred at rt for 1 h. The reaction mixture was poured into water and then extracted with EtOAc (100 mL×3). The combined organic layers were washed with brine (100 mL×3), dried over Na₂SO₄ and concentrated under reduced pressure. The residue was purified by flash column chromatography (eluting with PE/DCM=100:0 to 30:70) to afford tert-butyl 2-(2-(1-(4-methoxybenzyl)-6-oxo-5-(trifluoromethyl)-1,6-dihydropyridazin-3-yl)pyrrolidin-1-yl)acetate 1501 (800 mg, 60% purity, 35% yield) as a yellow oil.

LCMS (ESI) calcd for C₂₃H₂₈F₃N₃O₄[M+H]⁺ m/z 468.20, found 468.20.

### Preparation of 2-(2-(1-(4-methoxybenzyl)-6-oxo-5-(trifluoromethyl)-1,6-dihydropyridazin-3-yl)pyrrolidin-1-yl)acetic acid (1502)

A solution of tert-butyl 2-(2-(1-(4-methoxybenzyl)-6-oxo-5-(trifluoromethyl)-1,6-dihydropyridazin-3-yl)pyrrolidin-1-yl)acetate 1501 (800 mg, 1.71 mmol) in HCl-Dioxane (4 M, 20 mL) was stirred at rt for 1 h. The reaction solution was concentrated under reduced pressure and the residue was purified by C18 column (mobile phase: ACN—H₂O (0.1% FA), gradient: 10-95) to give 2-(2-(1-(4-methoxybenzyl)-6-oxo-5-(trifluoromethyl)-1,6-dihydropyridazin-3-yl)pyrrolidin-1-yl)acetic acid 1502 (200 mg, 90% purity, 25% yield) as a white solid.

LCMS (ESI) calcd for C₁₉H₂₀F₃N₃O₄[M+H]⁺ m/z 412.14, found 412.10.

### Preparation of 2-(4-methoxybenzyl)-6-(1-(2-oxo-2-(4-(5-(trifluoromethyl)pyrimidin-2-yl)piperazin-1-yl)ethyl)pyrrolidin-2-yl)-4-(trifluoromethyl)pyridazin-3(2H)-one (1503)

To a solution of 2-(2-(1-(4-methoxybenzyl)-6-oxo-5-(trifluoromethyl)-1,6-dihydropyridazin-3-yl)pyrrolidin-1-yl)acetic acid 1502 (200 mg, 0.49 mmol), 2-(piperazin-1-yl)-5-(trifluoromethyl)pyrimidine hydrochloride (197 mg, 0.73 mmol) in DCM (20 mL) were added DIPEA (251 mg, 1.94 mmol), T3P (50% wt in EtOAc, 618 mg, 0.97 mmol) at rt successively. The reaction mixture was stirred at rt for 1 h. The reaction solution was quenched with water (50 mL) and extracted with DCM (50 mL×2). The combined organic phase was concentrated and purified by flash column chromatography (eluting with DCM/MeOH=100:0 to 95:5) to afford 2-(4-methoxybenzyl)-6-(1-(2-oxo-2-(4-(5-(trifluoromethyl)pyrimidin-2-yl)piperazin-1-yl)ethyl)pyrrolidin-2-yl)-4-(trifluoromethyl)pyridazin-3(2H)-one 1503 (160 mg, 90% purity, 47% yield) as a white solid.

LCMS (ESI) calcd for C₂₈H₂₉F₆N₇O₃[M+H]⁺ m/z 626.22, found 626.25.

### Preparation of 6-(1-(2-oxo-2-(4-(5-(trifluoromethyl)pyrimidin-2-yl)piperazin-1-yl)ethyl)pyrrolidin-2-yl)-4-(trifluoromethyl)pyridazin-3(2H)-one (racemic Compound 24)

To a solution of 2-(4-methoxybenzyl)-6-(1-(2-oxo-2-(4-(5-(trifluoromethyl)pyrimidin-2-yl)piperazin-1-yl)ethyl)pyrrolidin-2-yl)-4-(trifluoromethyl)pyridazin-3(2H)-one 1503 (160 mg, 0.26 mmol) in TFA (10 mL) was added TfOH (1 mL) at rt. The reaction solution was stirred at 110° C. for 6 h. The mixture was adjusted to pH=8-9 with saturated aqueous NaHCO₃ at 0° C., then the aqueous layer was extracted with EtOAc (50 mL×3). The combined organic layers were concentrated under reduced pressure. The residue was purified by flash column chromatography (eluting with DCM/MeOH=100:0 to 95:5) to afford 6-(1-(2-oxo-2-(4-(5-(trifluoromethyl)pyrimidin-2-yl)piperazin-1-yl)ethyl)pyrrolidin-2-yl)-4-(trifluoromethyl)pyridazin-3(2H)-one racemic Compound 24 (70 mg, 95% purity, 51% yield) as a white solid.

Chiral resolution of 6-(1-(2-oxo-2-(4-(5-(trifluoromethyl)pyrimidin-2-yl)piperazin-1-yl)ethyl)pyrrolidin-2-yl)-4-(trifluoromethyl)pyridazin-3(2H)-one (Compound 24rac) Racemic compound 24 was separated by SFC (Column: Daicel Chiralpak IG SFC; 20 mm I.D.×250 mm, 5 μmm; Mobile phase: C₀₂/MeOH [0.1% NH3 (7 M Solution in MeOH)]=60/40) and concentrated under reduced pressure to afford the first fraction as compound 24a (26.6 mg, 99% purity, 100% ee, white solid) and the second fraction as compound 24b (19.0 mg, 99% purity, 100% ee, white solid).

### Compound 24a

¹H NMR (400 MHz, DMSO-d₆, ppm) δ: 13.49 (s, 1H), 8.73 (s, 2H), 7.81 (s, 1H), 3.98-3.81 (m, 2H), 3.76-3.64 (m, 2H), 3.64-3.50 (m, 3H), 3.50-3.42 (m, 1H), 3.42-3.35 (m, 1H), 3.29-3.12 (m, 3H), 2.58-2.51 (m, 1H), 2.23-2.08 (m, 1H), 1.95-1.71 (m, 3H).

LCMS (ESI) calcd for C₂₀H₂₁F₆N₇O₂[M+H]⁺ m/z 506.17, found 506.20.

### Compound 24b

¹H NMR (400 MHz, DMSO-d₆, ppm) δ: 13.48 (s, 1H), 8.73 (s, 2H), 7.81 (s, 1H), 3.96-3.85 (m, 2H), 3.75-3.62 (m, 2H), 3.61-3.50 (m, 3H), 3.50-3.43 (m, 1H), 3.42-3.34 (m, 1H), 3.30-3.14 (m, 3H), 2.58-2.51 (m, 1H), 2.23-2.06 (m, 1H), 1.95-1.72 (m, 3H).

LCMS (ESI) calcd for C₂₀H₂₁F₆N₇O₂[M+H]⁺ m/z 506.17, found 506.20.

### 7. Synthesis of 4-(1-(2-oxo-2-(4-(5-(trifluoromethyl)pyrimidin-2-yl)piperazin-1-yl)ethyl)pyrrolidin-2-yl)phthalazin-1(2H)-one (Compounds 38a and 38b)

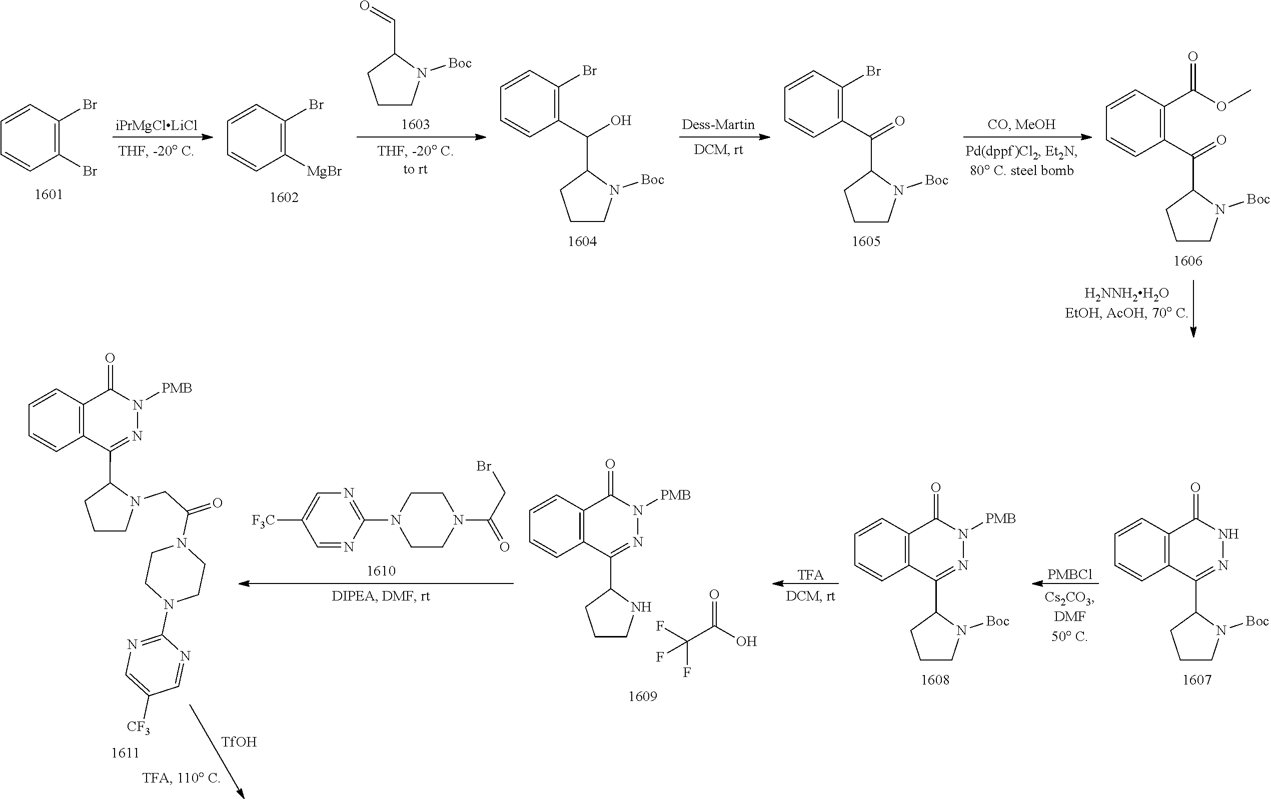

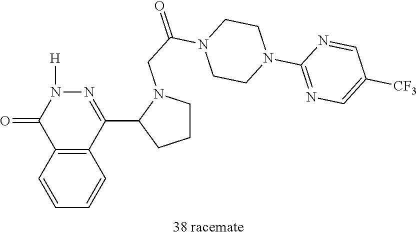 [SMILES: O=C(CN1CCCC1c1n[nH]c(=O)c2ccccc12)N1CCN(c2ncc(C(F)(F)F)cn2)CC1]

### Preparation of tert-butyl 2-((2-bromophenyl)(hydroxy)methyl)pyrrolidine-1-carboxylate (1604)

To a solution of 1,2-dibromobenzene 1601 (4.8 g, 0.0203 mol) in THF (100 mL) at −20° C. was added iPrMgCl·LiCl (16 mL, 0.0208 mol, 1.3 mol/L in THF). After stirring at −20° C. for 2 h, a solution of tert-butyl 2-formylpyrrolidine-1-carboxylate 1603 (4.88 g, 0.0243 mol) in THF (20 mL) was added and stirred at rt for 1 h. The reaction mixture was quenched with saturated NH₄Cl solution and extracted with EtOAc (100 mL×3). The combined organic layer was washed with brine, dried over Na₂SO₄, concentrated and purified with flash silica chromatography (eluting with DCM/PE, 0 to 64%) to obtain tert-butyl 2-((2-bromophenyl)(hydroxy)methyl)pyrrolidine-1-carboxylate 1604 (6.35 g, 90% purity, 78% yield) as a light yellow oil.

LCMS (ESI) calcd for C₁₆H₂₂BrNO₃ [M-t-Bu+H]⁺ m/z 300.02, found 299.88.

### Preparation of tert-butyl 2-(2-bromobenzoyl)pyrrolidine-1-carboxylate (1605)

Dess-Martin periodinane (12.64 g, 0.030 mol) was added to a solution of tert-butyl 2-((2-bromophenyl)(hydroxy)methyl)pyrrolidine-1-carboxylate 1604 (5.33 g, 0.015 mol) in DCM (100 mL). The mixture was stirred at rt for 2 h, quenched with saturated Na₂S₂O₃ solution and NaHCO₃ solution and extracted with DCM (20 mL×3). The combined organic layer was washed with brine, dried over Na₂SO₄, concentrated and purified with flash silica chromatography (eluting with EtOAc/PE, 0 to 17%) to obtain tert-butyl 2-(2-bromobenzoyl)pyrrolidine-1-carboxylate 1605 (4.8 g, 90% purity, 81% yield) as a colorless oil.

LCMS (ESI) calcd for C₁₆H₂₀BrNO₃ [M-t-Bu+H]⁺ m/z 298.01, found 297.79.

### Preparation of tert-butyl 2-(2-(methoxycarbonyl)benzoyl)pyrrolidine-1-carboxylate (1606)

A mixture of tert-butyl 2-(2-bromobenzoyl)pyrrolidine-1-carboxylate 1605 (1.0 g, 0.0028 mol), Pd(dppf)Cl₂ (0.2 g, 0.0002 mol) and Et₂N (0.6 g, 0.0056 mol) in MeOH (8 mL) was degassed with CO and heated at 80° C. for 16 h under CO atmosphere in a steel bomb. After cooling to rt, the mixture was concentrated and purified with flash silica chromatography (eluting with EtOAc/PE, 0 to 50%) to obtain tert-butyl 2-(2-(methoxycarbonyl)benzoyl)pyrrolidine-1-carboxylate 1606 (0.9 g, 28% purity, 28% yield) as a brown oil.

LCMS (ESI) calcd for C₁₈H₂₃NO₅ [M+Na]⁺ m/z 356.15, found 355.94.

### Preparation of tert-butyl 2-(4-oxo-3,4-dihydrophthalazin-1-yl)pyrrolidine-1-carboxylate (1607)

A mixture of tert-butyl 2-(2-(methoxycarbonyl)benzoyl)pyrrolidine-1-carboxylate 1606 (900 mg, 0.754 mmol, 28% purity) and H₂NNH₂·H₂O (888 mg, 22.165 mmol, 80% wt.) in EtOH (10 mL) and AcOH (10 mL) was heated at 70° C. for 1 h. After cooling to rt, the mixture was concentrated and purified with flash silica chromatography (eluting with EtOAc/PE, 0 to 71%) to obtain tert-butyl 2-(4-oxo-3,4-dihydrophthalazin-1-yl)pyrrolidine-1-carboxylate 1607 (150 mg, 90% purity, 56% yield) as a white solid.

LCMS (ESI) calcd for C₁₇H₂₁N₃O₃ [M+H]⁺ m/z 316.17, found 316.01.

### Preparation of tert-butyl 2-(3-(4-methoxybenzyl)-4-oxo-3,4-dihydrophthalazin-1-yl)pyrrolidine-1-carboxylate (1608)

A mixture of tert-butyl 2-(4-oxo-3,4-dihydrophthalazin-1-yl)pyrrolidine-1-carboxylate 1607 (140 mg, 0.442 mmol), PMBCl (83 mg, 0.531 mmol) and Cs₂CO₃ (288 mg, 0.885 mmol) in DMF (10 mL) was heated at 50° C. for 2 h. After cooling to rt, the mixture was diluted with water (80 mL) and extracted with EtOAc (50 mL×3). The combined organic layer was washed with brine, dried over Na₂SO₄, concentrated and purified with flash silica chromatography (eluting with EtOAc/PE, 0 to 27%) to obtain tert-butyl 2-(3-(4-methoxybenzyl)-4-oxo-3,4-dihydrophthalazin-1-yl)pyrrolidine-1-carboxylate 1608 (150 mg, 90% purity, 69% yield) as a colorless oil.

LCMS (ESI) calcd for C₂₅H₂₉N₃O₄[M+Na]⁺ m/z 458.21, found 458.08.

### Preparation of 2-(4-methoxybenzyl)-4-(pyrrolidin-2-yl)phthalazin-1(2H)-one 2,2,2-trifluoroacetate (1609)

TFA (2 mL) was added to a solution of tert-butyl 2-(3-(4-methoxybenzyl)-4-oxo-3,4-dihydrophthalazin-1-yl)pyrrolidine-1-carboxylate 1608 (135 mg, 0.309 mmol) in DCM (6 mL) at rt. The mixture was stirred at rt for 1 h, concentrated and evaporated with DCM 3 times to obtain 2-(4-methoxybenzyl)-4-(pyrrolidin-2-yl)phthalazin-1(2H)-one 2,2,2-trifluoroacetate 1609 (120 mg, 90% purity, 77% yield) as a yellow oil.

LCMS (ESI) calcd for C₂₀H₂₁N₃O₂ [M+H]⁺ m/z 336.17, found 336.05.

### Preparation of 2-(4-methoxybenzyl)-4-(1-(2-oxo-2-(4-(5-(trifluoromethyl)pyrimidin-2-yl)piperazin-1-yl)ethyl)pyrrolidin-2-yl)phthalazin-1(2H)-one (1611)

To a solution of 2-(4-methoxybenzyl)-4-(pyrrolidin-2-yl)phthalazin-1(2H)-one 2,2,2-trifluoroacetate 1609 (120 mg, 0.267 mmol) in DMF (20 mL) were added DIPEA (103 mg, 0.801 mmol) and 2-bromo-1-(4-(5-(trifluoromethyl)pyrimidin-2-yl)piperazin-1-yl)ethan-1-one 1610 (283 mg, 0.801 mmol). The mixture was stirred at rt for 1 h, diluted with water (100 mL) and extracted with EtOAc (50 mL×3). The combined organic layer was washed with brine, dried over Na₂SO₄, concentrated and purified with flash silica chromatography (eluting with MeOH/DCM, 0 to 5%) to obtain 2-(4-methoxybenzyl)-4-(1-(2-oxo-2-(4-(5-(trifluoromethyl)pyrimidin-2-yl)piperazin-1-yl)ethyl)pyrrolidin-2-yl)phthalazin-1(2H)-one 1611 (120 mg, 90% purity, 66% yield) as a yellow oil.

LCMS (ESI) calcd for C₃₁H₃₂F₃N₇O₃[M+H]⁺ m/z 608.26, found 608.25.

### Preparation of 4-(1-(2-oxo-2-(4-(5-(trifluoromethyl)pyrimidin-2-yl)piperazin-1-yl)ethyl)pyrrolidin-2-yl)phthalazin-1(2H)-one (38rac)

TfOH (1 mL) was added to a solution of 2-(4-methoxybenzyl)-4-(1-(2-oxo-2-(4-(5-(trifluoromethyl)pyrimidin-2-yl)piperazin-1-yl)ethyl)pyrrolidin-2-yl)phthalazin-1(2H)-one 1611 (120 mg, 0.197 mmol) in TFA (4 mL). The mixture was heated at 110° C. for 10 min, adjusted to pH 8.0 with saturated NaHCO₃ solution and extracted with DCM (100 mL×3). The combined organic layer was washed with brine, dried over Na₂SO₄, concentrated, purified with flash silica chromatography (eluting with EtOAc/PE, 0 to 100%) and prep-HPLC (Column: Gemini 5 μm C₁₈ 150×21.2 mm, mobile phase: ACN—H₂O (0.1% FA), gradient: 20-40) to obtain 4-(1-(2-oxo-2-(4-(5-(trifluoromethyl)pyrimidin-2-yl)piperazin-1-yl)ethyl)pyrrolidin-2-yl)phthalazin-1(2H)-one 38 racemate (40 mg, 99% purity, 41% yield) as a white solid.

### Chiral resolution of 4-(1-(2-oxo-2-(4-(5-(trifluoromethyl)pyrimidin-2-yl)piperazin-1-yl)ethyl)pyrrolidin-2-yl)phthalazin-1(2H)-one (38rac)

38 racemate was separated by SFC (Column: Daicel IH 250 mm×20 mm I.D., 5 μm; Mobile phase: CO₂/MeOH (0.1% NH₃)=70/30) and concentrated under reduced pressure to afford the first fraction as 38a (12.1 mg, 99% purity, 100% ee, white solid) and the second fraction as 38b (11.8 mg, 99% purity, 100% ee, white solid).

### Compound 38a

¹H NMR (400 MHz, DMSO-d₆, ppm) δ: 12.61 (s, 1H), 8.71 (s, 2H), 8.43-8.36 (m, 1H), 8.31-8.24 (m, 1H), 7.95-7.78 (m, 2H), 3.92-3.77 (m, 3H), 3.66-3.49 (m, 5H), 3.29-3.25 (m, 2H), 3.14-3.07 (m, 1H), 3.03-2.97 (m, 1H), 2.44-2.41 (m, 1H), 2.31-2.23 (m, 1H), 2.05-1.89 (m, 3H).

LCMS (ESI) calcd for C₂₃H₂₄F₃N₇O₂[M+H]⁺ m/z 488.20, found 488.20.

### Compound 38b

¹H NMR (400 MHz, DMSO-d₆, ppm) δ: 12.61 (s, 1H), 8.71 (s, 2H), 8.43-8.36 (m, 1H), 8.30-8.25 (m, 1H), 7.92-7.80 (m, 2H), 3.93-3.77 (m, 3H), 3.64-3.50 (m, 5H), 3.29-3.24 (m, 2H), 3.16-3.05 (m, 1H), 3.02-2.96 (m, 1H), 2.45-2.41 (m, 1H), 2.30-2.21 (m, 1H), 2.08-1.87 (m, 3H).

LCMS (ESI) calcd for C₂₃H₂₄F₃N₇O₂[M+H]⁺ m/z 488.20, found 488.20.

### 8. Synthesis of N-methyl-5-(4-(3-(4-(4-oxo-3,4-dihydrophthalazin-1-yl)piperidin-1-yl)propanoyl)piperazin-1-yl)picolinamide (Compound 48)

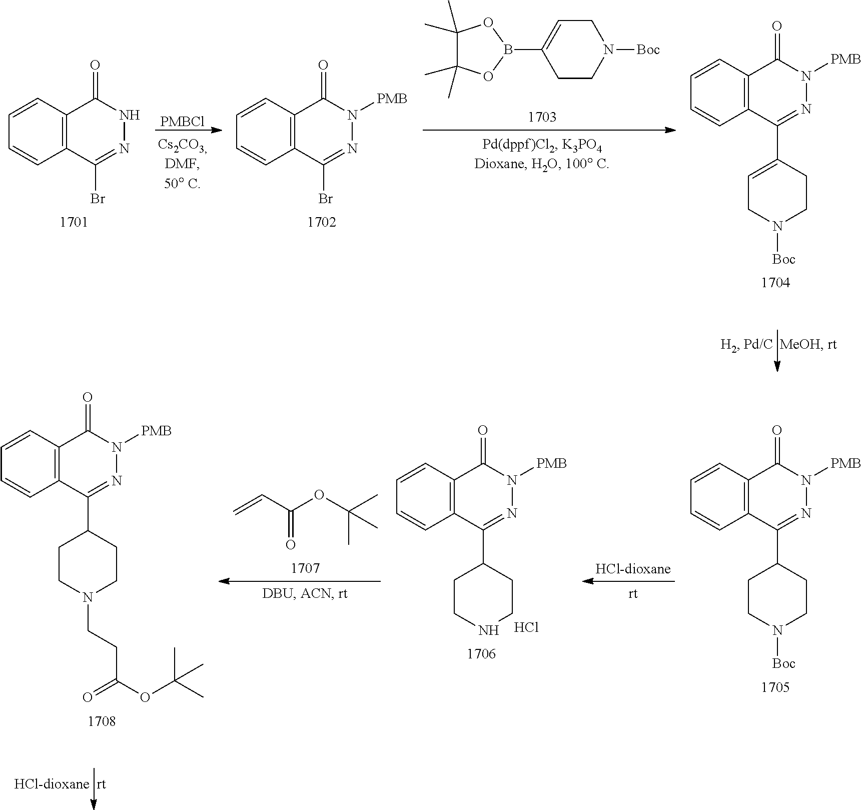

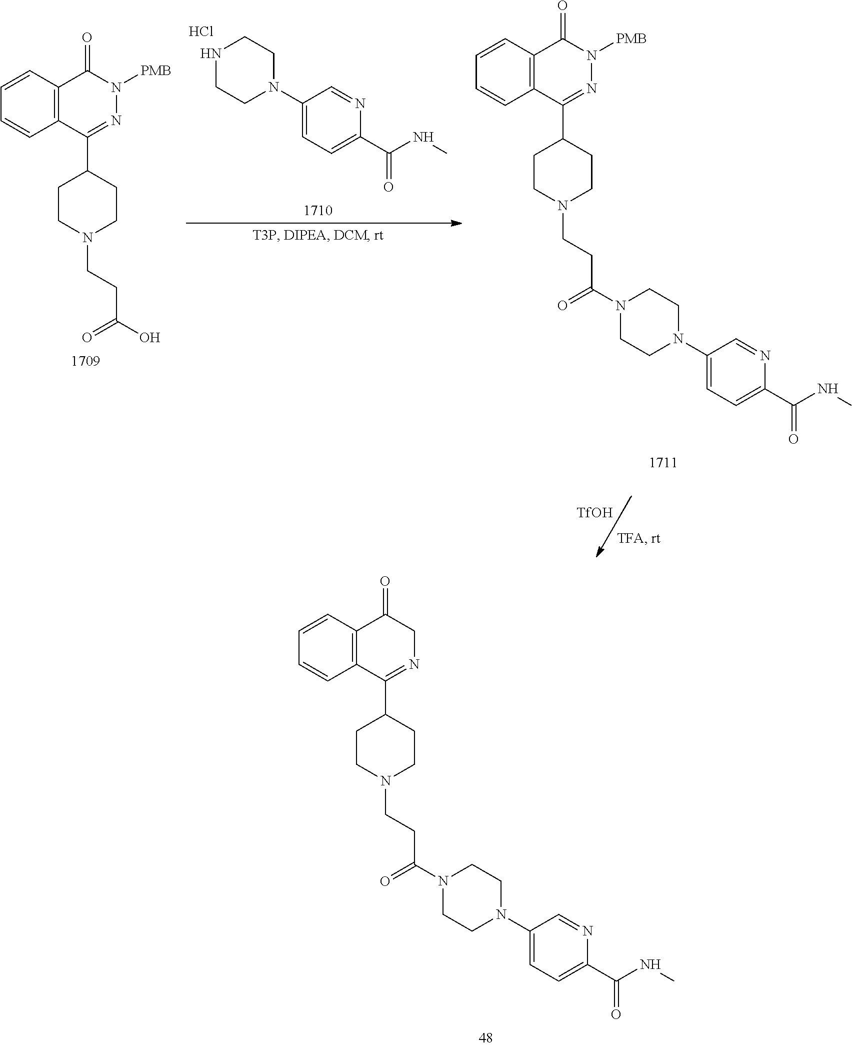

### Preparation of 4-bromo-2-(4-methoxybenzyl)phthalazin-1(2H)-one (1702)

A mixture of 4-bromophthalazin-1(2H)-one 1701 (1.0 g, 0.0044 mol), PMBCl (0.8 g, 0.0052 mol) and Cs₂CO₃ (2.9 g, 0.0088 mol) in DMF (20 mL) was heated at 50° C. for 2 h. After cooling to rt, the mixture was diluted with water and extracted with EtOAc (50 mL×3). The combined organic layer was washed with brine, dried over Na₂SO₄, concentrated and purified with flash silica chromatography (eluting with DCM/PE, 0 to 57%) to obtain 4-bromo-2-(4-methoxybenzyl)phthalazin-1(2H)-one 1702 (1.5 g, 90% purity, 88% yield) as a white solid.

LCMS (ESI) calcd for C₁₆H₁₃BrN₂O₂[M+H]⁺ m/z 345.02, found 344.90.

### Preparation of tert-butyl 4-(3-(4-methoxybenzyl)-4-oxo-3,4-dihydrophthalazin-1-yl)-3,6-dihydropyridine-1(2H)-carboxylate (1704)

A mixture of 4-bromo-2-(4-methoxybenzyl)phthalazin-1(2H)-one 1702 (1.4 g, 0.0041 mol), tert-butyl 4-(4,4,5,5-tetramethyl-1,3,2-dioxaborolan-2-yl)-3,6-dihydropyridine-1(2H)-carboxylate 1703 (1.4 g, 0.0045 mol), Pd(dppf)Cl₂ (0.3 g, 0.0004 mol) and K₃PO₄ (1.7 g, 0.0082 mol) in dioxane and H₂O (40 mL, 3:1) was degassed with N₂ and heated at 100° C. for 2 h under N₂ atmosphere. The resulting mixture was cooled, diluted with water and extracted with EtOAc (30 mL×3). The combined organic layer was washed with brine, dried over Na₂SO₄, concentrated, and purified with flash silica chromatography (eluting with EtOAc/PE, 0 to 28%) to obtain tert-butyl 4-(3-(4-methoxybenzyl)-4-oxo-3,4-dihydrophthalazin-1-yl)-3,6-dihydropyridine-1(2H)-carboxylate 1704 (1.8 g, 90% purity, 87% yield) as a yellow oil.

LCMS (ESI) calcd for C₂₆H₂₉N₃O₄ [M+H]⁺ m/z 448.22, found 448.02.

### Preparation of tert-butyl 4-(3-(4-methoxybenzyl)-4-oxo-3,4-dihydrophthalazin-1-yl)piperidine-1-carboxylate (1705)

A mixture of tert-butyl 4-(3-(4-methoxybenzyl)-4-oxo-3,4-dihydrophthalazin-1-yl)-3,6-dihydropyridine-1(2H)-carboxylate 1704 (1.8 g, 0.0040 mol) and Pd/C (1.3 g) in MeOH (50 mL) was degassed with H₂ and stirred at rt for 3 h under H₂ atmosphere. The mixture was filtered to remove Pd/C, and the filtrate was concentrated to obtain tert-butyl 4-(3-(4-methoxybenzyl)-4-oxo-3,4-dihydrophthalazin-1-yl)piperidine-1-carboxylate 1705 (1.8 g, 90% purity, 90% yield) as a yellow oil.

LCMS (ESI) calcd for C₂₆H₃₁N₃O₄ [M+H]⁺ m/z 450.24, found 450.20.

### Preparation of 2-(4-methoxybenzyl)-4-(piperidin-4-yl)phthalazin-1(2H)-one hydrochloride (1706)

A solution of tert-butyl 4-(3-(4-methoxybenzyl)-4-oxo-3,4-dihydrophthalazin-1-yl)piperidine-1-carboxylate 1705 (1.8 g, 0.0040 mol) in HCl-dioxane (30 mL, 4 M) was stirred at rt for 2 h and concentrated to obtain 2-(4-methoxybenzyl)-4-(piperidin-4-yl)phthalazin-1(2H)-one hydrochloride 1706 (1.3 g, 90% purity, 75% yield) as a grey solid.

LCMS (ESI) calcd for C₂₁H₂₃N₃O₂ [M+H]⁺ m/z 350.19, found 350.25.

### Preparation of tert-butyl 3-(4-(3-(4-methoxybenzyl)-4-oxo-3,4-dihydrophthalazin-1-yl)piperidin-1-yl)propanoate (1708)

To a solution of 2-(4-methoxybenzyl)-4-(piperidin-4-yl)phthalazin-1(2H)-one hydrochloride 1706 (750 mg, 1.944 mmol) in ACN (36 mL) were added DBU (592 mg, 3.887 mmol) and tert-butyl acrylate 1707 (498 mg, 3.887 mmol). The mixture was stirred at rt for 16 h, concentrated and purified with flash silica chromatography (eluting with EtOAc/PE, 0 to 42%) to obtain tert-butyl 3-(4-(3-(4-methoxybenzyl)-4-oxo-3,4-dihydrophthalazin-1-yl)piperidin-1-yl)propanoate 1708 (700 mg, 90% purity, 67% yield) as a white solid.

LCMS (ESI) calcd for C₂₈H₃₅N₃O₄ [M+H]⁺ m/z 478.27, found 478.25.

### Preparation of 3-(4-(3-(4-methoxybenzyl)-4-oxo-3,4-dihydrophthalazin-1-yl)piperidin-1-yl)propanoic acid (1709)

A solution of tert-butyl 3-(4-(3-(4-methoxybenzyl)-4-oxo-3,4-dihydrophthalazin-1-yl)piperidin-1-yl)propanoate 1708 (700 mg, 1.466 mmol) in HCl-dioxane (20 mL, 4 mol/L) was stirred at rt for 2 h and concentrated to obtain 3-(4-(3-(4-methoxybenzyl)-4-oxo-3,4-dihydrophthalazin-1-yl)piperidin-1-yl)propanoic acid 1709 (500 mg, 90% purity, 72% yield) as a white solid.

LCMS (ESI) calcd for C₂₄H₂₇N₃O₄ [M+H]⁺ m/z 422.21, found 422.04.

### Preparation of 5-(4-(3-(4-(3-(4-methoxybenzyl)-4-oxo-3,4-dihydrophthalazin-1-yl)piperidin-1-yl)propanoyl)piperazin-1-yl)-N-methylpicolinamide (1711)

To a solution of 3-(4-(3-(4-methoxybenzyl)-4-oxo-3,4-dihydrophthalazin-1-yl)piperidin-1-yl)propanoic acid 1709 (100 mg, 0.237 mmol) in DCM (15 mL) were added N-methyl-5-(piperazin-1-yl)picolinamide hydrochloride 1710 (73 mg, 0.285 mmol), DIPEA (123 mg, 0.949 mmol) and T3P (302 mg, 0.476 mmol, 50% in EtOAc). The mixture was stirred at rt for 1 h, concentrated and purified by flash silica chromatography (eluting with MeOH/DCM, 0 to 3%) to obtain 5-(4-(3-(4-(3-(4-methoxybenzyl)-4-oxo-3,4-dihydrophthalazin-1-yl)piperidin-1-yl)propanoyl)piperazin-1-yl)-N-methylpicolinamide 1711 (100 mg, 90% purity, 60% yield) as a white solid.

LCMS (ESI) calcd for C₃₅H₄₁N₇O₄ [M+H]⁺ m/z 624.33, found 624.30.

### Preparation of N-methyl-5-(4-(3-(4-(4-oxo-3,4-dihydrophthalazin-1-yl)piperidin-1-yl)propanoyl)piperazin-1-yl)picolinamide (48)

TfOH (1 mL) was added to a solution of 5-(4-(3-(4-(3-(4-methoxybenzyl)-4-oxo-3,4-dihydrophthalazin-1-yl)piperidin-1-yl)propanoyl)piperazin-1-yl)-N-methylpicolinamide 1711 (100 mg, 0.160 mmol) in TFA (4 mL). The mixture was stirred at rt for 30 min, adjusted to pH 8.0 with saturated NaHCO₃ solution and extracted with DCM (100 mL×3). The combined organic layer was washed with brine, dried over Na₂SO₄, concentrated and purified with prep-HPLC (Column: Gemini 5 μm C₁₈ 150×21.2 mm, mobile phase: ACN—H₂O (0.1% FA), gradient: 25-30) to afford N-methyl-5-(4-(3-(4-(4-oxo-3,4-dihydrophthalazin-1-yl)piperidin-1-yl)propanoyl)piperazin-1-yl)picolinamide 48 (0.16 FA salt, 10.4 mg, 98% purity, 12% yield) as a white solid.

¹H NMR (400 MHz, DMSO-d₆, ppm) δ: 12.50 (s, 1H), 8.42 (q, J=4.7 Hz, 1H), 8.32-8.24 (m, 2H), 8.22 (s, 0.16H), 8.11-8.03 (m, 1H), 7.98-7.91 (m, 1H), 7.89-7.81 (m, 2H), 7.41 (dd, J=8.8, 3.2 Hz, 1H), 3.72-3.57 (m, 4H), 3.45-3.35 (m, 4H), 3.25-3.14 (m, 1H), 3.06-2.96 (m, 2H), 2.78 (d, J=4.8 Hz, 3H), 2.66-2.55 (m, 4H), 2.25-2.14 (m, 2H), 1.92-1.82 (m, 2H), 1.81-1.68 (m, 2H). LCMS (ESI) calcd for C₂₇H₃₃N₇O₃ [M+H]⁺ m/z 504.27, found 504.15.

### 9. Synthesis of 5-(1-(2-oxo-2-(4-(5-(trifluoromethyl)pyrimidin-2-yl)piperazin-1-yl)ethyl)pyrrolidin-2-yl)-3-(trifluoromethyl)pyridin-2(1H)-one (Compound 60)

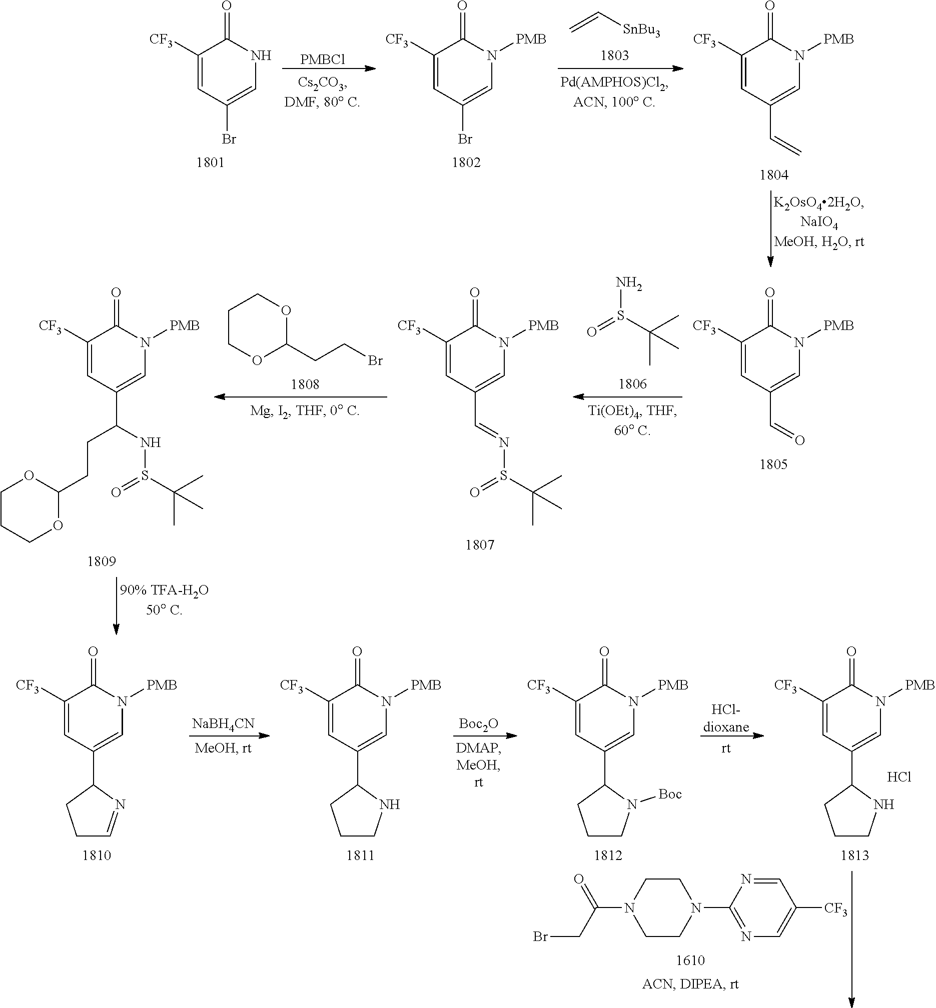

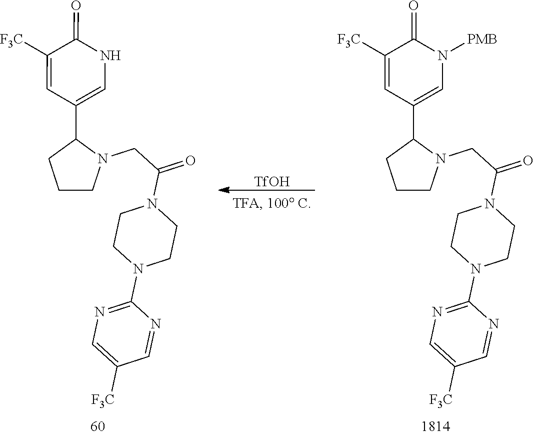

### Preparation of 5-bromo-1-(4-methoxybenzyl)-3-(trifluoromethyl)pyridin-2(1H)-one (1802)

A mixture of 5-bromo-3-(trifluoromethyl)pyridin-2(1H)-one 1801 (2.0 g, 0.0082 mol), PMBCl (1.9 g, 0.0123 mol) and Cs₂CO₃ (5.3 g, 0.0164 mol) in DMF (40 mL) was heated at 80° C. for 2 h. The mixture was diluted with water (300 mL) and extracted with EtOAc (100 mL×3). The combined organic layer was washed with brine, dried over Na₂SO₄, concentrated and purified with flash silica chromatography (eluting with EtOAc/PE, 0 to 18%) to obtain 5-bromo-1-(4-methoxybenzyl)-3-(trifluoromethyl)pyridin-2(1H)-one 1802 (2.3 g, 90% purity, 69% yield) as a white solid.

LCMS (ESI) calcd for C₁₄H₁₁BrF₃NO₂ [M+H]⁺ m/z 362.00, found 361.90.

### Preparation of 1-(4-methoxybenzyl)-3-(trifluoromethyl)-5-vinylpyridin-2(1H)-one (1804)

A mixture of 5-bromo-1-(4-methoxybenzyl)-3-(trifluoromethyl)pyridin-2(1H)-one 1802 (2.3 g, 0.0063 mol), tributyl(vinyl)stannane 1803 (4.0 g, 0.0126 mol) and Pd(AMPHOS)Cl₂ (0.2 g, 0.0003 mol) in ACN (60 mL) was degassed with N₂ and heated at 100° C. for 2 h under N₂ protection. The mixture was concentrated and purified with flash silica chromatography (eluting with EtOAc/PE, 0 to 20%) to obtain 1-(4-methoxybenzyl)-3-(trifluoromethyl)-5-vinylpyridin-2(1H)-one 1804 (2 g, 90% purity, 92% yield) as a yellow solid.

LCMS (ESI) calcd for C₁₆H₁₄F₃NO₂ [M+H]⁺ m/z 310.10, found 309.93.

### Preparation of 1-(4-methoxybenzyl)-6-oxo-5-(trifluoromethyl)-1,6-dihydropyridine-3-carbaldehyde (1805)

To a solution of 1-(4-methoxybenzyl)-3-(trifluoromethyl)-5-vinylpyridin-2(1H)-one 1804 (2.0 g, 0.0064 mol) in MeOH and H₂O (5:1, 42 mL) was added K₂OsO₄·2H₂O (0.2 g, 0.0006 mol). After stirring at rt for 10 min, NaIO₄ (5.5 g, 0.0256 mol) was added and the mixture was stirred at rt for 3 h. The mixture was filtered, and the filtrate was extracted with EtOAc (30 mL×3). The combined organic layer was washed with brine, dried over Na₂SO₄, concentrated, and purified with flash silica chromatography (eluting with EtOAc/PE, 0 to 40%) to obtain 1-(4-methoxybenzyl)-6-oxo-5-(trifluoromethyl)-1,6-dihydropyridine-3-carbaldehyde 1805 (1.0 g, 90% purity, 45% yield) as a white solid.

LCMS (ESI) calcd for C₁₅H₁₂F₃NO₃ [M+H]⁺ m/z 312.08, found 312.15.

### Preparation of (E)-N-((1-(4-methoxybenzyl)-6-oxo-5-(trifluoromethyl)-1,6-dihydropyridin-3-yl)methylene)-2-methylpropane-2-sulfinamide (1807)

To a solution of 1-(4-methoxybenzyl)-6-oxo-5-(trifluoromethyl)-1,6-dihydropyridine-3-carbaldehyde 1805 (1.0 g, 0.0032 mol) in THF (20 mL) were added 2-methylpropane-2-sulfinamide 1806 (0.8 g, 0.0064 mol) and Ti(OEt)₄ (1.5 g, 0.0064 mol). The mixture was heated at 60° C. for 2 h, concentrated and purified by flash silica chromatography (eluting with EtOAc/PE, 0 to 43%) to obtain (E)-N-((1-(4-methoxybenzyl)-6-oxo-5-(trifluoromethyl)-1,6-dihydropyridin-3-yl)methylene)-2-methylpropane-2-sulfinamide 1807 (1.0 g, 95% purity, 71% yield) as a white solid.

LCMS (ESI) calcd for C₁₉H₂₁F₃N₂O₃S [M+H]⁺ m/z 415.13, found 415.15.

### Preparation of N-(3-(1,3-dioxan-2-yl)-1-(1-(4-methoxybenzyl)-6-oxo-5-(trifluoromethyl)-1,6-dihydropyridin-3-yl)propyl)-2-methylpropane-2-sulfinamide (1809)

A 50 mL three-neck flask was charged with Mg (2.5 g), I₂ (0.1 g) and THF (10 mL) and degassed with N₂. A solution of 2-(2-bromoethyl)-1,3-dioxane 1808 (1.4 g, 0.0072 mol) in THF (10 mL) was added in 5 portions. When the first portion was added, a hair dryer was used to initiate the reaction, the remaining 4 portions were added dropwise to keep the solution refluxing. The mixture was then stirred for 30 min to obtain (2-(1,3-dioxan-2-yl)ethyl)magnesium bromide solution. A solution of (E)-N-((1-(4-methoxybenzyl)-6-oxo-5-(trifluoromethyl)-1,6-dihydropyridin-3-yl)methylene)-2-methylpropane-2-sulfinamide 1807 (1.0 g, 0.0024 mol) in THF (20 mL) was degassed with N₂, added (2-(1,3-dioxan-2-yl)ethyl)magnesium bromide solution and stirred at 0° C. for 30 min. The resulting mixture was quenched with saturated NH₄Cl solution and extracted with EtOAc (30 mL×3). The combined organic layer was washed with brine, dried over Na₂SO₄ and concentrated to obtain N-(3-(1,3-dioxan-2-yl)-1-(1-(4-methoxybenzyl)-6-oxo-5-(trifluoromethyl)-1,6-dihydropyridin-3-yl)propyl)-2-methylpropane-2-sulfinamide 1809 (1.0 g, 50% purity, 37% yield) as a yellow solid.

LCMS (ESI) calcd for C₂₅H₃₃F₃N₂O₅S [M+H]⁺ m/z 531.21, found 531.20.

### Preparation of tert-butyl 2-(1-(4-methoxybenzyl)-6-oxo-5-(trifluoromethyl)-1,6-dihydropyridin-3-yl)pyrrolidine-1-carboxylate (1812)

A solution of N-(3-(1,3-dioxan-2-yl)-1-(1-(4-methoxybenzyl)-6-oxo-5-(trifluoromethyl)-1,6-dihydropyridin-3-yl)propyl)-2-methylpropane-2-sulfinamide 1809 (1.0 g, 0.0019 mol) in 90% TFA-H₂O (18 mL) was heated at 50° C. for 1 h. After cooling to rt, the mixture was diluted with MeOH (90 mL), added NaBH₃CN (0.2 g, 0.0030 mol) and stirred at rt for 1 h. The resulting mixture was adjusted to pH 12.0 with 5 M aqueous NaOH solution, then added Boc₂O (0.8 g, 0.0038 mol) and DMAP (0.02 g, 0.0001 mol). After stirring at rt for 1 h, the mixture was extracted with EtOAc (50 mL×3). The combined organic layer was washed with brine, dried over Na₂SO₄, concentrated and purified with flash silica chromatography (eluting with EtOAc/PE, 0 to 17%) to obtain tert-butyl 2-(1-(4-methoxybenzyl)-6-oxo-5-(trifluoromethyl)-1,6-dihydropyridin-3-yl)pyrrolidine-1-carboxylate 1812 (0.4 g, 95% purity, 42% yield) as a yellow oil.

LCMS (ESI) calcd for C₂₃H₂₇F₃N₂O₄[M+H]⁺ m/z 453.20, found 453.06.

### Preparation of 1-(4-methoxybenzyl)-5-(pyrrolidin-2-yl)-3-(trifluoromethyl)pyridin-2(1H)-one hydrochloride (1813)

A solution of tert-butyl 2-(1-(4-methoxybenzyl)-6-oxo-5-(trifluoromethyl)-1,6-dihydropyridin-3-yl)pyrrolidine-1-carboxylate 1812 (400 mg, 0.880 mmol) in HCl-dioxane (10 mL, 4 M) was stirred at rt for 1 h and concentrated to obtain 1-(4-methoxybenzyl)-5-(pyrrolidin-2-yl)-3-(trifluoromethyl)pyridin-2(1H)-one hydrochloride 1813 (220 mg, 93% purity, 59% yield) as a yellow oil.

LCMS (ESI) calcd for C₁₈H₁₉F₃N₂O₂[M+H]⁺ m/z 353.15, found 352.99.

### Preparation of 1-(4-methoxybenzyl)-5-(1-(2-oxo-2-(4-(5-(trifluoromethyl)pyrimidin-2-yl)piperazin-1-yl)ethyl)pyrrolidin-2-yl)-3-(trifluoromethyl)pyridin-2(1H)-one (1814)

To a solution of 1-(4-methoxybenzyl)-5-(pyrrolidin-2-yl)-3-(trifluoromethyl)pyridin-2(1H)-one hydrochloride 1813 (100 mg, 0.256 mmol) in ACN (10 mL) were added DIPEA (66 mg, 0.513 mmol) and 2-bromo-1-(4-(5-(trifluoromethyl)pyrimidin-2-yl)piperazin-1-yl)ethan-1-one 1610 (109 mg, 0.308 mmol). The mixture was stirred at rt for 2 h, concentrated and purified with flash silica chromatography (eluting with EtOAc/PE, 0 to 96%) to obtain 1-(4-methoxybenzyl)-5-(1-(2-oxo-2-(4-(5-(trifluoromethyl)pyrimidin-2-yl)piperazin-1-yl)ethyl)pyrrolidin-2-yl)-3-(trifluoromethyl)pyridin-2(1H)-one 1814 (60 mg, 90% purity, 33% yield) as a yellow solid.

LCMS (ESI) calcd for C₂₉H₃₀F₆N₆O₃[M+H]⁺ m/z 625.24, found 625.12.

### Preparation of 5-(1-(2-oxo-2-(4-(5-(trifluoromethyl)pyrimidin-2-yl)piperazin-1-yl)ethyl)pyrrolidin-2-yl)-3-(trifluoromethyl)pyridin-2(1H)-one (60)

To a solution of 1-(4-methoxybenzyl)-5-(1-(2-oxo-2-(4-(5-(trifluoromethyl)pyrimidin-2-yl)piperazin-1-yl)ethyl)pyrrolidin-2-yl)-3-(trifluoromethyl)pyridin-2(1H)-one 1814 (60 mg, 0.096 mmol) in TFA (4 mL) was added TfOH (1 mL). The mixture was heated at 100° C. for 5 min, adjusted to pH 8.0 with saturated NaHCO₃ solution at ° C. and extracted with DCM (100 mL×3). The combined organic layer was washed with brine, dried over Na₂SO₄, concentrated, and purified with flash silica chromatography (eluting with MeOH/DCM, 0 to 5%) and prep-HPLC (Column: Gemini 5 μm C₁₈ 150×21.2 mm, mobile phase: ACN—H₂O (0.1% FA), gradient: 20-50) to obtain 5-(1-(2-oxo-2-(4-(5-(trifluoromethyl)pyrimidin-2-yl)piperazin-1-yl)ethyl)pyrrolidin-2-yl)-3-(trifluoromethyl)pyridin-2(1H)-one 60 (17.0 mg, 97% purity, 34% yield) as a white solid.

¹H NMR (400 MHz, DMSO-d₆, ppm) δ: 12.19 (s, 1H), 8.73 (s, 2H), 7.84-7.80 (m, 1H), 7.54 (s, 1H), 4.00-3.88 (m, 2H), 3.66-3.51 (m, 4H), 3.37-3.32 (m, 2H), 3.30-3.26 (m, 1H), 3.21-3.14 (m, 2H), 3.09-3.03 (m, 1H), 2.38-2.32 (m, 1H), 2.13-2.02 (m, 1H), 1.90-1.72 (m, 2H), 1.67-1.55 (m, 1H).

LCMS (ESI) calcd for C₂₁H₂₂F₆N₆O₂[M+H]⁺ m/z 505.18, found 505.72.

### 10. Synthesis of 4-chloro-6-(1-(2-oxo-2-(4-(5-(trifluoromethyl)pyrimidin-2-yl)piperazin-1-yl)ethyl)pyrrolidin-2-yl)pyridazin-3(2H)-one (Compound 68)

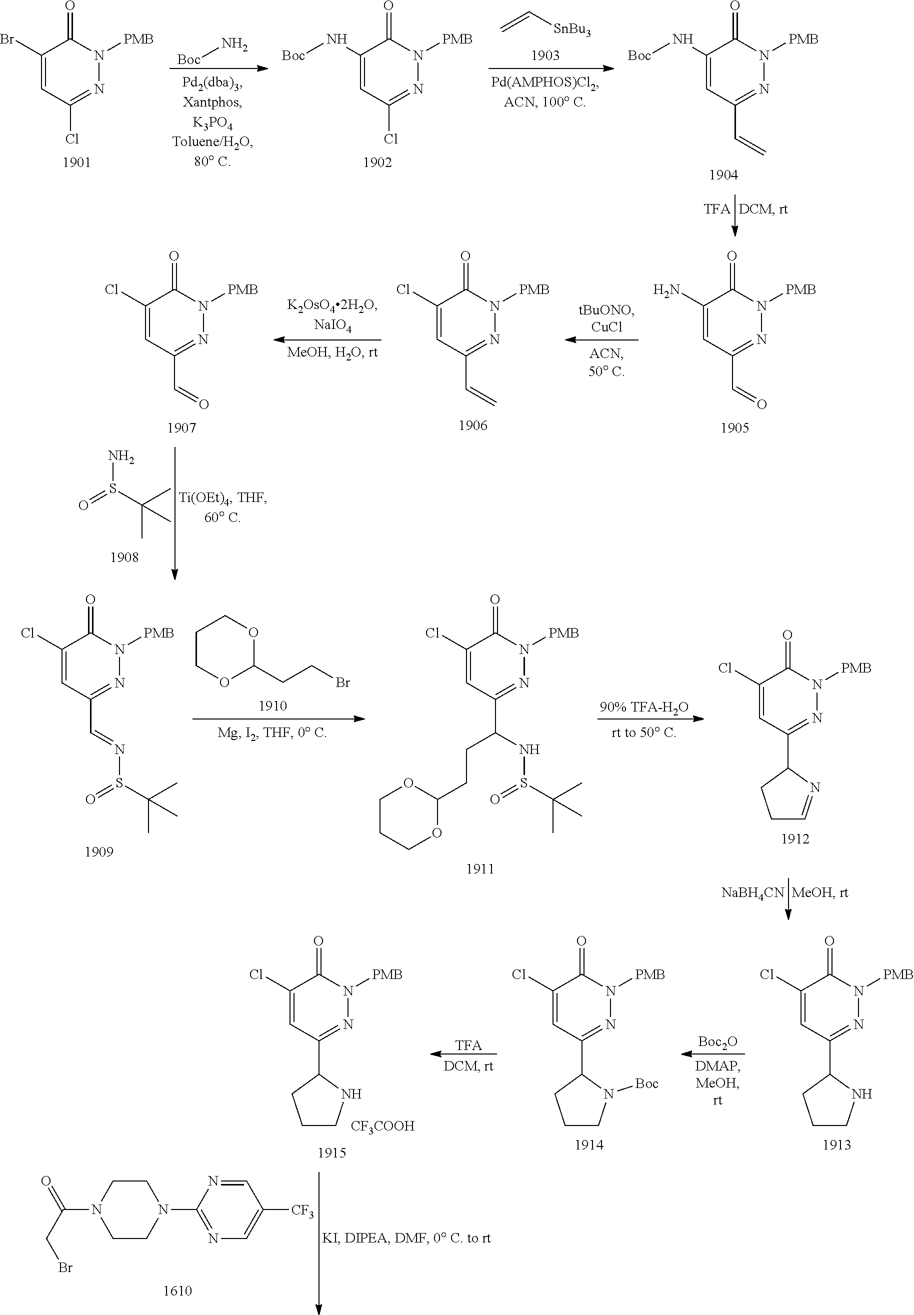

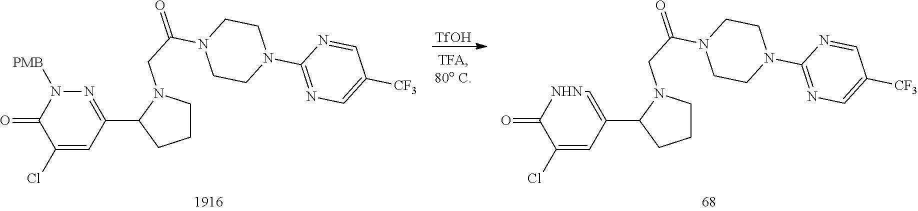

### Preparation of tert-butyl (6-chloro-2-(4-methoxybenzyl)-3-oxo-2,3-dihydropyridazin-4-yl)carbamate (1902)

To a solution of 4-bromo-6-chloro-2-(4-methoxybenzyl)pyridazin-3(2H)-one 1901 (6 g, 18.20 mmol) in toluene/H₂O=6:1 (140 mL) were added tert-butyl carbamate (2.4 g, 20.02 mmol), Pd(dba)₃ (840 mg, 0.9102 mmol), Xantphos (1.1 6, 1.90 mmol), and K₃PO₄ (7.7 g, 36.41 mmol) at rt. The reaction mixture was stirred at 80° C. for 2 h. The resulting mixture was concentrated under reduced pressure. The residue was purified by silica gel column (eluting with PE/EtOAc=100:0 to 50:50) to obtain tert-butyl (6-chloro-2-(4-methoxybenzyl)-3-oxo-2,3-dihydropyridazin-4-yl)carbamate 1902 (3.5 g, 90% purity, 40% yield) as yellow oil.

LCMS (ESI) calcd for C₁₇H₂₀ClN₃O₄[M+H]⁺ m/z 366.11, found m/z 366.00.

### Preparation of tert-butyl (2-(4-methoxybenzyl)-3-oxo-6-vinyl-2,3-dihydropyridazin-4-yl)carbamate (1904)

A mixture of tert-butyl (6-chloro-2-(4-methoxybenzyl)-3-oxo-2,3-dihydropyridazin-4-yl)carbamate 1902 (3.5 g, 0.0095 mol), tributyl(vinyl)stannane 1903 (3.6 g, 0.0114 mol) and Pd(AMPHOS)Cl₂ (0.68 g, 0.0009 mol) in ACN (60 mL) was degassed with N₂ and heated at 100° C. for 2 h under N₂. The mixture was concentrated and purified byflash silica chromatography (eluting with EtOAc/PE, 0 to 50%) to obtain tert-butyl (2-(4-methoxybenzyl)-3-oxo-6-vinyl-2,3-dihydropyridazin-4-yl)carbamate 1904 (1.8 g, 90% purity, 46% yield) as a yellow solid.

LCMS (ESI) calcd for C₁₉H₂₃N₃O₄ [M+H]⁺ m/z 358.17, found 358.10.

### Preparation of 4-amino-2-(4-methoxybenzyl)-6-vinylpyridazin-3(2H)-one (1905)

A suspension of tert-butyl (2-(4-methoxybenzyl)-3-oxo-6-vinyl-2,3-dihydropyridazin-4-yl)carbamate 1904 (1.8 g, 3.942 mmol) in DCM/TFA=1/1(20 mL) was stirred at rt for 30 min, concentrated to obtain 4-amino-2-(4-methoxybenzyl)-6-vinylpyridazin-3(2H)-one 1905 (1.5 g, 90% purity, 95% yield) as a yellow solid.

LCMS (ESI) calcd for C₁₄H₁₅N₃O₂ [M+H]⁺ m/z 258.12, found 258.15.

### Preparation of 4-chloro-2-(4-methoxybenzyl)-6-vinylpyridazin-3(2H)-one (1906)

To a solution of 4-amino-2-(4-methoxybenzyl)-6-vinylpyridazin-3(2H)-one 1905 (1500 mg, 5.835 mmol) in dry ACN (20 mL) was added tert-Butyl nitrite (900 mg, 8.752 mmol) and CuCl (900 mg, 8.752 mmol) at 0° C. The reaction mixture was stirred at 50° C. for 2 h. The reaction solution was concentrated under reduced pressure. The residue was purified by silica gel column (eluting with PE/EtOAc=100:0 to 50:50) to obtain 4-chloro-2-(4-methoxybenzyl)-6-vinylpyridazin-3(2H)-one 1906 (310 mg, 90% purity, 17% yield) as yellow oil.

LCMS (ESI) calcd for C₁₄H₁₃ClN₂O₂[M+H]⁺ m/z 277.07, found 276.87.

### Preparation of 5-chloro-1-(4-methoxybenzyl)-6-oxo-1,6-dihydropyridazine-3-carbaldehyde (1907)

To a solution of 4-chloro-2-(4-methoxybenzyl)-6-vinylpyridazin-3(2H)-one 1906 (310 mg, 1.191 mmol) in MeOH/H₂O (3:1, 20 mL) was added K₂OsO₄·2H₂O (41 mg, 0.119 mmol). After stirring at rt for 10 min, NaIO₄ (1197 mg, 5.5955 mmol) was added and stirred at rt for 4 h. The mixture was filtered, and the filtrate was extracted with EtOAc (30 mL×3). The combined organic layer was washed with brine, dried over Na₂SO₄, concentrated, and purified by flash silica chromatography (eluting with EtOAc/PE, 0 to 15%) to obtain 5-chloro-1-(4-methoxybenzyl)-6-oxo-1,6-dihydropyridazine-3-carbaldehyde 1907 (200 mg, 80% purity, 48% yield) as a yellow solid.

LCMS (ESI) calcd for C₁₃H₁₁ClN₂O₃[M+H]⁺ m/z 279.05, no MS signal found.

### Preparation of (E)-N-((5-chloro-1-(4-methoxybenzyl)-6-oxo-1,6-dihydropyridazin-3-yl)methylene)-2-methylpropane-2-sulfinamide (1909)

To a solution of 5-chloro-1-(4-methoxybenzyl)-6-oxo-1,6-dihydropyridazine-3-carbaldehyde 1907 (200 mg, 0.718 mmol) in THF (50 mL) were added 2-methylpropane-2-sulfinamide 1908 (174 mg, 1.436 mmol) and Ti(OEt)₄ (327 mg, 1.436 mmol). The mixture was heated at 60° C. for 2 h, concentrated and purified by flash silica chromatography (eluting with EtOAc/PE, 0 to 43%) to obtain (E)-N-((5-chloro-1-(4-methoxybenzyl)-6-oxo-1,6-dihydropyridazin-3-yl)methylene)-2-methylpropane-2-sulfinamide 1909 (150 mg, 90% purity, 49% yield) as a yellow solid.

LCMS (ESI) calcd for C₁₇H₂₀ClN₃O₃S [M+H]⁺ m/z 382.09, found 382.25.

### Preparation of N-(1-(5-chloro-1-(4-methoxybenzyl)-6-oxo-1,6-dihydropyridazin-3-yl)-3-(1,3-dioxan-2-yl)propyl)-2-methylpropane-2-sulfinamide (1911)

A 50 mL three-neck flask was charged with Mg (2.5 g), I₂ (0.1 g) and THF (10 mL), and then degassed with N₂. A solution of 2-(2-bromoethyl)-1,3-dioxane 1910 (1.4 g, 0.0072 mol) in THF (10 mL) was added to the mixture in 5 portions. When the first portion was added, a hair dryer was used to initiate reaction, and then the remaining 4 portions were added (keep refluxing). The mixture was stirred for another 30 min to obtain a (2-(1,3-dioxan-2-yl)ethyl)magnesium bromide solution. A solution of (E)-N-((5-chloro-1-(4-methoxybenzyl)-6-oxo-1,6-dihydropyridazin-3-yl)methylene)-2-methylpropane-2-sulfinamide 1909 (150 mg, 0.3928 mmol) in THF (20 mL) was degassed with N₂, added the (2-(1,3-dioxan-2-yl)ethyl)magnesium bromide solution obtained above and stirred at 0° C. for 30 min. The resulting mixture was quenched with saturated NH₄Cl solution and extracted with EtOAc (50 mL×3). The combined organic layer was washed with brine, dried over Na₂SO₄ and concentrated to obtain N-(1-(5-chloro-1-(4-methoxybenzyl)-6-oxo-1,6-dihydropyridazin-3-yl)-3-(1,3-dioxan-2-yl)propyl)-2-methylpropane-2-sulfinamide 1911 (70 mg, 90% purity, 38% yield) as a yellow solid.

LCMS (ESI) calcd for C₂₃H₃₂ClN₃O₅S [M+H]⁺ m/z 498.18, found 498.12.

### Preparation of tert-butyl 2-(5-chloro-1-(4-methoxybenzyl)-6-oxo-1,6-dihydropyridazin-3-yl)pyrrolidine-1-carboxylate (1914)

A solution of N-(1-(5-chloro-1-(4-methoxybenzyl)-6-oxo-1,6-dihydropyridazin-3-yl)-3-(1,3-dioxan-2-yl)propyl)-2-methylpropane-2-sulfinamide 1911 (70 mg, 0.1497 mmol) in 90% TFA-H₂O (10 mL) was stirred at 50° C. for 10 min. The mixture was diluted with MeOH (10 mL), added NaBH₃CN (20 mg, 0.3174 mmol) and stirred at rt for 1 h. The resulting mixture was adjusted to pH 12.0 with 5 M aqueous NaOH solution and then added Boc₂O (492 mg, 2.255 mmol) and DMAP (31 mg, 0.250 mmol). After stirring at rt for 18 h, the mixture was extracted with EtOAc (20 mL×3). The combined organic layer was washed with brine, dried over Na₂SO₄, concentrated and purified with flash silica chromatography (eluting with EtOAc/PE, 0 to 35%) to obtain tert-butyl 2-(5-chloro-1-(4-methoxybenzyl)-6-oxo-1,6-dihydropyridazin-3-yl)pyrrolidine-1-carboxylate 1914 (70 mg, 90% purity, 38% yield) as a yellow oil.

LCMS (ESI) calcd for C₂₁H₂₆ClN₃O₄[M+H]⁺ m/z 420.16, found 420.25.

### Preparation of 4-chloro-2-(4-methoxybenzyl)-6-(pyrrolidin-2-yl)pyridazin-3(2H)-one 2,2,2-trifluoroacetate (1915)

A solution of tert-butyl 2-(5-chloro-1-(4-methoxybenzyl)-6-oxo-1,6-dihydropyridazin-3-yl)pyrrolidine-1-carboxylate 1914 (70 mg, 0.1663 mmol) in DCM (5 mL) and TFA (5 mL) was stirred at rt for 30 min and concentrated to obtain 4-chloro-2-(4-methoxybenzyl)-6-(pyrrolidin-2-yl)pyridazin-3(2H)-one 2,2,2-trifluoroacetate 1915 (60 mg, 80% purity, 66% yield) as a yellow oil.

LCMS (ESI) calcd for C₁₆H₁₈ClN₃O₂ [M+H]⁺ m/z 320.11, found 320.15.

### Preparation of 4-chloro-2-(4-methoxybenzyl)-6-(1-(2-oxo-2-(4-(5-(trifluoromethyl)pyrimidin-2-yl)piperazin-1-yl)ethyl)pyrrolidin-2-yl)pyridazin-3(2H)-one (1916)

To a solution of 4-chloro-2-(4-methoxybenzyl)-6-(pyrrolidin-2-yl)pyridazin-3(2H)-one 2,2,2-trifluoroacetate 1915 (60 mg, 0.1383 mmol) in DMF (5 mL) at 0° C. were added DIPEA (48 mg, 0.3752 mmol), KI (31 mg, 0.1876 mmol) and 2-bromo-1-(4-(5-(trifluoromethyl)pyrimidin-2-yl)piperazin-1-yl)ethan-1-one 1610 (73 mg, 0.2063 mmol). The mixture was then stirred at rt for 2 h. The resulting reaction mixture was diluted with water and then extracted with EtOAc (30 mL×3). The combined organic layer was washed with brine, dried over Na₂SO₄, filtered, concentrated and purified with flash silica chromatography (eluting with MeOH/DCM, 0 to 3%) to obtain 4-chloro-2-(4-methoxybenzyl)-6-(1-(2-oxo-2-(4-(5-(trifluoromethyl)pyrimidin-2-yl)piperazin-1-yl)ethyl)pyrrolidin-2-yl)pyridazin-3(2H)-one 1916 (40 mg, 90% purity, 43% yield) as a white solid.

LCMS (ESI) calcd for C₂₇H₂₉ClF₃N₇O₃ [M+H]⁺ m/z 592.20, found 592.30.

### Preparation of 4-chloro-6-(1-(2-oxo-2-(4-(5-(trifluoromethyl)pyrimidin-2-yl)piperazin-1-yl)ethyl)pyrrolidin-2-yl)pyridazin-3(2H)-one (68)

To a solution of4-chloro-2-(4-methoxybenzyl)-6-(1-(2-oxo-2-(4-(5-(trifluoromethyl)pyrimidin-2-yl)piperazin-1-yl)ethyl)pyrrolidin-2-yl)pyridazin-3(2H)-one 1916 (40 mg, 0.0676 mmol) in TFA (5 mL) was added TfOH (0.2 mL). The mixture was heated at 80° C. for 5 min, adjusted to pH 8.0 with saturated NaHCO₃ solution at room temperature and extracted with DCM (100 mL×3). The combined organic layer was washed with brine, dried over Na₂SO₄, concentrated, and purified with prep-HPLC (Column: Gemini 5 μm C₁₈ 150×21.2 mm, mobile phase: ACN—H₂O (0.1% FA), gradient: 10-90) to obtain 4-chloro-6-(1-(2-oxo-2-(4-(5-(trifluoromethyl)pyrimidin-2-yl)piperazin-1-yl)ethyl)pyrrolidin-2-yl)pyridazin-3(2H)-one 68 (8.6 mg, 98% purity, 26% yield) as a white solid.

¹H NMR (400 MHz, DMSO-d₆, ppm) δ: 13.24 (s, 1H), 8.73 (s, 2H), 7.76 (s, 1H), 3.98-3.84 (m, 2H), 3.75-3.57 (m, 3H), 3.55-3.36 (m, 4H), 3.30-3.27 (m, 1H), 3.21-3.13 (m, 2H), 2.48-2.47 (m, 1H), 2.18-2.04 (m, 1H), 1.94-1.67 (m, 3H).

LCMS (ESI) calcd for C₁₉H₂₁ClF₃N₇O₂ [M+H]⁺ m/z 472.14, found 472.10.

### 11. Synthesis of 3-chloro-5-(1-(2-oxo-2-(4-(5-(trifluoromethyl)pyrimidin-2-yl)piperazin-1-yl)ethyl)pyrrolidin-2-yl)pyridin-2(1H)-one (Compounds 78a and 78b)

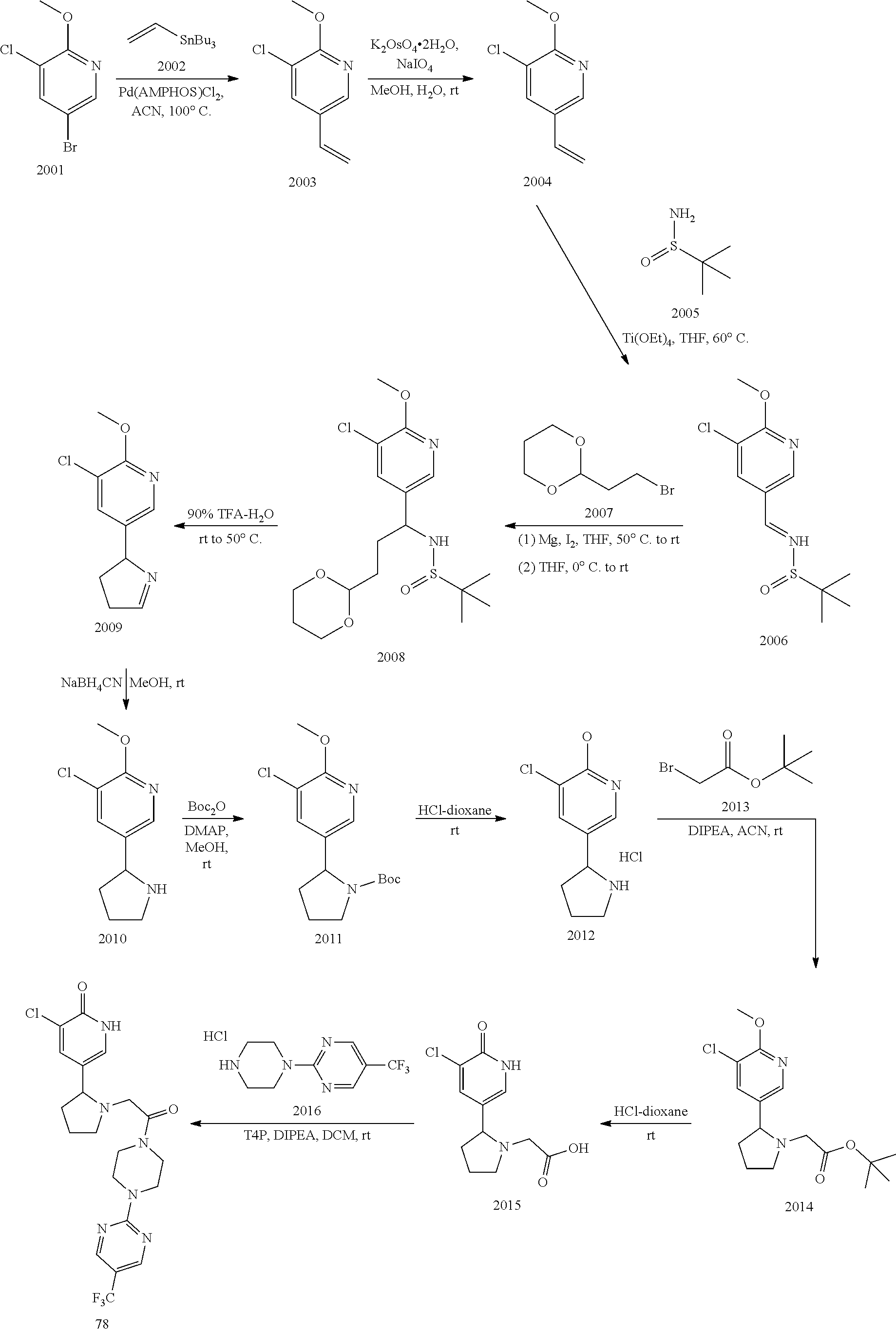

### Preparation of 3-chloro-2-methoxy-5-vinylpyridine (2003)

A mixture of 5-bromo-3-chloro-2-methoxypyridine 2001 (5.0 g, 0.0225 mol), tributyl(vinyl)stannane 2002 (14.3 g, 0.0450 mol) and Pd(AMPHOS)Cl₂ (1.6 g, 0.0023 mol) in ACN (50 mL) was degassed with N₂ and heated at 100° C. for 2 h under N₂ atmosphere. The mixture was concentrated and purified by flash silica chromatography (eluting with EtOAc/PE, 0 to 15%) to obtain 3-chloro-2-methoxy-5-vinylpyridine 2003 (4.0 g, 70% purity, 73% yield) as a yellow oil.

LCMS (ESI) calcd for C₈H₈ClNO [M+H]⁺ m/z 170.03, found 170.20.

### Preparation of 5-chloro-6-methoxynicotinaldehyde (2004)

To a solution of 3-chloro-2-methoxy-5-vinylpyridine 2003 (4.00 g, 0.0236 mol) in MeOH (350 mL) and H₂O (70 mL) were added K₂OsO₄·2H₂O (0.87 g, 0.0024 mol) and NaIO₄ (20.2 g, 0.0944 mol). The mixture was stirred at rt for 2 h, and then filtered to remove the white solid. The filtrate was washed with water and extracted with EtOAc (200 mL×3). The combined organic layer was washed with brine, dried over Na₂SO₄, concentrated, and purified by flash silica chromatography (eluting with EtOAc/PE, 0 to 25%) to obtain 5-chloro-6-methoxynicotinaldehyde 2004 (1.96 g, 90% purity, 43% yield) as a white solid.

LCMS (ESI) calcd for C₇H₆ClNO₂ [M+H]⁺ m/z 172.01, found 172.20.

### Preparation of (E)-N-((5-chloro-6-methoxypyridin-3-yl)methylene)-2-methylpropane-2-sulfinamide (2006)

To a solution of 5-chloro-6-methoxynicotinaldehyde 2004 (1.96 g, 0.0114 mol) in THF (30 mL) were added 2-methylpropane-2-sulfinamide 2005 (2.76 g, 0.0228 mol) and Ti(OEt)₄ (5.20 g, 0.0228 mol). The mixture was heated at 60° C. for 2 h, concentrated and purified by flash silica chromatography (eluting with EtOAc/PE, 0 to 25%) to obtain (E)-N-((5-chloro-6-methoxypyridin-3-yl)methylene)-2-methylpropane-2-sulfinamide 2006 (3.22 g, 90% purity, 92% yield) as a yellow solid.

LCMS (ESI) calcd for C₁₁H₁₅ClN₂O₂S [M+H]⁺ m/z 275.06, found 275.20.

### Preparation of N-(1-(5-chloro-6-methoxypyridin-3-yl)-3-(1,3-dioxan-2-yl)propyl)-2-methylpropane-2-sulfinamide (2008)

A 250 mL three-neck flask was charged with Mg (3.37 g, 0.1404 mol), I₂ (0.01 g, 0.00006 mol) in THF (20 mL) was degassed with N₂. A solution of 2-(2-bromoethyl)-1,3-dioxane 2007 (3.42 g, 0.018 mol) in THF (10 mL) was added in 5 portions. When the first portion was added, a hair dryer was used to initiate reaction, and the remaining 4 portions were added at 50° C. The mixture was stirred at 50° C. for 30 min to obtain (2-(1,3-dioxan-2-yl)ethyl)magnesium bromide solution. A solution of (E)-N-((5-chloro-6-methoxypyridin-3-yl)methylene)-2-methylpropane-2-sulfinamide 2006 (3.22 g, 0.0117 mol) in THF (10 mL) was degassed with N₂, the (2-(1,3-dioxan-2-yl)ethyl)magnesium bromide solution obtained above was added at 0° C. and the mixture was stirred at rt for 30 min. The resulting mixture was quenched with saturated NH₄Cl solution and extracted with EtOAc (200 mL×3). The combined organic layer was washed with brine, dried over Na₂SO₄ and concentrated to obtain N-(1-(5-chloro-6-methoxypyridin-3-yl)-3-(1,3-dioxan-2-yl)propyl)-2-methylpropane-2-sulfinamide 2008 (4.0 g, 90% purity, 78% yield) as a yellow oil.

LCMS (ESI) calcd for C₁₇H₂₇ClN₂O₄S [M+H]⁺ m/z 391.14, found 390.93.

### Preparation of tert-butyl 2-(5-chloro-6-methoxypyridin-3-yl)pyrrolidine-1-carboxylate (2011)

A solution of N-(1-(5-chloro-6-methoxypyridin-3-yl)-3-(1,3-dioxan-2-yl)propyl)-2-methylpropane-2-sulfinamide 2008 (4.0 g, 0.010 mol) in 90% TFA-H₂O (30 mL) was heated at 50° C. for 1 h. After cooling to rt, the mixture was diluted with MeOH (20 mL), NaBH₃CN (0.8 g, 0.013 mol) was added and the mixture was stirred at rt for 1 h. The resulting mixture was adjusted to pH 12.0 by addition of 5 M aqueous NaOH solution and then Boc₂O (9.2 g, 0.042 mol) and DMAP (0.6 g, 0.005 mol) were added. After stirring at rt for 1 h, the mixture was extracted with EtOAc (200 mL×3). The combined organic layer was washed with brine, dried over Na₂SO₄, concentrated and purified by flash silica chromatography (eluting with EtOAc/PE, 0 to 17%) to obtain tert-butyl 2-(5-chloro-6-methoxypyridin-3-yl)pyrrolidine-1-carboxylate 2011 (1.2 g, 70% purity, 26% yield) as a colorless oil.

LCMS (ESI) calcd for C₁₅H₂₁ClN₂O₃[M+H]⁺ m/z 313.12, found 313.25.

### Preparation of 3-chloro-2-methoxy-5-(pyrrolidin-2-yl)pyridine hydrochloride (2012)

A solution of tert-butyl 2-(5-chloro-6-methoxypyridin-3-yl)pyrrolidine-1-carboxylate 2011 (500 mg, 1.59 mmol) in 4 M HCl-dioxane (15 mL) was stirred at rt for 2 h and concentrated to obtain 3-chloro-2-methoxy-5-(pyrrolidin-2-yl)pyridine hydrochloride 2012 (330 mg, 90% purity, 74% yield) as a white solid.

LCMS (ESI) calcd for C₁₀H₁₃ClN₂O [M+H]⁺ m/z 213.07, found 213.25.

### Preparation of tert-butyl 2-(2-(5-chloro-6-methoxypyridin-3-yl)pyrrolidin-1-yl)acetate (2014)

To a solution of 3-chloro-2-methoxy-5-(pyrrolidin-2-yl)pyridine hydrochloride 2012 (330 mg, 1.32 mmol) in ACN (20 mL) were added DIPEA (514 mg, 3.97 mmol) and tert-butyl 2-bromoacetate 2013 (336 mg, 1.72 mmol). The mixture was stirred at rt for 2 h, diluted with water (100 mL) and extracted with EtOAc (100 mL×3). The combined organic layer was washed with brine, dried over Na₂SO₄, concentrated and purified with flash silica chromatography (eluting with EtOAc/PE, 0 to 21%) to obtain tert-butyl 2-(2-(5-chloro-6-methoxypyridin-3-yl)pyrrolidin-1-yl)acetate 2014 (260 mg, 90% purity, 54% yield) as a colorless oil.

LCMS (ESI) calcd for C₁₆H₂₃ClN₂O₃ [M+H]⁺ m/z 327.14, found 327.30.

### Preparation of 2-(2-(5-chloro-6-hydroxypyridin-3-yl)pyrrolidin-1-yl)acetic acid (2015)

A solution of tert-butyl 2-(2-(5-chloro-6-methoxypyridin-3-yl)pyrrolidin-1-yl)acetate 2014 (260 mg, 0.80 mmol) in HCl-dioxane (4 M, 10 mL) was stirred at rt for 16 h. The reaction solution was concentrated under reduced pressure to obtain 2-(2-(5-chloro-6-hydroxypyridin-3-yl)pyrrolidin-1-yl)acetic acid 2015 (180 mg, 80% purity, 70% yield) as white solid.

LCMS (ESI) calcd for C₁₁H₁₃ClN₂O₃[M+H]⁺ m/z 257.06, found 257.25.

### Preparation of 3-chloro-5-(1-(2-oxo-2-(4-(5-(trifluoromethyl)pyrimidin-2-yl)piperazin-1-yl)ethyl)pyrrolidin-2-yl)pyridin-2(1H)-one (78rac)

To a solution of 2-(2-(5-chloro-6-hydroxypyridin-3-yl)pyrrolidin-1-yl)acetic acid 2015 (180 mg, 0.70 mmol) and 2-(piperazin-1-yl)-5-(trifluoromethyl)pyrimidine hydrochloride 2016 (195 mg, 0.84 mmol) in DCM (5 mL) were added DIPEA (362 mg, 2.80 mmol) and T4P (1010 mg, 1.40 mmol, 50% wt in EtOAc). The reaction mixture was stirred at rt for 1 h. The reaction solution was quenched with water (15 mL) and extracted with DCM (20 mL×2). The organic phase was concentrated and purified by flash column chromatography (eluting with DCM/MeOH=100:0 to 95:5) to afford 3-chloro-5-(1-(2-oxo-2-(4-(5-(trifluoromethyl)pyrimidin-2-yl)piperazin-1-yl)ethyl)pyrrolidin-2-yl)pyridin-2(1H)-one, racemic mixture of 78a and 78b (260 mg, 99% purity, 77% yield) as white solid.

### Chiral resolution of 3-chloro-5-(1-(2-oxo-2-(4-(5-(trifluoromethyl)pyrimidin-2-yl)piperazin-1-yl)ethyl)pyrrolidin-2-yl)pyridin-2(1H)-one (78rac)

The racemic mixture of 78a and 78b was separated by SFC (Column: Daicel Chiralpak IC SFC; 20 mm×250 mm I.D., 5 μm; Mobile phase: CO₂/MeOH [0.1% NH₃(7 M Solution in MeOH)]=60/40) and concentrated under reduced pressure to afford the first fraction as 78a (109.0 mg, 99% purity, 100% ee, white solid) and the second fraction as 78b (114.6 mg, 99% purity, 100% ee, white solid).

### Compound 78a

¹H NMR (400 MHz, DMSO-d₆, ppm) δ:12.00 (s, 1H), 8.73 (s, 2H), 7.69 (d, J=2.0 Hz, 1H), 7.25 (d, J=1.2 Hz, 1H), 4.05-3.87 (m, 2H), 3.69-3.56 (m, 3H), 3.56-3.47 (m, 1H), 3.40-3.33 (m, 2H), 3.26-3.14 (m, 3H), 3.00 (d, J=13.6 Hz, 1H), 2.40-2.28 (m, 1H), 2.10-1.98 (m, 1H), 1.91-1.80 (m, 1H), 1.80-1.68 (m, 1H), 1.68-1.56 (m, 1H).

LCMS (ESI) calcd for C₂₀H₂₂ClF₃N₆O₂ [M+H]⁺ m/z 471.14, found 471.03.

### Compound 78b

¹H NMR (400 MHz, DMSO-d₆, ppm) δ:12.00 (s, 1H), 8.73 (s, 2H), 7.69 (d, J=1.2 Hz, 1H), 7.25 (s, 1H), 4.01-3.87 (m, 2H), 3.72-3.57 (m, 3H), 3.57-3.45 (m, 1H), 3.40-3.32 (m, 2H), 3.25-3.13 (m, 3H), 3.00 (d, J=13.6 Hz, 1H), 2.38-2.28 (m, 1H), 2.10-1.98 (m, 1H), 1.92-1.80 (m, 1H), 1.80-1.68 (m, 1H), 1.68-1.54 (m, 1H).

LCMS (ESI) calcd for C₂₀H₂₂ClF₃N₆O₂ [M+H]⁺ m/z 471.14, found 471.01.

### 12. Synthesis of 5-(1-(2-oxo-2-(4-(5-(trifluoromethyl)pyrimidin-2-yl)piperazin-1-yl)ethyl)pyrrolidin-2-yl)-3-(trifluoromethyl)pyrazin-2(1H)-one (Compounds 84a and 84b)

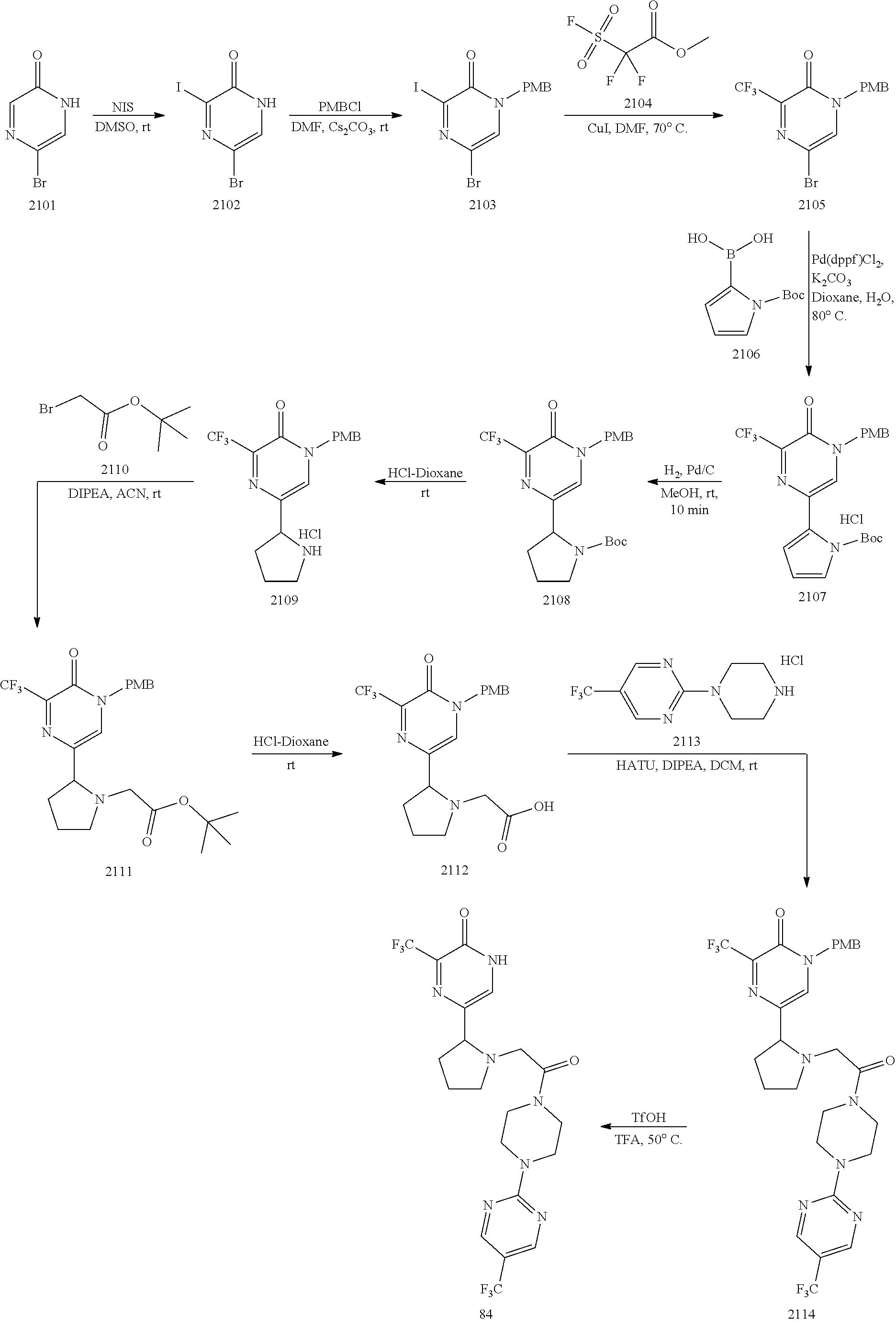

### Preparation of 5-bromo-3-iodopyrazin-2(1H)-one (2102)

To a solution of 5-bromopyrazin-2(1H)-one 2101 (4.5 g, 0.0257 mol) in DMSO (40 mL) was added NIS (6.9 g, 0.0308 mol) at rt. The reaction mixture was stirred at rt for 4 h. The resulting mixture was quenched with water and extracted with EtOAc (300 mL×3). The combined organic layer was washed with brine and concentrated under reduced pressure. The residue was purified by flash column chromatography (eluting with PE/EtOAc=100:0 to 50:50) to obtain 5-bromo-3-iodopyrazin-2(1H)-one 2102 (5.0 g, 80% purity, 51% yield) as a black solid.

LCMS (ESI) calcd for C₄H₂BrIN₂O [M+H]⁺ m/z 300.84, found 300.70.

### Preparation of 5-bromo-3-iodo-1-(4-methoxybenzyl)pyrazin-2(1H)-one (2103)

To a solution of 5-bromo-3-iodopyrazin-2(1H)-one 2102 (2.0 g, 0.00665 mol) in DMF (30 mL) were added Cs₂CO₃ (4.3 g, 0.0132 mol) and PMBCl (2.1 g, 0.0132 mol) at rt. After completion of addition, the reaction solution was stirred at rt for 2 h. The reaction mixture was added into cold water and then extracted with EtOAc (50 mL×3). The combined organic layer was washed with brine, dried over Na₂SO₄, and concentrated under reduced pressure. The residue was purified by flash silica chromatography (eluting with PE/EtOAc=100:0 to 50:50) to give 5-bromo-3-iodo-1-(4-methoxybenzyl)pyrazin-2(1H)-one 2103 (1.5 g, 90% purity, 48% yield) as a yellow oil.

LCMS (ESI) calcd for C₁₂H₁₀BrIN₂O₂[M+H]⁺ m/z 420.90, found 420.85.

### Preparation of 5-bromo-1-(4-methoxybenzyl)-3-(trifluoromethyl)pyrazin-2(1H)-one (2105)

To a solution of 5-bromo-3-iodo-1-(4-methoxybenzyl)pyrazin-2(1H)-one 2103 (1.40 g, 3.3 mmol) in DMF (20 mL) were added methyl 2,2-difluoro-2-(fluorosulfonyl)acetate 2104 (1.27 g, 6.6 mmol) and CuI (0.13 g, 0.7 mmol) at rt. The reaction mixture was stirred at 70° C. for 16 h. The reaction solution was quenched with water (200 mL) and extracted with EtOAc (200 mL×3). The organic phase was concentrated and purified by flash column chromatography (eluting with DCM/MeOH=100:0 to 95:5) to afford 5-bromo-1-(4-methoxybenzyl)-3-(trifluoromethyl)pyrazin-2(1H)-one 2105 (0.55 g, 90% purity, 42% yield) as a yellow oil.

LCMS (ESI) calcd for C₁₃H₁₀BrF₃N₂O₂ [M+H]⁺ m/z 362.99, no MS signal.

### Preparation of tert-butyl 2-(4-(4-methoxybenzyl)-5-oxo-6-(trifluoromethyl)-4,5-dihydropyrazin-2-yl)-1H-pyrrole-1-carboxylate (2107)

To a solution of 5-bromo-1-(4-methoxybenzyl)-3-(trifluoromethyl)pyrazin-2(1H)-one 2105 (1.10 g, 0.0030 mol) and (1-(tert-butoxycarbonyl)-1H-pyrrol-2-yl)boronic acid 2106 (1.27 g, 0.0060 mol) in 1,4-dioxane/H₂O (10:1, 50 mL) were added Pd(dppf)Cl₂ (0.22 g, 0.00030 mol) and K₂CO₃ (1.24 g, 0.0090 mol) at rt. The resulting mixture was stirred at 80° C. for 1 h. After cooling to rt, the reaction mixture was poured into water, then extracted with EtOAc (100 mL×3). The combined organic layers were washed with brine (100 mL×3), dried over Na₂SO₄, and concentrated under reduced pressure. The residue was purified by flash column chromatography (eluting with PE/EtOAc=100:0 to 50:50) to afford tert-butyl 2-(4-(4-methoxybenzyl)-5-oxo-6-(trifluoromethyl)-4,5-dihydropyrazin-2-yl)-1H-pyrrole-1-carboxylate 2107 (1.20 g, 90% purity, 80% yield) as a yellow oil.

LCMS (ESI) calcd for C₂₂H₂₂F₃N₃O₄[M+H]⁺ m/z 450.16, found 450.15.

### Preparation of tert-butyl 2-(4-(4-methoxybenzyl)-5-oxo-6-(trifluoromethyl)-4,5-dihydropyrazin-2-yl)-1H-pyrrole-1-carboxylate (2108)

To a solution of tert-butyl 2-(4-(4-methoxybenzyl)-5-oxo-6-(trifluoromethyl)-4,5-dihydropyrazin-2-yl)-1H-pyrrole-1-carboxylate 2107 (1200 mg, 2.67 mmol) in MeOH (30 mL) was added Pd/C (1200 mg, 10% wt.) at rt. The reaction mixture was degassed with H₂ and stirred at rt for 10 min under H₂ atmosphere. The reaction solution was filtered. The filtrate was concentrated and purified by flash column chromatography (eluting with PE/EtOAc=100:0 to 60:40) to obtain tert-butyl 2-(4-(4-methoxybenzyl)-5-oxo-6-(trifluoromethyl)-4,5-dihydropyrazin-2-yl)-1H-pyrrole-1-carboxylate 2108 (600 mg, 40% purity, 19% yield) as a yellow oil.

LCMS (ESI) calcd for C₂₂H₂₆F₃N₃O₄[M+H]⁺ m/z 454.19, found 454.15.

### Preparation of 1-(4-methoxybenzyl)-5-(pyrrolidin-2-yl)-3-(trifluoromethyl)pyrazin-2(1H)-one hydrochloride (2109)

A solution of tert-butyl 2-(4-(4-methoxybenzyl)-5-oxo-6-(trifluoromethyl)-4,5-dihydropyrazin-2-yl)pyrrolidine-1-carboxylate 2108 (600 mg, 40% purity, 0.53 mmol) in HCl in Dioxane (4 M, 30 mL) was stirred at rt for 1 h. The reaction solution was concentrated to afford 1-(4-methoxybenzyl)-5-(pyrrolidin-2-yl)-3-(trifluoromethyl)pyrazin-2(1H)-one hydrochloride 2109 (350 mg, 40% purity, 67% yield) as a yellow oil.

LCMS (ESI) calcd for C₁₇H₁₈F₃N₃O₂[M+H]⁺ m/z 354.14, found 354.20.

### Preparation of tert-butyl 2-(2-(4-(4-methoxybenzyl)-5-oxo-6-(trifluoromethyl)-4,5-dihydropyrazin-2-yl)pyrrolidin-1-yl)acetate (2111)

To a solution of 1-(4-methoxybenzyl)-5-(pyrrolidin-2-yl)-3-(trifluoromethyl)pyrazin-2(1H)-one hydrochloride 2109 (350 mg, 40% purity, 0.36 mmol) in ACN (20 mL) were added DIPEA (254 mg, 1.97 mmol) and tert-butyl 2-bromoacetate 2110 (385 mg, 1.97 mmol) at rt. The mixture was stirred at rt for 2 h. The reaction solution was quenched with water (50 mL) and extracted with DCM (80 mL×3). The organic phase was concentrated and purified by flash column chromatography (eluting with DCM/MeOH=100:0 to 95:5) to afford tert-butyl 2-(2-(4-(4-methoxybenzyl)-5-oxo-6-(trifluoromethyl)-4,5-dihydropyrazin-2-yl)pyrrolidin-1-yl)acetate 2111 (130 mg, 90% purity, 69% yield) as a yellow oil.

LCMS (ESI) calcd for C₂₃H₂₈F₃N₃O₄[M+H]⁺ m/z 468.20, found 468.25.

### Preparation of 2-(2-(4-(4-methoxybenzyl)-5-oxo-6-(trifluoromethyl)-4,5-dihydropyrazin-2-yl)pyrrolidin-1-yl)acetic acid (2112)

A solution of tert-butyl 2-(2-(4-(4-methoxybenzyl)-5-oxo-6-(trifluoromethyl)-4,5-dihydropyrazin-2-yl)pyrrolidin-1-yl)acetate 2111 (130 mg, 0.27 mmol) in HCl in Dioxane (4 M, 20 mL) was stirred at rt for 1 h. The reaction solution was concentrated to afford 2-(2-(4-(4-methoxybenzyl)-5-oxo-6-(trifluoromethyl)-4,5-dihydropyrazin-2-yl)pyrrolidin-1-yl)acetic acid 2112 (100 mg, 90% purity, 78% yield) as a yellow oil.

LCMS (ESI) calcd for C₁₉H₂₀F₃N₃O₄ [M+H]⁺ m/z 412.14, found 412.15.

### Preparation of 1-(4-methoxybenzyl)-5-(1-(2-oxo-2-(4-(5-(trifluoromethyl)pyrimidin-2-yl)piperazin-1-yl)ethyl)pyrrolidin-2-yl)-3-(trifluoromethyl)pyrazin-2(1H)-one (2114)

To a solution of 2-(2-(4-(4-methoxybenzyl)-5-oxo-6-(trifluoromethyl)-4,5-dihydropyrazin-2-yl)pyrrolidin-1-yl)acetic acid 2112 (100 mg, 0.24 mmol) in DMF (30 mL) were added 2-(piperazin-1-yl)-5-(trifluoromethyl)pyrimidine hydrochloride 2113 (108 mg, 0.40 mmol), DIPEA (117 mg, 0.90 mmol) and HATU (230 mg, 0.60 mmol) at rt. The reaction mixture was stirred at rt for 1 h. The reaction solution was quenched with water (50 mL) and extracted with EtOAc (80 mL×3). The organic phase was concentrated and purified by flash column chromatography (eluting with DCM/MeOH=100:0 to 95:5) to afford 1-(4-methoxybenzyl)-5-(1-(2-oxo-2-(4-(5-(trifluoromethyl)pyrimidin-2-yl)piperazin-1-yl)ethyl)pyrrolidin-2-yl)-3-(trifluoromethyl)pyrazin-2(1H)-one 2114 (110 mg, 90% purity, 65% yield) as a yellow oil.

LCMS (ESI) calcd for C₂₈H₂₉F₆N₇O₃[M+H]⁺ m/z 626.22, found 626.20.

### Preparation of 5-(1-(2-oxo-2-(4-(5-(trifluoromethyl)pyrimidin-2-yl)piperazin-1-yl)ethyl)pyrrolidin-2-yl)-3-(trifluoromethyl)pyrazin-2(1H)-one (84rac)

To a solution of 1-(4-methoxybenzyl)-5-(1-(2-oxo-2-(4-(5-(trifluoromethyl)pyrimidin-2-yl)piperazin-1-yl)ethyl)pyrrolidin-2-yl)-3-(trifluoromethyl)pyrazin-2(1H)-one 2114 (110 mg, 0.17 mmol) in TFA (5 mL) was added TfOH (0.5 mL) at rt. The reaction solution was stirred at 50° C. for 30 min. The mixture was adjusted to pH=8-9 with aqueous NaHCO₃ at 0° C., then extracted with EtOAc (100 mL×3). The combined organic layers were concentrated under reduced pressure. The residue was purified by flash column chromatography (eluting with DCM/MeOH=100:0 to 95:5) to afford 5-(1-(2-oxo-2-(4-(5-(trifluoromethyl)pyrimidin-2-yl)piperazin-1-yl)ethyl)pyrrolidin-2-yl)-3-(trifluoromethyl)pyrazin-2(1H)-one, 84a/84b racemic mixture (70 mg, 90% purity, 70% yield) as yellow oil.

### Chiral resolution of 5-(1-(2-oxo-2-(4-(5-(trifluoromethyl)pyrimidin-2-yl)piperazin-1-yl)ethyl)pyrrolidin-2-yl)-3-(trifluoromethyl)pyrazin-2(1H)-one (84rac)

84a/84b racemic mixture was separated by SFC (Column: Daicel IH 250 mm×20 mm I.D., 5 μm; Mobile phase: CO₂/MeOH (0.1% NH₃)=60/40) and concentrated under reduced pressure to afford the first fraction as 84a (36.9 mg, 99% purity, 100% ee, white solid) and the second fraction as 84b (30.0 mg, 99% purity, 100% ee, white solid).

### Compound 84a

¹H NMR (400 MHz, DMSO-d₆, ppm) δ: 13.03 (s, 1H), 8.73 (s, 2H), 7.73 (s, 1H), 3.99-3.82 (m, 2H), 3.70-3.45 (m, 6H), 3.41-3.34 (m, 1H), 3.26-3.21 (m, 1H), 3.19-3.07 (m, 2H), 2.54-2.51 (m, 1H), 2.11-1.99 (m, 1H), 1.88-1.73 (m, 3H).

LCMS (ESI) calcd for C₂₀H₂₁F₆N₇O₂[M+H]⁺ m/z 506.17, found 506.15.

Compound 84b 10 ¹H NMR (400 MHz, DMSO-d₆, ppm) δ: 13.02 (s, 1H), 8.73 (s, 2H), 7.74 (s, 1H), 3.98-3.81 (m, 2H), 3.68-3.45 (m, 6H), 3.41-3.34 (m, 1H), 3.28-3.22 (m, 1H), 3.20-3.07 (m, 2H), 2.54-2.51 (m, 1H), 2.14-2.02 (m, 1H), 1.87-1.71 (m, 3H).

LCMS (ESI) calcd for C₂₀H₂₁F₆N₇O₂[M+H]⁺ m/z 506.17, found 506.20.

### 13. Synthesis of (R)-6-(4,4-difluoro-1-(2-oxo-2-(4-(5-(trifluoromethyl)pyrimidin-2-yl)piperazin-1-yl)ethyl)pyrrolidin-2-yl)-4-(trifluoromethyl)pyridazin-3(2H)-one (Compound 91a)

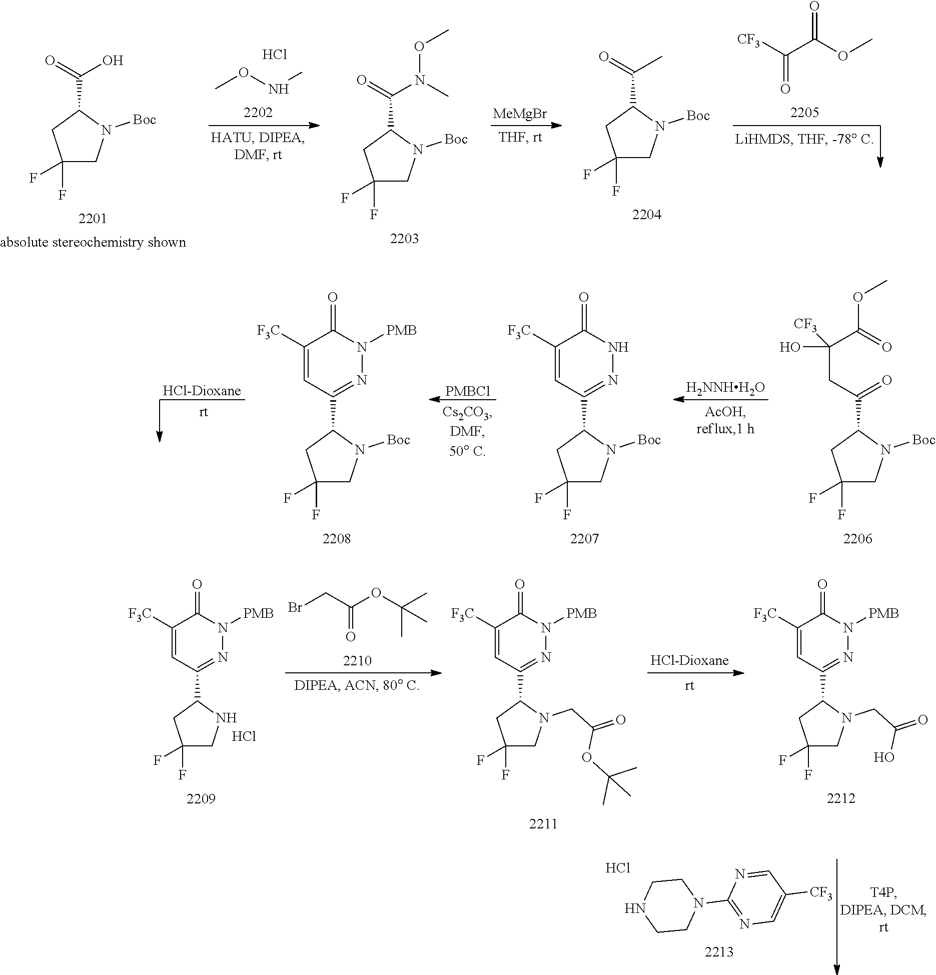

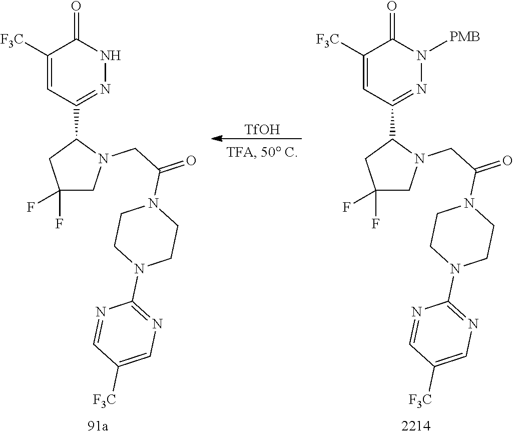

### Preparation of tert-butyl (R)-4,4-difluoro-2-(methoxy(methyl)carbamoyl)pyrrolidine-1-carboxylate (2203)

To a solution of (R)-1-(tert-butoxycarbonyl)-4,4-difluoropyrrolidine-2-carboxylic acid 2201 (2.0 g, 0.008 mol) and N,O-dimethylhydroxylamine hydrochloride 2202 (2.3 g, 0.023 mol) in DMF (30 mL) were added DIPEA (3.1 g, 0.024 mol) and HATU (6.0 g, 0.016 mol) at rt. The reaction mixture was stirred at rt for 1 h. The reaction solution was quenched with water (200 mL) and extracted with EtOAc (100 mL×3). The organic phase was concentrated, and the residue was purified by flash column chromatography (eluting with DCM/MeOH=100:0 to 95:5) to afford tert-butyl (R)-4,4-difluoro-2-(methoxy(methyl)carbamoyl)pyrrolidine-1-carboxylate 2203 (2 g, 90% purity, 77% yield) as yellow oil.

LCMS (ESI) calcd for C₁₂H₂₀F₂N₂O₄[M+Na]⁺ m/z 317.14, found 317.15.

### Preparation of tert-butyl (R)-2-acetyl-4,4-difluoropyrrolidine-1-carboxylate (2204)

To a solution of tert-butyl (R)-4,4-difluoro-2-(methoxy(methyl)carbamoyl)pyrrolidine-1-carboxylate 2203 (2 g, 0.0068 mol) in THF (20 mL) was added MeMgBr (1 M, 13.6 mL, 0.0136 mol) at rt. The resulting mixture was stirred at rt for 1 h. Water (100 mL) was added, and the mixture was extracted EtOAc (150 mL×2). The combined organic layers were dried over Na₂SO₄, concentrated in vacuo and the residue was purified by flash column chromatography (eluting with PE/EtOAc=100:0 to 60:40) to afford tert-butyl (R)-2-acetyl-4,4-difluoropyrrolidine-1-carboxylate 2204 (1.5 g, 90% purity, 79% yield) as a yellow oil.

LCMS (ESI) calcd for C₁₁H₁₇F₂NO₃ [M-t-Bu+H]⁺ m/z 194.12, found 194.15.

### Preparation of tert-butyl (2R)-4,4-difluoro-2-(4,4,4-trifluoro-3-hydroxy-3-(methoxycarbonyl)butanoyl)pyrrolidine-1-carboxylate (2206)

To a solution of tert-butyl (R)-2-acetyl-4,4-difluoropyrrolidine-1-carboxylate 2204 (1.5 g, 0.0060 mol) in THF (20 mL) was added LiHMDS (1 M, 6 mL, 0.0060 mol) at −78° C. The resulting mixture was stirred at −78° C. for 0.5 h. To the reaction mixture was added methyl 3,3,3-trifluoro-2-oxopropanoate 2205 (1.2 g, 0.0078 mol) at −78° C. and then stirred at −78° C. for 0.5 h. The reaction mixture was poured into water, then extracted with EtOAc (80 mL×3). The combined organic layers were concentrated under reduced pressure. The residue was purified by flash column chromatography (eluting with PE/EtOAc=100:0 to 60:40) to afford tert-butyl (2R)-4,4-difluoro-2-(4,4,4-trifluoro-3-hydroxy-3-(methoxycarbonyl)butanoyl)pyrrolidine-1-carboxylate 2206 (1.2 g, 40% purity, 20% yield) as a yellow oil.

LCMS (ESI) calcd for C₁₅H₂₀F₅NO₆ [M+Na]⁺ m/z 428.12, found 428.10.

### Preparation of tert-butyl (R)-4,4-difluoro-2-(6-oxo-5-(trifluoromethyl)-1,6-dihydropyridazin-3-yl)pyrrolidine-1-carboxylate (2207)

To a solution of tert-butyl (2R)-4,4-difluoro-2-(4,4,4-trifluoro-3-hydroxy-3-(methoxycarbonyl)butanoyl)pyrrolidine-1-carboxylate 2206 (1.2 g, 0.0029 mol) in AcOH (10 mL) was added H₂NNH₂·H₂O (2 mL, 80% wt.) at rt. The reaction mixture was stirred at 100° C. for 1 h. The reaction mixture was concentrated under reduced pressure to give crude. The crude was purified by flash column chromatography (eluting with PE/EtOAc=100:0 to 50:50) to afford tert-butyl (R)-4,4-difluoro-2-(6-oxo-5-(trifluoromethyl)-1,6-dihydropyridazin-3-yl)pyrrolidine-1-carboxylate 2207 (0.32 g, 90% purity, 27% yield) as a yellow oil.

LCMS (ESI) calcd for C₁₄H₁₆F₅N₃O₃[M+H]⁺ m/z 370.11, found 370.05.

### Preparation of tert-butyl (R)-4,4-difluoro-2-(1-(4-methoxybenzyl)-6-oxo-5-(trifluoromethyl)-1,6-dihydropyridazin-3-yl)pyrrolidine-1-carboxylate (2208)

To a solution of tert-butyl (R)-4,4-difluoro-2-(6-oxo-5-(trifluoromethyl)-1,6-dihydropyridazin-3-yl)pyrrolidine-1-carboxylate 2207 (320 mg, 0.8642 mmol) in DMF (10 mL) were added Cs₂CO₃ (563 mg, 1.7284 mmol) and PMBCl (203 mg, 1.2963 mmol) at rt. The mixture was stirred at 50° C. for 1 h. The resulting mixture was added into cold water and then extracted with EtOAc (50 mL×3). The combined organic layer was washed with brine, dried over Na₂SO₄, and concentrated under reduced pressure. The residue was purified by flash silica chromatography (eluting with PE/EtOAc=100:0 to 50:50) to give tert-butyl (R)-4,4-difluoro-2-(1-(4-methoxybenzyl)-6-oxo-5-(trifluoromethyl)-1,6-dihydropyridazin-3-yl)pyrrolidine-1-carboxylate 2208 (250 mg, 90% purity, 53% yield) as a yellow oil.

LCMS (ESI) calcd for C₂₂H₂₄F₅N₃O₄[M+H]⁺ m/z 490.17, found 490.10.

### Preparation of (R)-6-(4,4-difluoropyrrolidin-2-yl)-2-(4-methoxybenzyl)-4-(trifluoromethyl)pyridazin-3(2H)-one hydrochloride (2209)

A solution of tert-butyl (R)-4,4-difluoro-2-(1-(4-methoxybenzyl)-6-oxo-5-(trifluoromethyl)-1,6-dihydropyridazin-3-yl)pyrrolidine-1-carboxylate 2208 (250 mg, 0.5097 mmol) in HCl-dioxane (4 M, 10 mL) was stirred at rt for 1 h. The reaction mixture was concentrated under reduced pressure to give (R)-6-(4,4-difluoropyrrolidin-2-yl)-2-(4-methoxybenzyl)-4-(trifluoromethyl)pyridazin-3(2H)-one hydrochloride 2209 (200 mg, 90% purity, 82% yield) as a yellow oil.

LCMS (ESI) calcd for C₁₇H₁₆F₅N₃O₂ [M+H]⁺ m/z 390.12, found 390.10.

### Preparation of tert-butyl (R)-2-(4,4-difluoro-2-(1-(4-methoxybenzyl)-6-oxo-5-(trifluoromethyl)-1,6-dihydropyridazin-3-yl)pyrrolidin-1-yl)acetate (2211)

To a solution of (R)-6-(4,4-difluoropyrrolidin-2-yl)-2-(4-methoxybenzyl)-4-(trifluoromethyl)pyridazin-3(2H)-one hydrochloride 2209 (200 mg, 0.470 mmol) in ACN (20 mL) were added tert-butyl 2-bromoacetate 2210 (458 mg, 2.349 mmol) and DIPEA (303 mg, 2.348 mmol) at rt. The mixture was stirred at 80° C. for 2 h. The resulting mixture was added into water and then extracted with EtOAc (50 mL×3). The combined organic layer was washed with brine, dried over Na₂SO₄, and concentrated under reduced pressure. The residue was purified by flash silica chromatography (eluting with PE/EtOAc=100:0 to 50:50) to give tert-butyl (R)-2-(4,4-difluoro-2-(1-(4-methoxybenzyl)-6-oxo-5-(trifluoromethyl)-1,6-dihydropyridazin-3-yl)pyrrolidin-1-yl)acetate 2211 (200 mg, 90% purity, 76% yield) as a yellow oil.

LCMS (ESI) calcd for C₂₃H₂₆F₅N₃O₄[M+H]⁺ m/z 504.18, found 504.20.

### Preparation of (R)-2-(4,4-difluoro-2-(1-(4-methoxybenzyl)-6-oxo-5-(trifluoromethyl)-1,6-dihydropyridazin-3-yl)pyrrolidin-1-yl)acetic acid (2212)

A solution of tert-butyl (R)-2-(4,4-difluoro-2-(1-(4-methoxybenzyl)-6-oxo-5-(trifluoromethyl)-1,6-dihydropyridazin-3-yl)pyrrolidin-1-yl)acetate 2211 (200 mg, 0.3972 mmol) in HCl-dioxane (10 mL, 4 M) was stirred at rt for 1 h. The resulting mixture was concentrated under reduced pressure to give (R)-2-(4,4-difluoro-2-(1-(4-methoxybenzyl)-6-oxo-5-(trifluoromethyl)-1,6-dihydropyridazin-3-yl)pyrrolidin-1-yl)acetic acid 2212 (150 mg, 90% purity, 75% yield) as a yellow oil.

LCMS (ESI) calcd for C₁₉H₁₈F₅N₃O₄[M+H]⁺ m/z 448.12, found 448.15.

### Preparation of (R)-6-(4,4-difluoro-1-(2-oxo-2-(4-(5-(trifluoromethyl)pyrimidin-2-yl)piperazin-1-yl)ethyl)pyrrolidin-2-yl)-2-(4-methoxybenzyl)-4-(trifluoromethyl)pyridazin-3(2H)-one (2214)

To a solution of (R)-2-(4,4-difluoro-2-(1-(4-methoxybenzyl)-6-oxo-5-(trifluoromethyl)-1,6-dihydropyridazin-3-yl)pyrrolidin-1-yl)acetic acid 2212 (150 mg, 0.3353 mmol) in DCM (20 mL) were added 2-(piperazin-1-yl)-5-(trifluoromethyl)pyrimidine hydrochloride 2213 (78 mg, 0.3353 mmol), DIPEA (130 mg, 1.0059 mmol) and T4P (483 mg, 0.6706 mmol, 50% wt. in EtOAc) at rt. The reaction mixture stirred at rt for 1 h. The resulting mixture was added into water and then extracted with DCM (50 mL×3). The combined organic layer was washed with brine, dried over Na₂SO₄, and concentrated under reduced pressure. The residue was purified by flash silica chromatography (eluting with DCM/MeOH=100:0 to 90:10) to give (R)-6-(4,4-difluoro-1-(2-oxo-2-(4-(5-(trifluoromethyl)pyrimidin-2-yl)piperazin-1-yl)ethyl)pyrrolidin-2-yl)-2-(4-methoxybenzyl)-4-(trifluoromethyl)pyridazin-3(2H)-one 2214 (200 mg, 90% purity, 81% yield) as a yellow oil.

LCMS (ESI) calcd for C₂₈H₂₇F₈N₇O₃[M+H]⁺ m/z 662.20, found 662.10.

### Preparation of (R)-6-(4,4-difluoro-1-(2-oxo-2-(4-(5-(trifluoromethyl)pyrimidin-2-yl)piperazin-1-yl)ethyl)pyrrolidin-2-yl)-4-(trifluoromethyl)pyridazin-3(2H)-one (91a)

To a solution of (R)-6-(4,4-difluoro-1-(2-oxo-2-(4-(5-(trifluoromethyl)pyrimidin-2-yl)piperazin-1-yl)ethyl)pyrrolidin-2-yl)-2-(4-methoxybenzyl)-4-(trifluoromethyl)pyridazin-3(2H)-one 2214 (200 mg, 0.3023 mmol) in TFA (3 mL) was added TfOH (0.5 mL) at rt. After completion of addition, the reaction solution was stirred at 50° C. for 10 min. The residue was diluted with DCM (50 mL) and then adjusted pH to 8 with saturated aqueous NaHCO₃ at 0° C. The basified solution was extracted with DCM (100 mL×3). The combined organic layer was washed with brine, dried over Na₂SO₄, and concentrated under reduced pressure. The crude product was purified by flash silica chromatography (eluting with DCM/MeOH=100:0 to 90:10) and then separated by SFC resolution (Column: (R,R)-Whelk-O1 250 mm×20 mm I.D., 5 μm; Mobile phase: CO₂/MeOH (0.1% NH₃)=70/30) to give the first fraction as (R)-6-(4,4-difluoro-1-(2-oxo-2-(4-(5-(trifluoromethyl)pyrimidin-2-yl)piperazin-1-yl)ethyl)pyrrolidin-2-yl)-4-(trifluoromethyl)pyridazin-3(2H)-one 91a (41.5 mg, 99% purity, 25% yield, 100% ee, white solid).

Note: S-enantiomer was observed and removed by chiral separation.

¹H NMR (400 MHz, DMSO-d₆, ppm) δ: 13.61 (s, 1H), 8.73 (s, 2H), 7.85 (s, 1H), 4.11 (t, J=8.2 Hz, 1H), 3.91-3.81 (m, 2H), 3.80-3.69 (m, 2H), 3.62-3.45 (m, 4H), 3.43-3.33 (m, 3H), 3.21-3.08 (m, 1H), 2.77-2.63 (m, 1H), 2.48-2.41 (m, 1H).

LCMS (ESI) calcd for C₂₀H₁₉F₈N₇O₂[M+H]⁺ m/z 542.15, found 542.10.

### 14. Synthesis of (S)-6-(4,4-difluoro-1-(2-oxo-2-(4-(5-(trifluoromethyl)pyrimidin-2-yl)piperazin-1-yl)ethyl)pyrrolidin-2-yl)-4-(trifluoromethyl)pyridazin-3(2H)-one (Compound 91b)

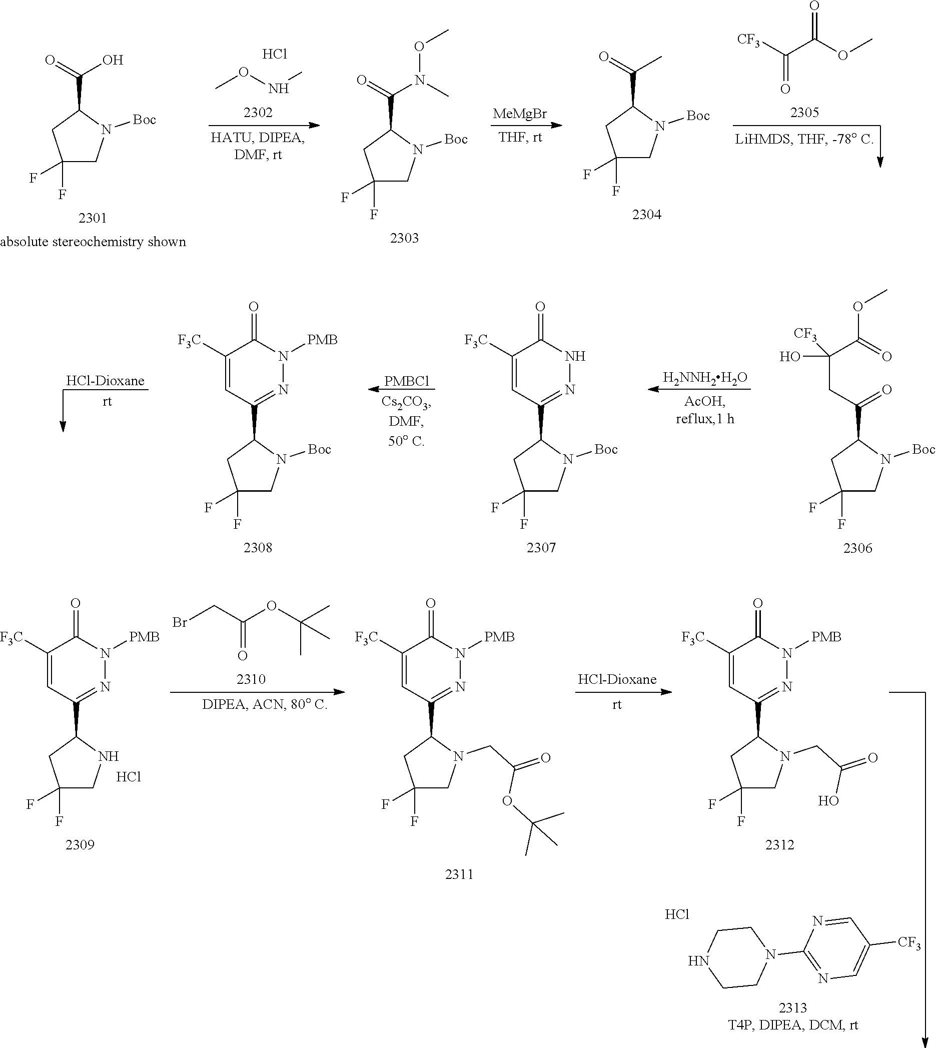

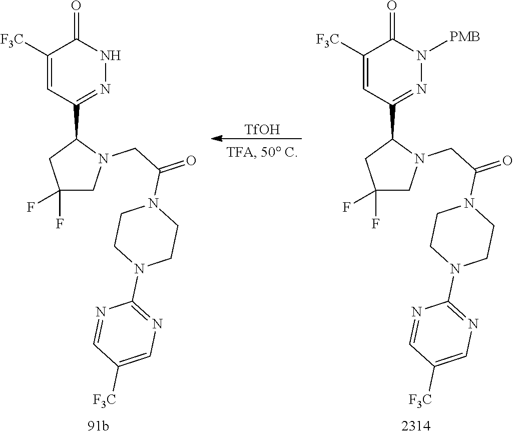

### Preparation of tert-butyl (S)-4,4-difluoro-2-(methoxy(methyl)carbamoyl)pyrrolidine-1-carboxylate (2303)

To a solution of (S)-1-(tert-butoxycarbonyl)-4,4-difluoropyrrolidine-2-carboxylic acid 2301 (2.0 g, 0.008 mol) and N,O-dimethylhydroxylamine hydrochloride 2302 (2.5 g, 0.010 mol) in DMF (20 mL) were added DIPEA (3.8 g, 0.030 mol) and HATU (7.5 g, 0.020 mol) at rt. The reaction mixture was stirred at rt for 1 h. The reaction solution was quenched with water (200 mL) and extracted with EtOAc (200 mL×3). The organic phase was concentrated, and the residue was purified by flash column chromatography (eluting with DCM/MeOH=100:0 to 95:5) to afford tert-butyl (S)-4,4-difluoro-2-(methoxy(methyl)carbamoyl)pyrrolidine-1-carboxylate 2303 (2.5 g, 90% purity, 76% yield) as a yellow oil.

LCMS (ESI) calcd for C₁₂H₂₀F₂N₂O₄[M+H]⁺ m/z 295.30, found 295.35.

### Preparation of tert-butyl (S)-2-acetyl-4,4-difluoropyrrolidine-1-carboxylate (2304)

To a solution of tert-butyl (S)-4,4-difluoro-2-(methoxy(methyl)carbamoyl)pyrrolidine-1-carboxylate 2303 (2.5 g, 0.0085 mol) in THF (50 mL) was added MeMgBr (1 M, 17 mL, 0.017 mol) at rt. The resulting mixture was stirred at rt for 1 h. Water (100 mL) was added, and the mixture was extracted EtOAc (150 mL×2). The combined organic layers were dried over Na₂SO₄, concentrated in vacuo and the residue was purified by flash column chromatography (eluting with PE/EtOAc=100:0 to 60:40) to afford tert-butyl (S)-2-acetyl-4,4-difluoropyrrolidine-1-carboxylate 2304 (2.0 g, 90% purity, 84% yield) as a yellow oil.

LCMS (ESI) calcd for C₁₁H₁₇F₂NO₃ [M-Boc+H]⁺ m/z 150.12, found 150.00.

### Preparation of tert-butyl (2S)-4,4-difluoro-2-(4,4,4-trifluoro-3-hydroxy-3-(methoxycarbonyl)butanoyl)pyrrolidine-1-carboxylate (2306)

To a solution of tert-butyl (S)-2-acetyl-4,4-difluoropyrrolidine-1-carboxylate 2304 (2.0 g, 0.0080 mol) in THF (20 mL) was added LiHMDS (1 M, 9.6 mL, 0.0096 mol) at −78° C. The resulting mixture was stirred at −78° C. for 15 min. To the reaction mixture was added methyl 3,3,3-trifluoro-2-oxopropanoate 2305 (1.5 g, 0.010 mol) at −78° C. and then stirred at −78° C. for 15 min. The reaction mixture was poured into water. The aqueous layer was extracted with EtOAc (80 mL×3). The combined organic layers were concentrated under reduced pressure. The residue was purified by flash column chromatography (eluting with PE/EtOAc=100:0 to 60:40) to afford tert-butyl (2S)-4,4-difluoro-2-(4,4,4-trifluoro-3-hydroxy-3-(methoxycarbonyl)butanoyl)pyrrolidine-1-carboxylate 2306 (2.0 g, 50% purity, 31% yield) as a yellow oil.

LCMS (ESI) calcd for C₁₅H₂₀F₅NO₆ [M+Na]⁺ m/z 428.12, found 428.15.

### Preparation of tert-butyl (S)-4,4-difluoro-2-(6-oxo-5-(trifluoromethyl)-1,6-dihydropyridazin-3-yl)pyrrolidine-1-carboxylate (2307)

To a solution of tert-butyl (2S)-4,4-difluoro-2-(4,4,4-trifluoro-3-hydroxy-3-(methoxycarbonyl)butanoyl)pyrrolidine-1-carboxylate 2306 (2.0 g, 0.0049 mol) in AcOH (20 mL) was added H₂NNH₂·H₂O (5 mL, 80% wt.) at rt. The reaction mixture was stirred at 80° C. for 1 h. The reaction mixture was concentrated under reduced pressure to give crude. The crude was purified by flash column chromatography (eluting with PE/EtOAc=100:0 to 40:60) to afford tert-butyl (S)-4,4-difluoro-2-(6-oxo-5-(trifluoromethyl)-1,6-dihydropyridazin-3-yl)pyrrolidine-1-carboxylate 2307 (2.0 g, 50% purity, 55% yield) as a yellow oil.

LCMS (ESI) calcd for C₁₄H₁₆F₅N₃O₃[M+H]⁺ m/z 370.11, found 370.05.

### Preparation of tert-butyl (S)-4,4-difluoro-2-(1-(4-methoxybenzyl)-6-oxo-5-(trifluoromethyl)-1,6-dihydropyridazin-3-yl)pyrrolidine-1-carboxylate (2308)

To a solution of tert-butyl (S)-4,4-difluoro-2-(6-oxo-5-(trifluoromethyl)-1,6-dihydropyridazin-3-yl)pyrrolidine-1-carboxylate 2307 (2.0 g, 0.0054 mol) in DMF (30 mL) were added Cs₂CO₃ (3.5 g, 0.0108 mol) and PMBCl (1.3 g, 0.0081 mol) at rt. The mixture was stirred at 50° C. for 1 h. The resulting mixture was added into cold water and then extracted with EtOAc (100 mL×3). The combined organic layer was washed with brine, dried over Na₂SO₄ and concentrated under reduced pressure. The residue was purified by flash silica chromatography (eluting with PE/EtOAc=100:0 to 50:50) to give tert-butyl (S)-4,4-difluoro-2-(1-(4-methoxybenzyl)-6-oxo-5-(trifluoromethyl)-1,6-dihydropyridazin-3-yl)pyrrolidine-1-carboxylate 2308 (1.2 g, 90% purity, 40% yield) as a yellow oil.

LCMS (ESI) calcd for C₂₂H₂₄F₅N₃O₄[M+H]⁺ m/z 490.17, found 490.25.

### Preparation of (S)-6-(4,4-difluoropyrrolidin-2-yl)-2-(4-methoxybenzyl)-4-(trifluoromethyl)pyridazin-3(2H)-one hydrochloride (2309)

A solution of tert-butyl (S)-4,4-difluoro-2-(1-(4-methoxybenzyl)-6-oxo-5-(trifluoromethyl)-1,6-dihydropyridazin-3-yl)pyrrolidine-1-carboxylate 2308 (500 mg, 1.019 mmol) in HCl-dioxane (4 M, 20 mL) was stirred at rt for 1 h. The reaction mixture was concentrated under reduced pressure to give (S)-6-(4,4-difluoropyrrolidin-2-yl)-2-(4-methoxybenzyl)-4-(trifluoromethyl)pyridazin-3(2H)-one hydrochloride 2309 (350 mg, 90% purity, 72% yield) as a yellow oil.

LCMS (ESI) calcd for C₁₇H₁₆F₅N₃O₂ [M+H]⁺ m/z 390.12, found 390.15.

### Preparation of tert-butyl (S)-2-(4,4-difluoro-2-(1-(4-methoxybenzyl)-6-oxo-5-(trifluoromethyl)-1,6-dihydropyridazin-3-yl)pyrrolidin-1-yl)acetate (2311)

To a solution of (S)-6-(4,4-difluoropyrrolidin-2-yl)-2-(4-methoxybenzyl)-4-(trifluoromethyl)pyridazin-3(2H)-one hydrochloride 2309 (350 mg, 0.822 mmol) in ACN (30 mL) were added tert-butyl 2-bromoacetate 2310 (321 mg, 1.644 mmol) and DIPEA (212 mg, 1.644 mmol) at rt. The mixture was stirred at 80° C. for 1 h. The resulting mixture was added into water and then extracted with EtOAc (50 mL×3). The combined organic layer was washed with brine, dried over Na₂SO₄, and concentrated under reduced pressure. The residue was purified by flash silica chromatography (eluting with PE/EtOAc=100:0 to 50:50) to give tert-butyl (S)-2-(4,4-difluoro-2-(1-(4-methoxybenzyl)-6-oxo-5-(trifluoromethyl)-1,6-dihydropyridazin-3-yl)pyrrolidin-1-yl)acetate 2311 (400 mg, 90% purity, 86% yield) as a yellow oil.

LCMS (ESI) calcd for C₂₃H₂₆F₅N₃O₄[M+H]⁺ m/z 504.18, found 504.15.

### Preparation of (S)-2-(4,4-difluoro-2-(1-(4-methoxybenzyl)-6-oxo-5-(trifluoromethyl)-1,6-dihydropyridazin-3-yl)pyrrolidin-1-yl)acetic acid (2312)

A solution of tert-butyl (S)-2-(4,4-difluoro-2-(1-(4-methoxybenzyl)-6-oxo-5-(trifluoromethyl)-1,6-dihydropyridazin-3-yl)pyrrolidin-1-yl)acetate 2311 (290 mg, 0.576 mmol) in HCl-dioxane (20 mL, 4 M) was stirred at rt for 4 h. The resulting mixture was concentrated under reduced pressure to give (S)-2-(4,4-difluoro-2-(1-(4-methoxybenzyl)-6-oxo-5-(trifluoromethyl)-1,6-dihydropyridazin-3-yl)pyrrolidin-1-yl)acetic acid 2312 (220 mg, 90% purity, 76% yield) as a yellow oil.

LCMS (ESI) calcd for C₁₉H₁₈F₅N₃O₄[M+H]⁺ m/z 448.12, found 448.15.

### Preparation of (S)-6-(4,4-difluoro-1-(2-oxo-2-(4-(5-(trifluoromethyl)pyrimidin-2-yl)piperazin-1-yl)ethyl)pyrrolidin-2-yl)-2-(4-methoxybenzyl)-4-(trifluoromethyl)pyridazin-3(2H)-one (2314)

To a solution of (S)-2-(4,4-difluoro-2-(1-(4-methoxybenzyl)-6-oxo-5-(trifluoromethyl)-1,6-dihydropyridazin-3-yl)pyrrolidin-1-yl)acetic acid 2312 (220 mg, 0.492 mmol) in DCM (20 mL) were added 2-(piperazin-1-yl)-5-(trifluoromethyl)pyrimidine hydrochloride 2313 (133 mg, 0.492 mmol), DIPEA (190 mg, 1.475 mmol) and T4P (708 mg, 0.984 mmol, 50% wt. in EtOAc) at rt. The reaction mixture stirred at rt for 1 h. The resulting mixture was added into water and then extracted with EtOAc (50 mL×3). The combined organic layer was washed with brine, dried over Na₂SO₄, and concentrated under reduced pressure. The residue was purified by flash silica chromatography (eluting with DCM/MeOH=100:0 to 95:5) to give (S)-6-(4,4-difluoro-1-(2-oxo-2-(4-(5-(trifluoromethyl)pyrimidin-2-yl)piperazin-1-yl)ethyl)pyrrolidin-2-yl)-2-(4-methoxybenzyl)-4-(trifluoromethyl)pyridazin-3(2H)-one 2314 (200 mg, 90% purity, 55% yield) as a yellow oil.

LCMS (ESI) calcd for C₂₈H₂₇F₈N₇O₃[M+H]⁺ m/z 662.20, found 662.15.

### Preparation of (S)-6-(4,4-difluoro-1-(2-oxo-2-(4-(5-(trifluoromethyl)pyrimidin-2-yl)piperazin-1-yl)ethyl)pyrrolidin-2-yl)-4-(trifluoromethyl)pyridazin-3(2H)-one (91b)

To a solution of (S)-6-(4,4-difluoro-1-(2-oxo-2-(4-(5-(trifluoromethyl)pyrimidin-2-yl)piperazin-1-yl)ethyl)pyrrolidin-2-yl)-2-(4-methoxybenzyl)-4-(trifluoromethyl)pyridazin-3(2H)-one 2314 (200 mg, 0.3023 mmol) in TFA (3 mL) was added TfOH (0.5 mL) at rt. After completion of addition, the reaction solution was stirred at 50° C. for 10 min. The residue was diluted with DCM (50 mL) and then adjusted pH to 8 with saturated aqueous NaHCO₃ at 0° C. The basified solution was extracted with DCM (30 mL×3). The combined organic layer was washed with brine, dried over Na₂SO₄, and concentrated under reduced pressure. The crude product was purified by flash silica chromatography (eluting with DCM/MeOH=100:0 to 90:10) and then separated by SFC resolution (Column: (R,R)-Whelk-O1 250 mm×20 mm I.D., 5 μm; Mobile phase: CO₂/MeOH (0.1% NH₃)=70/30) to give the second fraction as (S)-6-(4,4-difluoro-1-(2-oxo-2-(4-(5-(trifluoromethyl)pyrimidin-2-yl)piperazin-1-yl)ethyl)pyrrolidin-2-yl)-4-(trifluoromethyl)pyridazin-3(2H)-one 91b (43.1 mg, 99% purity, 26% yield, 100% ee, white solid).

Note: R-enantiomer was observed and removed by chiral separation.

¹H NMR (400 MHz, DMSO-d₆, ppm) δ: 13.62 (s, 1H), 8.73 (s, 2H), 7.85 (s, 1H), 4.11 (t, J=8.4 Hz, 1H), 3.91-3.81 (m, 2H), 3.78-3.70 (m, 2H), 3.59-3.45 (m, 4H), 3.42-3.34 (m, 3H), 3.21-3.10 (m, 1H), 2.77-2.64 (m, 1H), 2.46-2.42 (m, 1H).

LCMS (ESI) calcd for C₂₀H₁₉F₈N₇O₂[M+H]⁺ m/z 542.15, found 542.35.

### 15. Synthesis of 3-oxo-6-(1-(2-oxo-2-(4-(5-(trifluoromethyl)pyrimidin-2-yl)piperazin-1-yl)ethyl)pyrrolidin-2-yl)-2,3-dihydropyridazine-4-carbonitrile (Compounds 98a and 98b)

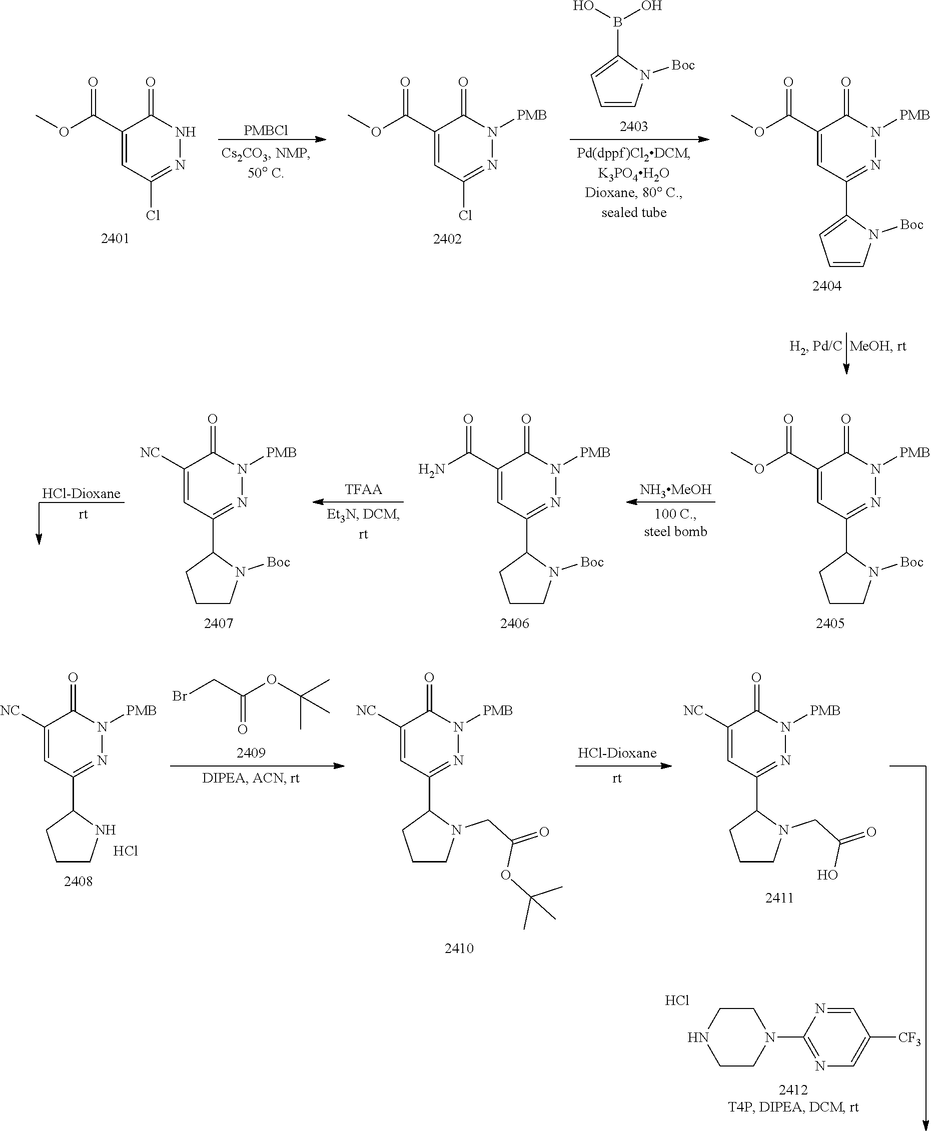

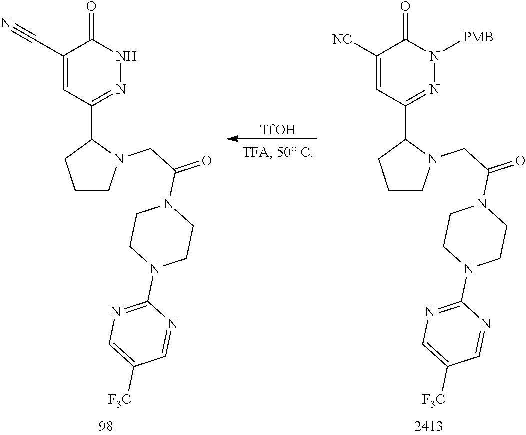

### Preparation of methyl 6-chloro-2-(4-methoxybenzyl)-3-oxo-2,3-dihydropyridazine-4-carboxylate (2402)

To a solution of methyl 6-chloro-3-oxo-2,3-dihydropyridazine-4-carboxylate 2401 (2 g, 0.0106 mol) in NMP (50 mL) was added PMBCl (2.0 g, 0.0127 mol) and Cs₂CO₃ (4 g, 0.0127 mol) at rt. The resulting mixture was stirred at 50° C. for 1 h. After cooling to room temperature, the reaction mixture was poured into water and then extracted with EtOAc (200 mL×3). The combined organic layers were washed with brine (100 mL×4), dried over Na₂SO₄ and concentrated under reduced pressure. The residue was purified by flash column chromatography (eluting with PE/EtOAc=100:0 to 50:50) to afford methyl 6-chloro-2-(4-methoxybenzyl)-3-oxo-2,3-dihydropyridazine-4-carboxylate 2402 (2.6 g, 90% purity, 72% yield) as a yellow oil.

LCMS (ESI) calcd for C₁₄H₁₃ClN₂O₄[M+Na]⁺ m/z 309.06, found m/z 309.20.

### Preparation of methyl 6-(1-(tert-butoxycarbonyl)-1H-pyrrol-2-yl)-2-(4-methoxybenzyl)-3-oxo-2,3-dihydropyridazine-4-carboxylate (2404)

To a solution of methyl 6-chloro-2-(4-methoxybenzyl)-3-oxo-2,3-dihydropyridazine-4-carboxylate 2402 (2.6 g, 0.0084 mol) in dioxane (30 mL) were added (1-(tert-butoxycarbonyl)-1H-pyrrol-2-yl)boronic acid 2403 (2.9 g, 0.014 mol), K₃PO₄·H₂O (3.86 g, 0.017 mol) and Pd(dppf)Cl₂-DCM (0.34 g, 0.00042 mol) at rt. The reaction mixture was stirred in a sealed tube at 80° C. for 16 h. The resulting mixture was concentrated under reduced pressure. The residue was purified by silica gel column (eluting with PE/EtOAc=100:0 to 50:50) to obtain methyl 6-(1-(tert-butoxycarbonyl)-1H-pyrrol-2-yl)-2-(4-methoxybenzyl)-3-oxo-2,3-dihydropyridazine-4-carboxylate 2404 (1.3 g, 90% purity, 32% yield) as a yellow oil.

LCMS (ESI) calcd for C₂₃H₂₅N₃O₆ [M+H]⁺ m/z 440.17, found m/z 440.20.

### Preparation of methyl 6-(1-(tert-butoxycarbonyl)pyrrolidin-2-yl)-2-(4-methoxybenzyl)-3-oxo-2,3-dihydropyridazine-4-carboxylate (2405)

To a solution of methyl 6-(1-(tert-butoxycarbonyl)-1H-pyrrol-2-yl)-2-(4-methoxybenzyl)-3-oxo-2,3-dihydropyridazine-4-carboxylate 2404 (1.2 g, 2.7 mmol) in MeOH (5 mL) was added Pd/C (1.2 g) at rt. The mixture was evacuated and backfilled with hydrogen three times and then charged with hydrogen. The resulting mixture was stirred at rt for 2 hours. The resulting mixture was filtered through celite. The filtrate was concentrated under vacuum to give crude methyl 6-(1-(tert-butoxycarbonyl)pyrrolidin-2-yl)-2-(4-methoxybenzyl)-3-oxo-2,3-dihydropyridazine-4-carboxylate 2405 (1 g, 90% purity 74% yield) which was used directly in next step without further purification.

LCMS (ESI) calcd for C₂₃H₂₉N₃O₆ [M+H]⁺ m/z 444.21, found 444.20.

### Preparation of tert-butyl 2-(5-carbamoyl-1-(4-methoxybenzyl)-6-oxo-1,6-dihydropyridazin-3-yl)pyrrolidine-1-carboxylate (2406)

A solution of methyl 6-(1-(tert-butoxycarbonyl)pyrrolidin-2-yl)-2-(4-methoxybenzyl)-3-oxo-2,3-dihydropyridazine-4-carboxylate 2405 (1 g, 0.0022 mol) in NH₃-MeOH (20 mL, 7 M) in a steel bomb was stirred at 100° C. for 1 h. The resulting mixture was concentrated under reduced pressure. The residue was purified by silica gel column (eluting with PE/EtOAc=100:0 to 50:50) to obtain tert-butyl 2-(5-carbamoyl-1-(4-methoxybenzyl)-6-oxo-1,6-dihydropyridazin-3-yl)pyrrolidine-1-carboxylate 2406 (0.6 g, 80% purity, 50% yield) as a yellow oil.

LCMS (ESI) calcd for C₂₂H₂₈N₄O₅ [M+H]⁺ m/z 429.21, found 429.20.

### Preparation of tert-butyl 2-(5-cyano-1-(4-methoxybenzyl)-6-oxo-1,6-dihydropyridazin-3-yl)pyrrolidine-1-carboxylate (2407)

To a suspension of tert-butyl 2-(5-carbamoyl-1-(4-methoxybenzyl)-6-oxo-1,6-dihydropyridazin-3-yl)pyrrolidine-1-carboxylate 2406 (600 mg, 1.397 mmol) and Et₃N (424 mg, 4.191 mmol) in DCM (10 mL) was added TFAA (440 mg, 2.095 mmol) at 0° C. Then the reaction mixture was stirred at rt for 2 h. The resulting mixture was concentrated under reduced pressure. The residue was purified by flash chromatography (eluting with DCM/MeOH=100:0 to 90:10) to obtain tert-butyl 2-(5-cyano-1-(4-methoxybenzyl)-6-oxo-1,6-dihydropyridazin-3-yl)pyrrolidine-1-carboxylate 2407 (400 mg, 90% purity, 62% yield) as a yellow oil.

LCMS (ESI) calcd for C₂₂H₂₆N₄O₄ [M+H]⁺ m/z 411.20, found 411.05.

### Preparation of 2-(4-methoxybenzyl)-3-oxo-6-(pyrrolidin-2-yl)-2,3-dihydropyridazine-4-carbonitrile hydrochloride (2408)

A suspension of tert-butyl 2-(5-cyano-1-(4-methoxybenzyl)-6-oxo-1,6-dihydropyridazin-3-yl)pyrrolidine-1-carboxylate 2407 (400 mg, 0.9721 mmol) in HCl-dioxane (4 M, 10 mL) was stirred at rt for 30 min, concentrated and evaporated with DCM (20 mL) to obtain 2-(4-methoxybenzyl)-3-oxo-6-(pyrrolidin-2-yl)-2,3-dihydropyridazine-4-carbonitrile hydrochloride 2408 (350 mg, 80% purity, 83% yield) as a yellow oil.

LCMS (ESI) calcd for C₁₇H₁₈N₄O₂[M+H]⁺ m/z 311.14, found 311.00.

### Preparation of tert-butyl 2-(2-(5-cyano-1-(4-methoxybenzyl)-6-oxo-1,6-dihydropyridazin-3-yl)pyrrolidin-1-yl)acetate (2410)

To a solution of 2-(4-methoxybenzyl)-3-oxo-6-(pyrrolidin-2-yl)-2,3-dihydropyridazine-4-carbonitrile hydrochloride 2408 (350 mg, 1.0092 mmol) in ACN (10 mL) were added tert-butyl 2-bromoacetate 2409 (295 mg, 1.5138 mmol) and DIPEA (195 mg, 1.5138 mmol). After completion of addition, the reaction solution was stirred at rt for 2 h. Water was added to quench the reaction. The obtained solution was extracted with EtOAc (50 mL×4). The combined organic phase was washed with brine, dried over Na₂SO₄, and concentrated under reduced pressure. The residue was purified by flash chromatography (eluting with PE/EtOAc=100:0 to 50:50) to give tert-butyl 2-(2-(5-cyano-1-(4-methoxybenzyl)-6-oxo-1,6-dihydropyridazin-3-yl)pyrrolidin-1-yl)acetate 2410 (300 mg, 90% purity, 63% yield) as a yellow solid.

LCMS (ESI) calcd for C₂₃H₂₈N₄O₄ [M+H]⁺ m/z 425.21, found 425.15.

### Preparation of 2-(2-(5-cyano-1-(4-methoxybenzyl)-6-oxo-1,6-dihydropyridazin-3-yl)pyrrolidin-1-yl)acetic acid (2411)

A suspension of tert-butyl 2-(2-(5-cyano-1-(4-methoxybenzyl)-6-oxo-1,6-dihydropyridazin-3-yl)pyrrolidin-1-yl)acetate 2410 (150 mg, 0.3534 mmol) in HCl-dioxane (4 M, 5 mL) was stirred at rt for 30 min, concentrated and evaporated with DCM (20 mL). The crude product was dried in vacuo to obtain 2-(2-(5-cyano-1-(4-methoxybenzyl)-6-oxo-1,6-dihydropyridazin-3-yl)pyrrolidin-1-yl)acetic acid 2411 (120 mg, 90% purity, 82% yield) as a yellow oil.

LCMS (ESI) calcd for C₁₉H₂₀N₄O₄ [M+H]⁺ m/z 369.15, found 369.10.

### Preparation of 2-(4-methoxybenzyl)-3-oxo-6-(1-(2-oxo-2-(4-(5-(trifluoromethyl)pyrimidin-2-yl)piperazin-1-yl)ethyl)pyrrolidin-2-yl)-2,3-dihydropyridazine-4-carbonitrile (2413)

To a solution of 2-(2-(5-cyano-1-(4-methoxybenzyl)-6-oxo-1,6-dihydropyridazin-3-yl)pyrrolidin-1-yl)acetic acid 2411 (120 mg, 0.3257 mmol) and 2-(piperazin-1-yl)-5-(trifluoromethyl)pyrimidine hydrochloride 2412 (105 mg, 0.3908 mmol) in DCM (5 mL) were added T4P (50% wt in EtOAc, 352 mg, 0.4885 mmol) and DIPEA (63 mg, 0.4885 mmol). The mixture was kept stirring at rt for 1 h. The resulting mixture was quenched with water and extracted with DCM (20 mL×3). The combined organic phase was concentrated under reduced pressure. The residue was purified by silica gel column (eluting with DCM/MeOH=100:0 to 90:10) to obtain 2-(4-methoxybenzyl)-3-oxo-6-(1-(2-oxo-2-(4-(5-(trifluoromethyl)pyrimidin-2-yl)piperazin-1-yl)ethyl)pyrrolidin-2-yl)-2,3-dihydropyridazine-4-carbonitrile 2413 (100 mg, 90% purity, 47% yield) as a white solid.

LCMS (ESI) calcd for C₂₈H₂₉F₃N₈O₃[M+H]⁺ m/z 583.23, found 583.20.

### Preparation of 3-oxo-6-(1-(2-oxo-2-(4-(5-(trifluoromethyl)pyrimidin-2-yl)piperazin-1-yl)ethyl)pyrrolidin-2-yl)-2,3-dihydropyridazine-4-carbonitrile (98rac)

TfOH (0.2 mL) was added to a solution of 2-(4-methoxybenzyl)-3-oxo-6-(1-(2-oxo-2-(4-(5-(trifluoromethyl)pyrimidin-2-yl)piperazin-1-yl)ethyl)pyrrolidin-2-yl)-2,3-dihydropyridazine-4-carbonitrile 2413 (100 mg, 0.1717 mmol) in TFA (5 mL). The mixture was stirred at 50° C. for 30 min, adjusted to pH 8.0 with saturated NaHCO₃ solution at 50° C. and extracted with DCM (100 mL×3). The combined organic layer was concentrated and purified by C₁₈ column (mobile phase: ACN—H₂O (0.05% NH₃), gradient: 15%-60%) to obtain 3-oxo-6-(1-(2-oxo-2-(4-(5-(trifluoromethyl)pyrimidin-2-yl)piperazin-1-yl)ethyl)pyrrolidin-2-yl)-2,3-dihydropyridazine-4-carbonitrile 98a/98b racemic mixture (30 mg, 95% purity, 35% yield) as a white solid.

### Chiral resolution of 3-oxo-6-(1-(2-oxo-2-(4-(5-(trifluoromethyl)pyrimidin-2-yl)piperazin-1-yl)ethyl)pyrrolidin-2-yl)-2,3-dihydropyridazine-4-carbonitrile (98rac)

98a/98b racemic mixture was separated by SFC (Column: Daicel IH 250 mm×20 mm I.D., 5 μm; Mobile phase: CO₂/MeOH (0.1% NH₃)=60/40) and concentrated under reduced pressure to afford the first fraction as 98a (11.3 mg, 98.52% purity, 100% ee, yellow solid) and the second fraction as 98b (11.8 mg, 98.40% purity, 100% ee, yellow solid).

### Compound 98a

¹H NMR (400 MHz, DMSO-d₆, ppm) δ: 13.56 (s, 1H), 8.73 (s, 2H), 8.21 (s, 1H), 3.99-3.82 (m, 2H), 3.79-3.65 (m, 2H), 3.63-3.34 (m, 6H), 3.25-3.13 (m, 2H), 2.58-2.52 (m, 1H), 2.21-2.07 (m, 1H), 2.01-1.69 (m, 3H).

LCMS (ESI) calcd for C₂₀H₂₁F₃N₈O₂[M+H]⁺ m/z 463.17, found 463.20.

### Compound 98b

¹H NMR (400 MHz, DMSO-d₆, ppm) δ: 13.58 (s, 1H), 8.73 (s, 2H), 8.21 (s, 1H), 3.98-3.83 (m, 2H), 3.79-3.65 (m, 2H), 3.63-3.33 (m, 6H), 3.25-3.13 (m, 2H), 2.58-2.52 (m, 1H), 2.20-2.06 (m, 1H), 1.98-1.68 (m, 3H).

LCMS (ESI) calcd for C₂₀H₂₁F₃N₈O₂[M+H]⁺ m/z 463.17, found 463.15

### 16. Synthesis of 6-methyl-5-(1-(2-oxo-2-(4-(5-(trifluoromethyl)pyrimidin-2-yl)piperazin-1-yl)ethyl)pyrrolidin-2-yl)-3-(trifluoromethyl)pyridin-2(1H)-one (Compounds 102a and 102b)

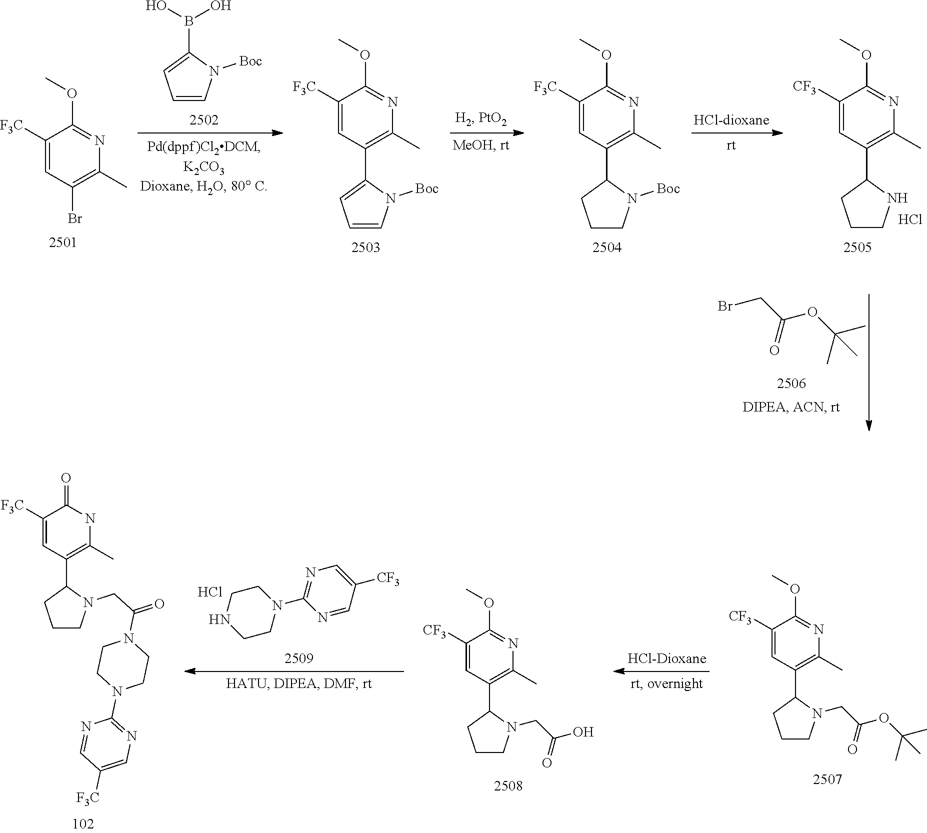

### Preparation of tert-butyl 2-(6-methoxy-2-methyl-5-(trifluoromethyl)pyridin-3-yl)-1H-pyrrole-1-carboxylate (2503)

To a solution of 3-bromo-6-methoxy-2-methyl-5-(trifluoromethyl)pyridine 2501 (500 mg, 1.85 mmol) and (1-(tert-butoxycarbonyl)-1H-pyrrol-2-yl)boronic acid 2502 (589 mg, 2.78 mmol) in dioxane/H₂O=5:1 (100 mL) was added K₂CO₃ (768 mg, 5.55 mmol) and Pd(dppf)Cl₂-DCM (136 mg, 0.19 mmol) under N₂. The mixture was heated at 80° C. for 2 hours. The resulting mixture was diluted with water (100 mL) and extracted with EtOAc (100 mL×3). The combined organic phases were washed with brine, dried over sodium sulfate, concentrated, and purified by silica gel column chromatography (eluting with EtOAc/PE, 0% to 50%) to give tert-butyl 2-(6-methoxy-2-methyl-5-(trifluoromethyl)pyridin-3-yl)-1H-pyrrole-1-carboxylate 2503 (300 mg, 90% purity, 40% yield) as a white solid.

LCMS (ESI) calcd for C₁₇H₁₉F₃N₂O₃[M-Boc+H]⁺ m/z 257.13, found 257.05.

### Preparation of tert-butyl 2-(6-methoxy-2-methyl-5-(trifluoromethyl)pyridin-3-yl)pyrrolidine-1-carboxylate (2504)

To a solution of tert-butyl 2-(6-methoxy-2-methyl-5-(trifluoromethyl)pyridin-3-yl)-1H-pyrrole-1-carboxylate 2503 (300 mg, 0.84 mmol) in MeOH (10 mL) was added PtO₂ (191 mg). The mixture was evacuated and backfilled with hydrogen three times and then charged with hydrogen. The resulting mixture was stirred at room temperature for 6 hours. Then the mixture was filtered through celite. The filtrate was concentrated under vacuum and purified by silica gel column chromatography (eluting with EtOAc/PE, 0% to 50%) to give tert-butyl 2-(6-methoxy-2-methyl-5-(trifluoromethyl)pyridin-3-yl)pyrrolidine-1-carboxylate 2504 (200 mg, 90% purity, 59% yield) as a white solid.

LCMS (ESI) calcd for C₁₇H₂₃F₃N₂O₃[M+H]⁺ m/z 361.17, found 361.05.

### Preparation of 2-methoxy-6-methyl-5-(pyrrolidin-2-yl)-3-(trifluoromethyl)pyridine hydrochloride (2505)

A solution of tert-butyl 2-(6-methoxy-2-methyl-5-(trifluoromethyl)pyridin-3-yl)pyrrolidine-1-carboxylate 2504 (200 mg, 0.55 mmol) in HCl dioxane solution (4 M, 10 mL) was stirred at room temperature for 2 hours. The mixture was concentrated under reduced pressure to provide crude 2-methoxy-6-methyl-5-(pyrrolidin-2-yl)-3-(trifluoromethyl)pyridine hydrochloride 2505 (140 mg, 90% yield, 87% yield) as a white solid.

LCMS (ESI) calcd for C₁₂H₁₅F₃N₂O [M+H]⁺ m/z 261.11, found 261.10.

### Preparation of tert-butyl 2-(2-(6-methoxy-2-methyl-5-(trifluoromethyl)pyridin-3-yl)pyrrolidin-1-yl)acetate (2507)

To a solution of 2-methoxy-6-methyl-5-(pyrrolidin-2-yl)-3-(trifluoromethyl)pyridine hydrochloride 2505 (140 mg, 0.54 mmol) in ACN (10 mL) was added tert-butyl 2-bromoacetate 2506 (157 mg, 0.80 mmol) and DIPEA (208 mg, 1.61 mmol). The mixture was stirred at rt for 2 hours. The resulting mixture was concentrated, and purified by silica gel column chromatography (eluting with EtOAc/PE, 0% to 50%) to give tert-butyl 2-(2-(6-methoxy-2-methyl-5-(trifluoromethyl)pyridin-3-yl)pyrrolidin-1-yl)acetate 2507 (150 mg, 90% purity, 67% yield) as a white solid. LCMS (ESI) calcd for C₁₈H₂₅F₃N₂O₃[M+H]⁺ m/z 375.18, found 375.15.

### Preparation of 2-(2-(2-methyl-6-oxo-5-(trifluoromethyl)-1,6-dihydropyridin-3-yl)pyrrolidin-1-yl)acetic acid (2508)

A solution of tert-butyl 2-(2-(6-methoxy-2-methyl-5-(trifluoromethyl)pyridin-3-yl)pyrrolidin-1-yl)acetate 2507 (150 mg, 0.40 mmol) in HCl dioxane solution (4 M, 10 mL) was stirred at room temperature overnight. The mixture was concentrated to provide crude 2-(2-(2-methyl-6-oxo-5-(trifluoromethyl)-1,6-dihydropyridin-3-yl)pyrrolidin-1-yl)acetic acid 2508 (110 mg, 90% purity, 77% yield) as a white solid.

LCMS (ESI) calcd for C₁₃H₁₅F₃N₂O₃[M+H]⁺ m/z 305.10, found 305.15.

### Preparation of 6-methyl-5-(1-(2-oxo-2-(4-(5-(trifluoromethyl)pyrimidin-2-yl)piperazin-1-yl)ethyl)pyrrolidin-2-yl)-3-(trifluoromethyl)pyridin-2(1H)-one (102rac)

To a solution of 2-(2-(2-methyl-6-oxo-5-(trifluoromethyl)-1,6-dihydropyridin-3-yl)pyrrolidin-1-yl)acetic acid 2508 (100 mg, 0.31 mmol) and 2-(piperazin-1-yl)-5-(trifluoromethyl)pyrimidine hydrochloride 2509 (101 mg, 0.38 mmol) in DMF (10 mL) was added HATU (238 mg, 0.63 mmol) and DIPEA (202 mg, 1.57 mmol). The mixture was stirred at rt for 2 hours. The resulting mixture was diluted with water (50 mL) and extracted with EtOAc (50 mL×3). The combined organic phases were washed with brine, dried over sodium sulfate, concentrated, and purified by silica gel column chromatography (eluting with MeOH/DCM, 0% to 10%) to give 6-methyl-5-(1-(2-oxo-2-(4-(5-(trifluoromethyl)pyrimidin-2-yl)piperazin-1-yl)ethyl)pyrrolidin-2-yl)-3-(trifluoromethyl)pyridin-2(1H)-one racemic mixture of 102a/102b (120 mg, 90% purity, 64% yield) as a white solid.

### Chiral resolution of 6-methyl-5-(1-(2-oxo-2-(4-(5-(trifluoromethyl)pyrimidin-2-yl)piperazin-1-yl)ethyl)pyrrolidin-2-yl)-3-(trifluoromethyl)pyridin-2(1H)-one (102rac)

The racemic mixture of 102a/102b was separated by SFC (Column: DAICEL IH 20 mm I.D.×25 0 mm, 5 μm; Mobile phase: CO₂/MeOH [0.1% NH₃ (7 M Solution in MeOH)]=80/20) and concentrated under reduced pressure to afford the first fraction as 102a (52.1 mg, 99.62% purity, ee %: 100, white solid) and the second fraction as 102b (55.7 mg, 99.88% purity, ee %: 100, white solid).

### Compound 102a

¹H NMR (400 MHz, DMSO-d₆, ppm) δ: 12.11 (s, 1H), 8.73 (s, 2H), 7.78 (s, 1H), 4.02-3.90 (m, 2H), 3.69-3.52 (m, 4H), 3.51-3.44 (m, 1H), 3.31-3.25 (m, 2H), 3.22-3.17 (m, 1H), 3.16-3.06 (m, 2H), 2.39-2.33 (m, 1H), 2.28 (s, 3H), 2.14-2.03 (m, 1H), 1.89-1.72 (m, 2H), 1.57-1.46 (m, 1H).

LCMS (ESI) calcd for C₂₂H₂₄F₆N₆O₂[M+H]⁺ m/z 519.19, found 519.15.

### Compound 102b

¹H NMR (400 MHz, DMSO-d₆, ppm) δ: 12.11 (s, 1H), 8.73 (s, 2H), 7.78 (s, 1H), 4.01-3.91 (m, 2H), 3.69-3.53 (m, 4H), 3.51-3.44 (m, 1H), 3.31-3.25 (m, 2H), 3.22-3.16 (m, 1H), 3.15-3.06 (m, 2H), 2.38-2.32 (m, 1H), 2.28 (s, 3H), 2.15-2.05 (m, 1H), 1.89-1.72 (m, 2H), 1.58-1.45 (m, 1H).

LCMS (ESI) calcd for C₂₂H₂₄F₆N₆O₂[M+H]⁺ m/z 519.19, found 519.20.

### 17. Synthesis of 6-(2-(2-oxo-2-(4-(5-(trifluoromethyl)pyrimidin-2-yl)piperazin-1-yl)ethyl)isoindolin-1-yl)-4-(trifluoromethyl)pyridazin-3(2H)-one (Compounds 104a and 104b)

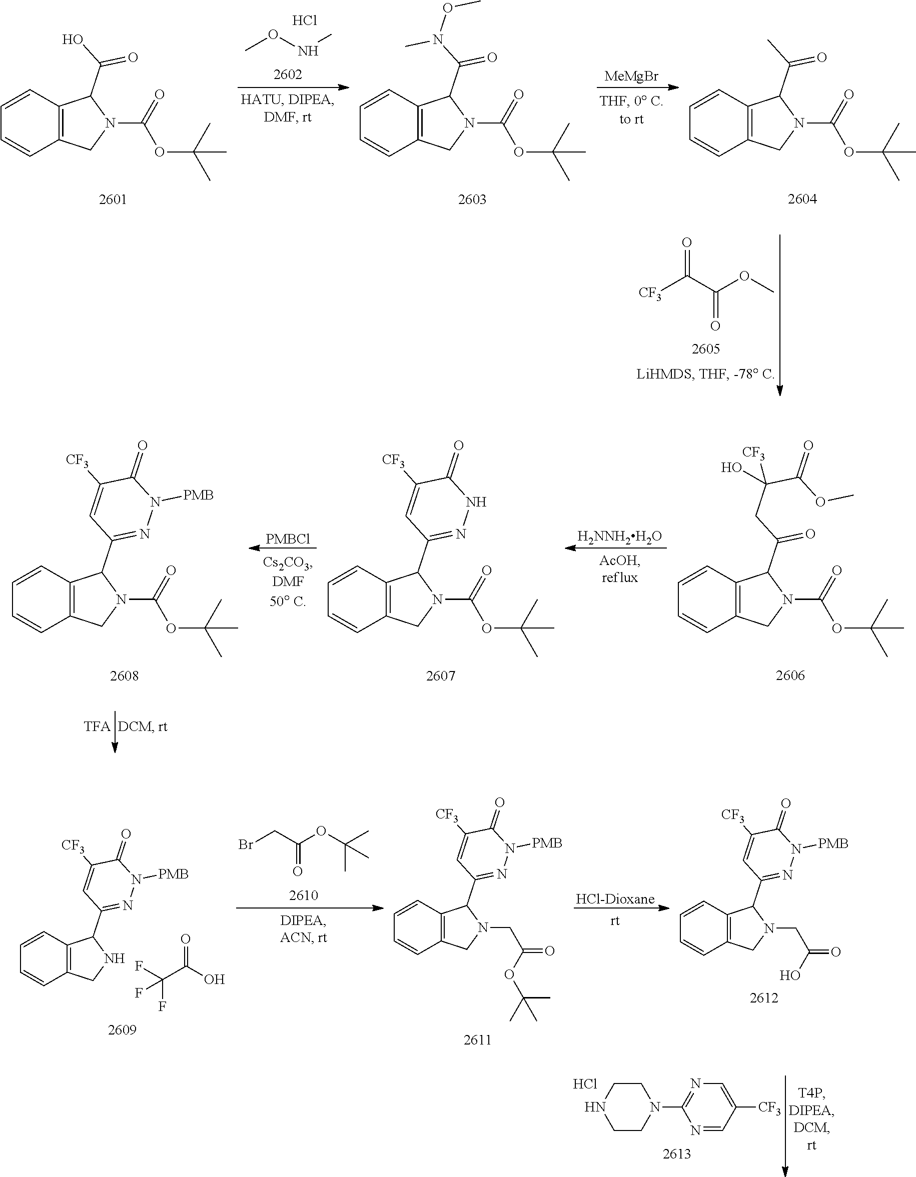

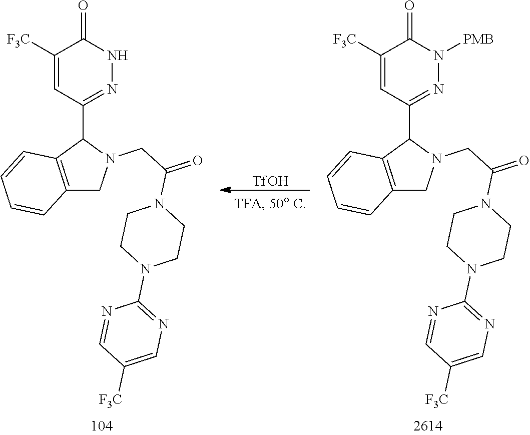

### Preparation of tert-butyl 1-(methoxy(methyl)carbamoyl)isoindoline-2-carboxylate (2603)

To a solution of 2-(tert-butoxycarbonyl)isoindoline-1-carboxylic acid 2601 (5.0 g, 0.019 mol) in DMF (100 mL) were added N,O-dimethylhydroxylamine hydrochloride 2602 (5.6 g, 0.057 mol), DIPEA (12.3 g, 0.095 mol) and HATU (14.5 g, 0.038 mol) at rt. The mixture was stirred at rt for 2 h. The reaction mixture was added into cold water and then extracted with EtOAc (200 mL×3). The combined organic layer was washed with brine, dried over Na₂SO₄, and concentrated under reduced pressure. The residue was purified by flash silica chromatography (eluting with PE/EtOAc=100:0 to 50:50) to give tert-butyl 1-(methoxy(methyl)carbamoyl)isoindoline-2-carboxylate 2603 (5 g, 90% purity, 77% yield) as a yellow oil.

LCMS (ESI) calcd for C₁₆H₂₂N₂O₄ [M+H]⁺ m/z 307.16, found 307.20.

### Preparation of tert-butyl 1-acetylisoindoline-2-carboxylate (2604)

To a solution of tert-butyl 1-(methoxy(methyl)carbamoyl)isoindoline-2-carboxylate 2603 (5 g, 0.0163 mol) in THF (50 mL) was added MeMgBr (1 M, 32 mL, 0.0320 mol) at 0° C. The mixture was stirred at rt for 1 h. The reaction mixture was added into cold water and then extracted with EtOAc (200 mL×3). The combined organic layer was washed with brine, dried over Na₂SO₄, and concentrated under reduced pressure. The residue was purified by flash silica chromatography (eluting with PE/EtOAc=100:0 to 50:50) to give tert-butyl 1-acetylisoindoline-2-carboxylate 2604 (2 g, 90% purity, 42% yield) as a yellow oil.

LCMS (ESI) calcd for C₁₅H₁₉NO₃ [M-t-Bu+H]⁺ m/z 206.14, found 206.20.

### Preparation of tert-butyl 1-(4,4,4-trifluoro-3-hydroxy-3-(methoxycarbonyl)butanoyl)isoindoline-2-carboxylate (2606)

To a solution of tert-butyl 1-acetylisoindoline-2-carboxylate 2604 (2.0 g, 0.0077 mol) in THF (30 mL) was added LiHMDS in THF (1 M, 7.7 mL, 0.0077 mol) at −78° C. The mixture was stirred at −78° C. for 10 min, then methyl 3,3,3-trifluoro-2-oxopropanoate 2605 (1.8 g, 0.0115 mol) was added at the same temperature. The mixture was stirred at −78° C. for 10 min. The reaction mixture was quenched with water (50 mL) and extracted with EtOAc (20 mL×3). The combined organic layers were washed with water and brine, dried over Na₂SO₄, concentrated to afford crude tert-butyl 1-(4,4,4-trifluoro-3-hydroxy-3-(methoxycarbonyl)butanoyl)isoindoline-2-carboxylate 2606 (2 g, 80% purity, 49% yield) as a yellow oil.

LCMS (ESI) calcd for C₁₉H₂₂F₃NO₆ [M-t-Bu+H]⁺ m/z 362.14, found 362.10.

### Preparation of tert-butyl 1-(6-oxo-5-(trifluoromethyl)-1,6-dihydropyridazin-3-yl)isoindoline-2-carboxylate (2607)

To a solution of tert-butyl 1-(4,4,4-trifluoro-3-hydroxy-3-(methoxycarbonyl)butanoyl)isoindoline-2-carboxylate 2606 (2.0 g, 0.0048 mol) in AcOH (30 mL) was added NH₂NH₂—H₂O (1.4 g, 0.023 mol, 80% wt.) at rt. The mixture was stirred at 100° C. for 1 h. The reaction mixture was concentrated to dryness, and purified by column chromatography on silica gel (eluting with MeOH/DCM, 0 to 10%) to afford tert-butyl 1-(6-oxo-5-(trifluoromethyl)-1,6-dihydropyridazin-3-yl)isoindoline-2-carboxylate 2607 (1.0 g, 80% purity, 43% yield) as a yellow oil.

LCMS (ESI) calcd for C₁₈H₁₈F₃N₃O₃[M+H]⁺ m/z 382.13, found 382.10.

### Preparation of tert-butyl 1-(1-(4-methoxybenzyl)-6-oxo-5-(trifluoromethyl)-1,6-dihydropyridazin-3-yl)isoindoline-2-carboxylate (2608)

To a solution of tert-butyl 1-(6-oxo-5-(trifluoromethyl)-1,6-dihydropyridazin-3-yl)isoindoline-2-carboxylate 2607 (1.0 g, 0.0026 mol) in DMF (20 mL) were added Cs₂CO₃ (1.7 g, 0.0052 mol) and PMBCl (0.6 g, 0.0039 mol) at rt. The mixture was stirred at 50° C. for 1 h. The reaction mixture was quenched with water (100 mL) and extracted with EtOAc (50 mL×3). The combined organic layers were washed with water and brine, dried over Na₂SO₄, concentrated, and the residue was purified by column chromatography on silica gel (eluting with EtOAc/PE, 0 to 80%) to afford tert-butyl 1-(1-(4-methoxybenzyl)-6-oxo-5-(trifluoromethyl)-1,6-dihydropyridazin-3-yl)isoindoline-2-carboxylate 2608 (1 g, 90% purity, 69% yield) as a yellow oil.

LCMS (ESI) calcd for C₂₆H₂₆F₃N₃O₄[M+H]⁺ m/z 502.19, found 502.15.

### Preparation of 6-(isoindolin-1-yl)-2-(4-methoxybenzyl)-4-(trifluoromethyl)pyridazin-3(2H)-one 2,2,2-trifluoroacetate (2609)

A solution of tert-butyl 1-(1-(4-methoxybenzyl)-6-oxo-5-(trifluoromethyl)-1,6-dihydropyridazin-3-yl)isoindoline-2-carboxylate 2608 (500 mg, 0.9950 mmol) in DCM (5 mL) and TFA (5 mL) was stirred at rt for 1 h. The mixture was concentrated to give 6-(isoindolin-1-yl)-2-(4-methoxybenzyl)-4-(trifluoromethyl)pyridazin-3(2H)-one 2,2,2-trifluoroacetate 2609 (350 mg, 90% purity, 61% yield) as a yellow oil.

LCMS (ESI) calcd for C₂₁H₁₈F₃N₃O₂[M+H]⁺ m/z 402.14, found 402.10.

### Preparation of tert-butyl 2-(1-(1-(4-methoxybenzyl)-6-oxo-5-(trifluoromethyl)-1,6-dihydropyridazin-3-yl)isoindolin-2-yl)acetate (2611)

To a solution of 6-(isoindolin-1-yl)-2-(4-methoxybenzyl)-4-(trifluoromethyl)pyridazin-3(2H)-one 2,2,2-trifluoroacetate 2609 (350 mg, 0.68 mmol) in ACN (20 mL) were added DIPEA (337 mg, 2.61 mmol) and tert-butyl 2-bromoacetate 2610 (254 mg, 1.30 mmol) at rt. The mixture was stirred at rt for 2 h. The mixture was concentrated and purified by column chromatography on silica gel (eluting with EtOAc/PE, 0 to 100%) to afford tert-butyl 2-(1-(1-(4-methoxybenzyl)-6-oxo-5-(trifluoromethyl)-1,6-dihydropyridazin-3-yl)isoindolin-2-yl)acetate 2611 (280 mg, 90% purity, 71% yield) as a yellow oil.

LCMS (ESI) calcd for C₂₇H₂₈F₃N₃O₄[M+H]⁺ m/z 516.20, found 516.15.

### Preparation of 2-(1-(1-(4-methoxybenzyl)-6-oxo-5-(trifluoromethyl)-1,6-dihydropyridazin-3-yl)isoindolin-2-yl)acetic acid (2612)

A solution of tert-butyl 2-(1-(1-(4-methoxybenzyl)-6-oxo-5-(trifluoromethyl)-1,6-dihydropyridazin-3-yl)isoindolin-2-yl)acetate 2611(280 mg, 0.543 mmol) in HCl-dioxane (4 M, 30 mL) was stirred at rt for 1 h. The mixture was concentrated to afford 2-(1-(1-(4-methoxybenzyl)-6-oxo-5-(trifluoromethyl)-1,6-dihydropyridazin-3-yl)isoindolin-2-yl)acetic acid 2612 (250 mg, 90% purity, 100% yield) as a yellow oil.

LCMS (ESI) calcd for C₂₃H₂₀F₃N₃O₄ [M+H]⁺ m/z 460.14, found 460.10.

### Preparation of 2-(4-methoxybenzyl)-6-(2-(2-oxo-2-(4-(5-(trifluoromethyl)pyrimidin-2-yl)piperazin-1-yl)ethyl)isoindolin-1-yl)-4-(trifluoromethyl)pyridazin-3(2H)-one (2614)

To a solution of 2-(1-(1-(4-methoxybenzyl)-6-oxo-5-(trifluoromethyl)-1,6-dihydropyridazin-3-yl)isoindolin-2-yl)acetic acid 2612 (250 mg, 0.543 mmol) and 2-(piperazin-1-yl)-5-(trifluoromethyl)pyrimidine hydrochloride 2613 (219 mg, 0.815 mmol) in DCM (30 mL) were added DIPEA (210 mg, 1.629 mmol) and T4P (782 mg, 1.086 mmol, 50% wt. in EtOAc) at rt. The mixture was stirred at rt for 2 h. The mixture was concentrated and the residue was purified by column chromatography on silica gel (eluting with DCM/MeOH, 0 to 10%) to afford 2-(4-methoxybenzyl)-6-(2-(2-oxo-2-(4-(5-(trifluoromethyl)pyrimidin-2-yl)piperazin-1-yl)ethyl)isoindolin-1-yl)-4-(trifluoromethyl)pyridazin-3(2H)-one 2614 (180 mg, 90% purity, 44% yield) as a yellow oil.

LCMS (ESI) calcd for C₃₂H₂₉F₆N₇O₃[M+H]⁺ m/z 674.22, found 674.10.

### Preparation of 6-(2-(2-oxo-2-(4-(5-(trifluoromethyl)pyrimidin-2-yl)piperazin-1-yl)ethyl)isoindolin-1-yl)-4-(trifluoromethyl)pyridazin-3(2H)-one (104rac)

To a solution of 2-(4-methoxybenzyl)-6-(2-(2-oxo-2-(4-(5-(trifluoromethyl)pyrimidin-2-yl)piperazin-1-yl)ethyl)isoindolin-1-yl)-4-(trifluoromethyl)pyridazin-3(2H)-one 2614 (180 mg, 0.2672 mmol) in TFA (3 mL) was added TfOH (0.5 mL) at rt. The reaction solution was stirred at 50° C. for 10 min. The mixture was adjusted to pH=8-9 with aqueous NaHCO₃ at 0° C., then extracted with EtOAc (50 mL×3). The combined organic layers were concentrated under reduced pressure. The residue was purified with flash silica chromatography (eluting with MeOH/DCM, 0 to 10%) and prep-HPLC (Gemini 5 μm C₁₈ 150×21.2 mm, mobile phase: ACN—H₂O (0.1% FA), gradient: 30-70) to afford 6-(2-(2-oxo-2-(4-(5-(trifluoromethyl)pyrimidin-2-yl)piperazin-1-yl)ethyl)isoindolin-1-yl)-4-(trifluoromethyl)pyridazin-3(2H)-one 104a/104b racemic mixture (60 mg, 95% purity, 38% yield) as white solid.

### Chiral resolution of 6-(2-(2-oxo-2-(4-(5-(trifluoromethyl)pyrimidin-2-yl)piperazin-1-yl)ethyl)isoindolin-1-yl)-4-(trifluoromethyl)pyridazin-3(2H)-one (104rac)

104a/104b racemic mixture was separated by SFC (Column: Daicel IH 250 mm×20 mm I.D., 5 μm; Mobile phase: CO₂/MeOH (0.1% NH₃)=80/20) and concentrated under reduced pressure to afford the first fraction as 104a (20.8 mg, 99.71% purity, 100% ee, yellow solid) and the second fraction as 104b (18.4 mg, 99.28% purity, 100% ee, yellow solid).

### Compound 104a

¹H NMR (400 MHz, DMSO-d₆, ppm) δ: 13.68 (s, 1H), 8.73 (s, 2H), 7.49 (s, 1H), 7.38-7.28 (m, 2H), 7.23 (t, J=7.0 Hz, 1H), 6.98 (d, J=7.4 Hz, 1H), 5.21-5.14 (m, 1H), 4.48-4.39 (m, 1H), 4.20-4.10 (m, 1H), 3.91-3.83 (m, 2H), 3.82-3.71 (m, 3H), 3.69-3.50 (m, 3H), 3.47-3.36 (m, 2H).

LCMS (ESI) calcd for C₂₄H₂₁F₆N₇O₂[M+H]⁺ m/z 554.17, found 554.25.

### Compound 104b

¹H NMR (400 MHz, DMSO-d₆, ppm) δ: 13.68 (s, 1H), 8.73 (s, 2H), 7.49 (s, 1H), 7.37-7.28 (m, 2H), 7.23 (t, J=7.0 Hz, 1H), 6.98 (d, J=7.2 Hz, 1H), 5.20-5.15 (m, 1H), 4.47-4.40 (m, 1H), 4.18-4.10 (m, 1H), 3.92-3.83 (m, 2H), 3.83-3.71 (m, 3H), 3.69-3.49 (m, 3H), 3.47-3.36 (m, 2H).

LCMS (ESI) calcd for C₂₄H₂₁F₆N₇O₂[M+H]⁺ m/z 554.17, found 554.20.

### 18. Synthesis of 6-(4-methoxy-1-(2-oxo-2-(4-(5-(trifluoromethyl)pyrimidin-2-yl)piperazin-1-yl)ethyl)pyrrolidin-2-yl)-4-(trifluoromethyl)pyridazin-3(2H)-one (Compound 138)

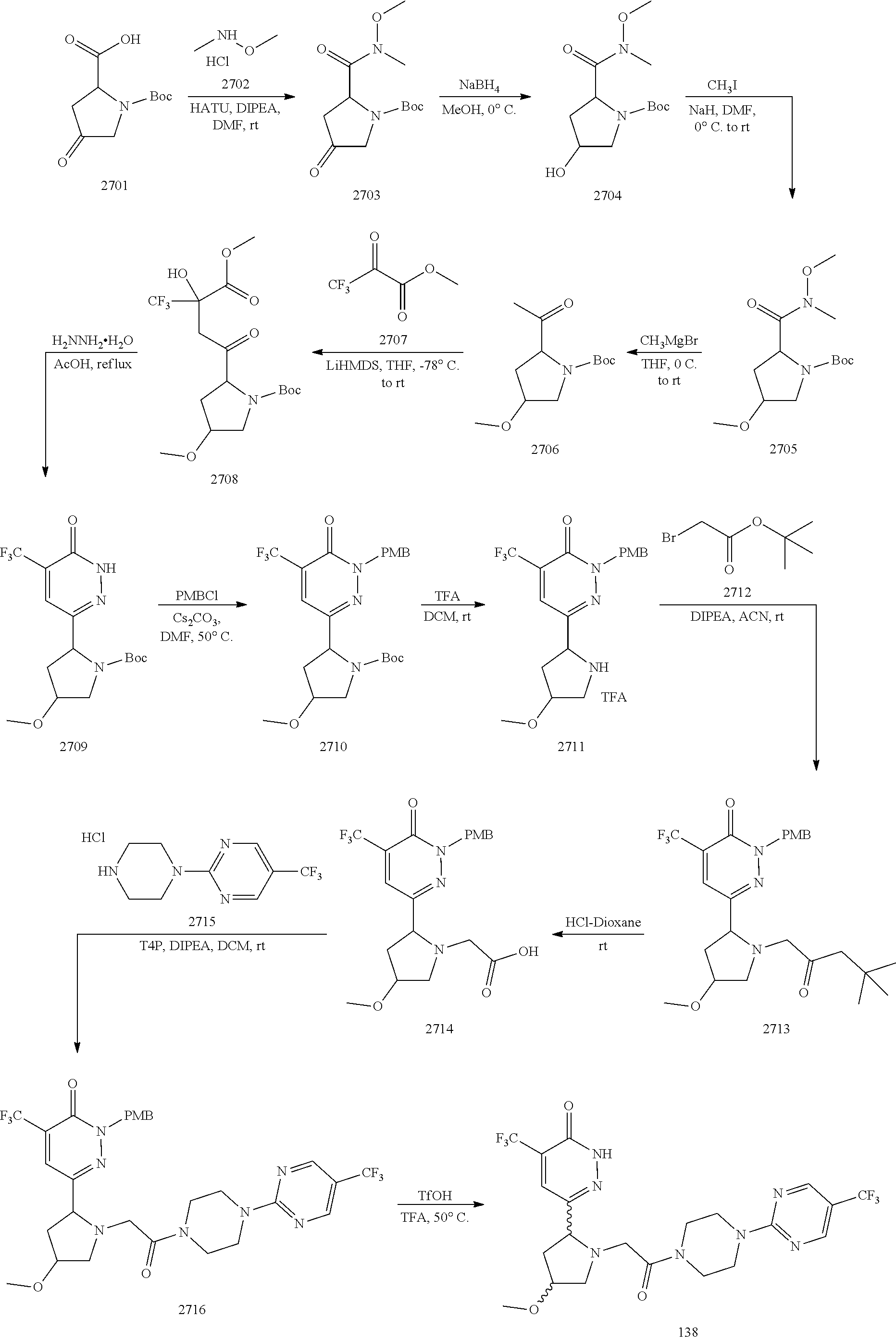

### Preparation of tert-butyl 2-(methoxy(methyl)carbamoyl)-4-oxopyrrolidine-1-carboxylate (2703)

To a solution of 1-(tert-butoxycarbonyl)-4-oxopyrrolidine-2-carboxylic acid 2701 (25 g, 0.11 mol) and N,O-dimethylhydroxylamine hydrochloride 2702 (12.7 g, 0.13 mol) in DMF (500 mL) was added HATU (82.6 g, 0.22 mol) and DIPEA (70.2 g, 0.54 mol). The mixture was stirred at rt for 2 hours. The resulting mixture was diluted with water (1000 mL) and extracted with EtOAc (500 mL×3). The combined organic phases were washed with brine, dried over sodium sulfate, concentrated, and purified by silica gel column chromatography (eluting with EtOAc/PE, 0% to 50%) to give tert-butyl 2-(methoxy(methyl)carbamoyl)-4-oxopyrrolidine-1-carboxylate 2703 (8 g, 90% purity, 24% yield) as a yellow oil.

LCMS (ESI) calcd for C₁₂H₂₀N₂O₅ [M-Boc+H]⁺ m/z 173.14, found 173.20.

### Preparation of tert-butyl 4-hydroxy-2-(methoxy(methyl)carbamoyl)pyrrolidine-1-carboxylate (2704)

To a solution of tert-butyl 2-(methoxy(methyl)carbamoyl)-4-oxopyrrolidine-1-carboxylate 2703 (8 g, 0.029 mol) in MeOH (200 mL) was added NaBH₄ (4.43 g, 0.117 mol) at 0° C. The mixture was stirred at 0° C. for 1 h. The resulting mixture was diluted with water (500 mL) and extracted with EtOAc (200 mL×4). The combined organic phases were washed with brine, dried over sodium sulfate, concentrated, and purified by silica gel column chromatography (eluting with MeOH/DCM, 0% to 5%) to give tert-butyl 4-hydroxy-2-(methoxy(methyl)carbamoyl)pyrrolidine-1-carboxylate 2704 (4 g, 90% purity, 44% yield) as a yellow oil.

LCMS (ESI) calcd for C₁₂H₂₂N₂O₅ [M+H]⁺ m/z 275.15, found 275.20.

### Preparation of tert-butyl 4-methoxy-2-(methoxy(methyl)carbamoyl)pyrrolidine-1-carboxylate (2705)

To a solution of tert-butyl 4-hydroxy-2-(methoxy(methyl)carbamoyl)pyrrolidine-1-carboxylate 2704 (4 g, 0.015 mol) in DMF (80 mL) was added NaH (1.2 g, 60% wt, 0.029 mol). The mixture was stirred at 0° C. for 30 min. Then CH₃I (4.1 g, 0.029 mol) was added to the mixture at 0° C. The mixture was then stirred at rt for 2 hours. The resulting mixture was diluted with water (200 mL) and extracted with EtOAc (100 mL×3). The combined organic phases were washed with brine, dried over sodium sulfate, concentrated, and purified by silica gel column chromatography (eluting with MeOH/DCM, 0% to 5%) to give tert-butyl 4-methoxy-2-(methoxy(methyl)carbamoyl)pyrrolidine-1-carboxylate 2705 (2.5 g, 90% purity, 53% yield) as a yellow oil.

LCMS (ESI) calcd for C₁₃H₂₄N₂O₅ [M+H]⁺ m/z 289.17, found 289.20.

### Preparation of tert-butyl 2-acetyl-4-methoxypyrrolidine-1-carboxylate (2706)

To a solution of tert-butyl 4-methoxy-2-(methoxy(methyl)carbamoyl)pyrrolidine-1-carboxylate 2705 (2.5 g, 0.0086 mol) in THF (100 mL) was added CH₃MgBr (2.9 mL, 3 M in 2-MeTHF, 0.0087 mol) at 0° C. under N₂. The mixture was stirred at rt for 2 hours. The resulting mixture was diluted with water (100 mL) and extracted with EtOAc (100 mL×3). The combined organic phases were washed with brine, dried over sodium sulfate, concentrated, and purified by silica gel column chromatography (eluting with MeOH/DCM, 0% to 10%) to give tert-butyl 2-acetyl-4-methoxypyrrolidine-1-carboxylate 2706 (1.2 g, 90% purity, 51% yield) as a yellow oil.

LCMS (ESI) calcd for C₁₂H₂₁NO₄ [M-Boc+H]⁺ m/z 144.15, found 144.20.

### Preparation of tert-butyl 4-methoxy-2-(4,4,4-trifluoro-3-hydroxy-3-(methoxycarbonyl)butanoyl)pyrrolidine-1-carboxylate (2708)

To a solution of tert-butyl 2-acetyl-4-methoxypyrrolidine-1-carboxylate 2706 (1.2 g, 0.0049 mol) in dry THF (50 mL) was added LiHMDS (10 mL, 1 M in THF, 0.010 mmol) dropwise at −78° C. under an atmosphere of N₂. After addition, the solution was stirred at −78° C. for 30 minutes. Then methyl 3,3,3-trifluoro-2-oxopropanoate 2707 (1.15 g, 0.0074 mol) was added dropwise. The resulting solution was slowly warmed to room temperature and stirred for 2 hours. The final mixture was quenched with saturated aqueous NH₄Cl solution (150 mL) and extracted with EtOAc (100 mL×3). The combined organic phases were washed with brine, dried with sodium sulfate, concentrated, and purified by silica gel column chromatography (eluting with MeOH/DCM, 0% to 10%) to give tert-butyl 4-methoxy-2-(4,4,4-trifluoro-3-hydroxy-3-(methoxycarbonyl)butanoyl)pyrrolidine-1-carboxylate 2708 (1 g, 50% purity, 24% yield) as a yellow oil.

LCMS (ESI) calcd for C₁₆H₂₄F₃NO₇ [M-Boc+H]⁺ m/z 300.15, found 299.89.

### Preparation of tert-butyl 4-methoxy-2-(6-oxo-5-(trifluoromethyl)-1,6-dihydropyridazin-3-yl)pyrrolidine-1-carboxylate (2709)

To a solution of tert-butyl 4-methoxy-2-(4,4,4-trifluoro-3-hydroxy-3-(methoxycarbonyl)butanoyl)pyrrolidine-1-carboxylate 2708 (1 g, 0.0025 mol) in AcOH (20 mL) was added H₂NNH₂·H₂O (1 mL, 80% wt in H₂O). The mixture was stirred at 80° C. for 2 hours. The resulting mixture was concentrated, and purified by silica gel column chromatography (eluting with MeOH/DCM, 0% to 10%) to give tert-butyl 4-methoxy-2-(6-oxo-5-(trifluoromethyl)-1,6-dihydropyridazin-3-yl)pyrrolidine-1-carboxylate 2709 (0.4 g, 50% purity, 22% yield) as a yellow oil.

LCMS (ESI) calcd for C₁₅H₂₀F₃N₃O₄[M+H]⁺ m/z 364.14, found 364.15.

### Preparation of tert-butyl 4-methoxy-2-(1-(4-methoxybenzyl)-6-oxo-5-(trifluoromethyl)-1,6-dihydropyridazin-3-yl)pyrrolidine-1-carboxylate (2710)

To a solution of tert-butyl 4-methoxy-2-(6-oxo-5-(trifluoromethyl)-1,6-dihydropyridazin-3-yl)pyrrolidine-1-carboxylate 2709 (400 mg, 1.1 mmol) in DMF (20 mL) was added PMBCl (258 mg, 1.7 mmol) and Cs₂CO₃ (1073 mg, 3.3 mmol). The mixture was heated at 50° C. for 2 hours. The resulting mixture was diluted with water (50 mL) and extracted with EtOAc (20 mL×3). The combined organic phases were washed with brine, dried over sodium sulfate, concentrated, and purified by silica gel column chromatography (eluting with EtOAc/PE, 0 to 50%) to give tert-butyl 4-methoxy-2-(1-(4-methoxybenzyl)-6-oxo-5-(trifluoromethyl)-1,6-dihydropyridazin-3-yl)pyrrolidine-1-carboxylate 2710 (150 mg, 90% purity, 25% yield) as a yellow oil.

LCMS (ESI) calcd for C₂₃H₂₈F₃N₃O₅[M+H]⁺ m/z 484.20, found 484.08.

### Preparation of 2-(4-methoxybenzyl)-6-(4-methoxypyrrolidin-2-yl)-4-(trifluoromethyl)pyridazin-3(2H)-one 2,2,2-trifluoroacetate (2711)

To a solution of tert-butyl 4-methoxy-2-(1-(4-methoxybenzyl)-6-oxo-5-(trifluoromethyl)-1,6-dihydropyridazin-3-yl)pyrrolidine-1-carboxylate 2710 (150 mg, 0.28 mmol) in DCM (10 mL) was added TFA (2 mL). The mixture was stirred at room temperature for 2 hours. The mixture was concentrated to provide crude 2-(4-methoxybenzyl)-6-(4-methoxypyrrolidin-2-yl)-4-(trifluoromethyl)pyridazin-3(2H)-one 2,2,2-trifluoroacetate 2711 (120 mg, 90% purity, 77% yield) as a yellow oil.

LCMS (ESI) calcd for C₁₈H₂₀F₃N₃O₃[M+H]⁺ m/z 384.15, found 384.02.

### Preparation of tert-butyl 2-(4-methoxy-2-(1-(4-methoxybenzyl)-6-oxo-5-(trifluoromethyl)-1,6-dihydropyridazin-3-yl)pyrrolidin-1-yl)acetate (2713)

To a solution of 2-(4-methoxybenzyl)-6-(4-methoxypyrrolidin-2-yl)-4-(trifluoromethyl)pyridazin-3(2H)-one 2,2,2-trifluoroacetate 2711 (120 mg, 0.22 mmol) in ACN (10 mL) was added tert-butyl 2-bromoacetate 2712 (122 mg, 0.63 mmol) and DIPEA (121 mg, 0.94 mmol). The mixture was stirred at rt for 2 hours. The resulting mixture was concentrated, and purified by silica gel column chromatography (eluting with EtOAc/PE, 0 to 50%) to give tert-butyl 2-(4-methoxy-2-(1-(4-methoxybenzyl)-6-oxo-5-(trifluoromethyl)-1,6-dihydropyridazin-3-yl)pyrrolidin-1-yl)acetate 2713 (110 mg, 90% purity, 91% yield) as a white solid.

LCMS (ESI) calcd for C₂₄H₃₀F₃N₃O₅[M+H]⁺ m/z 498.21, found 498.15.

### Preparation of 2-(4-methoxy-2-(1-(4-methoxybenzyl)-6-oxo-5-(trifluoromethyl)-1,6-dihydropyridazin-3-yl)pyrrolidin-1-yl)acetic acid (2714)

A solution of tert-butyl 2-(4-methoxy-2-(1-(4-methoxybenzyl)-6-oxo-5-(trifluoromethyl)-1,6-dihydropyridazin-3-yl)pyrrolidin-1-yl)acetate 2713 (110 mg, 0.20 mmol) in HCl dioxane solution (4 M, 10 mL) was stirred at room temperature for 2 hours. The mixture was concentrated to provide crude 2-(4-methoxy-2-(1-(4-methoxybenzyl)-6-oxo-5-(trifluoromethyl)-1,6-dihydropyridazin-3-yl)pyrrolidin-1-yl)acetic acid 2714 (90 mg, 90% purity, 92% yield) as a colorless oil.

LCMS (ESI) calcd for C₂₀H₂₂F₃N₃O₅[M+H]⁺ m/z 442.15, found 442.10.

### Preparation of 6-(4-methoxy-1-(2-oxo-2-(4-(5-(trifluoromethyl)pyrimidin-2-yl)piperazin-1-yl)ethyl)pyrrolidin-2-yl)-2-(4-methoxybenzyl)-4-(trifluoromethyl)pyridazin-3(2H)-one (2716)

To a solution of 2-(4-methoxy-2-(1-(4-methoxybenzyl)-6-oxo-5-(trifluoromethyl)-1,6-dihydropyridazin-3-yl)pyrrolidin-1-yl)acetic acid 2714 (90 mg, 0.20 mmol), 2-(piperazin-1-yl)-5-(trifluoromethyl)pyrimidine hydrochloride 2715 (66 mg, 0.24 mmol) and DIPEA (132 mg, 1.02 mmol) in DCM (10 mL) was added T4P (294 mg, 0.41 mmol, 50% wt in EtOAc). The mixture was stirred at rt for 2 hours. The resulting mixture was concentrated, and purified by silica gel column chromatography (eluting with MeOH/DCM, 0% to 10%) to give 6-(4-methoxy-1-(2-oxo-2-(4-(5-(trifluoromethyl)pyrimidin-2-yl)piperazin-1-yl)ethyl)pyrrolidin-2-yl)-2-(4-methoxybenzyl)-4-(trifluoromethyl)pyridazin-3(2H)-one 2716 (110 mg, 90% purity, 74% yield) as a white solid.

LCMS (ESI) calcd for C₂₉H₃₁F₆N₇O₄[M+H]⁺ m/z 656.23, found 656.15.

### Preparation of 6-(4-methoxy-1-(2-oxo-2-(4-(5-(trifluoromethyl)pyrimidin-2-yl)piperazin-1-yl)ethyl)pyrrolidin-2-yl)-4-(trifluoromethyl)pyridazin-3(2H)-one (138cis-1/138trans-1/138cis-2/138trans-2 mixture)

To a solution of 6-(4-methoxy-1-(2-oxo-2-(4-(5-(trifluoromethyl)pyrimidin-2-yl)piperazin-1-yl)ethyl)pyrrolidin-2-yl)-2-(4-methoxybenzyl)-4-(trifluoromethyl)pyridazin-3(2H)-one 2716 (110 mg, 0.17 mmol) in TFA (5 mL) was added TfOH (1 mL) at rt. After completion of addition, the reaction solution was stirred at 50° C. for 30 min. The residue was cooled to rt and diluted with DCM (10 mL), then adjusted pH to 8 with saturated aqueous NaHCO₃ at 0° C. The basified solution was extracted with DCM (50 mL×3). The combined organic layer was washed with brine, dried over Na₂SO₄, and concentrated under reduced pressure. The residue was purified by silica gel column chromatography (eluting with MeOH/DCM, 0 to 10%) to give 6-(4-methoxy-1-(2-oxo-2-(4-(5-(trifluoromethyl)pyrimidin-2-yl)piperazin-1-yl)ethyl)pyrrolidin-2-yl)-4-(trifluoromethyl)pyridazin-3(2H)-one 138cis-1/138trans-1/138cis-2/138trans-2 mixture (40 mg, 95% purity, 43% yield) as a white solid.

### Chiral resolution of 6-(4-methoxy-1-(2-oxo-2-(4-(5-(trifluoromethyl)pyrimidin-2-yl)piperazin-1-yl)ethyl)pyrrolidin-2-yl)-4-(trifluoromethyl)pyridazin-3(2H)-one (138cis-1/138trans-1/138cis-2/138trans-2 mixture)

138cis-1/138trans-1/138cis-2/138trans-2 mixture was separated by SFC (Column: DAICEL IH 20 mm×250 mmL I.D, 5 μm; Mobile phase: CO₂/MeOH [0.1% NH₃ (7 M Solution in MeOH)]=80/20) and concentrated under reduced pressure to afford the first fraction as 138cis-1 (2.1 mg, 96.75% purity, ee %: 100, white solid, Cis-assumed according to COSY and NOE), the second fraction as 138trans-1 (7.9 mg, 99.31% purity, ee %: 100, white solid, Trans-assumed according to COSY and NOE), the third fraction as 138cis-2 (2.8 mg, 98.80% purity, ee %: 93, white solid, Cis-assumed) and the last fraction as 138trans-2 (11.3 mg, 99.92% purity, ee %: 100, white solid, Trans-assumed).

138cis-1

¹H NMR (400 MHz, DMSO-d₆, ppm) δ: 13.52 (s, 1H), 8.73 (s, 2H), 7.85 (s, 1H), 4.04-3.97 (m, 1H), 3.94-3.83 (m, 2H), 3.75-3.63 (m, 3H), 3.61-3.54 (m, 1H), 3.54-3.44 (m, 3H), 3.40-3.34 (m, 1H), 3.29-3.25 (m, 1H), 3.25-3.19 (m, 4H), 2.48-2.47 (m, 1H), 2.10-1.96 (m, 2H).

LCMS (ESI) calcd for C₂₁H₂₃F₆N₇O₃[M+H]⁺ m/z 536.18, found 536.25.

138trans-1

¹H NMR (400 MHz, DMSO-d₆, ppm) δ: 13.40 (s, 1H), 8.73 (s, 2H), 7.96 (s, 1H), 3.97-3.88 (m, 3H), 3.75-3.59 (m, 4H), 3.54-3.46 (m, 2H), 3.36-3.33 (m, 1H), 3.29-3.23 (m, 3H), 3.21 (s, 3H), 2.77-2.72 (m, 1H), 2.47-2.43 (m, 1H), 1.85-1.76 (m, 1H).

LCMS (ESI) calcd for C₂₁H₂₃F₆N₇O₃[M+H]⁺ m/z 536.18, found 536.07.

138cis-2

¹H NMR (400 MHz, DMSO-d₆, ppm) δ: 13.52 (s, 1H), 8.73 (s, 2H), 7.85 (s, 1H), 4.04-3.98 (m, 1H), 3.96-3.83 (m, 2H), 3.76-3.64 (m, 3H), 3.60-3.54 (m, 1H), 3.53-3.45 (m, 3H), 3.40-3.35 (m, 1H), 3.30-3.26 (m, 1H), 3.25-3.19 (m, 4H), 2.48-2.46 (m, 1H), 2.16-1.94 (m, 2H).

LCMS (ESI) calcd for C₂₁H₂₃F₆N₇O₃[M+H]⁺ m/z 536.18, found 536.10.

138trans-2

¹H NMR (400 MHz, DMSO-d₆, ppm) δ: 13.40 (s, 1H), 8.73 (s, 2H), 7.96 (s, 1H), 3.96-3.88 (m, 3H), 3.75-3.59 (m, 4H), 3.54-3.47 (m, 2H), 3.38-3.33 (m, 1H), 3.30-3.23 (m, 3H), 3.21 (s, 3H), 2.78-2.72 (m, 1H), 2.48-2.44 (m, 1H), 1.84-1.77 (m, 1H).

LCMS (ESI) calcd for C₂₁H₂₃F₆N₇O₃[M+H]⁺ m/z 536.18, found 536.12.

### Assays

Exemplary compounds of the invention were prepared and tested to determine their effect as PARP1 and PARP7 inhibitors. Typical assays are described below.

### PARP1 Biochemical Dissociation-Enhanced Lanthanide Fluorescence Immunoassay (DELFIA Assay)

Optiplate HB 384-well plates were coated with anti-FLAG antibody, supplied as a 4 mg/ml solution, using a Na₂CO₃/HCO₃ coating buffer at pH 9.6, overnight at 4° C., in order to achieve a final immobilisation per well of 0.3 g. Wells were then washed 3×5 min in coating wash buffer (PBS/0.05% Tween (v/v)), and blocked with 2% BSA (w/v) in coating wash buffer overnight at 4° C. Prior to assay, wells were washed 3×5 min in coating wash buffer. For the assay 20 μl of 2.5 nM recombinant full length human N-terminally FLAG-tagged PARP1 was added to each well of the 384-well plate for 30 min at room temperature followed by addition of 50 nL of compound solution in DMSO using pintool technology. Following incubation for 30 min at room temperature, 5 μl of 10 μM biotin-NAD+ and 10 nM activation DNA (sequence shown below) in solution in 20 mM HEPES (pH 7.5), 100 mM NaCl, 2 mM DTT, 0.1% BSA (w/v), 0.02% Tween (v/v) assay buffer. Auto-PARylation proceeded for 2 h at room temperature prior to the addition of 5 μl of 12 mM NAD⁺ quenching solution. After 30 min at room temperature, assay solution was removed and following washing 5 times for 3 min, 100 μl of a 1:1000 dilution of DELFIA Eu-N1 Streptavidin reagent was added. Plates were then incubated for 30 min at room temperature. Reaction mixture was removed and plates washed times for 3 min prior to the addition of 25 μl DELFIA enhancement solution. Following incubation for 30 min at room temperature, fluorescence was measured on a Pherastar FS (Ex337 nm, Em620 nm; integration start 60 μs; integration time 400 μs).

Typically compounds were tested from 20 μM at 3-fold dilution intervals in 12-point concentration-response curves to determine IC₅₀ values. Data was analysed using ActivityBase software and replicate values for the low (without enzyme, 0.2% DMSO) and high (0.2% DMSO) % controls were averaged and the data obtained from the test compounds expressed as a % of 100% using the below formulae:

$$\% value = 100 - ( 100 * ( ( high control - unknown ) / ( high control - low control ) )$$

% data was fitted to a non-linear regression equation (log inhibitor vs response-variable slope 4-parameters) to obtain IC₅₀ values.

The IC₅₀ values for a variety of test compounds are shown in Table 1.

### Activation DNA Sequences

```
Duplex Sequences
SEQ ID NO: 1
5′-ACCCTGCTGTGGGC/ideoxyU/GGAGAACAAGGTGAT-3′
SEQ ID NO: 2
5′-ATCACCTTGTTCTCCAHGCCCACAGCAGGGT-3
SEQ ID NO: 3
    5′-ACCCTGCTGTGGGCGGAGAACAAGGTGAT-3
            | || | |||||||||||||||
3′-TGGGACGACACCCGHACCTCTTGTTCCACTA-5′
PARP7 biochemical dissociation-enhanced lanthanide
fluorescence immunoassay (DELFIA assay)
```

Optiplate HB 384-well plates were coated with anti-FLAG antibody, supplied as a 4 mg/ml solution, using a Na₂CO₃/HCO₃ coating buffer at pH 9.6, overnight at 4° C., in order to achieve a final immobilisation per well of 0.3 μg. Wells were then washed 3× in coating wash buffer (PBS/0.05% Tween (v/v)), blocked with 2% BSA (w/v) in coating wash buffer and washed 3 further times prior to assay. For the assay 20 μl of 12.5-37.5 nM recombinant human Flag-tagged PARP7 (amino acids 456-657) was added to each well of the 384-well plate for 30 min at room temperature. 50 nl of test compound in DMSO was added using pintool technology and plates were incubated for a further 30 min at room temperature. 5 μl of 15 μM biotin-NAD⁺ assay solution in 20 mM HEPES (pH 7.5), 100 mM NaCl, 2 mM DTT, 0.1% BSA (w/v), 0.02% Tween (v/v) assay buffer was then added and MARylation proceeded for 2-3 h at room temperature prior to the addition of 5 μl of 12 mM NAD⁺ quenching solution. After 30 min at room temperature, assay solution was removed and following washing 5 times, 100 μl of a 1:1000 dilution of DELFIA Eu-N1 Streptavidin reagent was added. Plates were then incubated for 30 min at room temperature. Reaction mixture was removed and plates washed 5 times prior to the addition of 25 μl DELFIA enhancement solution. Following incubation for 30 min at room temperature, fluorescence was read on either an Envision or Pherastar FS (Ex337 nm, Em620 nm).

Typically compounds were tested from 10-20 μM at 0.5 log intervals in 10-12-point concentration-response curves to determine IC₅₀ values. Data was analysed using ActivityBase software and replicate values for the low (without enzyme, 0.2% DMSO) and high (0.2% DMSO) % controls were averaged and the data obtained from the test compounds expressed as a % of 100% using the below formulae:

$$\% activity = 100 * ( value - low control ) / ( high control - low control ) )$$

% activity data was fitted with 4-parameter non-linear regression equation to obtain IC50 values.

The IC50 values for a variety of test compounds are shown in Tables 1 and 1A.

TABLE 1
Results of Parp1 and Parp7 assays for selected compounds

| | PARP1 | PARP7 |
|---|---|---|
| Compound | activity | activity |
| 1a | ++ | + |
| 1b | +++ | ++ |
| 2a | NT | ++ |
| 2b | NT | +++ |
| 3a | NT | +++ |
| 3b | NT | ++ |
| 4a | ++ | + |
| 4b | ++ | ++ |
| 5rac | +++ | ++++ |
| 6a | +++ | ++ |
| 6b | ++ | + |
| 7a | ++ | ++ |
| 7b | ++ | ++ |
| 8a | ++ | ++ |
| 8b | ++ | + |
| 9a | ++ | ++ |
| 9b | + | + |
| 10a | ++ | + |
| 10b | +++ | +++ |
| 11a | ++ | ++ |
| 11b | ++ | + |
| 12rac | ++ | ++ |
| 13a | ++ | + |
| 13b | ++ | ++ |
| 14a | +++ | +++ |
| 14b | ++ | +++ |
| 15a | ++ | ++ |
| 15b | ++ | + |
| 16rac | ++ | ++ |
| 17rac | ++ | ++ |
| 18a | +++ | ++++ |
| 18b | +++ | +++ |
| 19rac | ++ | +++ |
| 20rac | ++ | ++ |
| 21rac | +++ | +++ |
| 22rac | ++ | ++ |
| 23rac | ++ | ++ |
| 24a | + | + |
| 24b | +++ | ++++ |
| 25a | +++ | +++ |
| 25b | +++ | ++ |
| 26rac | + | ++ |
| 27a | +++ | + |
| 27b | +++ | +++ |
| 28a | ++ | + |
| 28b | ++ | + |
| 29a | ++ | + |
| 29b | ++ | − |
| 30rac | ++ | ++ |
| 31rac | ++ | + |
| 32a | ++ | ++ |
| 32b | − | − |

TABLE 1A

| | PARP1 | PARP7 |
|---|---|---|
| Compound | activity | activity |
| 33a | − | ++ |
| 33b | ++ | ++ |
| 34a | ++ | +++ |
| 34b | + | + |
| 35a | +++ | ++++ |
| 35b | + | ++ |
| 36a | +++ | ++++ |
| 36b | + | ++ |
| 37a | +++ | ++ |
| 37b | + | + |
| 38a | ++ | + |
| 38b | + | + |
| 39rac | +++ | ++++ |
| 40a | +++ | ++++ |
| 40b | + | + |
| 41a | +++ | ++++ |
| 41b | + | + |
| 42a | + | ++ |
| 42b | +++ | ++++ |
| 43a | +++ | ++++ |
| 43b | + | + |
| 44rac | +++ | ++++ |
| 45a | +++ | ++++ |
| 45b | + | + |
| 46a | +++ | ++++ |
| 46b | ++ | + |
| 47rac | +++ | ++++ |
| 48 | ++++ | − |
| 49 | +++ | − |
| 50 | +++ | − |
| 51 | +++ | + |
| 52rac | ++++ | ++++ |
| 53rac | ++ | +++ |
| 54a | ++ | ++++ |
| 54b | − | + |
| 55a | + | + |
| 55b | ++ | ++++ |
| 56a | ++ | + |
| 56b | ++ | + |
| 57a | ++ | ++++ |
| 57b | + | ++ |
| 58rac | ++ | ++++ |
| 59 | +++ | − |
| 60rac | ++ | ++++ |
| 60a | +++ | ++++ |
| 60b | ++ | +++ |
| 61 | ++ | − |
| 62 | ++ | ++ |
| 63a | +++ | +++ |
| 63b | − | + |
| 64 | ++ | + |
| 65rac | +++ | +++ |
| 66a | ++ | ++ |
| 66b | − | + |
| 67rac | ++ | +++ |
| 68rac | ++ | ++++ |
| 69a | +++ | ++++ |
| 69b | + | + |
| 70a | + | +++ |
| 70b | − | + |
| 71a | + | + |
| 71b | +++ | +++ |
| 72a | ++ | +++ |
| 72b | ++++ | ++++ |
| 73a | ++ | + |
| 73b | + | + |
| 74a | +++ | ++++ |
| 74b | + | ++ |
| 75a | +++ | ++++ |
| 75b | − | + |
| 76a | + | ++ |
| 76b | ++ | ++++ |
| 77a | + | ++ |
| 77b | ++ | ++++ |
| 78a | ++ | ++++ |
| 78b | − | ++ |
| 79a | +++ | ++++ |
| 79b | + | + |
| 80a | + | ++ |
| 80b | +++ | ++++ |
| 81a | + | ++ |
| 81b | +++ | ++++ |
| 82a | +++ | ++++ |
| 82b | + | ++ |
| 83a | ++ | ++++ |
| 83b | − | ++ |
| 84a | + | +++ |
| 84b | + | ++ |
| 85a | ++++ | ++++ |
| 85b | + | ++ |
| 86 | ++ | + |
| 87a | ++ | + |
| 87b | +++ | +++ |
| 88a | ++ | ++ |
| 88b | ++ | ++ |
| 89a | ++ | ++++ |
| 89b | + | ++ |
| 90a | + | − |
| 90b | + | + |
| 91a | ++ | ++++ |
| 91b | + | ++ |
| 93a | ++++ | ++++ |
| 93b | + | ++ |
| 94a | +++ | +++ |
| 94b | + | + |
| 95a | ++++ | ++++ |
| 95b | + | ++ |
| 96a | ++ | ++ |
| 96b | ++++ | ++++ |
| 97a | ++ | ++++ |
| 97b | + | ++ |
| 98a | ++ | ++++ |
| 98b | + | + |
| 99a | ++++ | ++++ |
| 99b | + | ++ |
| 100a | +++ | ++++ |
| 100b | ++ | + |
| 101a | ++ | − |
| 101b | ++ | ++ |
| 102a | ++ | ++++ |
| 102b | + | +++ |
| 103 | ++ | + |
| 104a | ++ | ++++ |
| 104b | + | ++ |
| 105a | + | +++ |
| 105b | + | +++ |
| 106a | ++ | − |
| 106b | + | + |
| 107a | + | ++ |
| 107b | − | ++ |
| 108a | +++ | ++++ |
| 108b | + | ++ |
| 109a | +++ | ++++ |
| 109b | − | + |
| 110a | ++ | + |
| 110b | + | ++ |
| 111a | +++ | ++++ |
| 111b | − | + |
| 112a | +++ | ++++ |
| 112b | + | ++ |
| 113rac | +++ | ++++ |
| 114a | ++ | ++ |
| 114b | + | − |
| 115rac | − | ++ |
| 116a | ++ | +++ |
| 116b | + | + |
| 117a | ++++ | ++++ |
| 117b | + | ++ |
| 118a | ++++ | ++++ |
| 118b | + | + |
| 119a | ++ | ++++ |
| 119b | − | + |
| 120a | + | +++ |
| 120b | − | + |
| 121a | ++ | +++ |
| 121b | + | + |
| 122a | ++ | ++++ |
| 122b | ++ | ++ |
| 123a | +++ | ++ |
| 123b | − | − |
| 124a | +++ | +++ |
| 124b | + | + |
| 125a | ++ | ++++ |
| 125b | + | + |
| 126a | +++ | ++++ |
| 126b | + | ++ |
| 127a | +++ | ++ |
| 127b | + | + |
| 128a | ++++ | ++++ |
| 128b | + | ++ |
| 129a | ++ | +++ |
| 129b | − | − |
| 130a | +++ | ++++ |
| 130b | + | + |
| 138cis1 | +++ | ++++ |
| 138trans1 | ++ | ++++ |
| 138cis2 | + | + |
| 138trans2 | − | + |
| 143a | ++ | ++++ |
| 143b | + | + |
| 144a | +++ | ++++ |
| 144b | + | + |
| 145a | +++ | ++++ |
| 145b | − | − |
| 146a | ++ | ++++ |
| 146b | − | + |
| 147a | +++ | ++++ |
| 147b | − | ++ |
| 148a | +++ | ++++ |
| 148b | + | +++ |
| 149a | +++ | ++++ |
| 149b | − | ++ |
| 150a | +++ | ++++ |
| 150b | + | +++ |

Key: − indicates IC₅₀ value above 10 μM
+ indicates IC₅₀ value above 1 μM up to 10 μM
++ indicates IC₅₀ value above 100 nM up to 1 μM
+++ indicates IC₅₀ value above 10 nM up to 100 nM
++++ indicates IC₅₀ value of 10 nM or less

1. A compound, that is a PARP7 and/or a PARP1 inhibitor compound, having a formula: 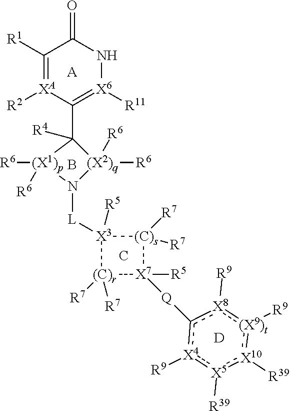
wherein:
X^{A} is selected from C and N;
when X^{A} is C:
R¹ and R² together form a 4 to 7 membered ring together with the atoms of ring A to which they are attached; or
R¹ is selected from a C₁ to C₆ alkyl, alkoxy or haloalkyl group, a C₃ to C₆ cycloalkyl or heterocyclic group, a halogen group, and —CN; and R² is selected from H, a C₁ to C₃ alkyl group, a C₁ to C₃ fluoroalkyl group, and a halogen group;
when X^{A} is N:
R¹ is selected from a C₁ to C₆ alkyl, alkoxy or haloalkyl group, a C₃ to C₆ cycloalkyl or heterocyclic group, a halogen group, and —CN; and
R² is absent;
X⁶ is independently selected from C and N;
when X⁶ is N, R¹¹ is absent;
when X⁶ is C, R¹¹ is selected from H, —CN, a C₁ to C₃ alkyl group, and a C₁ to C₃ haloalkyl group;
p is selected from 0, 1, 2, 3, 4 and 5;
q is selected from 0, 1 and 2, with the proviso that p+q is 2, 3, 4 or 5;
each X¹ is independently selected from C, N, O and S;
each X² is independently selected from C, N, O and S;
ring B is a saturated ring or an unsaturated ring having exactly one double bond;
R⁴ is selected from H, deuterium, a halogen, a C₁-C₆ alkyl group, a linear or branched C₁-C₆ halogenated alkyl group, an —OH group, a linear or branched C₁-C₆ alcohol group, an NH₂ group, a C₁-C₆ amino group, a C₁-C₆ alkoxy group, a C₁-C₆ haloalkoxy group, a C₁-C₆ fluoroalkoxy group, an aryloxy group, a haloaryloxy group, and an arylalkoxy group, or R⁴ and an R⁶ together represent a C₁ to C₃ alkylene group;
each R⁶ is independently present or absent depending on the valency of the X¹ or X² atom to which it is attached, and each R⁶ is when present independently selected from H, deuterium, a halogen, a C₁-C₆ alkyl group, a linear or branched C₁-C₆ halogenated alkyl group, an —OH group, a linear or branched C₁-C₆ alcohol group, an NH₂ group, a C₁-C₆ amino group, a C₁-C₆ alkoxy group, a C₁-C₆ haloalkoxy group, a C₁-C₆ fluoroalkoxy group, an aryloxy group, a haloaryloxy group, and an arylalkoxy group; or a pair of R⁶ groups attached to different atoms together represent a —CH₂— or —CH₂CH₂— group which forms a ring with ring B atoms; or R⁴ and an R⁶ together represent a C₁ to C₃ alkylene group;
X³ and X⁷ are each independently selected from C and N;
each bond between the X³ and/or the X⁷, and/or the carbon atoms in ring C may be a single bond or a double bond depending on the number of bonds to, and the valence of, the X³ atom, the X⁷ atom and the carbon atoms;
each R⁵ is independently present or absent depending on the number of bonds to, and the valency of, the X³ or X⁷ atom to which that R⁵ is attached, and each R⁵ is when present independently selected from H, deuterium, a halogen, a C₁-C₆ alkyl group, a linear or branched C₁-C₆ halogenated alkyl group, an —OH group, a linear or branched C₁-C₆ alcohol group, an NH₂ group, a C₁-C₆ amino group, a C₁-C₆ alkoxy group, a C₁-C₆ haloalkoxy group, a C₁-C₆ fluoroalkoxy group, an aryloxy group, a haloaryloxy group, and an arylalkoxy group;
each R⁷ is independently present or absent depending on the number of bonds to the carbon atom to which that R⁷ is attached, and each R⁷ is when present independently selected from H, deuterium, a halogen, a C₁-C₆ alkyl group, a linear or branched C₁-C₆ halogenated alkyl group, an —OH group, a linear or branched C₁-C₆ alcohol group, an NH₂ group, a C₁-C₆ amino group, a C₁-C₆ alkoxy group, a C₁-C₆ haloalkoxy group, a C₁-C₆ fluoroalkoxy group, an aryloxy group, a haloaryloxy group, and an arylalkoxy group, or a pair of R⁷ groups attached to different atoms together represent a —CH₂— or —CH₂CH₂— group which forms a ring with ring C atoms;
r is selected from 0, 1, 2, 3, 4 and 5;
s is selected from 0, 1, 2, 3, 4 and 5, with the proviso that r+s is 2, 3, 4 or 5;
each of X⁴, X⁵, X⁸, X⁹ and X¹⁰ is independently selected from C, N, O and S;
tis 0 or 1;
ring D is an aromatic ring, and includes 2 or 3 double bonds depending on the size of t and the nature of X⁴, X⁵, X⁸, X⁹ and X¹⁰;
one of R³⁹ is an R⁹ and one of R³⁹ is an R³;
R³ being selected from a halogenated C₁ to C₃ alkyl group, a halogenated C₁ to C₃ alkoxy group, a halogen group, a —CN group, and an amide group;
each R⁹ is independently present or absent depending on the number of bonds to, and the valency of, the X⁴, X⁵, X⁸, X⁹ or X¹⁰ atom to which that R⁹ is attached, and each R⁹ is when present independently selected from H, a C₁-C₃ alkyl group, a C₁-C₃ fluoroalkyl group, —CN, and a halogen group;
Q is a bond or a linker selected from —O—, —NH—, —N(Me)-, —CH2- and C═O,
and
L is selected from a group having one of the following structures: 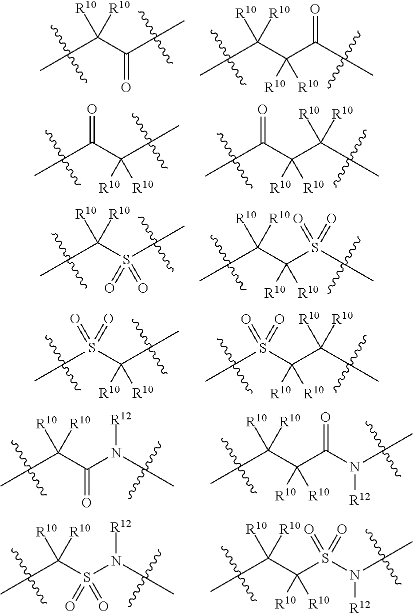
wherein:
each R¹⁰ is independently selected from H, a halogen, a C₁-C₆ alkyl group, a linear or branched C₁-C₆ halogenated alkyl group, an NH₂ group, a C₁-C₆ amino group, an —OH group, a linear or branched C₁-C₆ alcohol group, and a C₁-C₆ alkoxy group; and
R¹² is selected from H, a C₁-C₆ alkyl group, and a linear or branched C₁-C₆ halogenated alkyl group;
or L and R⁵ together represent: 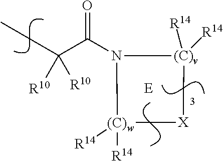
wherein:
ring E is spiro to ring C;
each R¹⁰ is independently selected from H, a halogen, a C₁-C₆ alkyl group, a linear or branched C₁-C₆ halogenated alkyl group, an NH₂ group, a C₁-C₆ amino group, an —OH group, a linear or branched C₁-C₆ alcohol group, and a C₁-C₆ alkoxy group;
v is 1 or 2;
w is 1 or 2; and
each R¹⁴ is H.

2-14. (canceled)

15. A compound according to claim 1, wherein ring B is selected from:

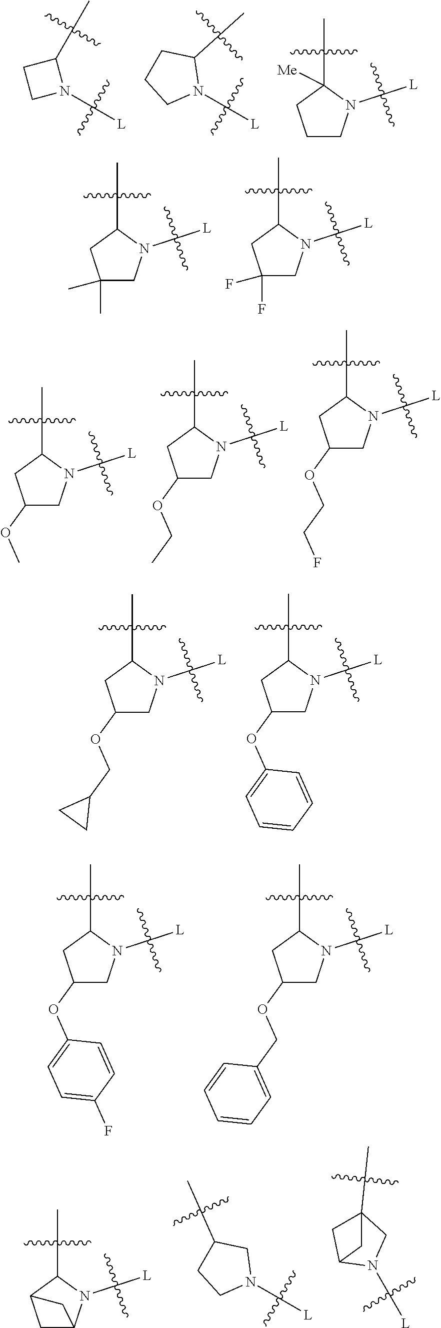

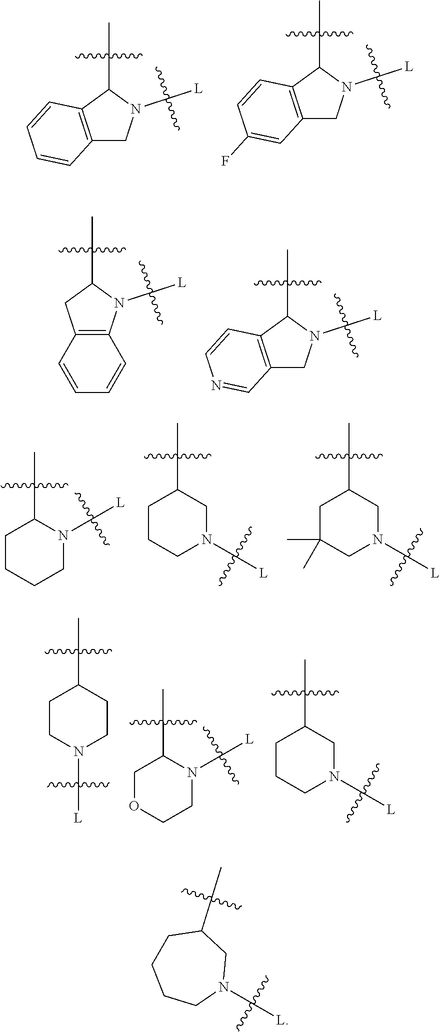

16-20. (canceled)

21. A compound according to claim 1, wherein L is selected from:

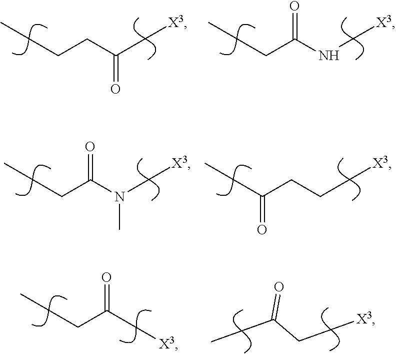

22. (canceled)

23. A compound according to claim 1, wherein L and R⁵ together represent:

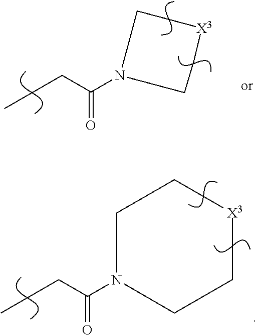

24-26. (canceled)

27. A compound according to claim 1, wherein ring C has a structure selected from:

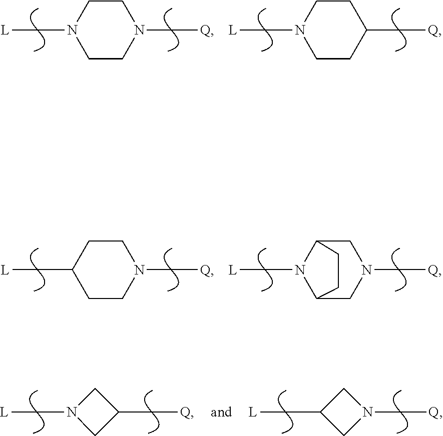

28. A compound according to claim 1, wherein ring D is selected from: 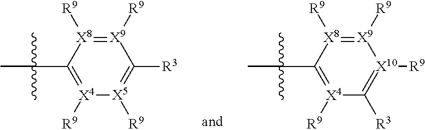
wherein X⁴, X⁵, X⁸, X⁹, and X¹⁰ are each independently selected from C and N;
or wherein ring D has the following structure: 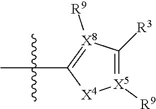
wherein X⁵ and X⁸ are each independently selected from C and N, and X⁴ is selected from O and S;
or wherein ring D has the following structure: 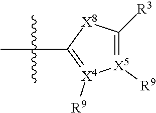
wherein X⁴ and X⁵ are each independently selected from C and N, and X⁸ is selected from O and S,
or wherein ring D is selected from: 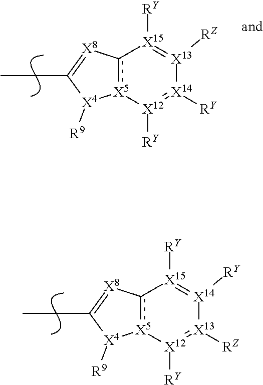
wherein:
X⁴ is selected from N, O, and S,
when X⁴ is O or S:
R⁹ is absent;
X⁵, X¹⁰, X¹², X¹³, X¹⁴, and X¹⁵ are each independently selected from C and N;
each R^{Y} is:
i) absent when the corresponding one of X¹², X¹⁴, and X¹⁵ is an N;
or
ii) H when the corresponding one of X¹², X¹⁴, and X¹⁵ is a C;
R^{Z} is:
i) absent when X¹³ is N; or
ii) selected from H, —CN, —CF₃, and a halogen when X¹³ is C.

29-38. (canceled)

39. A compound according to claim 1, wherein ring D is selected from:

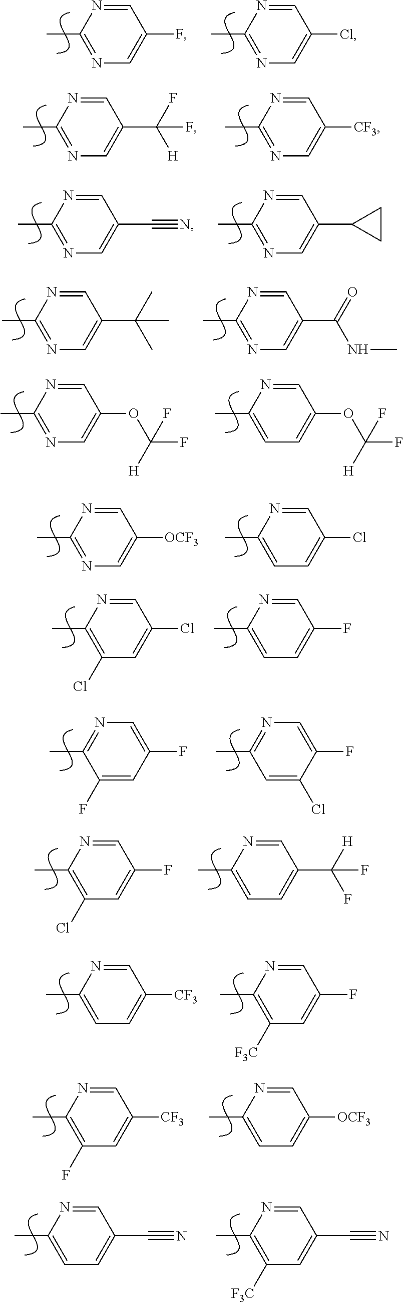

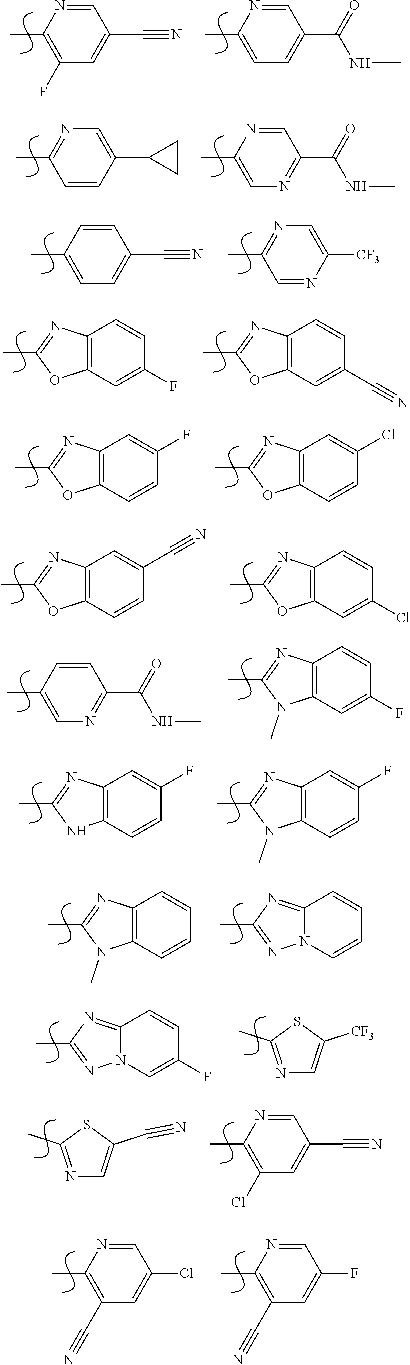

40. A compound according to claim 1, having the following formula: 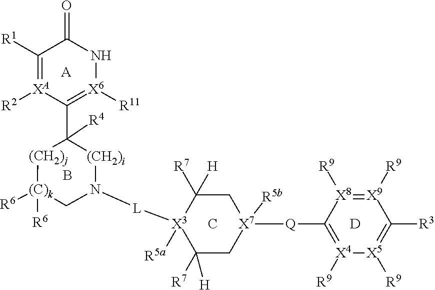
wherein:
R¹ is selected from —CH₃, —CH₂—CH₃, a cyclopropyl group, CF₃, C₁, and —CN;
i is 0, 1 or 2;
j is 0, 1, or 2;
k is 0 or 1;
with the proviso that i, j and k sum to 1, 2, 3, or 4; and
R⁴:
i) is selected from H and methyl group, and each R⁶ is independently selected from H, F, a methyl group, a C1 to C4 alkoxy group, a C₁ to C₄ haloalkoxy group, —OPh, —OCH₂Ph, 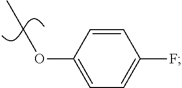
or
ii) is fused with one R⁶ to form a —CH₂— group bridging ring B, and the other R⁶ is H;
L is selected from: 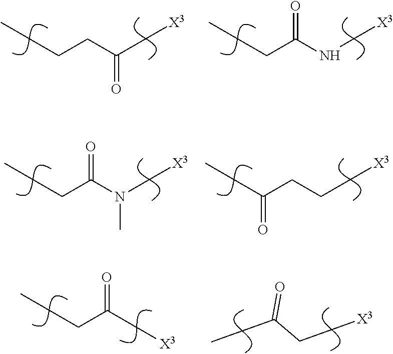
X³ and X⁷ are each selected from C and N, with the proviso that at least one of X³ and X⁷ is N;
when X³ is C, R^{5a} is H;
when X³ is N, R^{5a} is absent;
when X⁷ is C, R^{5b} is H and Q is a bond or a linker selected from —O—, —NH—, and —N(CH₃)—;
when X⁷ is N, R^{5b} is absent and Q is a bond;
each R⁷ is H, or the R⁷ groups are fused to form a —CH₂—CH₂— group bridging ring C;
X⁴, X⁵, X⁸, and X⁹ are each selected from C and N, with the proviso that no more than two of X⁴, X⁵, X⁸, and X⁹ are N;
when X⁴, X⁵, X⁸, or X⁹ is an N, the corresponding R⁹ is absent;
when X⁴, X⁵, X⁸, or X⁹ is a C, the corresponding R⁹ is selected from H, F, Cl, —CN, and —CF₃; and
R³ is selected from —F; —Cl; —CF₃; —CHF₂, —OCF₂H; —OCF₃; —CN;

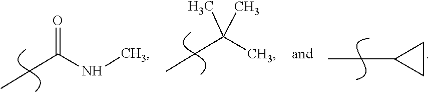

41. A compound according to claim 1, which compound comprises the following formula: 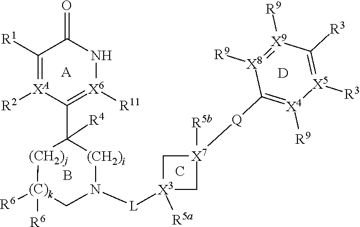
wherein:
R¹ is selected from —CH₃, —CH₂—CH₃, a cyclopropyl group, CF₃, Cl, and —CN;
i is 0, 1 or 2;
j is 0, 1, or 2;
k is 0 or 1;
with the proviso that i, j and k sum to 1, 2, 3, or 4; and
R⁴:
i) is selected from H and methyl group, and each R⁶ is independently selected from H, F, a methyl group, a C1 to C4 alkoxy group, a C₁ to C₄ haloalkoxy group, —OPh; —OCH₂Ph; 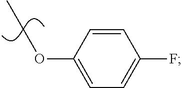
or
ii) is fused with one R⁶ to form a —CH₂— group bridging ring B, and the other R⁶ is H;
L is selected from: 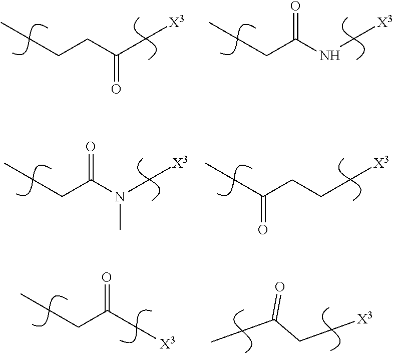
one of X³ and X⁷ is C, and the other of X³ and X⁷ is N;
when X³ is C, R^{5a} is H;
when X³ is N, R^{5a} is absent;
when X⁷ is C, R^{5b} is H and Q is a bond or a linker selected from —O—, —NH—, and —N(CH₃)—;
when X⁷ is N, R^{5b} is absent and Q is a bond;
X⁴, X⁵, X⁸, and X⁹ are each selected from C and N, with the proviso that no more than two of X⁴, X⁵, X⁸, and X⁹ are N;
when X⁴, X⁵, X⁸, or X⁹ is an N, the corresponding R⁹ is absent;
when X⁴, X⁵, X⁸, or X⁹ is a C, the corresponding R⁹ is selected from H, F, Cl, —CN, and —CF₃; and
R³ is selected from —F; —Cl; —CF₃; —CHF₂, —OCF₂H; —OCF₃; —CN;

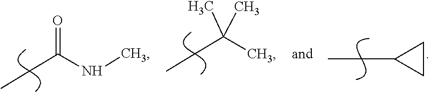

42. A compound according to claim 1, having the following formula: 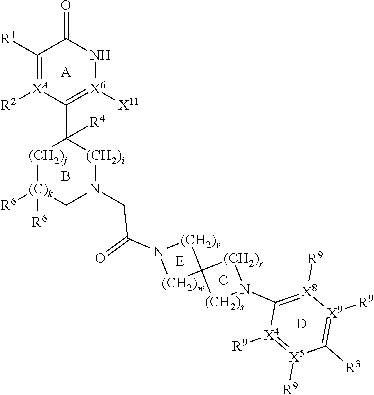
wherein:
R¹ is selected from —CH₃, —CH₂—CH₃, a cyclopropyl group, CF₃, Cl, and —CN;
i is 0, 1 or 2;
j is 0, 1, or 2;
k is 0 or 1;
with the proviso that i, j and k sum to 1, 2, 3, or 4;
R⁴:
i) is selected from H and methyl group, and each R⁶ is independently selected from H, F, a methyl group, a C1 to C4 alkoxy group, a C₁ to C₄ haloalkoxy group, —OPh, —OCH₂Ph, 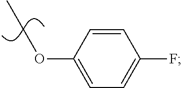
or
ii) is fused with one R⁶ to form a —CH₂— group bridging ring B, and the other R⁶ is H;
v is 1 or 2;
w is 1 or 2;
r is 1 or 2;
s is 1 or 2;
X⁴, X⁵, X⁸, and X⁹ are each selected from C and N, with the proviso that no more than two of X⁴, X⁵, X⁸, and X⁹ are N;
when X⁴, X⁵, X⁸, or X⁹ is an N, the corresponding R⁹ is absent;
when X⁴, X⁵, X⁸, or X⁹ is a C, the corresponding R⁹ is selected from H, F, Cl, —CN, and —CF₃; and
R³ is selected from —F; —Cl; —CF₃; —CHF₂, —OCF₂H; —OCF₃; —CN;

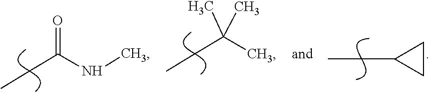

43. A compound according to claim 1, having the following formula: 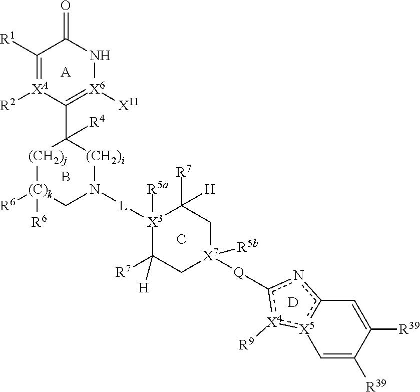
wherein:
R¹ is selected from —CH₃, —CH₂—CH₃, a cyclopropyl group, CF₃, Cl, and —CN;
i is 0, 1 or 2;
j is 0, 1, or 2;
k is 0 or 1;
with the proviso that i, j and k sum to 1, 2, 3, or 4;
R⁴:
i) is selected from H and methyl group, and each R⁶ is independently selected from H, F, a methyl group, a C1 to C4 alkoxy group, a C₁ to C₄ haloalkoxy group, —OPh; —OCH₂Ph; 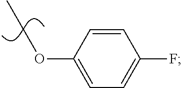
or
ii) is fused with one R⁶ to form a —CH₂— group bridging ring B, and the other R⁶ is H;
L is selected from: 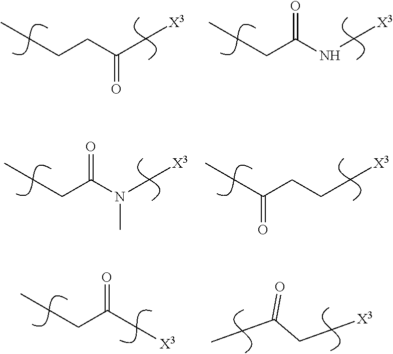
X³ and X⁷ are each selected from C and N, with the proviso that at least one of X³ and X⁷ is N;
when X³ is C, R^{5a} is H;
when X³ is N, R^{5a} is absent;
when X⁷ is C, R^{5b} is H, and Q is a bond or a linker selected from —O—, —NH—, and —N(CH₃)—;
when X⁷ is N, R^{5b} is absent, and Q is a bond;
each R⁷ is H, or the R⁷ groups are fused to form a —CH₂—CH₂— group bridging ring C;
ring D is an aromatic ring;
X⁴ is selected from N and O;
when X⁴ is O, R⁹ is absent;
X⁵ is selected from C and N;
when X⁴ is N and X⁵ is N, R⁹ is absent;
when X⁴ is N and X⁵ is C, R⁹ is H or a methyl group;
each R³⁹ is selected from H, F, Cl, and —CN, with the proviso that at least one R³⁹ is H.

44. A compound according to claim 1, having the following formula: 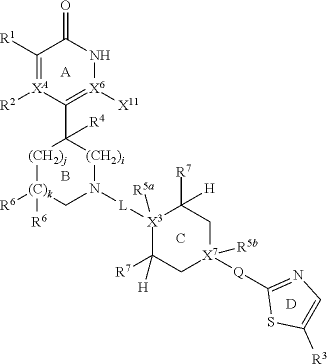
wherein:
R¹ is selected from —CH₃, —CH₂—CH₃, a cyclopropyl group, CF₃, Cl, and —CN;
i is 0, 1 or 2;
j is 0, 1, or 2;
k is 0 or 1;
with the proviso that i, j and k sum to 1, 2, 3, or 4;
R⁴:
i) is selected from H and methyl group, and each R⁶ is independently selected from H, F, a methyl group, a C1 to C4 alkoxy group, a C₁ to C₄ haloalkoxy group, —OPh; —OCH₂Ph; 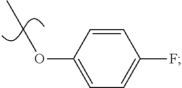
or
ii) is fused with one R⁶ to form a —CH₂— group bridging ring B, and the other R⁶ is H;
L is selected from: 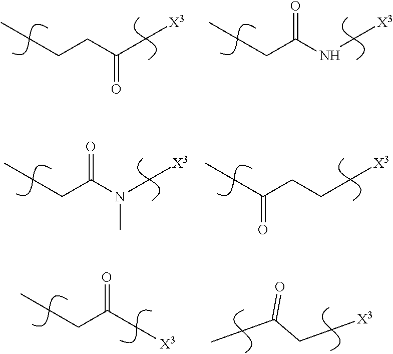
X³ and X⁷ are each selected from C and N, with the proviso that at least one of X³ and X⁷ is N;
when X³ is C, R^{5a} is H;
when X³ is N, R^{5a} is absent;
when X⁷ is C, R^{5b} is H and Q is a bond or a linker selected from —O—, —NH—, and —N(CH₃)—;
when X⁷ is N, R^{5b} is absent and Q is a bond;
each R⁷ is H, or the R⁷ groups are fused to form a —CH₂—CH₂— group bridging ring C; and
R³ is selected from —F; —Cl; —CF₃; —CHF₂; —OCF₂H; —OCF₃; —CN;

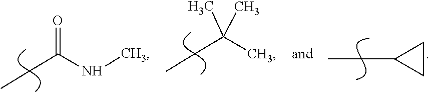

45. (canceled)

46. A compound according to claim 1, having the following formula: 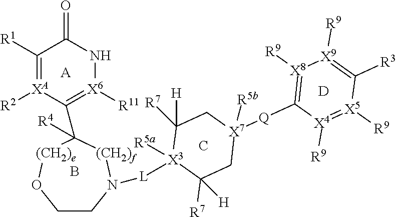
wherein:
R¹ is selected from —CH₃, —CH₂—CH₃, a cyclopropyl group, CF₃, Cl, and —CN;
R⁴ is H or a methyl group;
e is 1 and f is 0, or e is 0 and f is 1;
L is selected from: 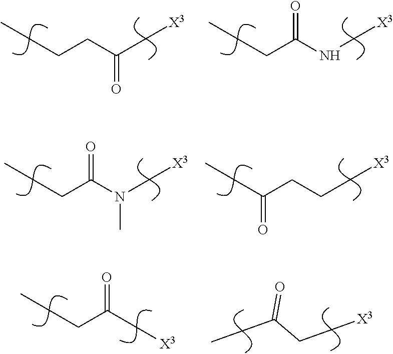
X³ and X⁷ are each selected from C and N, with the proviso that at least one of X³ and X⁷ is N;
when X³ is C, R^{5a} is H;
when X³ is N, R^{5a} is absent;
when X⁷ is C, R^{5b} is H, and Q is a bond or a linker selected from —O—, —NH—, and —N(CH₃)—;
when X⁷ is N, R^{5b} is absent, and Q is a bond;
each R⁷ is H, or the R⁷ groups are fused to form a —CH₂—CH₂— group bridging ring C;
X⁴, X⁵, X⁸, and X⁹ are each selected from C and N, with the proviso that no more than two of X⁴, X⁵, X⁸, and X⁹ are N;
when X⁴, X⁵, X⁸, or X⁹ is an N, the corresponding R⁹ is absent;
when X⁴, X⁵, X⁸, or X⁹ is a C, the corresponding R⁹ is selected from H, F, Cl, —CN, and —CF₃;
and
R³ is selected from —F; —Cl; —CF₃; —CHF₂; —OCF₂H; —OCF₃; —CN;

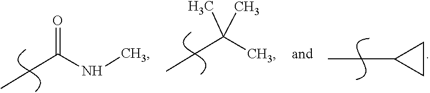

47. A compound according to claim 1, having the following formula: 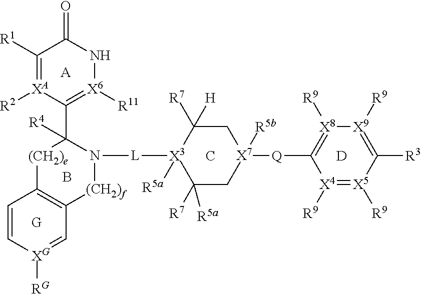
wherein:
R¹ is selected from —CH₃, —CH₂—CH₃, a cyclopropyl group, CF₃, Cl, and —CN
R⁴ is H or a methyl group;
e is 1 and f is 0, or e is 0 and f is 1;
X^{G} is selected from C and N;
when X^{G} is C, R^{G} is selected from H and F;
when X^{G} is N, R^{G} is absent;
L is selected from: 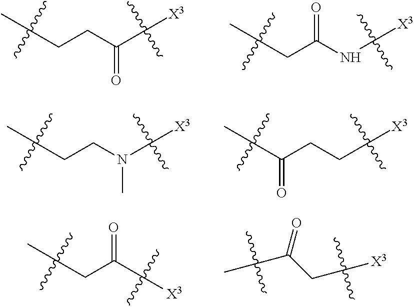
X³ and X⁷ are each selected from C and N, with the proviso that at least one of X³ and X⁷ is N;
when X³ is C, R^{5a} is H;
when X³ is N, R^{5a} is absent;
when X⁷ is C, R^{5b} is H, and Q is a bond or a linker selected from —O—, —NH— and —N(CH₃)—;
when X⁷ is N, R^{5b} is absent, and Q is a bond;
each R⁷ is H, or the R⁷ groups are fused to form a —CH₂—CH₂— group bridging ring C;
X⁴, X⁵, X⁸, and X⁹ are each selected from C and N, with the proviso that no more than two of X⁴, X⁵, X⁸, and X⁹ are N;
when X⁴, X⁵, X⁸, or X⁹ is an N, the corresponding R⁹ is absent;
when X⁴, X⁵, X⁸, or X⁹ is a C, the corresponding R⁹ is selected from H, F, Cl, —CN, and —CF₃;
and
R³ is selected from —F; —Cl; —CF₃; —CHF₂; —OCF₂H; —OCF₃; —CN;

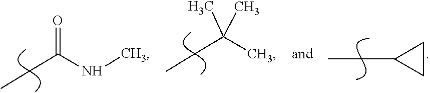

48. A compound according to claim 1, having the following formula: 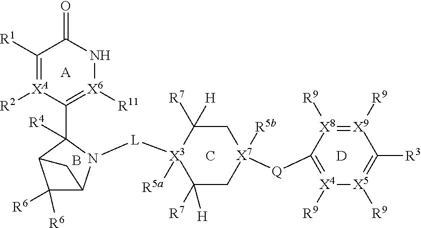
wherein:
R¹ is selected from —CH₃, —CH₂—CH₃, a cyclopropyl group, CF₃, Cl, and —CN
R⁴ is selected from H and methyl group;
each R⁶ is independently selected from H, F, a methyl group, a C1 to C4 alkoxy group, a C₁ to C₄ haloalkoxy group, —OPh, —OCH₂, and 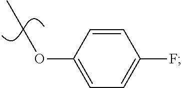
L is selected from: 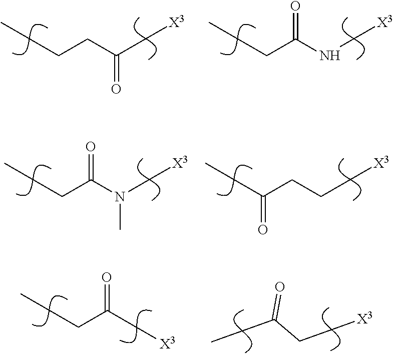
X³ and X⁷ are each selected from C and N, with the proviso that at least one of X³ and X⁷ is N;
when X³ is C, R^{5a} is H;
when X³ is N, R^{5a} is absent;
when X⁷ is C, R^{5b} is H and Q is a bond or a linker selected from —O—, —NH—, and —N(CH₃)—;
when X⁷ is N, R^{5b} is absent and Q is a bond;
each R⁷ is H, or the R⁷ groups are fused to form a —CH₂—CH₂— group bridging ring C;
X⁴, X⁵, X⁸, and X⁹ are each selected from C and N, with the proviso that no more than two of X⁴, X⁵, X⁸, and X⁹ are N;
when X⁴, X⁵, X⁸, or X⁹ is an N, the corresponding R⁹ is absent;
when X⁴, X⁵, X⁸, or X⁹ is a C, the corresponding R⁹ is selected from H, F, Cl, —CN, and —CF₃; and
R³ is selected from —F; —Cl; —CF₃; —CHF₂, —OCF₂H; —OCF₃; —CN;

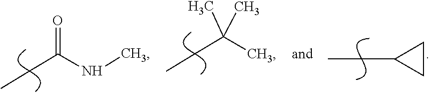

49-55. (canceled)

56. A compound according to claim 1, which compound comprises the following formula: 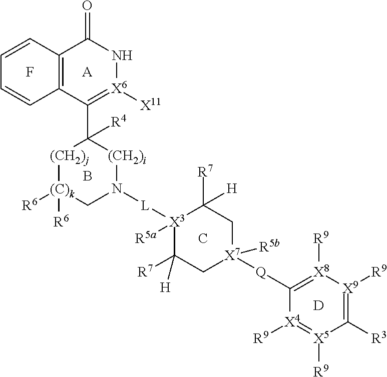
wherein:
is 0, 1 or2;
j is 0, 1, or 2;
k is 0 or 1;
with the proviso that i, j and k sum to 1, 2, 3, or 4;
R⁴:
i) is selected from H and methyl group, and each R⁶ is independently selected from H, F, a methyl group, a C1 to C4 alkoxy group; a C₁ to C₄ haloalkoxy group; —OPh; —OCH₂Ph; 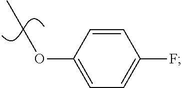
or
ii) is fused with one R⁶ to form a —CH₂— group bridging ring B, and the other R⁶ is H;
L is selected from: 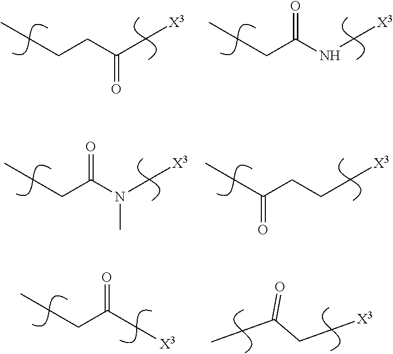
X³ and X⁷ are each selected from C and N, with the proviso that at least one of X³ and X⁷ is N;
when X³ is C, R^{5a} is H;
when X³ is N, R^{5a} is absent;
when X⁷ is C, R^{5b} is H, and Q is a bond or a linker selected from —O—, —NH— and —N(CH₃)—;
when X⁷ is N, R^{5b} is absent, and Q is a bond;
each R⁷ is H, or the R⁷ groups are fused to form a —CH₂—CH₂— group bridging ring C;
X⁴, X⁵, X⁸, and X⁹ are each selected from C and N, with the proviso that no more than two of X⁴, X⁵, X⁸, and X⁹ are N;
when X⁴, X⁵, X⁸, or X⁹ is an N, the corresponding R⁹ is absent;
when X⁴, X⁵, X⁸, or X⁹ is a C, the corresponding R⁹ is selected from H, F, Cl, —CN, and —CF₃; and
R³ is selected from —F; —Cl; —CF₃; —CHF₂, —OCF₂H; —OCF₃; —CN;

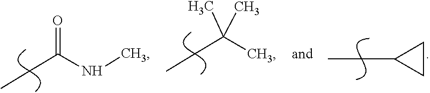

57-60. (canceled)

61. A compound according to claim 1, having a formula selected from:

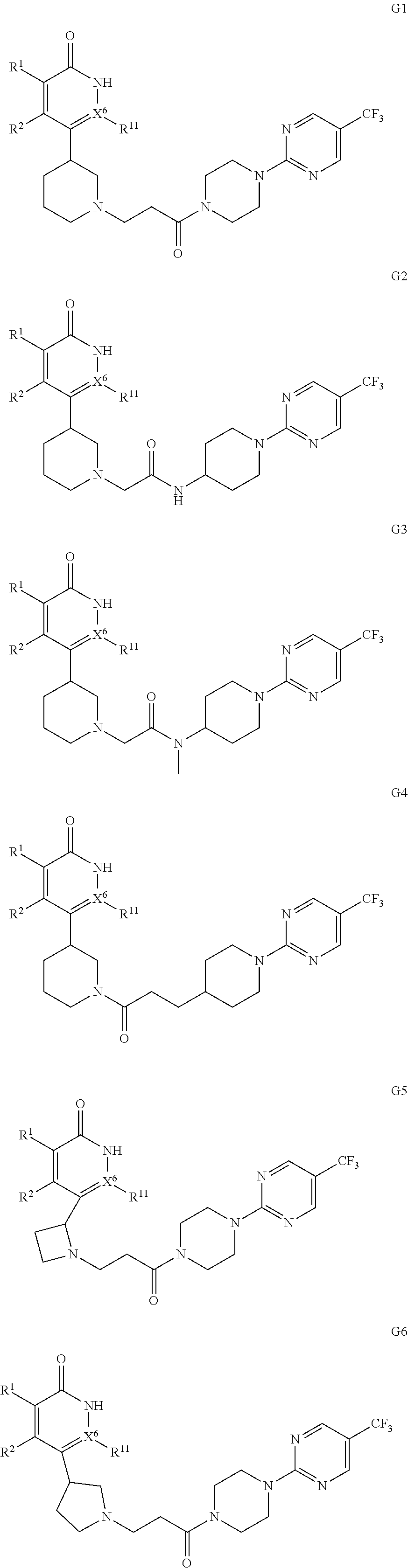

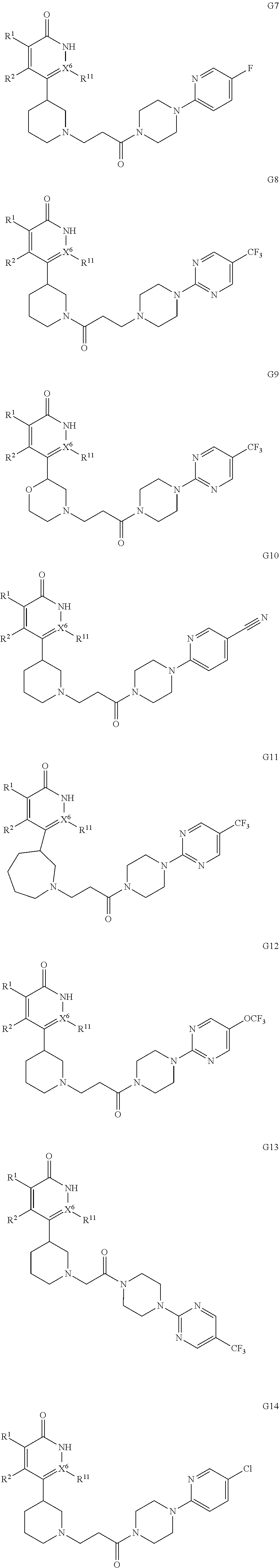

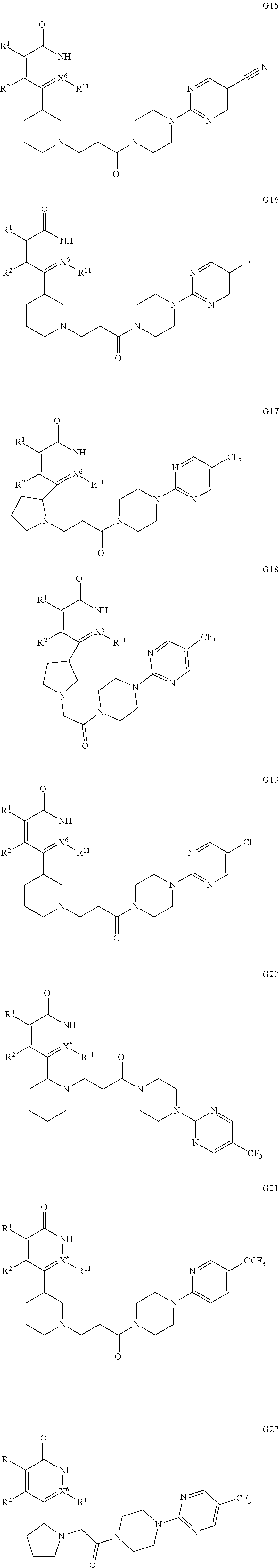

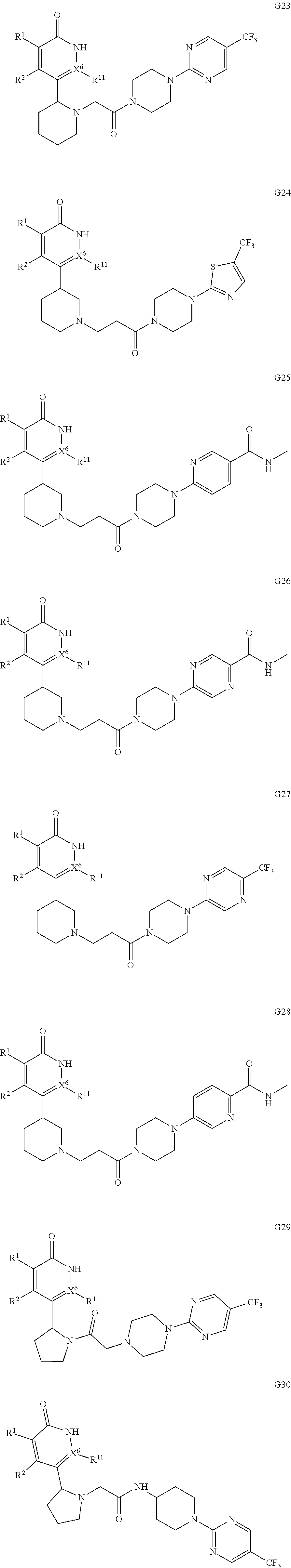

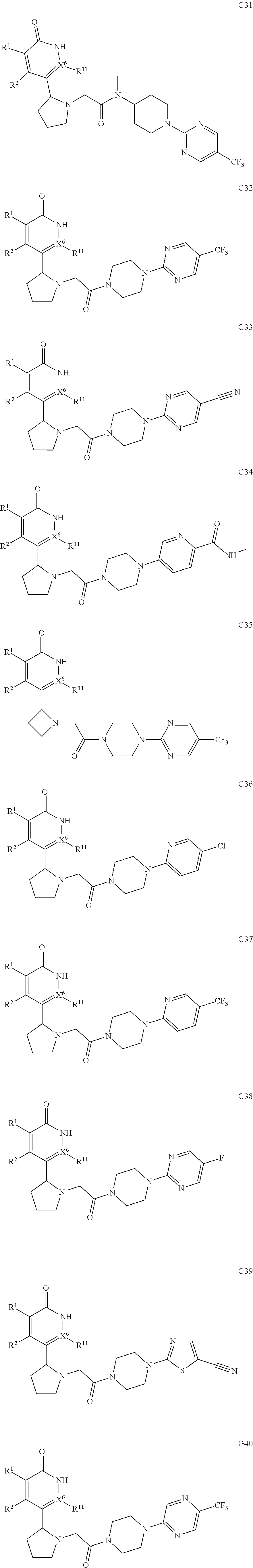

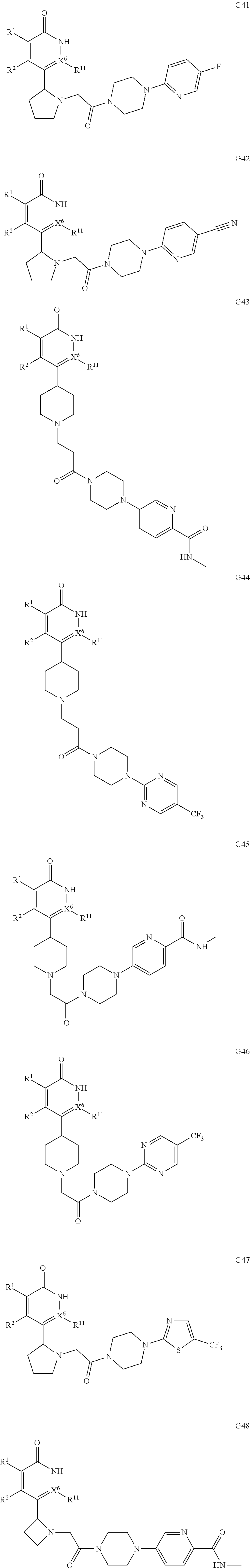

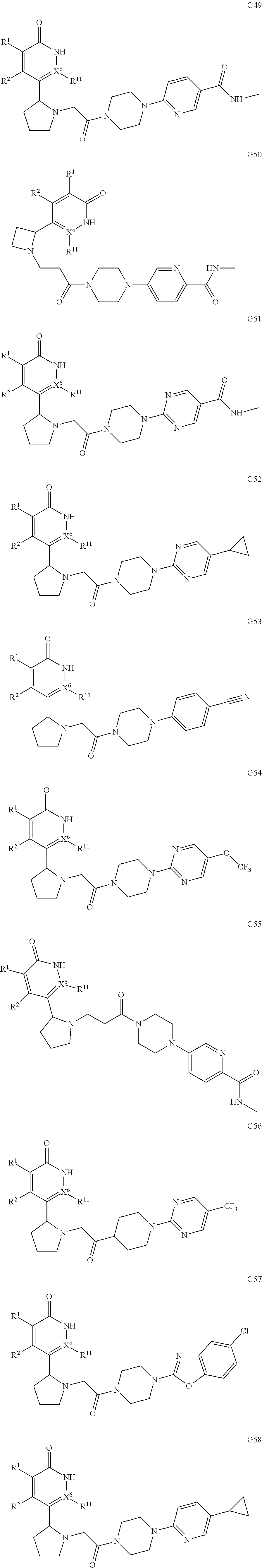

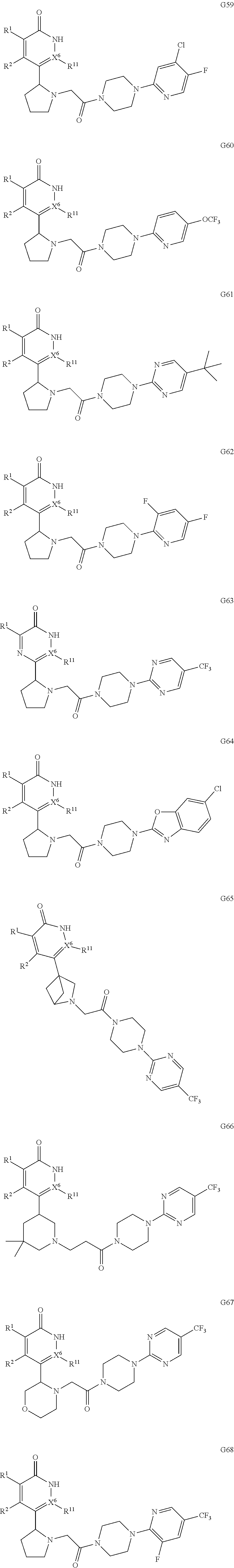

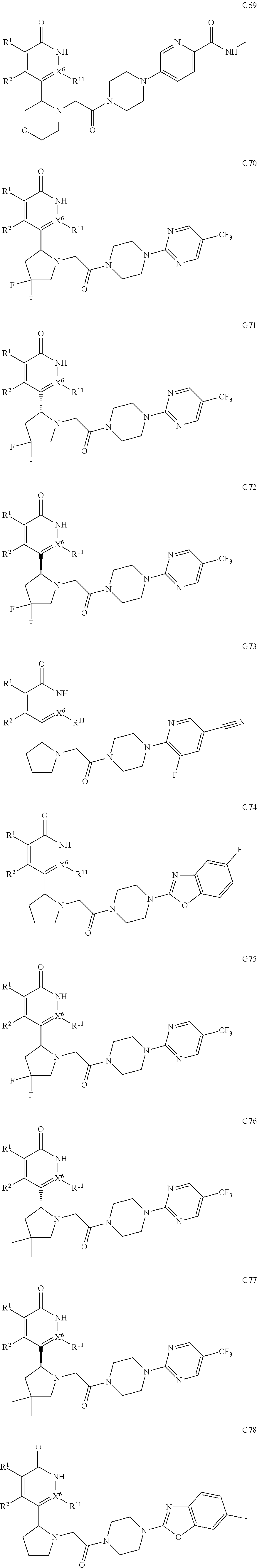

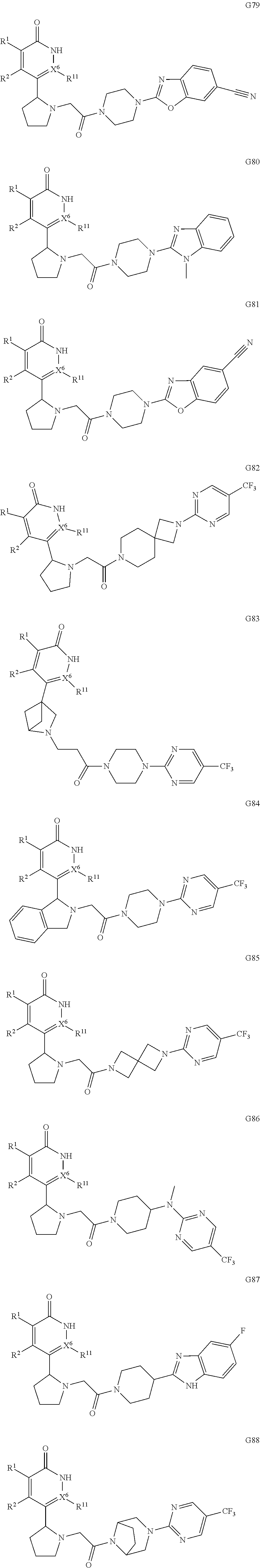

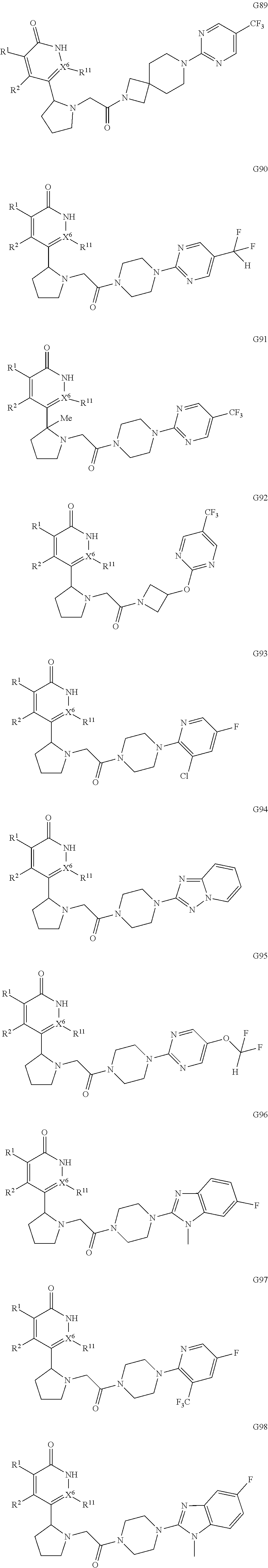

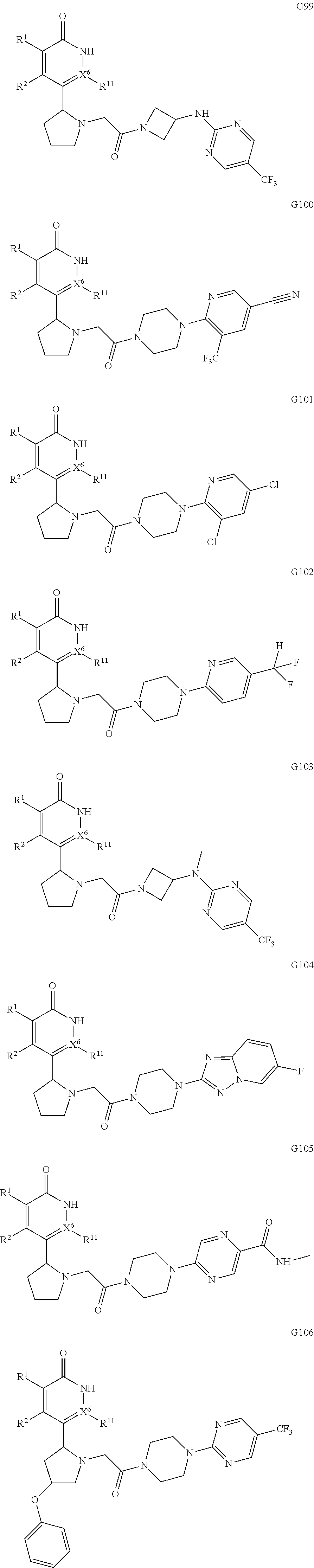

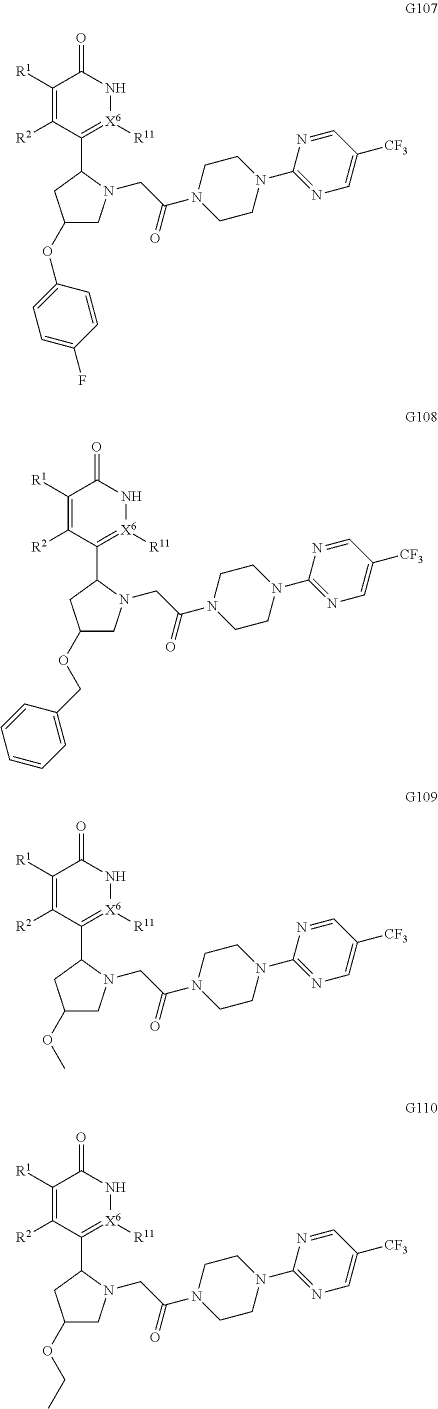

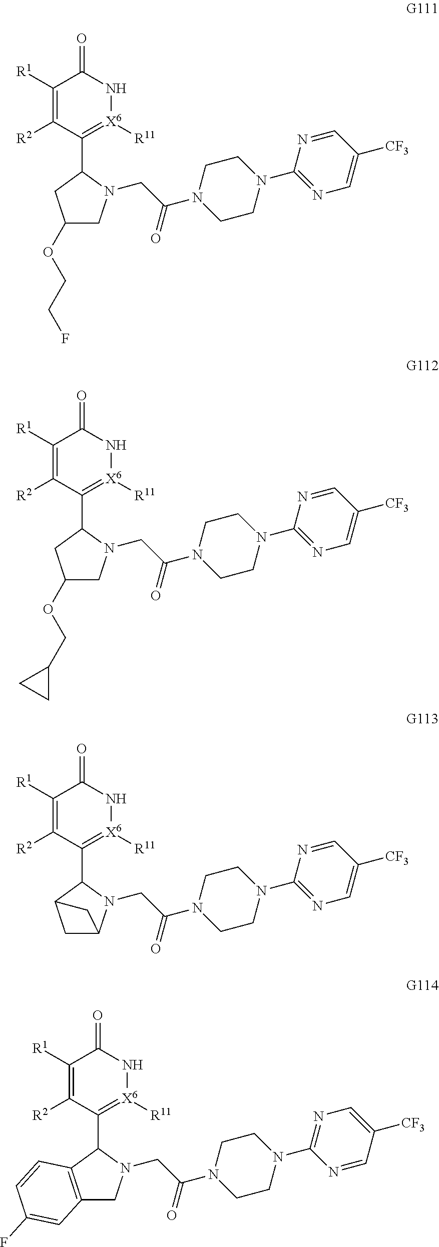

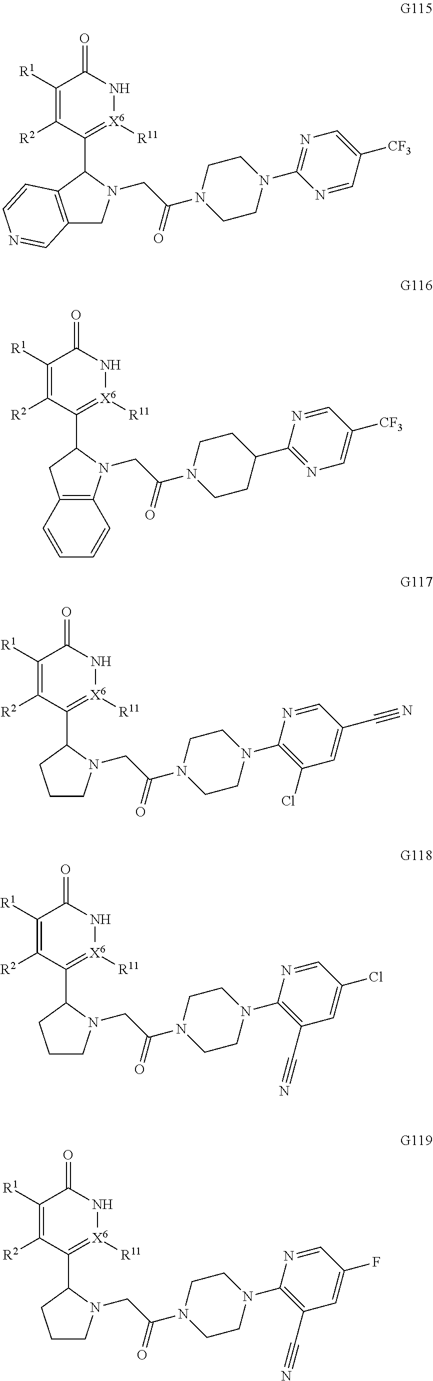

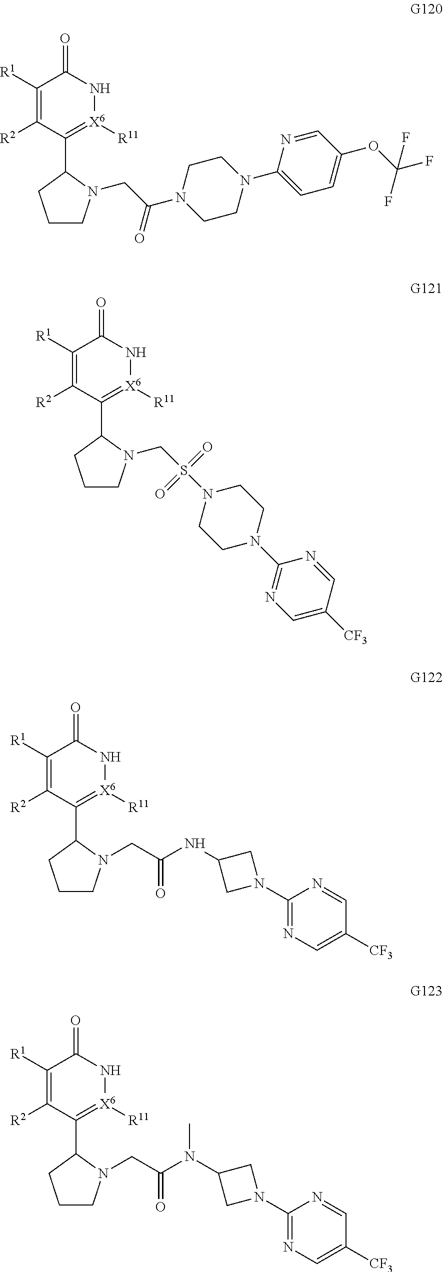

62. A compound according to claim 1, having a formula selected from:

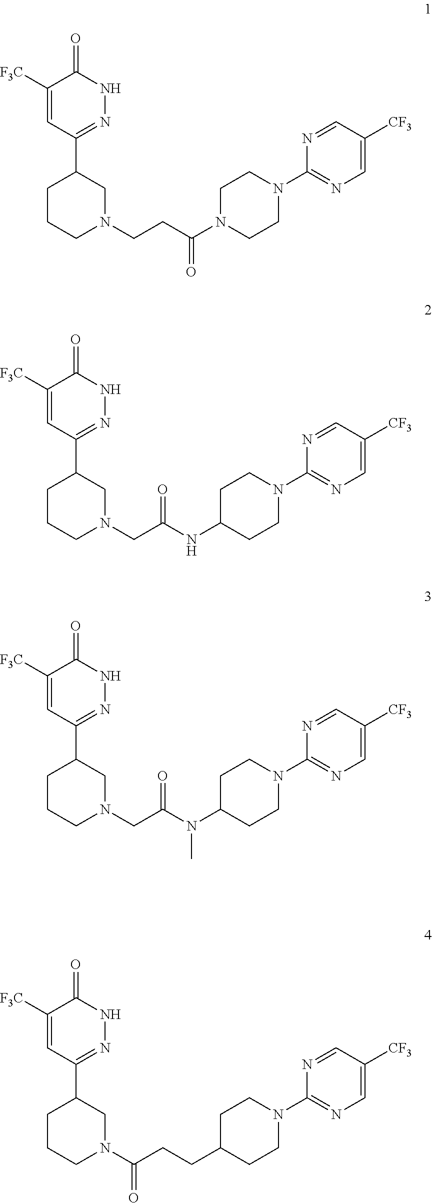 [SMILES: O=C(CCN1CCCC(c2cc(C(F)(F)F)c(=O)[nH]n2)C1)N1CCN(c2ncc(C(F)(F)F)cn2)CC1 ; O=C(CN1CCCC(c2cc(C(F)(F)F)c(=O)[nH]n2)C1)NC1CCN(c2ncc(C(F)(F)F)cn2)CC1 ; CN(C(=O)CN1CCCC(c2cc(C(F)(F)F)c(=O)[nH]n2)C1)C1CCN(c2ncc(C(F)(F)F)cn2)CC1 ; O=C(CCC1CCN(c2ncc(C(F)(F)F)cn2)CC1)N1CCCC(c2cc(C(F)(F)F)c(=O)[nH]n2)C1]

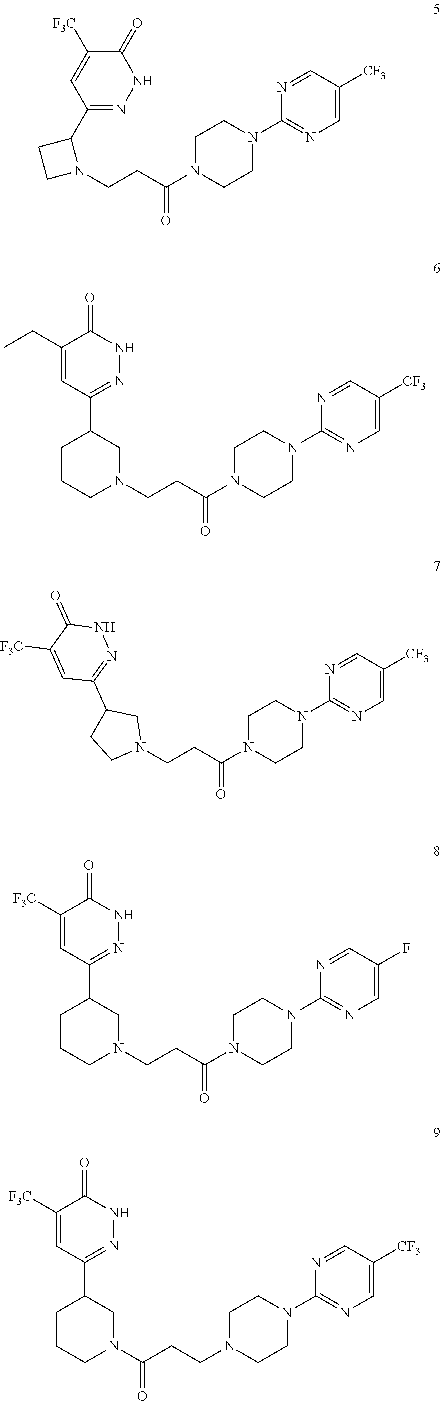 [SMILES: O=C(CCN1CCC1c1cc(C(F)(F)F)c(=O)[nH]n1)N1CCN(c2ncc(C(F)(F)F)cn2)CC1 ; CCc1cc(C2CCCN(CCC(=O)N3CCN(c4ncc(C(F)(F)F)cn4)CC3)C2)n[nH]c1=O ; O=C(CCN1CCC(c2cc(C(F)(F)F)c(=O)[nH]n2)C1)N1CCN(c2ncc(C(F)(F)F)cn2)CC1 ; O=C(CCN1CCCC(c2cc(C(F)(F)F)c(=O)[nH]n2)C1)N1CCN(c2ncc(F)cn2)CC1 ; O=C(CCN1CCN(c2ncc(C(F)(F)F)cn2)CC1)N1CCCC(c2cc(C(F)(F)F)c(=O)[nH]n2)C1]

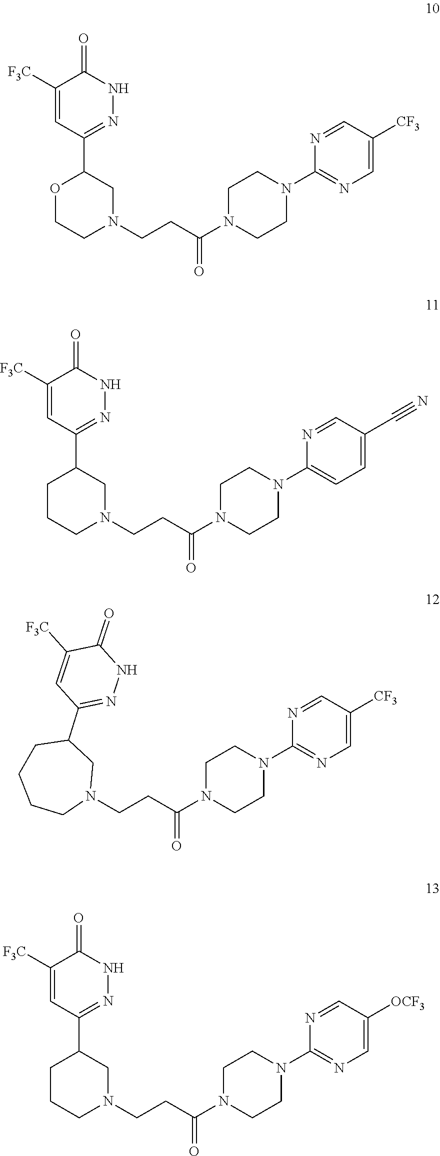 [SMILES: O=C(CCN1CCOC(c2cc(C(F)(F)F)c(=O)[nH]n2)C1)N1CCN(c2ncc(C(F)(F)F)cn2)CC1 ; N#Cc1ccc(N2CCN(C(=O)CCN3CCCC(c4cc(C(F)(F)F)c(=O)[nH]n4)C3)CC2)nc1 ; O=C(CCN1CCCCC(c2cc(C(F)(F)F)c(=O)[nH]n2)C1)N1CCN(c2ncc(C(F)(F)F)cn2)CC1 ; O=C(CCN1CCCC(c2cc(C(F)(F)F)c(=O)[nH]n2)C1)N1CCN(c2ncc(OC(F)(F)F)cn2)CC1]

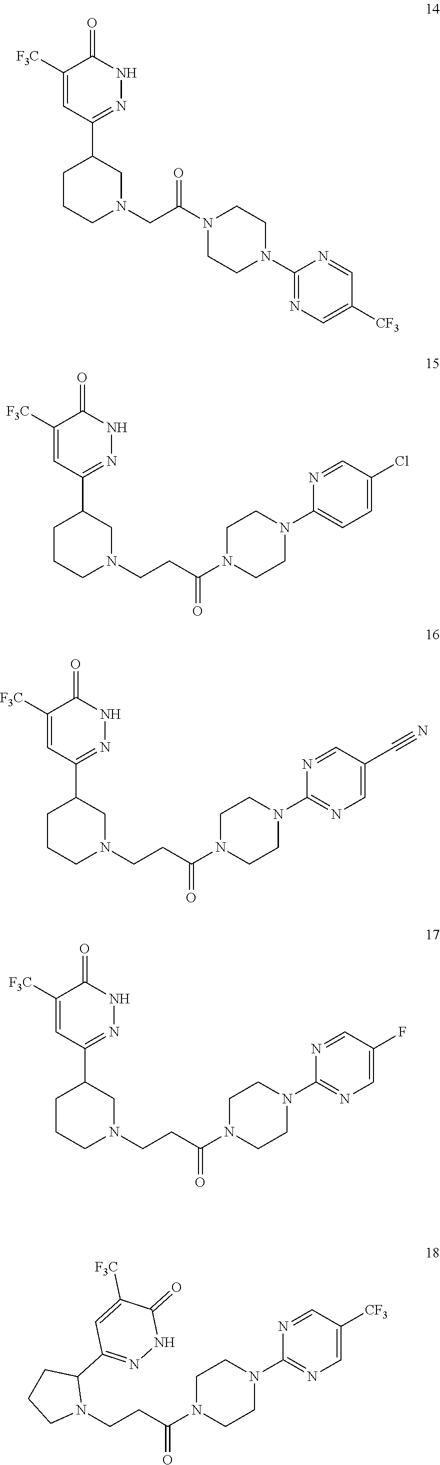 [SMILES: O=C(CN1CCCC(c2cc(C(F)(F)F)c(=O)[nH]n2)C1)N1CCN(c2ncc(C(F)(F)F)cn2)CC1 ; O=C(CCN1CCCC(c2cc(C(F)(F)F)c(=O)[nH]n2)C1)N1CCN(c2ccc(Cl)cn2)CC1 ; N#Cc1cnc(N2CCN(C(=O)CCN3CCCC(c4cc(C(F)(F)F)c(=O)[nH]n4)C3)CC2)nc1 ; O=C(CCN1CCCC(c2cc(C(F)(F)F)c(=O)[nH]n2)C1)N1CCN(c2ncc(F)cn2)CC1 ; O=C(CCN1CCCC1c1cc(C(F)(F)F)c(=O)[nH]n1)N1CCN(c2ncc(C(F)(F)F)cn2)CC1]

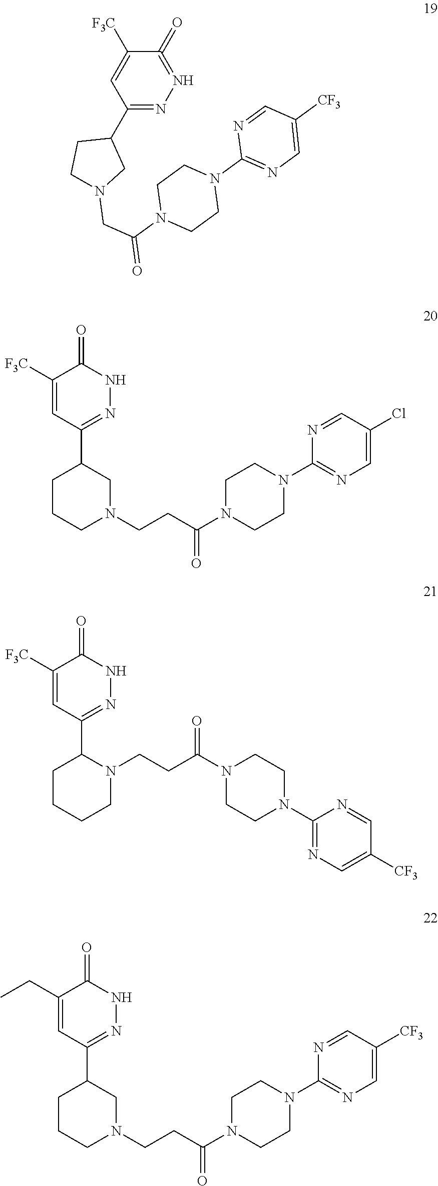

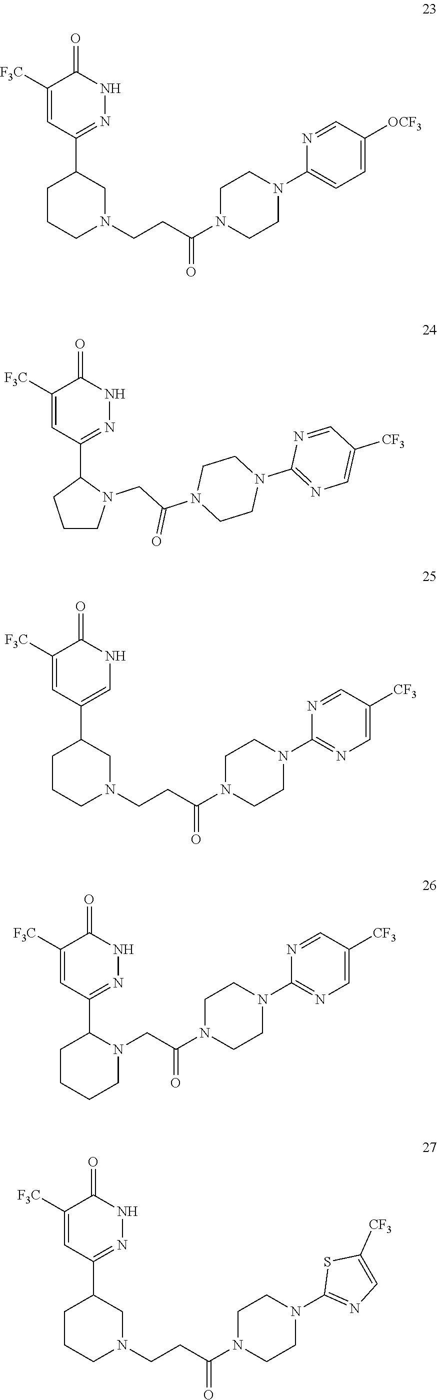 [SMILES: O=C(CCN1CCCC(c2cc(C(F)(F)F)c(=O)[nH]n2)C1)N1CCN(c2ccc(OC(F)(F)F)cn2)CC1 ; O=C(CN1CCCC1c1cc(C(F)(F)F)c(=O)[nH]n1)N1CCN(c2ncc(C(F)(F)F)cn2)CC1 ; O=C(CCN1CCCC(c2c[nH]c(=O)c(C(F)(F)F)c2)C1)N1CCN(c2ncc(C(F)(F)F)cn2)CC1 ; O=C(CN1CCCCC1c1cc(C(F)(F)F)c(=O)[nH]n1)N1CCN(c2ncc(C(F)(F)F)cn2)CC1 ; O=C(CCN1CCCC(c2cc(C(F)(F)F)c(=O)[nH]n2)C1)N1CCN(c2ncc(C(F)(F)F)s2)CC1]

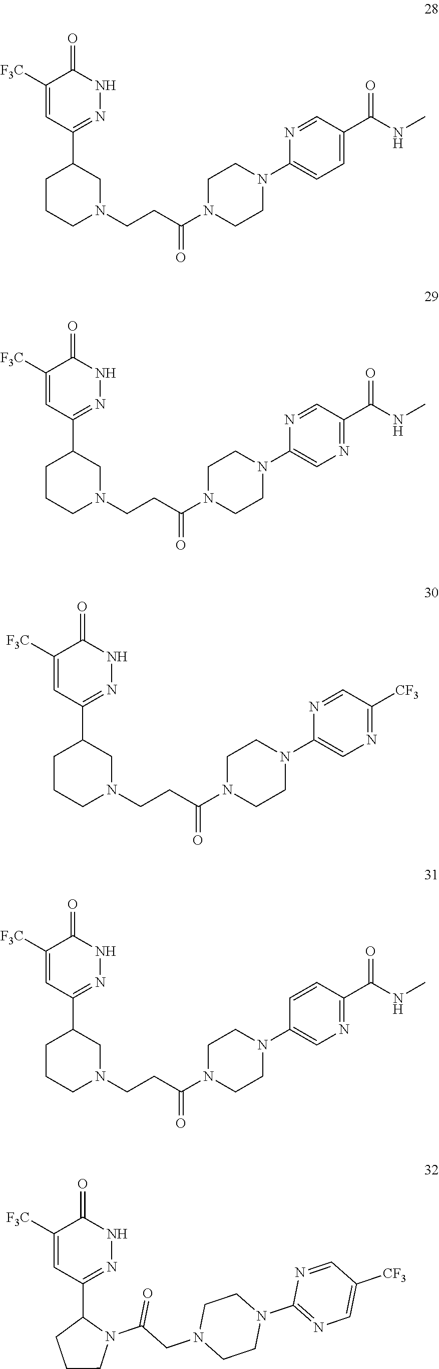 [SMILES: CNC(=O)c1ccc(N2CCN(C(=O)CCN3CCCC(c4cc(C(F)(F)F)c(=O)[nH]n4)C3)CC2)nc1 ; CNC(=O)c1cnc(N2CCN(C(=O)CCN3CCCC(c4cc(C(F)(F)F)c(=O)[nH]n4)C3)CC2)cn1 ; O=C(CCN1CCCC(c2cc(C(F)(F)F)c(=O)[nH]n2)C1)N1CCN(c2cnc(C(F)(F)F)cn2)CC1 ; CNC(=O)c1ccc(N2CCN(C(=O)CCN3CCCC(c4cc(C(F)(F)F)c(=O)[nH]n4)C3)CC2)cn1 ; O=C(CN1CCN(c2ncc(C(F)(F)F)cn2)CC1)N1CCCC1c1cc(C(F)(F)F)c(=O)[nH]n1]

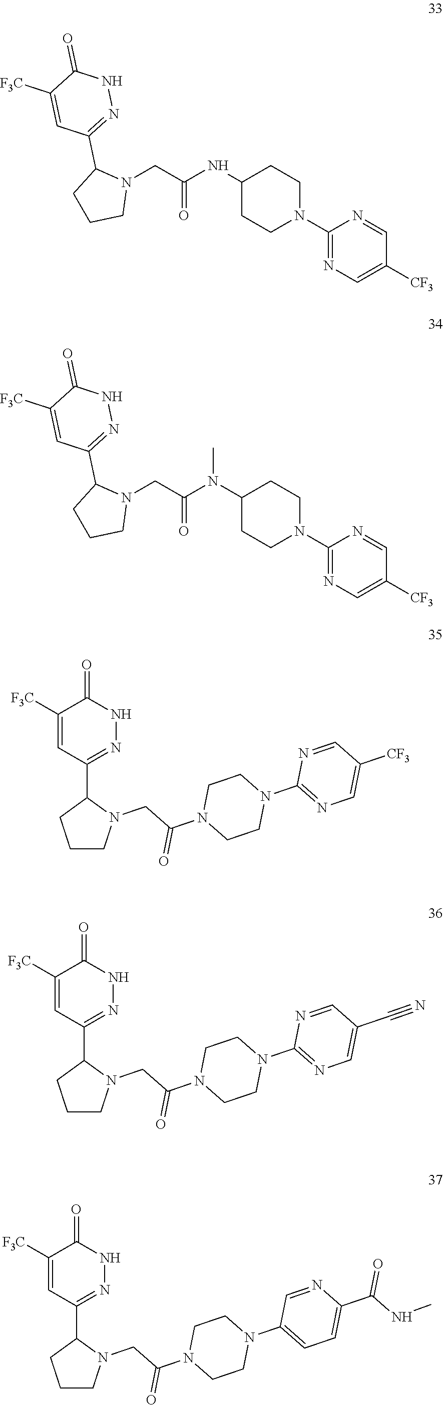 [SMILES: O=C(CN1CCCC1c1cc(C(F)(F)F)c(=O)[nH]n1)NC1CCN(c2ncc(C(F)(F)F)cn2)CC1 ; CN(C(=O)CN1CCCC1c1cc(C(F)(F)F)c(=O)[nH]n1)C1CCN(c2ncc(C(F)(F)F)cn2)CC1 ; O=C(CN1CCCC1c1cc(C(F)(F)F)c(=O)[nH]n1)N1CCN(c2ncc(C(F)(F)F)cn2)CC1 ; N#Cc1cnc(N2CCN(C(=O)CN3CCCC3c3cc(C(F)(F)F)c(=O)[nH]n3)CC2)nc1 ; CNC(=O)c1ccc(N2CCN(C(=O)CN3CCCC3c3cc(C(F)(F)F)c(=O)[nH]n3)CC2)cn1]

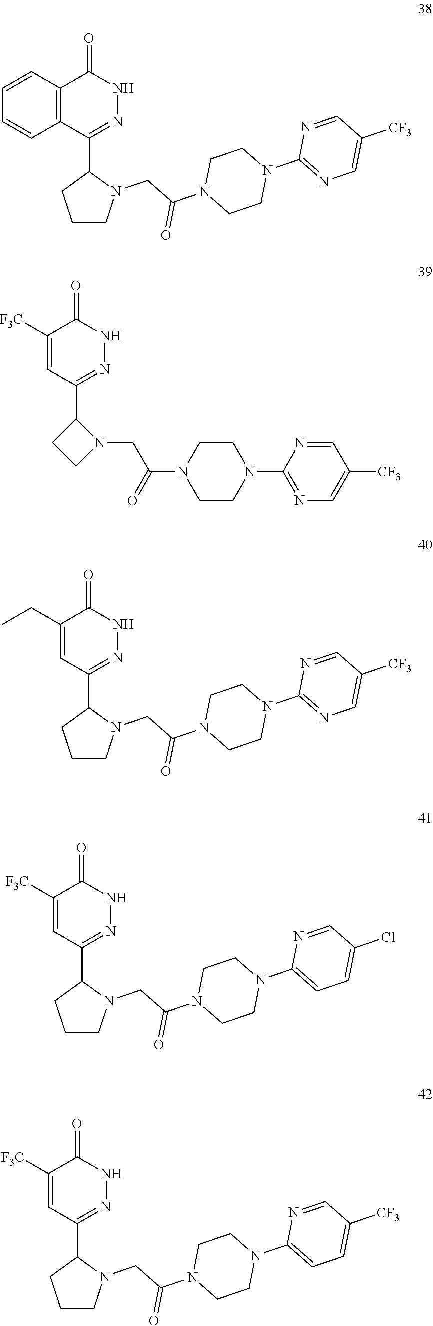 [SMILES: O=C(CN1CCCC1c1n[nH]c(=O)c2ccccc12)N1CCN(c2ncc(C(F)(F)F)cn2)CC1 ; O=C(CN1CCC1c1cc(C(F)(F)F)c(=O)[nH]n1)N1CCN(c2ncc(C(F)(F)F)cn2)CC1 ; CCc1cc(C2CCCN2CC(=O)N2CCN(c3ncc(C(F)(F)F)cn3)CC2)n[nH]c1=O ; O=C(CN1CCCC1c1cc(C(F)(F)F)c(=O)[nH]n1)N1CCN(c2ccc(Cl)cn2)CC1 ; O=C(CN1CCCC1c1cc(C(F)(F)F)c(=O)[nH]n1)N1CCN(c2ccc(C(F)(F)F)cn2)CC1]

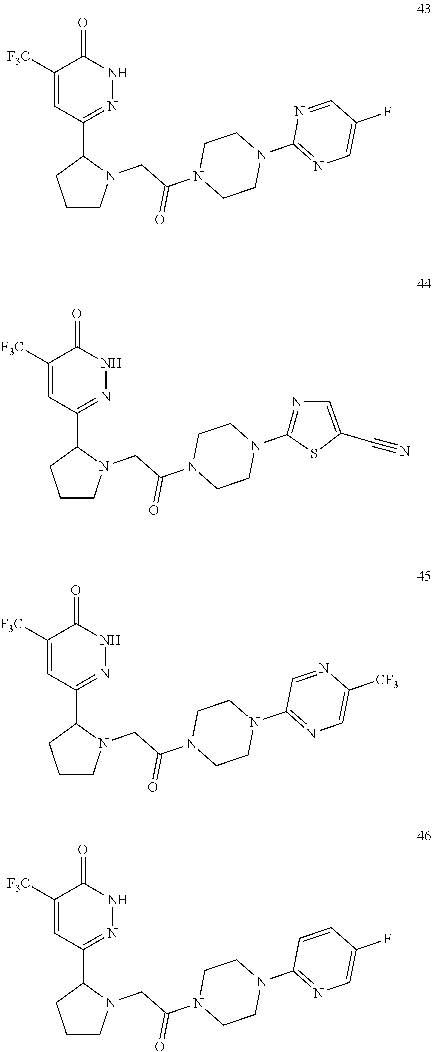 [SMILES: O=C(CN1CCCC1c1cc(C(F)(F)F)c(=O)[nH]n1)N1CCN(c2ncc(F)cn2)CC1 ; N#Cc1cnc(N2CCN(C(=O)CN3CCCC3c3cc(C(F)(F)F)c(=O)[nH]n3)CC2)s1 ; O=C(CN1CCCC1c1cc(C(F)(F)F)c(=O)[nH]n1)N1CCN(c2cnc(C(F)(F)F)cn2)CC1 ; O=C(CN1CCCC1c1cc(C(F)(F)F)c(=O)[nH]n1)N1CCN(c2ccc(F)cn2)CC1]

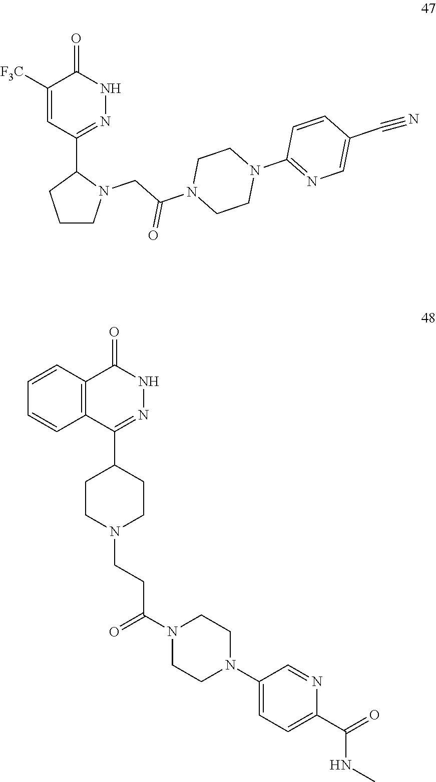 [SMILES: N#Cc1ccc(N2CCN(C(=O)CN3CCCC3c3cc(C(F)(F)F)c(=O)[nH]n3)CC2)nc1 ; CNC(=O)c1ccc(N2CCN(C(=O)CCN3CCC(c4n[nH]c(=O)c5ccccc45)CC3)CC2)cn1]

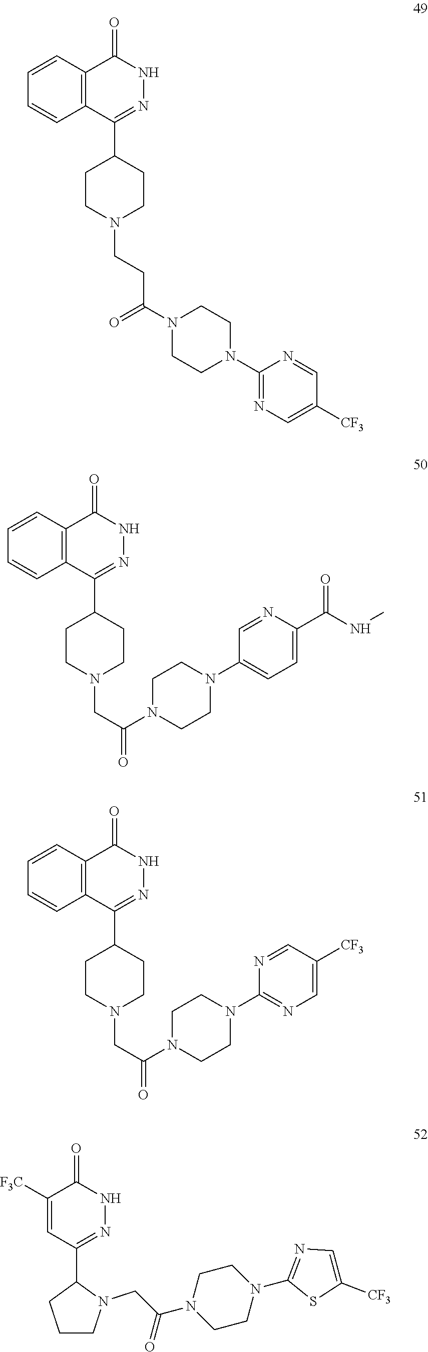 [SMILES: O=C(CCN1CCC(c2n[nH]c(=O)c3ccccc23)CC1)N1CCN(c2ncc(C(F)(F)F)cn2)CC1 ; CNC(=O)c1ccc(N2CCN(C(=O)CN3CCC(c4n[nH]c(=O)c5ccccc45)CC3)CC2)cn1 ; O=C(CN1CCC(c2n[nH]c(=O)c3ccccc23)CC1)N1CCN(c2ncc(C(F)(F)F)cn2)CC1 ; O=C(CN1CCCC1c1cc(C(F)(F)F)c(=O)[nH]n1)N1CCN(c2ncc(C(F)(F)F)s2)CC1]

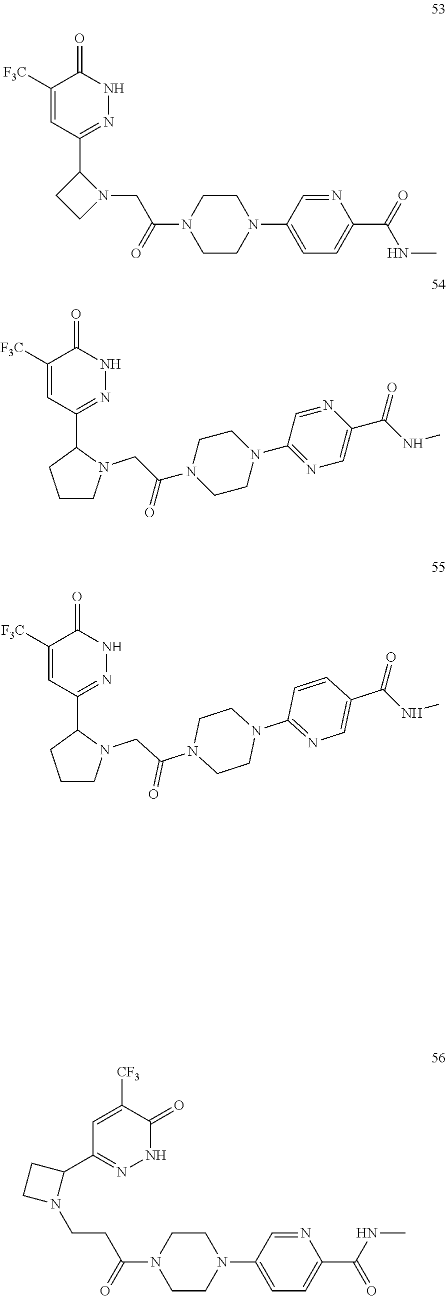 [SMILES: CNC(=O)c1ccc(N2CCN(C(=O)CN3CCC3c3cc(C(F)(F)F)c(=O)[nH]n3)CC2)cn1 ; CNC(=O)c1cnc(N2CCN(C(=O)CN3CCCC3c3cc(C(F)(F)F)c(=O)[nH]n3)CC2)cn1 ; CNC(=O)c1ccc(N2CCN(C(=O)CN3CCCC3c3cc(C(F)(F)F)c(=O)[nH]n3)CC2)nc1 ; CNC(=O)c1ccc(N2CCN(C(=O)CCN3CCC3c3cc(C(F)(F)F)c(=O)[nH]n3)CC2)cn1]

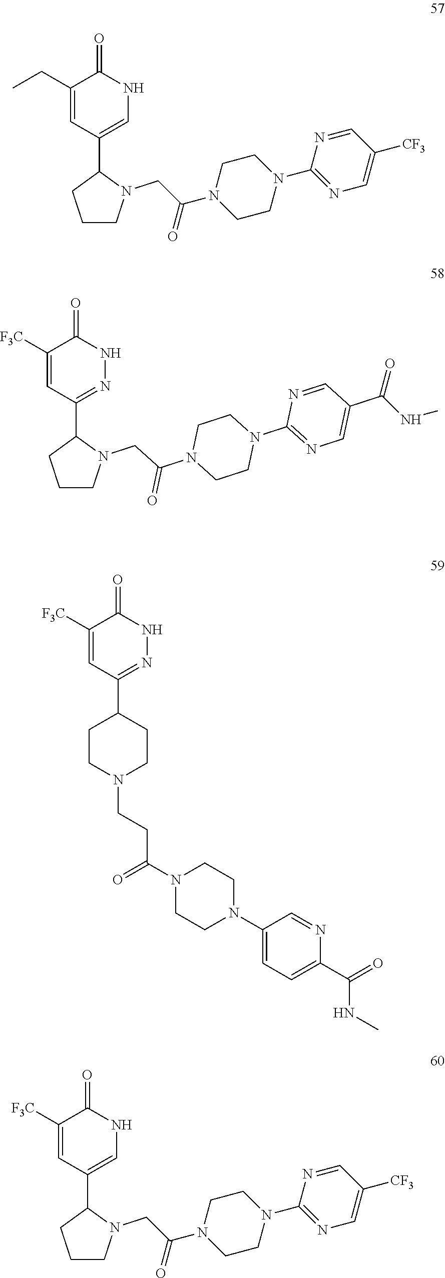 [SMILES: CCc1cc(C2CCCN2CC(=O)N2CCN(c3ncc(C(F)(F)F)cn3)CC2)c[nH]c1=O ; CNC(=O)c1cnc(N2CCN(C(=O)CN3CCCC3c3cc(C(F)(F)F)c(=O)[nH]n3)CC2)nc1 ; CNC(=O)c1ccc(N2CCN(C(=O)CCN3CCC(c4cc(C(F)(F)F)c(=O)[nH]n4)CC3)CC2)cn1 ; O=C(CN1CCCC1c1c[nH]c(=O)c(C(F)(F)F)c1)N1CCN(c2ncc(C(F)(F)F)cn2)CC1]

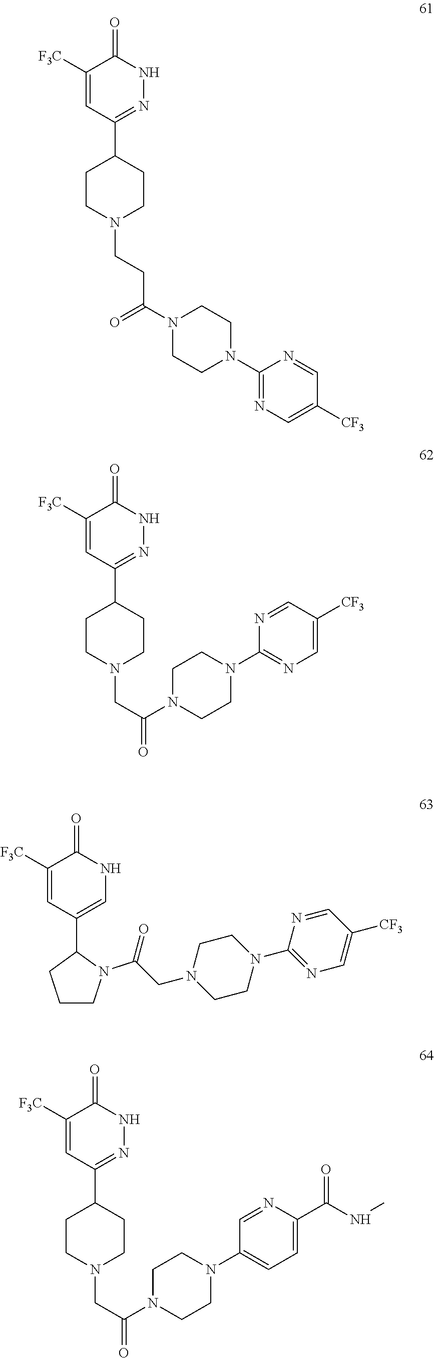 [SMILES: O=C(CCN1CCC(c2cc(C(F)(F)F)c(=O)[nH]n2)CC1)N1CCN(c2ncc(C(F)(F)F)cn2)CC1 ; O=C(CN1CCC(c2cc(C(F)(F)F)c(=O)[nH]n2)CC1)N1CCN(c2ncc(C(F)(F)F)cn2)CC1 ; O=C(CN1CCN(c2ncc(C(F)(F)F)cn2)CC1)N1CCCC1c1c[nH]c(=O)c(C(F)(F)F)c1 ; CNC(=O)c1ccc(N2CCN(C(=O)CN3CCC(c4cc(C(F)(F)F)c(=O)[nH]n4)CC3)CC2)cn1]

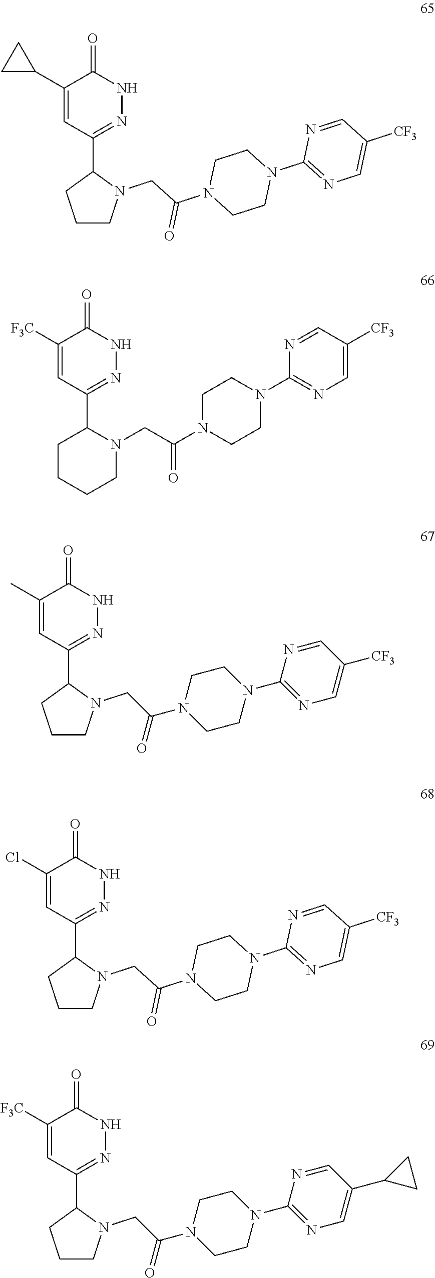 [SMILES: O=C(CN1CCCC1c1cc(C2CC2)c(=O)[nH]n1)N1CCN(c2ncc(C(F)(F)F)cn2)CC1 ; O=C(CN1CCCCC1c1cc(C(F)(F)F)c(=O)[nH]n1)N1CCN(c2ncc(C(F)(F)F)cn2)CC1 ; Cc1cc(C2CCCN2CC(=O)N2CCN(c3ncc(C(F)(F)F)cn3)CC2)n[nH]c1=O ; O=C(CN1CCCC1c1cc(Cl)c(=O)[nH]n1)N1CCN(c2ncc(C(F)(F)F)cn2)CC1 ; O=C(CN1CCCC1c1cc(C(F)(F)F)c(=O)[nH]n1)N1CCN(c2ncc(C3CC3)cn2)CC1]

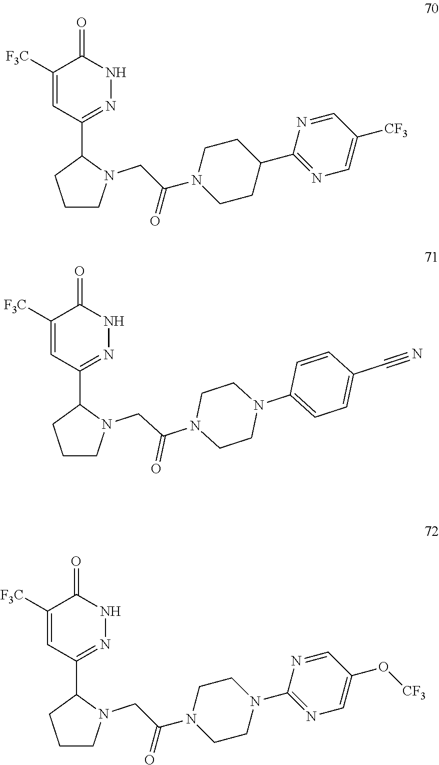 [SMILES: O=C(CN1CCCC1c1cc(C(F)(F)F)c(=O)[nH]n1)N1CCC(c2ncc(C(F)(F)F)cn2)CC1 ; N#Cc1ccc(N2CCN(C(=O)CN3CCCC3c3cc(C(F)(F)F)c(=O)[nH]n3)CC2)cc1 ; O=C(CN1CCCC1c1cc(C(F)(F)F)c(=O)[nH]n1)N1CCN(c2ncc(OC(F)(F)F)cn2)CC1]

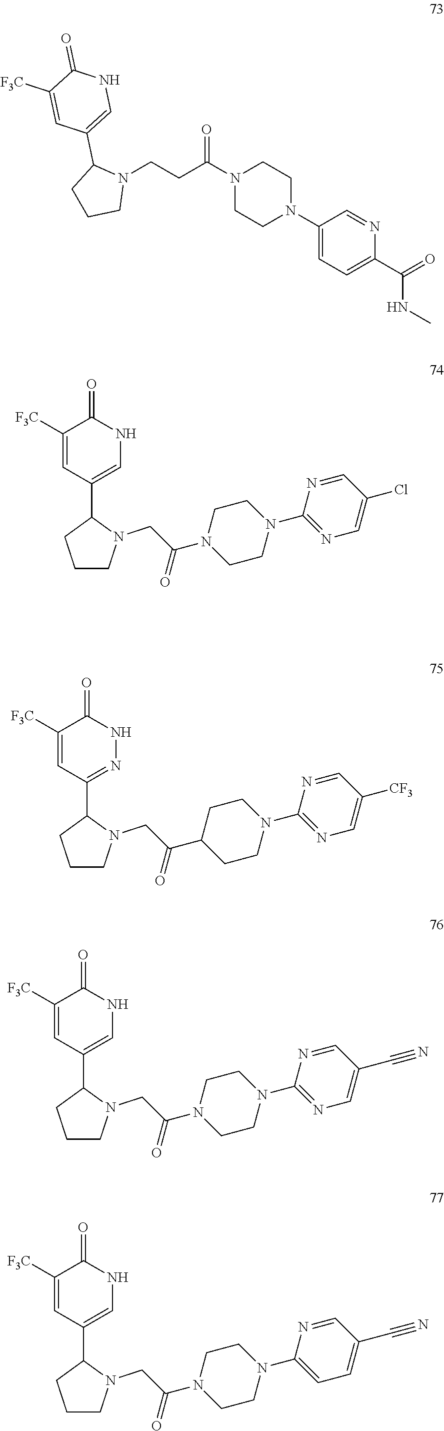 [SMILES: CNC(=O)c1ccc(N2CCN(C(=O)CCN3CCCC3c3c[nH]c(=O)c(C(F)(F)F)c3)CC2)cn1 ; O=C(CN1CCCC1c1c[nH]c(=O)c(C(F)(F)F)c1)N1CCN(c2ncc(Cl)cn2)CC1 ; O=C(CN1CCCC1c1cc(C(F)(F)F)c(=O)[nH]n1)C1CCN(c2ncc(C(F)(F)F)cn2)CC1 ; N#Cc1cnc(N2CCN(C(=O)CN3CCCC3c3c[nH]c(=O)c(C(F)(F)F)c3)CC2)nc1 ; N#Cc1ccc(N2CCN(C(=O)CN3CCCC3c3c[nH]c(=O)c(C(F)(F)F)c3)CC2)nc1]

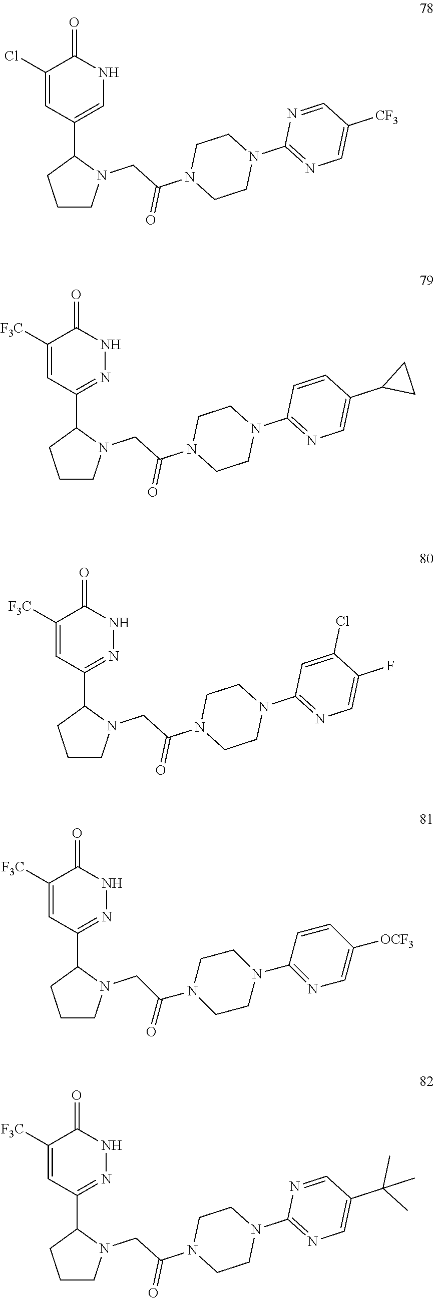 [SMILES: O=C(CN1CCCC1c1c[nH]c(=O)c(Cl)c1)N1CCN(c2ncc(C(F)(F)F)cn2)CC1 ; O=C(CN1CCCC1c1cc(C(F)(F)F)c(=O)[nH]n1)N1CCN(c2ccc(C3CC3)cn2)CC1 ; O=C(CN1CCCC1c1cc(C(F)(F)F)c(=O)[nH]n1)N1CCN(c2cc(Cl)c(F)cn2)CC1 ; O=C(CN1CCCC1c1cc(C(F)(F)F)c(=O)[nH]n1)N1CCN(c2ccc(OC(F)(F)F)cn2)CC1 ; CC(C)(C)c1cnc(N2CCN(C(=O)CN3CCCC3c3cc(C(F)(F)F)c(=O)[nH]n3)CC2)nc1]

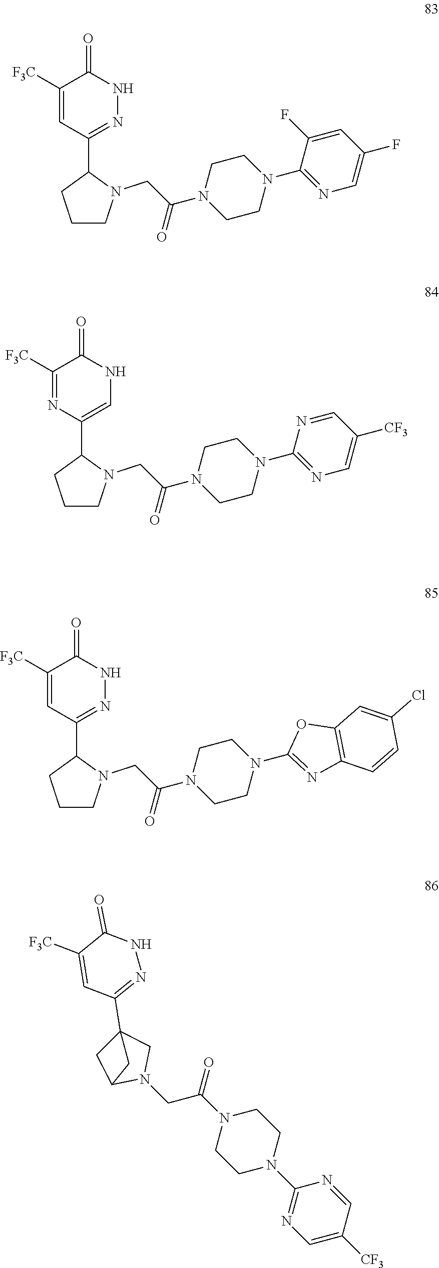 [SMILES: O=C(CN1CCCC1c1cc(C(F)(F)F)c(=O)[nH]n1)N1CCN(c2ncc(F)cc2F)CC1 ; O=C(CN1CCCC1c1c[nH]c(=O)c(C(F)(F)F)n1)N1CCN(c2ncc(C(F)(F)F)cn2)CC1 ; O=C(CN1CCCC1c1cc(C(F)(F)F)c(=O)[nH]n1)N1CCN(c2nc3ccc(Cl)cc3o2)CC1 ; O=C(CN1CC2(c3cc(C(F)(F)F)c(=O)[nH]n3)CC1C2)N1CCN(c2ncc(C(F)(F)F)cn2)CC1]

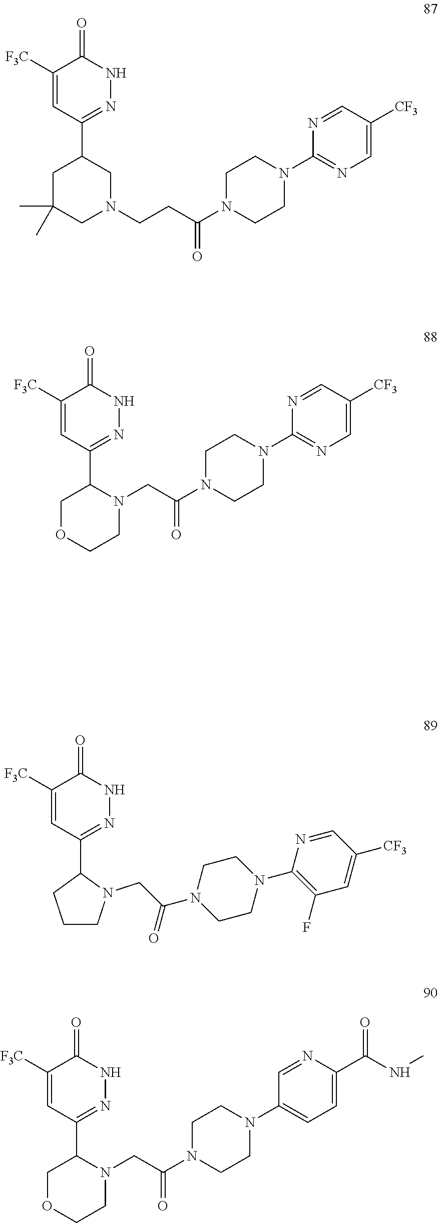 [SMILES: CC1(C)CC(c2cc(C(F)(F)F)c(=O)[nH]n2)CN(CCC(=O)N2CCN(c3ncc(C(F)(F)F)cn3)CC2)C1 ; O=C(CN1CCOCC1c1cc(C(F)(F)F)c(=O)[nH]n1)N1CCN(c2ncc(C(F)(F)F)cn2)CC1 ; O=C(CN1CCCC1c1cc(C(F)(F)F)c(=O)[nH]n1)N1CCN(c2ncc(C(F)(F)F)cc2F)CC1 ; CNC(=O)c1ccc(N2CCN(C(=O)CN3CCOCC3c3cc(C(F)(F)F)c(=O)[nH]n3)CC2)cn1]

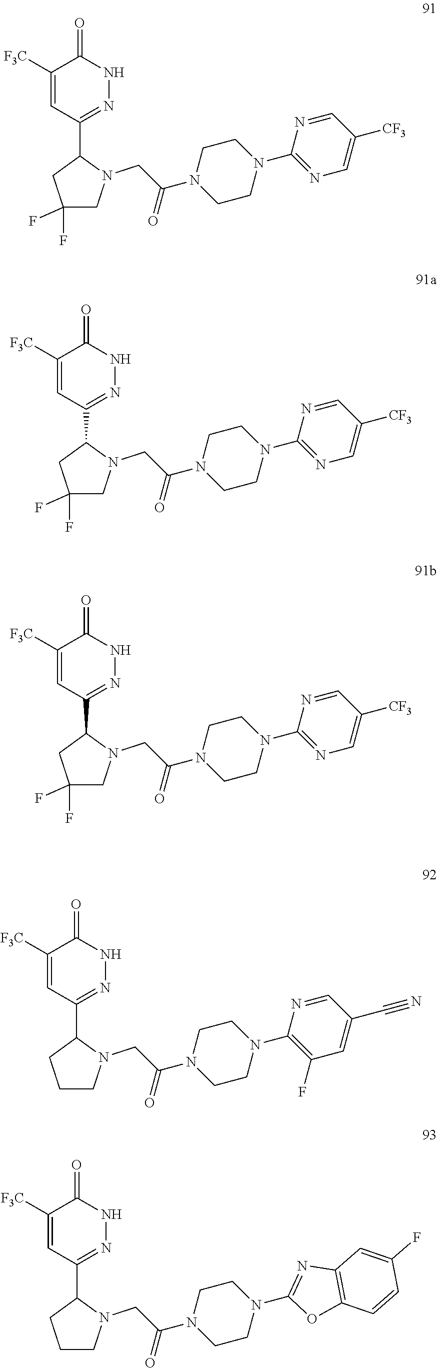 [SMILES: O=C(CN1CC(F)(F)CC1c1cc(C(F)(F)F)c(=O)[nH]n1)N1CCN(c2ncc(C(F)(F)F)cn2)CC1 ; O=C(CN1CC(F)(F)C[C@@H]1c1cc(C(F)(F)F)c(=O)[nH]n1)N1CCN(c2ncc(C(F)(F)F)cn2)CC1 ; O=C(CN1CC(F)(F)C[C@H]1c1cc(C(F)(F)F)c(=O)[nH]n1)N1CCN(c2ncc(C(F)(F)F)cn2)CC1 ; N#Cc1cnc(N2CCN(C(=O)CN3CCCC3c3cc(C(F)(F)F)c(=O)[nH]n3)CC2)c(F)c1 ; O=C(CN1CCCC1c1cc(C(F)(F)F)c(=O)[nH]n1)N1CCN(c2nc3cc(F)ccc3o2)CC1]

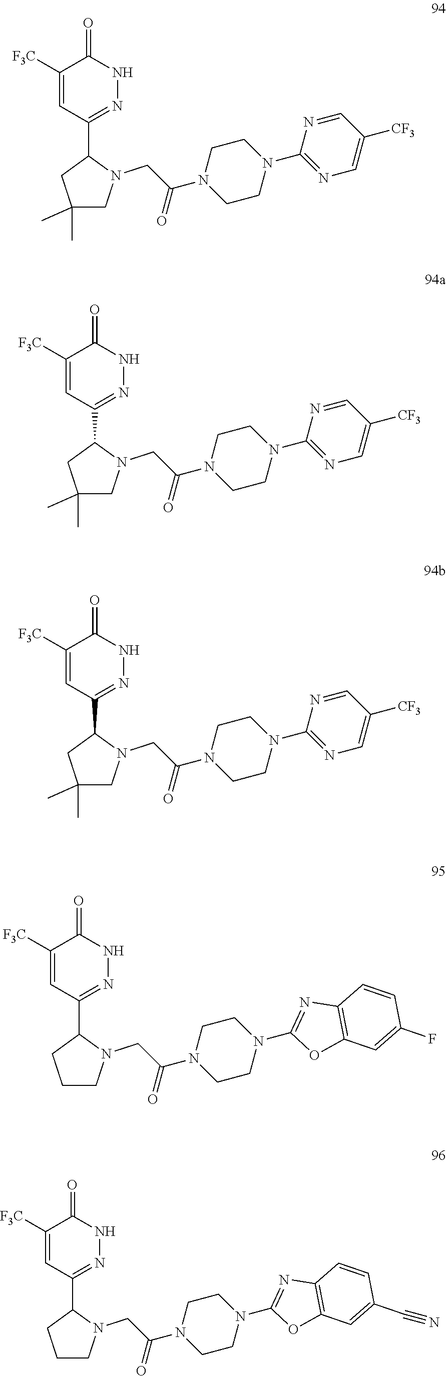 [SMILES: CC1(C)CC(c2cc(C(F)(F)F)c(=O)[nH]n2)N(CC(=O)N2CCN(c3ncc(C(F)(F)F)cn3)CC2)C1 ; CC1(C)C[C@H](c2cc(C(F)(F)F)c(=O)[nH]n2)N(CC(=O)N2CCN(c3ncc(C(F)(F)F)cn3)CC2)C1 ; CC1(C)C[C@@H](c2cc(C(F)(F)F)c(=O)[nH]n2)N(CC(=O)N2CCN(c3ncc(C(F)(F)F)cn3)CC2)C1 ; O=C(CN1CCCC1c1cc(C(F)(F)F)c(=O)[nH]n1)N1CCN(c2nc3ccc(F)cc3o2)CC1 ; N#Cc1ccc2nc(N3CCN(C(=O)CN4CCCC4c4cc(C(F)(F)F)c(=O)[nH]n4)CC3)oc2c1]

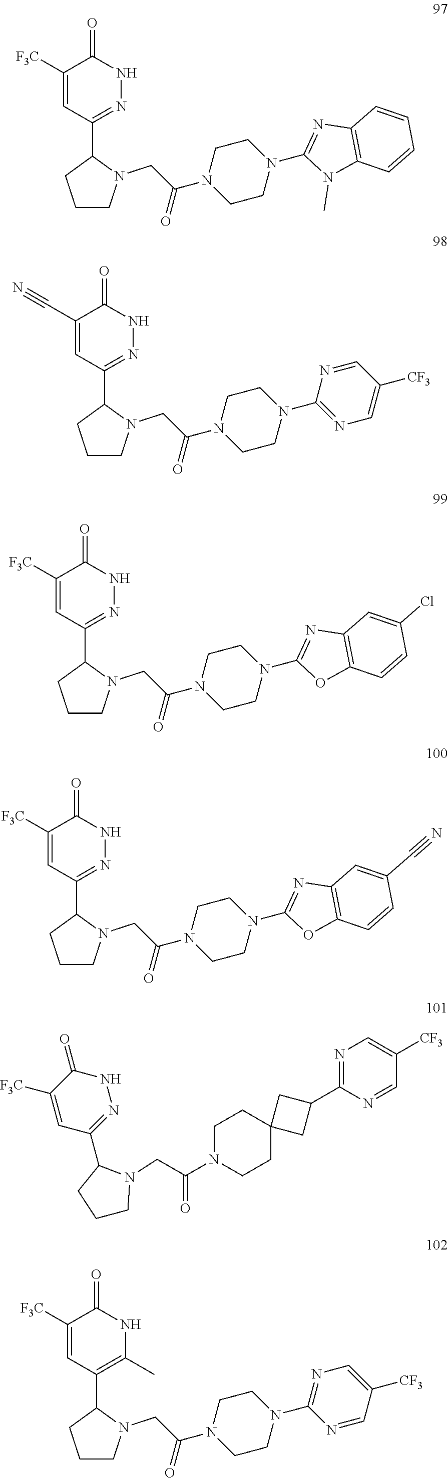 [SMILES: Cn1c(N2CCN(C(=O)CN3CCCC3c3cc(C(F)(F)F)c(=O)[nH]n3)CC2)nc2ccccc21 ; N#Cc1cc(C2CCCN2CC(=O)N2CCN(c3ncc(C(F)(F)F)cn3)CC2)n[nH]c1=O ; O=C(CN1CCCC1c1cc(C(F)(F)F)c(=O)[nH]n1)N1CCN(c2nc3cc(Cl)ccc3o2)CC1 ; N#Cc1ccc2oc(N3CCN(C(=O)CN4CCCC4c4cc(C(F)(F)F)c(=O)[nH]n4)CC3)nc2c1 ; O=C(CN1CCCC1c1cc(C(F)(F)F)c(=O)[nH]n1)N1CCC2(CC1)CC(c1ncc(C(F)(F)F)cn1)C2 ; Cc1[nH]c(=O)c(C(F)(F)F)cc1C1CCCN1CC(=O)N1CCN(c2ncc(C(F)(F)F)cn2)CC1]

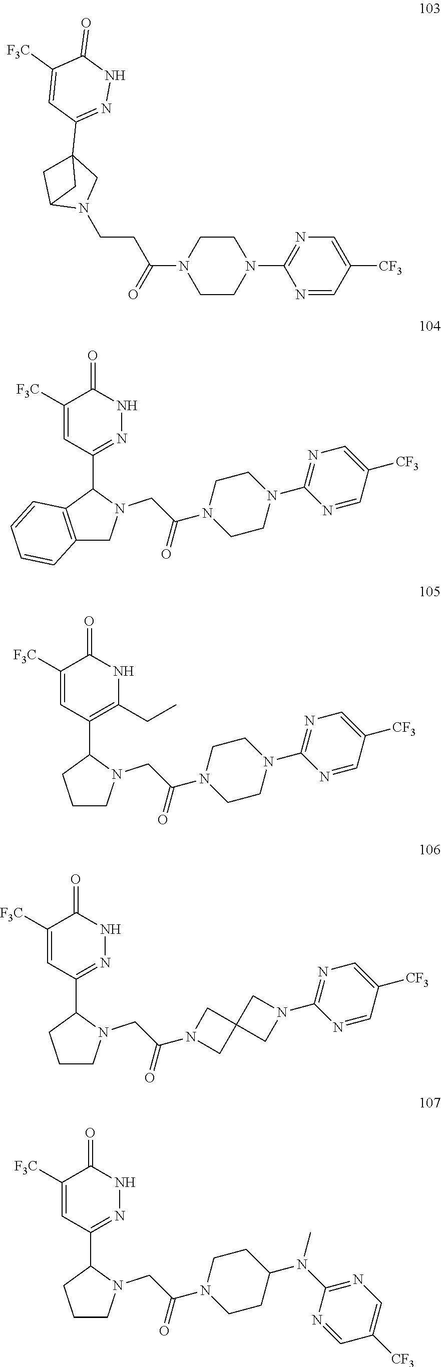 [SMILES: O=C(CCN1CC2(c3cc(C(F)(F)F)c(=O)[nH]n3)CC1C2)N1CCN(c2ncc(C(F)(F)F)cn2)CC1 ; O=C(CN1Cc2ccccc2C1c1cc(C(F)(F)F)c(=O)[nH]n1)N1CCN(c2ncc(C(F)(F)F)cn2)CC1 ; CCc1[nH]c(=O)c(C(F)(F)F)cc1C1CCCN1CC(=O)N1CCN(c2ncc(C(F)(F)F)cn2)CC1 ; O=C(CN1CCCC1c1cc(C(F)(F)F)c(=O)[nH]n1)N1CC2(C1)CN(c1ncc(C(F)(F)F)cn1)C2 ; CN(c1ncc(C(F)(F)F)cn1)C1CCN(C(=O)CN2CCCC2c2cc(C(F)(F)F)c(=O)[nH]n2)CC1]

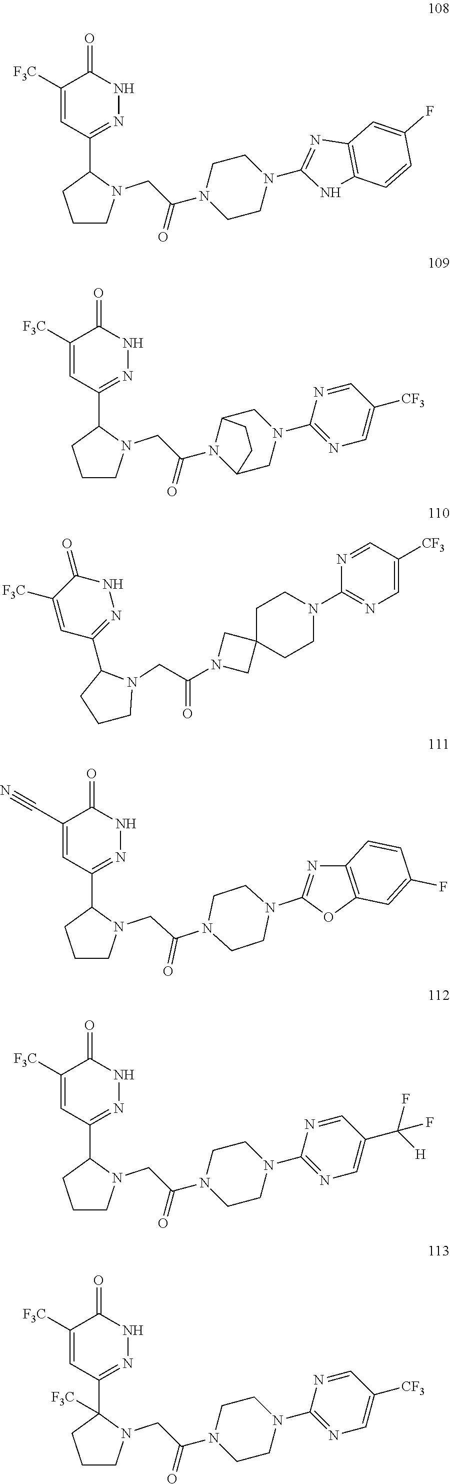 [SMILES: O=C(CN1CCCC1c1cc(C(F)(F)F)c(=O)[nH]n1)N1CCN(c2nc3cc(F)ccc3[nH]2)CC1 ; O=C(CN1CCCC1c1cc(C(F)(F)F)c(=O)[nH]n1)N1C2CCC1CN(c1ncc(C(F)(F)F)cn1)C2 ; O=C(CN1CCCC1c1cc(C(F)(F)F)c(=O)[nH]n1)N1CC2(CCN(c3ncc(C(F)(F)F)cn3)CC2)C1 ; N#Cc1cc(C2CCCN2CC(=O)N2CCN(c3nc4ccc(F)cc4o3)CC2)n[nH]c1=O ; O=C(CN1CCCC1c1cc(C(F)(F)F)c(=O)[nH]n1)N1CCN(c2ncc(C(F)F)cn2)CC1 ; O=C(CN1CCCC1(c1cc(C(F)(F)F)c(=O)[nH]n1)C(F)(F)F)N1CCN(c2ncc(C(F)(F)F)cn2)CC1]

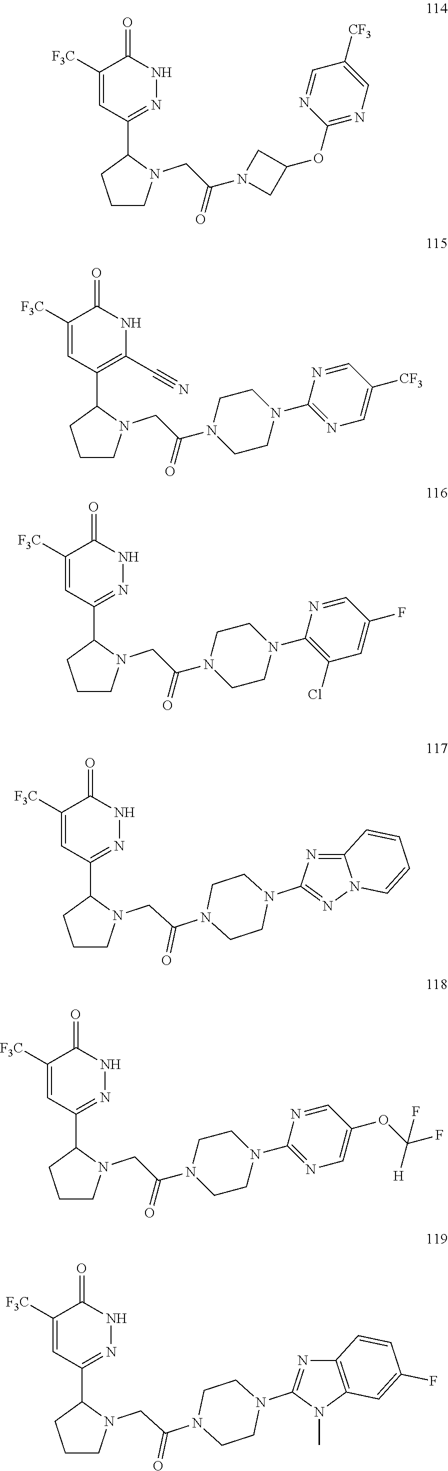 [SMILES: O=C(CN1CCCC1c1cc(C(F)(F)F)c(=O)[nH]n1)N1CC(Oc2ncc(C(F)(F)F)cn2)C1 ; N#Cc1[nH]c(=O)c(C(F)(F)F)cc1C1CCCN1CC(=O)N1CCN(c2ncc(C(F)(F)F)cn2)CC1 ; O=C(CN1CCCC1c1cc(C(F)(F)F)c(=O)[nH]n1)N1CCN(c2ncc(F)cc2Cl)CC1 ; O=C(CN1CCCC1c1cc(C(F)(F)F)c(=O)[nH]n1)N1CCN(c2nc3ccccn3n2)CC1 ; O=C(CN1CCCC1c1cc(C(F)(F)F)c(=O)[nH]n1)N1CCN(c2ncc(OC(F)F)cn2)CC1 ; Cn1c(N2CCN(C(=O)CN3CCCC3c3cc(C(F)(F)F)c(=O)[nH]n3)CC2)nc2ccc(F)cc21]

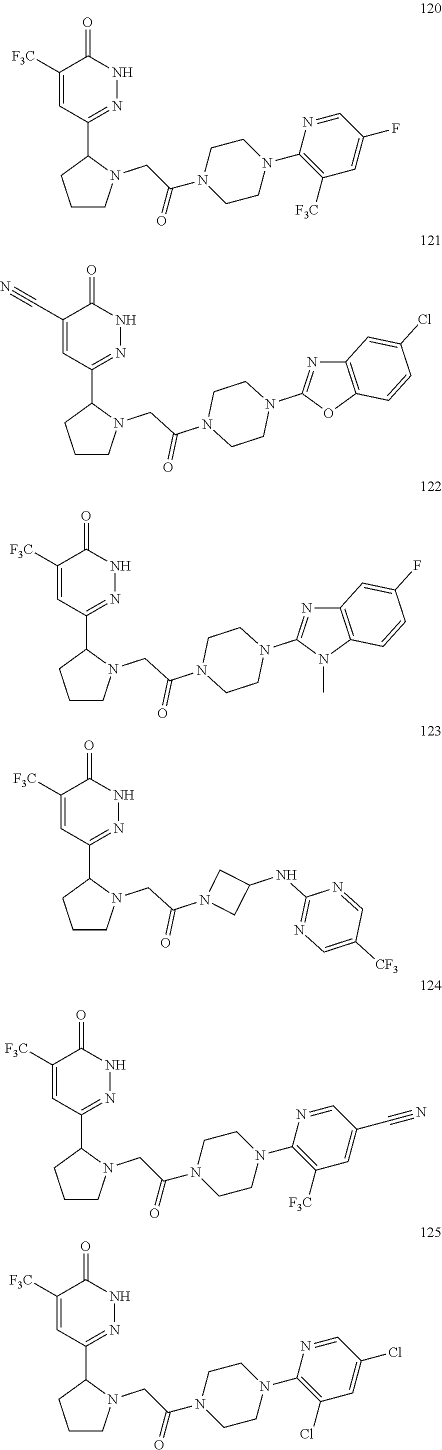

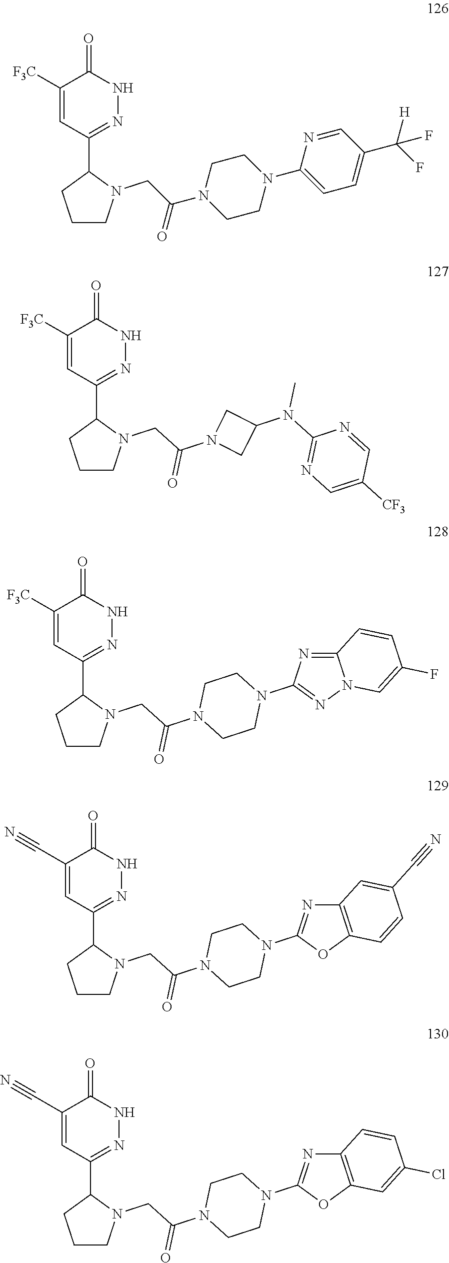 [SMILES: O=C(CN1CCCC1c1cc(C(F)(F)F)c(=O)[nH]n1)N1CCN(c2ccc(C(F)F)cn2)CC1 ; CN(c1ncc(C(F)(F)F)cn1)C1CN(C(=O)CN2CCCC2c2cc(C(F)(F)F)c(=O)[nH]n2)C1 ; O=C(CN1CCCC1c1cc(C(F)(F)F)c(=O)[nH]n1)N1CCN(c2nc3ccc(F)cn3n2)CC1 ; N#Cc1ccc2oc(N3CCN(C(=O)CN4CCCC4c4cc(C#N)c(=O)[nH]n4)CC3)nc2c1 ; N#Cc1cc(C2CCCN2CC(=O)N2CCN(c3nc4ccc(Cl)cc4o3)CC2)n[nH]c1=O]

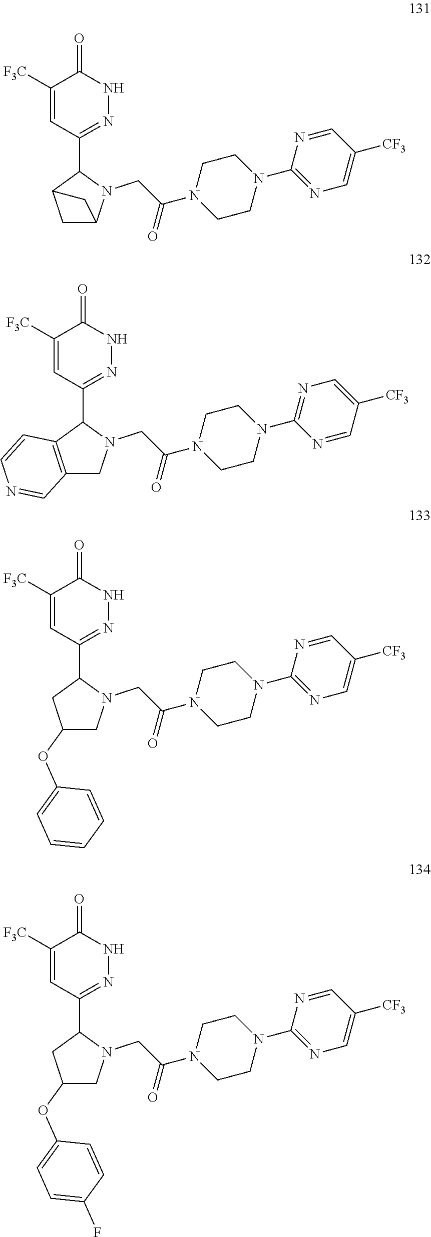 [SMILES: O=C(CN1C2CC(C2)C1c1cc(C(F)(F)F)c(=O)[nH]n1)N1CCN(c2ncc(C(F)(F)F)cn2)CC1 ; O=C(CN1Cc2cnccc2C1c1cc(C(F)(F)F)c(=O)[nH]n1)N1CCN(c2ncc(C(F)(F)F)cn2)CC1 ; O=C(CN1CC(Oc2ccccc2)CC1c1cc(C(F)(F)F)c(=O)[nH]n1)N1CCN(c2ncc(C(F)(F)F)cn2)CC1 ; O=C(CN1CC(Oc2ccc(F)cc2)CC1c1cc(C(F)(F)F)c(=O)[nH]n1)N1CCN(c2ncc(C(F)(F)F)cn2)CC1]

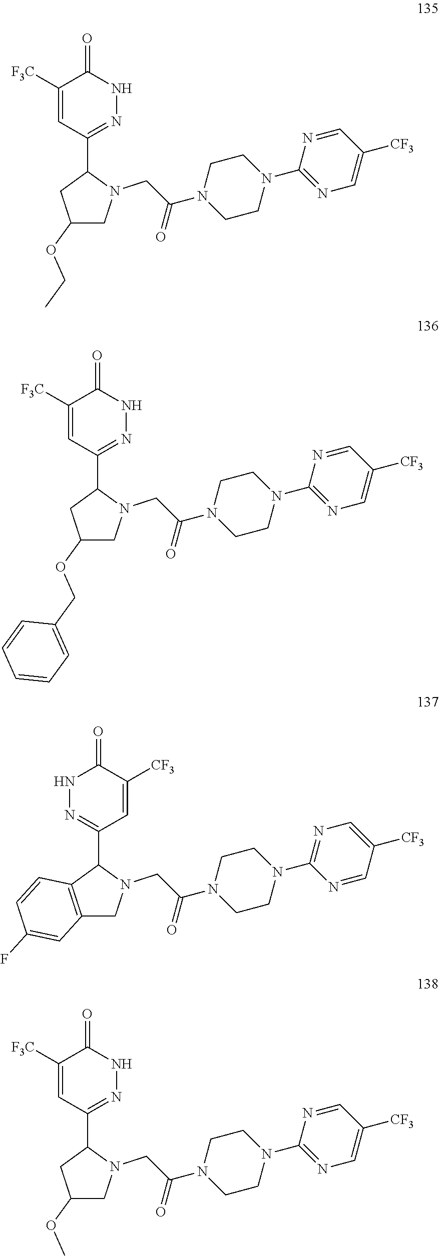 [SMILES: CCOC1CC(c2cc(C(F)(F)F)c(=O)[nH]n2)N(CC(=O)N2CCN(c3ncc(C(F)(F)F)cn3)CC2)C1 ; O=C(CN1CC(OCc2ccccc2)CC1c1cc(C(F)(F)F)c(=O)[nH]n1)N1CCN(c2ncc(C(F)(F)F)cn2)CC1 ; O=C(CN1Cc2cc(F)ccc2C1c1cc(C(F)(F)F)c(=O)[nH]n1)N1CCN(c2ncc(C(F)(F)F)cn2)CC1 ; COC1CC(c2cc(C(F)(F)F)c(=O)[nH]n2)N(CC(=O)N2CCN(c3ncc(C(F)(F)F)cn3)CC2)C1]

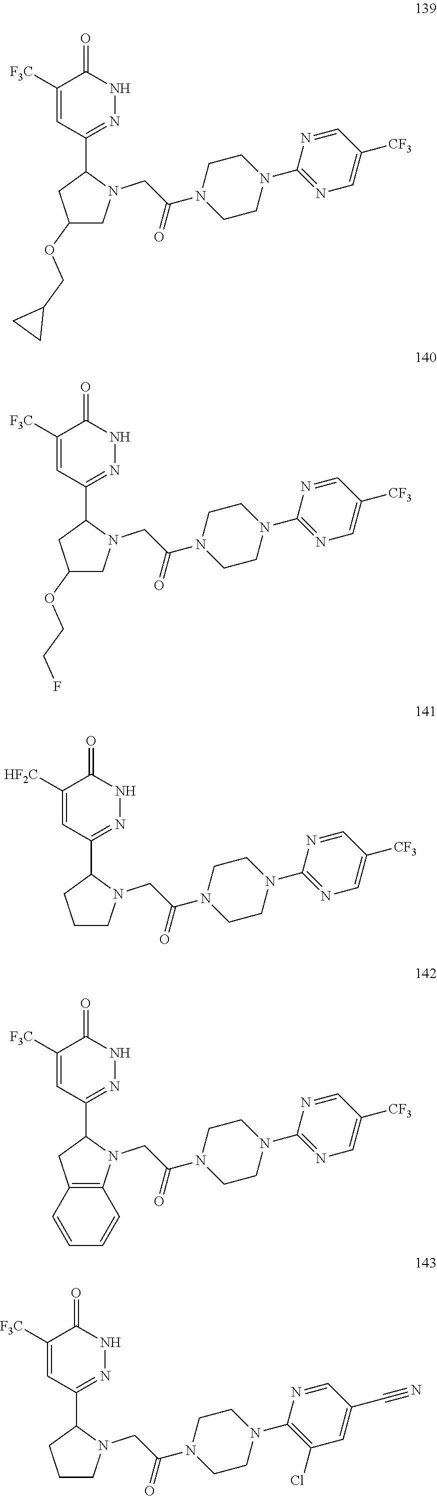

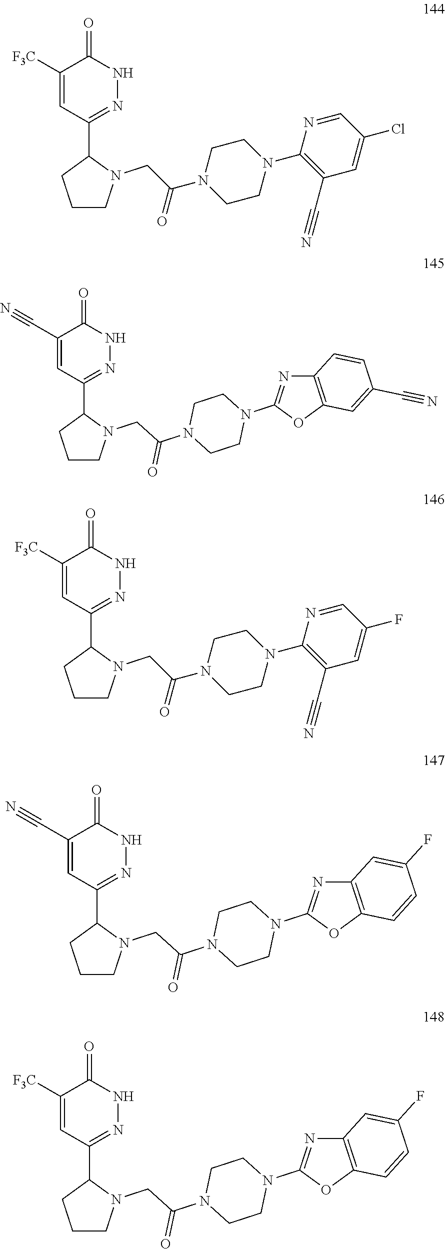 [SMILES: N#Cc1cc(Cl)cnc1N1CCN(C(=O)CN2CCCC2c2cc(C(F)(F)F)c(=O)[nH]n2)CC1 ; N#Cc1ccc2nc(N3CCN(C(=O)CN4CCCC4c4cc(C#N)c(=O)[nH]n4)CC3)oc2c1 ; N#Cc1cc(F)cnc1N1CCN(C(=O)CN2CCCC2c2cc(C(F)(F)F)c(=O)[nH]n2)CC1 ; N#Cc1cc(C2CCCN2CC(=O)N2CCN(c3nc4cc(F)ccc4o3)CC2)n[nH]c1=O ; O=C(CN1CCCC1c1cc(C(F)(F)F)c(=O)[nH]n1)N1CCN(c2nc3cc(F)ccc3o2)CC1]

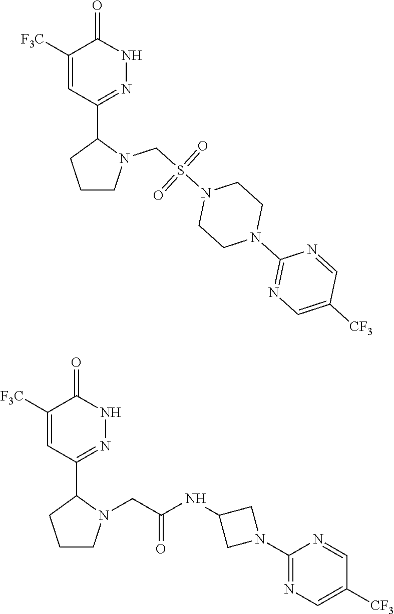 [SMILES: O=c1[nH]nc(C2CCCN2CS(=O)(=O)N2CCN(c3ncc(C(F)(F)F)cn3)CC2)cc1C(F)(F)F ; O=C(CN1CCCC1c1cc(C(F)(F)F)c(=O)[nH]n1)NC1CN(c2ncc(C(F)(F)F)cn2)C1]

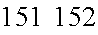

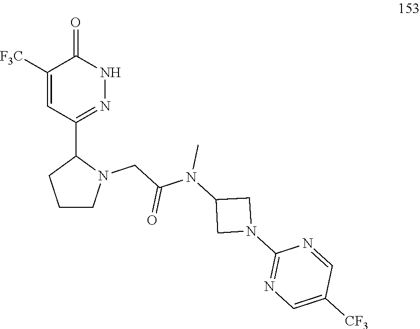 [SMILES: CN(C(=O)CN1CCCC1c1cc(C(F)(F)F)c(=O)[nH]n1)C1CN(c2ncc(C(F)(F)F)cn2)C1]

63-78. (canceled)

79. A method of treating a disease and/or a condition and/or a disorder, which method comprises administering to a patient a compound of claim 1.

80. (canceled)

81. A method according to claim 79, wherein the disease is a cancer, and wherein the method further comprises administering to the patient a further agent for treating a cancer wherein the further agent for treating cancer is selected from anti-microtubule agents, platinum coordination complexes, alkylating agents, antibiotic agents, topoisomerase II inhibitors, antimetabolites, topoisomerase I inhibitors, senolytic agents, hormones and hormone analogues, signal transduction Pathway inhibitors, DNA damage repair pathway inhibitors, non-receptor tyrosine kinase angiogenesis inhibitors, antibody-drug conjugates, anti-androgen therapies, immunotherapeutic agents, an oncolytic virus, an immune stimulatory antibody, a novel adjuvant, a peptide, a cytokine, a chimeric antigen receptor T cell therapy, a small molecule immune modulator, tumour microenvironment modulators; anti-angiogenic agents; receptor tyrosine kinase inhibitors, cell growth inhibitors, proapoptotic agents and cell cycle signalling inhibitors.

82-88. (canceled)
